(19) United States
(12) Patent Application Publication
(10) Pub. No.: US 2004/0029220 A1
(43) Pub. Date: Feb. 12, 2004

(54) Novel proteins and nucleic acids encoding same

(76) Inventors: Corine A.M. Vernet, North Branford, CT (US); Elma R. Fernandes, Branford, CT (US); Valerie Gerlach, Branford, CT (US); Richard A. Shimkets, West Haven, CT (US); Uriel M. Malyankar, Branford, CT (US); Ferenc L. Boldog, North Haven, CT (US); Bryan D. Zerhusen, Branford, CT (US); Kimberly A. Spytek, New Haven, CT (US); Kumud Majumder, Stamford, CT (US); Velizar T. Tchernev, Branford, CT (US); Muralidhara Padigaru, Branford, CT (US); Meera Patturajan, Branford, CT (US); Catherine E. Burgess, Wethersfield, CT (US); Esha A. Gangolli, Branford, CT (US); Glennda Smithson, Branford, CT (US); Luca Rastelli, Guilford, CT (US); John R. MacDougall, Hamden, CT (US); Raymond J. Taupier JR., East Haven, CT (US); William M. Grosse, Branford, CT (US); Edward S. Szekeres JR., Wallingford, CT (US); John P. Alsobrook II, Madison, CT (US); David W. Anderson, Branford, CT (US); Xiaojia (Sasha) Guo, Branford, CT (US); Li Li, Branford, CT (US); Mei Zhong, Branford, CT (US)

Correspondence Address: Ivor R. Elrifi, Ph.D., Esq., Mintz Levin Cohn Ferris Glovsky & Popeo, P.C., One Financial Center, Boston, MA, 02111, US

(21) Appl. No.: 10/174,333

(22) Filed: Jun. 18, 2002

Related U.S. Application Data
(63) Continuation-in-part of application No. 09/842,758, filed on Apr. 25, 2001.
(60) Provisional application No. 60/298,994, filed on Jun. 18, 2001.
(60) Provisional application No. 60/386,837, filed on Jun. 7, 2002.
(60) Provisional application No. 60/200,158, filed on Apr. 26, 2000.
(60) Provisional application No. 60/200,613, filed on Apr. 28, 2000.
(60) Provisional application No. 60/200,780, filed on Apr. 28, 2000.
(60) Provisional application No. 60/201,006, filed on May 1, 2000.
(60) Provisional application No. 60/201,007, filed on May 1, 2000.
(60) Provisional application No. 60/201,236, filed on May 1, 2000.
(60) Provisional application No. 60/201,238, filed on May 1, 2000.
(60) Provisional application No. 60/201,186, filed on May 2, 2000.
(60) Provisional application No. 60/201,474, filed on May 3, 2000.
(60) Provisional application No. 60/201,508, filed on May 3, 2000.
(60) Provisional application No. 60/220,591, filed on Jul. 25, 2000.
(60) Provisional application No. 60/232,678, filed on Sep. 15, 2000.
(60) Provisional application No. 60/263,217, filed on Jan. 22, 2001.
(60) Provisional application No. 60/265,160, filed on Jan. 30, 2001.
(60) Provisional application No. 60/269,531, filed on Feb. 16, 2001.

(51) Int. Cl. C07K 14/705; C07H 21/04; C12P 21/02; C12N 5/06

(52) U.S. Cl. 435/69.1; 435/320.1; 435/325; 530/350; 536/23.2

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/298,994, filed on Jun. 18, 2001; U.S. Ser. No. ______, filed on Jun. 7, 2002; and U.S. Ser. No. 09/842,758, filed on Apr. 25, 2001, which claims priority from U.S. Ser. No. 60/200,158, filed Apr. 26, 2000; U.S. Ser. No. 60/200,613, filed Apr. 28, 2000; U.S. Ser. No. 60/200,780, filed Apr. 28, 2000; U.S. Ser. No. 60/201,006, filed May 1, 2000; U.S. Ser. No. 60/201,007, filed May 1, 2000; U.S. Ser. No. 60/201,236, filed May 1, 2000; U.S. Ser. No. 60/201,238, filed May 1, 2000; U.S. Ser. No. 60/201,186, filed May 2, 2000; U.S. Ser. No. 60/201,474, filed May 3, 2000; U.S. Ser. No. 60/201,508, filed on May 3, 2001; U.S. Ser. No. 60/220,591, filed on Jul. 25, 2000; U.S. Ser. No. 60/232,678 filed Sep. 15, 2000; U.S. Ser. No. 60/263,217 filed Jan. 22, 2001; U.S. Ser. No. 60/265,160, filed Jan. 30, 2001; and U.S. Ser. No. 60/269,531, filed Feb. 16, 2001, each of which is incorporated by reference in its entirety.

### BACKGROUND OF THE INVENTION

The invention generally relates to nucleic acids and polypeptides. More particularly, the invention relates to nucleic acids encoding novel molecules (MOL) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as MOLX, or MOL1, MOL2, MOL3, MOL4, MOL5, MOL6, MOL7, MOL8, MOL9, and MIL10 nucleic acids and polypeptides. These nucleic acids and polypeptides, as well as derivatives, homologs, analogs and fragments thereof, will hereinafter be collectively designated as “MOLX” nucleic acid or polypeptide sequences.

In one aspect, the invention provides an isolated MOLX nucleic acid molecule encoding a MOLX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176. In some embodiments, the MOLX nucleic acid molecule will hybridize under stringent conditions to a nucleic acid sequence complementary to a nucleic acid molecule that includes a protein-coding sequence of a MOLX nucleic acid sequence. The invention also includes an isolated nucleic acid that encodes a MOLX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176.

Also included in the invention is an oligonucleotide, e.g., an oligonucleotide which includes at least 6 contiguous nucleotides of a MOLX nucleic acid (e.g., SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176) or a complement of said oligonucleotide.

Also included in the invention are substantially purified MOLX polypeptides (SEQ ID NOS:2, 4, 6, 8, 10, 12, 14, 16, 18, 20, 22, 24, 26, 28, 30, 115, 117, 119, 122, 124, 127, 129, 131, 133, 135, 137, 139,141, 143, 145, 147, 149, 151, 153, 155, 157, 159, 161, 163, 165, 167, 169, 171, 173, 175, and 177). In certain embodiments, the MOLX polypeptides include an amino acid sequence that is substantially identical to the amino acid sequence of a human MOLX polypeptide.

The invention also features antibodies that immunoselectively bind to MOLX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a MOLX nucleic acid, a MOLX polypeptide, or an antibody specific for a MOLX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a MOLX nucleic acid, under conditions allowing for expression of the MOLX polypeptide encoded by the DNA. If desired, the MOLX polypeptide can then be recovered.

In another aspect, the invention includes a method of detecting the presence of a MOLX polypeptide in a sample. In the method, a sample is contacted with a compound that selectively binds to the polypeptide under conditions allowing for formation of a complex between the polypeptide and the compound. The complex is detected, if present, thereby identifying the MOLX polypeptide within the sample.

The invention also includes methods to identify specific cell or tissue types based on their expression of a MOLX.

Also included in the invention is a method of detecting the presence of a MOLX nucleic acid molecule in a sample by contacting the sample with a MOLX nucleic acid probe or primer, and detecting whether the nucleic acid probe or primer bound to a MOLX nucleic acid molecule in the sample.

In a further aspect, the invention provides a method for modulating the activity of a MOLX polypeptide by contacting a cell sample that includes the MOLX polypeptide with a compound that binds to the MOLX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a MOLX nucleic acid, a MOLX polypeptide, or a MOLX-specific antibody, or biologically-active derivatives or fragments thereof.

For example, the compositions of the present invention will have efficacy for treatment of patients suffering from: Cancer including pancreatic cancer, adenoma, brain tumor, colon cancer breast cancer, prostate cancer, testis cancer, neurological disorders including age-related disorders, Alzheimer's disease, Stroke, Parkinson's disease, Huntington's disease, Cerebral palsy, Epilepsy, Behavioral disorders, Addiction, Anxiety, Pain, nephropathy, neurodegenerative disorders, Aneurysms, Fibromuscular dysplasia, metabolic disorders including, failure to thrive, nutritional edema, hypoproteinemia, trypsinogen deficiency disease, chronic and heriditary pancreatitis, enterkinase defieciency, Hypercholesterolemia, Obesity, Diabetes, cardiac disorders inclusing tachycardia, erythroderma, long QT syndrome, heart block, Ataxia-telangiectasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Larsen syndrome, night blindness, brugada syndrome, Von Hippel-Lindau (VHL) syndrome, Tuberous sclerosis, Hypercalceimia, Cirrhosis, angiogenesis and wound healing, blood pressure regulation, Trauma, Tuberous sclerosis, Fertility, Hirschsprung's disease, Renal artery stenosis, Interstitial nephritis, Glomerulonephritis, Polycystic kidney disease, Renal tubular acidosis, IgA nephropathy, immune disorders including cell-mediated immunity disorders, Leukodystrophies, inflammation, Hyperthyroidism, Hypothyroidism, Lesch-Nyhan syndrome, Multiple sclerosis, Transplantation, lung diseases, including asthma, Emphysema, immundeficiencies, Crohn's disease, Scleroderma, Appendicitis, Autoimmune diseases, Systemic lupus erythematosus, developmental disorders, neural tube defects, modulation of apoptosis, viral, bacterial, and parasitic infections and/or other pathologies and disorders of the like.

The polypeptides can be used as immunogens to produce antibodies specific for the invention, and as vaccines. They can also be used to screen for potential agonist and antagonist compounds. For example, a cDNA encoding MOLX may be useful in gene therapy, and MOLX may be useful when administered to a subject in need thereof. By way of nonlimiting example, the compositions of the present invention will have efficacy for treatment of patients suffering from Cancer including pancreatic cancer, adenoma, brain tumor, colon cancer breast cancer, prostate cancer, testis cancer, neurological disorders including age-related disorders, Alzheimer's disease, Stroke, Parkinson's disease, Huntington's disease, Cerebral palsy, Epilepsy, Behavioral disorders, Addiction, Anxiety, Pain, nephropathy, neurodegenerative disorders, Aneurysms, Fibromuscular dysplasia, metabolic disorders including. failure to thrive, nutritional edema, hypoproteinemia, trypsinogen deficiency disease, chronic and heriditary pancreatitis, enterkinase defieciency, Hypercholesterolemia, Obesity, Diabetes, cardiac disorders inclusing tachycardia, erythroderma, long QT syndrome, heart block, Ataxia-telangiectasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Larsen syndrome, night blindness, brugada syndrome, Von Hippel-Lindau (VHL) syndrome, Tuberous sclerosis, Hypercalceimia, Cirrhosis, angiogenesis and wound healing, blood pressure regulation, Trauma, Tuberous sclerosis, Fertility, Hirschsprung's disease, Renal artery stenosis, Interstitial nephritis, Glomerulonephritis, Polycystic kidney disease, Renal tubular acidosis, IgA nephropathy, immune disorders including cell-mediated immunity disorders, Leukodystrophies, inflammation, Hyperthyroidism, Hypothyroidism, Lesch-Nyhan syndrome, Multiple sclerosis, Transplantation, lung diseases, including asthma, Emphysema, immundeficiencies, Crohn's disease, Scleroderma, Appendicitis, Autoimmune diseases, Systemic lupus erythematosus, developmental disorders, neural tube defects, modulation of apoptosis, viral, bacterial, and parasitic infections and/or other pathologies and disorders.

The invention further includes a method for screening for a modulator of disorders or syndromes including, e.g., Cancer including pancreatic cancer, adenoma, brain tumor, colon cancer breast cancer, prostate cancer, testis cancer, neurological disorders including age-related disorders, Alzheimer's disease, Stroke, Parkinson's disease, Huntington's disease, Cerebral palsy, Epilepsy, Behavioral disorders, Addiction, Anxiety, Pain, nephropathy, neurodegenerative disorders, Aneurysms, Fibromuscular dysplasia, metabolic disorders including. failure to thrive, nutritional edema, hypoproteinemia, trypsinogen deficiency disease, chronic and heriditary pancreatitis, enterkinase defieciency, Hypercholesterolemia, Obesity, Diabetes, cardiac disorders inclusing tachycardia, erythroderma, long QT syndrome, heart block, Ataxia-telangiectasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Larsen syndrome, night blindness, brugada syndrome, Von Hippel-Lindau (VHL) syndrome, Tuberous sclerosis, Hypercalceimia, Cirrhosis, angiogenesis and wound healing, blood pressure regulation, Trauma, Tuberous sclerosis, Fertility, Hirschsprung's disease, Renal artery stenosis, Interstitial nephritis, Glomerulonephritis, Polycystic kidney disease, Renal tubular acidosis, IgA nephropathy, immune disorders including cell-mediated immunity disorders, Leukodystrophies, inflammation, Hyperthyroidism, Hypothyroidism, Lesch-Nyhan syndrome, Multiple sclerosis, Transplantation, lung diseases, including asthma, Emphysema, immundeficiencies, Crohn's disease, Scleroderma, Appendicitis, Autoimmune diseases, Systemic lupus erythematosus, developmental disorders, neural tube defects, modulation of apoptosis, viral, bacterial, and parasitic infections or other disorders related to cell signal processing and metabolic pathway modulation. The method includes contacting a test compound with a MOLX polypeptide and determining if the test compound binds to said MOLX polypeptide. Binding of the test compound to the MOLX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., Cancer including pancreatic cancer, adenoma, brain tumor, colon cancer breast cancer, prostate cancer, testis cancer, neurological disorders including age-related disorders, Alzheimer's disease, Stroke, Parkinson's disease, Huntington's disease, Cerebral palsy, Epilepsy, Behavioral disorders, Addiction, Anxiety, Pain, nephropathy, neurodegenerative disorders, Aneurysms, Fibromuscular dysplasia, metabolic disorders including. failure to thrive, nutritional edema, hypoproteinemia, trypsinogen deficiency disease, chronic and heriditary pancreatitis, enterkinase defieciency, Hypercholesterolemia, Obesity, Diabetes, cardiac disorders inclusing tachycardia, erythroderma, long QT syndrome, heart block, Ataxia-telangiectasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Larsen syndrome, night blindness, brugada syndrome, Von Hippel-Lindau (VHL) syndrome, Tuberous sclerosis, Hypercalceimia, Cirrhosis, angiogenesis and wound healing, blood pressure regulation, Trauma, Tuberous sclerosis, Fertility, Hirschsprung's disease, Renal artery stenosis, Interstitial nephritis, Glomerulonephritis, Polycystic kidney disease, Renal tubular acidosis, IgA nephropathy, immune disorders including cell-mediated immunity disorders, Leukodystrophies, inflammation, Hyperthyroidism, Hypothyroidism, Lesch-Nyhan syndrome, Multiple sclerosis, Transplantation, lung diseases, including asthma, Emphysema, immundeficiencies, Crohn's disease, Scleroderma, Appendicitis, Autoimmune diseases, Systemic lupus erythematosus, developmental disorders, neural tube defects, modulation of apoptosis, viral, bacterial, and parasitic infections or other disorders related to cell signal processing and metabolic pathway modulation by administering a test compound to a test animal at increased risk for the aforementioned disorders or syndromes. The test animal expresses a recombinant polypeptide encoded by a MOLX nucleic acid. Expression or activity of MOLX polypeptide is then measured in the test animal, as is expression or activity of the protein in a control animal which recombinantly-expresses MOLX polypeptide and is not at increased risk for the disorder or syndrome. Next, the expression of MOLX polypeptide in both the test animal and the control animal is compared. A change in the activity of MOLX polypeptide in the test animal relative to the control animal indicates the test compound is a modulator of latency of the disorder or syndrome.

In yet another aspect, the invention includes a method for determining the presence of or predisposition to a disease associated with altered levels of a MOLX polypeptide, a MOLX nucleic acid, or both, in a subject (e.g., a human subject). The method includes measuring the amount of the MOLX polypeptide in a test sample from the subject and comparing the amount of the polypeptide in the test sample to the amount of the MOLX polypeptide present in a control sample. An alteration in the level of the MOLX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a MOLX polypeptide, a MOLX nucleic acid, or a MOLX-specific antibody to a subject (e.g., a human subject), in an amount sufficient to alleviate or prevent the pathological condition. In preferred embodiments, the disorder, includes, e.g., Cancer including pancreatic cancer, adenoma, brain tumor, colon cancer breast cancer, prostate cancer, testis cancer, neurological disorders including age-related disorders, Alzheimer's disease, Stroke, Parkinson's disease, Huntington's disease, Cerebral palsy, Epilepsy, Behavioral disorders, Addiction, Anxiety, Pain, nephropathy, neurodegenerative disorders, Aneurysms, Fibromuscular dysplasia, metabolic disorders including, failure to thrive, nutritional edema, hypoproteinemia, trypsinogen deficiency disease, chronic and heriditary pancreatitis, enterkinase defieciency, Hypercholesterolemia, Obesity, Diabetes, cardiac disorders inclusing tachycardia, erythroderma, long QT syndrome, heart block, Ataxia-telangiectasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Larsen syndrome, night blindness, brugada syndrome, Von Hippel-Lindau (VHL) syndrome, Tuberous sclerosis, Hypercalceimia, Cirrhosis, angiogenesis and wound healing, blood pressure regulation, Trauma, Tuberous sclerosis, Fertility, Hirschsprung's disease, Renal artery stenosis, Interstitial nephritis, Glomerulonephritis, Polycystic kidney disease, Renal tubular acidosis, IgA nephropathy, immune disorders including cell-mediated immunity disorders, Leukodystrophies, inflammation, Hyperthyroidism, Hypothyroidism, Lesch-Nyhan syndrome, Multiple sclerosis, Transplantation, lung diseases, including asthma, Emphysema, immundeficiencies, Crohn's disease, Scleroderma, Appendicitis, Autoimmune diseases, Systemic lupus erythematosus, developmental disorders, neural tube defects, modulation of apoptosis, viral, bacterial, and parasitic infections.

In yet another aspect, the invention can be used in a method to identity the cellular receptors and downstream effectors of the invention by any one of a number of techniques commonly employed in the art. These include but are not limited to the two-hybrid, system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are referred to individually as MOL1, MOL2, MOL3, MOL4, MOL5, MOL6, MOL7, MOL8, MOL9, and MOL10. The nucleic acids, and their encoded polypeptides, are collectively designated herein as “MOLX”.

The novel MOLX nucleic acids of the invention include the nucleic acids whose sequences are provided in Tables 1A, 2A, 3A, 4A, 5A, 6A, 6D, 7A, 8A, 8D, 9A, 9D, 9F, and 10A. inclusive (“Tables 1A-10A”), or a fragment, derivative, analog or homolog thereof. The novel MOLX proteins of the invention include the protein fragments whose sequences are provided in Tables 1B, 2B, 3B, 4B 5B, 6B, 6E, 7B, 8B, 8E, 9B, 9E, 9G and 10B inclusive (“Tables 1B-10B”). The individual MOLX nucleic acids and proteins are described below. Within the scope of this invention is a method of using these nucleic acids and peptides in the treatment or prevention of a disorder related to cell signaling or metabolic pathway modulation.

MOL1

MOL1a

A disclosed interleukin-1 receptor/Toll-like nucleic acid of 1050 nucleotides, MOL1a, is shown in Table 1A. The disclosed MOL1a open reading frame (“ORF”) begins at the ATG initiation codon at nucleotides 1-3, shown in bold in Table 1A. The encoded polypeptide is alternatively referred to herein as MOL1a or as GM_{—}79960178. The disclosed MOL1a ORF terminates at a TGA codon at nucleotides 3043-3045. As shown in Table 1A the start and stop codons are in bold letters.

TABLE 1A
MOL1a nucleotide sequence.

(SEQ ID NO:1)

ATGCTGGCCATGACCCTGGCCCTGGGTACCTTGCCTGCCTTCCTACCCTGTGAGCTCCAGCCCCACGGCCTGGTGA
ACTGCAACTGGCTGTTCCTGAAGTCTGTGCCCCACTTCTCCATGGCAGCACCCCGTGGCAATGTCACCAGCCTTTC
CTTGTCCTCCAACCGCATCCACCACCTCCATGATTCTGACTTTGCCCACCTGCCCAGCCTGCGGCATCTCAACCTC
AAGTGGAACTGCCCGCCGGTTGGCCTCAGCCCCATGCACTTCCCCTGCCACATGACCATCGAGCCCAGCACCTTCT
TGGCTGTGCCCACCCTGGAAGAGCTAAACCTGAGCTACAACATCATCATGACTGTGCCTGCGCTGCCCAAATCCCT
CATATCCCTGTCCCTCAGCCATACCAACATCCTGATGCTAGACTCTGCCAGCCTCGCCGGCCTGCATGCCCTGCGC
TTCCTATTCATGGACGGCAACTGTTATTACAAGAACCCCTGCAGGCAGGCACTGGAGGTGGCCCCGGGTGCCCTCC
TTGGCCTGGGCAACCTCACCCACCTGTCACTCAAGTACAACAACCTCACTGTGGTGCCCCGCAACCTGCCTTCCAG
CCTGGAGTATCTGCTGTTGTCCTACAACCGCATCGTCAAACTGGCGCCTGAGGACCTGGCCAATCTGACCGCCCTG
CGTGTGCTCGATGTGGGCGGAAATTGCCGCCGCTGCGACCACGCTCCCAACCCCTGCATGGAGTGCCCTCGTCACT
TCCCCCAGCTACATCCCGATACCTTCAGCCACCTGAGCCGTCTTGAAGGCCTGGTGTTGAAGGACAGTTCTCTCTC
CTGGCTGAATGCCAGTTGGTTCCGTGGGCTGGGAAACCTCCGAGTGCTGGACCTGAGTGAGAACTTCCTCTACAAA
TGCATCACTAAAACCAAGGCCTTCCAGGGCCTAACACAGCTGCGCAAGCTTAACCTGTCCTTCAATTACCAAAAGA
GGGTGTCCTTTGCCCACCTGTCTCTGGCCCCTTCCTTCGGGAGCCTGGTCGCCCTGAAGGAGCTGGACATGCACGG
CATCTTCTTCCGCTCACTCGATGAGACCACGCTCCGGCCACTGGCCCGCCTGCCCATGCTCCAGACTCTGCGTCTG
CAGATGAACTTCATCAACCAGGCCCAGCTCGGCATCTTCAGGGCCTTCCCTGGCCTGCGCTACGTGGACCTGTCGG
ACAACCGCATCAGCGGAGCTTCGGAGCTGACAGCCACCATGGGGGAGGCAGATGGAGGGGAGAAGGTCTGGCTGCA
GCCTGGGGACCTTGCTCCGGCCCCAGTGGACACTCCCAGCTCTGAAGACTTCAGGCCCAACTGCAGCACCCTCAAC
TTCACCTTGGATCTGTCACGGAACAACCTGGTGACCGTGCAGCCGGAGATGTTTGCCCAGCTCTCGCACCTGCAGT
GCCTGCGCCTGAGCCACAACTGCATCTCGCAGGCAGTCAATGGCTCCCAGTTCCTGCCGCTGACCGGTCTGCAGGT
GCTAGACCTGTCCCACAATAAGCTGGACCTCTACCACGAGCACTCATTCACGGAGCTACCACGACTGGAGGCCCTG
GACCTCAGCTACAACAGCCAGCCCTTTGGCATGCAGGGCGTGGGCCACAACTTCAGCTTCGTGGCTCACCTGCGCA
CCCTGCGCCACCTCAGCCTGGCCCACAACAACATCCACAGCCAAGTGTCCCAGCAGCTCTGCAGTACGTCGCTGCG
GGCCCTGGACTTCAGCGGCAATGCACTGGGCCATATGTGGGCCGAGGGAGACCTCTATCTGCACTTCTTCCAAGGC
CTGAGCGGTTTGATCTGGCTGGACTTGTCCCAGAACCGCCTGCACACCCTCCTGCCCCAAACCCTGCGCAACCTCC
CCAAGAGCCTACAGGTGCTGCGTCTCCGTGACAATTACCTGGCCTTCTTTAAGTGGTGGAGCCTCCACTTCCTGCC
CAAACTGGAAGTCCTCGACCTGGCAGGAAACCAGCTGAAGGCCCTGACCAATGGCAGCCTGCCTGCTGGCACCCGG
CTCCGGAGGCTGGATGTCAGCTGCAACAGCATCAGCTTCGTGGCCCCCGGCTTCTTTTCCAAGGCCAAGGAGCTGC
GAGAGCTCAACCTTAGCGCCAACGCCCTCAAGACAGTGGACCACTCCTGGTTTGGGCCCCTGGCGAGTGCCCTGCA
AATACTAGATGTAAGCGCCAACCCTCTGCACTGCGCCTGTGGGGCGGCCTTTATGGACTTCCTGCTGGAGGTGCAG
GCTGCCGTGCCCGGTCTGCCCAGCCGGGTGAAGTGTGGCAGTCCGGGCCAGCTCCAGGGCCTCAGCATCTTTGCAC
AGGACCTGCGCCTCTGCCTGGATGAGGCCCTCTCCTGGGACTGTTTCGCCCTCTCGCTGCTGGCTGTGGCTCTGGG
CCTGGGTGTGCCCATGCTGCATCACCTCTGTGGCTGGGACCTCTGGTACTGCTTCCACCTGTGCCTGGCCTGGCTT
CCCTGGCGGGGGCGGCAAAGTGGGCGAGATGAGGATGCCCTGCCCTACGATGCCTTCGTGGTCTTCGACAAAACGC
AGAGCGCAGTGGCAGACTGGGTGTACAACGAGCTTCGGGGGCAGCTGGAGGAGTGCCGTGGGCGCTGGGCACTCCG
CCTGTGCCTGGAGGAACGCGACTGGCTGCCTGGCAAAACCCTCTTTGAGAACCTGTGGGCCTCGGTCTATGGCAGC
CGCAAGACGCTGTTTGTGCTGGCCCACACGGACCGGGTCAGTGGTCTCTTGCGCGCCAGCTTCCTGCTGGCCCAGC
AGCGCCTGCTGGAGGACCGCAAGGACGTCGTGGTGCTGGTGATCCTGAGCCCTGACGGCCGCCGCTCCCGCTATGT
GCGGCTGCGCCAGCGCCTCTGCCGCCAGAGTGTCCTCCTCTGGCCCCACCAGCCCAGTGGTCAGCGCAGCTTCTGG
GCCCAGCTGGGCATGGCCCTGACCAGGGACAACCACCACTTCTATAACCGGAACTTCTGCCAGGGACCCACGGCCG
AATAG

A disclosed encoded MOL1a protein has 346 amino acid residues, referred to as the MOL1a protein. The MOL1a protein was analyzed for signal peptide prediction and cellular localization. SignalP results predict that MOL1a is cleaved between position 41 and 42 of SEQ ID NO:2. Psort and Hydropathy profiles also predict that MOL1a contains a signal peptide and is likely to be localized in the plasma membrane (Certainty=0.4600). The disclosed MOL1a polypeptide sequence is presented in Table 1B using the one-letter amino acid code.

TABLE 1B
Encoded MOL1a protein sequence.

(SEQ ID NO:2)

MLAMTLALGTLPAFLPCELQPHGLVNCNWLFLKSVPHFSMAAPRGNVTSLSLSSNRIHHLHDSDFAHLPSLRHLNLK
WNCPPVGLSPMHFPCHMTIEPSTFLAVPTLEELNLSYNNIMTVPALPKSLISLSLSHTNILMLDSASLAGLHALRFL
FMDGNCYYKNPCRQALEVAPGALLGLGNLTHLSLKYNNLTVVPRNLPSSLEYLLLSYNRIVKLAPEDLANLTALRVL
DVGGNCRRCDHAPNPCMECPRHFPQLHPDTFSHLSRLEGLVLKDSSLSWLNASWFRGLGNLRVLDLSENFLYKCITK
TKAFQGLTQLRKLNLSFNYQKRVSFAHLSLAPSFGSLVALKELDMHGIFFRSLDETTLRPLARLPMLQTLRLQMNFI
NQAQLGIFRAFPGLRYVDLSDNRISGASELTATMGEADGGEKVWLQPGDLAPAPVDTPSSEDFRPNCSTLNFTLDLS
RNNLVTVQPEMFAQLSHLQCLRLSHNCISQAVNGSQFLPLTGLQVLDLSHNKLDLYHEHSFTELPRLEALDLSYNSQ
PFGMQGVGHNFSFVAHLRTLRHLSLAHNNIHSQVSQQLCSTSLRALDFSGNALGHMWAEGDLYLHFFQGLSGLIWLD
LSQNRLHTLLPQTLRNLPKSLQVLRLRDNYLAFFKWWSLHFLPKLEVLDLAGNQLKALTNGSLPAGTRLRRLDVSCN
SISFVAPGFFSKAKELRELNLSANALKTVDHSWFGPLASALQILDVSANPLHCACGAAFMDFLLEVQAAVPGLPSRV
KCGSPGQLQGLSIFAQDLRLCLDEALSWDCFALSLLAVALGLGVPMLHHLCGWDLWYCFHLCLAWLPWRGRQSGRDE
DALPYDAFVVFDKTQSAVADWVYNELRGQLEECRGRWALRLCLEERDWLPGKTLFENLWASVYGSRKTLFVLAHTDR
VSGLLRASFLLAQQRLLEDRKDVVVLVILSPDGRRSRYVRLRQRLCRQSVLLWPHQPSGQRSFWAQLGMALTRDNHH
FYNRNFCQGPTAE

MOL1a was initially identified on chromosome 3 with a TblastN analysis of a proprietary sequence file for a G-protein coupled receptor probe or homolog, which was run against the Genomic Daily Files made available by GenBank. A proprietary software program (GenScan™) was used to further predict the nucleic acid sequence and the selection of exons. The resulting sequences were further modified by means of similarities using BLAST searches. The sequences were then manually corrected for apparent inconsistencies, thereby obtaining the sequences encoding the full-length protein.

A region of the MOL1a nucleic acid sequence has 690 of 1203 bases (57%) identical to a Homo sapiens Toll Receptor mRNA (GENBANK-ID: AL137451), with an E-value of 5.7×10⁻⁸. In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the MOL1a BLAST analysis, e.g., the Homo sapiens MOL, matched the Query MOL1a sequence purely by chance is 5.7×10⁻⁸.

A BLASTX search was performed against public protein databases. The full amino acid sequence of the protein of the invention was found to have 342 of 900 amino acid residues (38%) identical to, and 493 of 900 residues (54%) positive with, the 1049 amino acid residue Toll-like Receptor 7 protein from Homo sapiens (ptnr:SPTREMBL-ACC:AAF60188).

MOL1b

A disclosed Toll receptor 9-like nucleic acid of 3118 nucleotides, MOL1b, is shown in Table 1C. The disclosed MOL1b open reading frame (“ORF”) begins at the ATG initiation codon at nucleotides 18-20, shown in bold in Table 1C. The encoded polypeptide is alternatively referred to herein as MOL1b or as CG54674-02. The disclosed MOL1b ORF terminates at a TAG codon at nucleotides 3114-3116. As shown in Table 1C the start and stop codons are in bold letters.

TABLE 1C
MOL1b nucleotide sequence.

(SEQ ID NO:114)

CCCCTCGCACCCCCAGCATGGGTTTCTGCCGCAGCGCCCTGCACCCGCTGTCTCTCCTGGTGCAGGCCATCATGCTG
GCCATGACCCTGGCCCTGGGTACCTTGCCTGCCTTCCTACCCTGTGAGCTCCAGCCCCACGGCCTGGTGAACTGCAA
CTGGCTGTTCCTGAAGTCTGTGCCCCACTTCTCCATGGCAGCACCCCGTGGCAATGTCACCAGCCTTTCCTTGTCCT
CCAACCGCATCCACCACCTCCATGATTCTGACTTTGCCCACCTGCCCAGCCTGCGGCATCTCAACCTCAAGTGGAAC
TGCCCGCCGGTTGGCCTCAGCCCCATGCACTTCCCCTGCCACATGACCATCGAGCCCAGCACCTTCTTGGCTGTGCC
CACCCTGGAAGAGCTAAACCTGAGCTACAACAACATCATGACTGTGCCTGCGCTGCCCAAATCCCTCATATCCCTGT
CCCTCAGCCATACCAACATCCTGATGCTAGACTCTGCCAGCCTCGCCGGCCTGCATGCCCTGCGCTTCCTATTCATG
GACGGCAACTGTTATTACAAGAACCCCTGCAGGCAGGCACTGGAGGTGGCCCCGGGTGCCCTCCTTGGCCTGGGCAA
CCTCACCCACCTGTCACTCAAGTACAACAACCTCACTGTGGTGCCCCGCAACCTGCCTTCCAGCCTGGAGTATCTGC
TGTTGTCCTACAACCGCATCGTCAAACTGGCGCCTGAGGACCTGGCCAATCTGACCGCCCTGCGTGTGCTCGATGTG
GGCGGAAATTGCCGCCGCTGCGACCACGCTCCCAACCCCTGCATGGAGTGCCCTCGTCACTTCCCCCAGCTACATCC
CGATACCTTCAGCCACCTGAGCCGTCTTGAAGGCCTGGTGTTGAAGGACAGTTCTCTCTCCTGGCTGAATGCCAGTT
GGTTCCGTGGGCTGGGAAACCTCCGAGTGCTGGACCTGAGTGAGAACTTCCTCTACAAATGCATCACTAAAACCAAG
GCCTTCCAGGGCCTAACACAGCTGCGCAAGCTTAACCTGTCCTTCAATTACCAAAAGAGGGTGTCCTTTGCCCACCT
GTCTCTGGCCCCTTCCTTCGGGAGCCTGGTCGCCCTGAAGGAGCTGGACATGCACGGCATCTTCTTCCGCTCACTCG
ATGAGACCACGCTCCGGCCACTGGCCCGCCTGCCCATGCTCCAGACTCTGCGTCTGCAGATGAACTTCATCAACCAG
GCCCAGCTCGGCATCTTCAGGGCCTTCCCTGGCCTGCGCTACGTGGACCTGTCGGACAACCGCATCAGCGGAGCTTC
GGAGCTGACAGCCACCATGGGGGAGGCAGATGGAGGGGAGAAGGTCTGGCTGCAGCCTGGGGACCTTGCTCCGGCCC
CAGTGGACACTCCCAGCTCTGAAGACTTCAGGCCCAACTGCAGCACCCTCAACTTCGCCTTGGATCTGTCACGGAAC
AACCTGGTGACCGTGCAGCCGGAGATGTTTGCCCAGCTCTCGCACCTGCAGTGCCTGCGCCTGAGCCACAACTGCAT
CTCGCAGGCAGTCAATGGCTCCCAGTTCCTGCCGCTGACCGGTCTGCAGGTGCTAGACCTGTCCCACAATAAGCTGG
ACCTCTACCACGAGCACTCATTCACGGAGCTACCACGACTGGAGGCCCTGGACCTCAGCTACAACAGCCAGCCCTTT
GGCATGCAGGGCGTGGGCCACAACTTCAGCTTCGTGGCTCACCTGCGCACCCTGCGCCACCTCAGCCTGGCCCACAA
CAACATCCACAGCCAAGTGTCCCAGCAGCTCTGCAGTACGTCGCTGCGGGCCCTGGACTTCAGCGGCAATGCACTGG
GCCATATGTGGGCCGAGGGAGACCTCTATCTGCACTTCTTCCAAGGCCTGAGCGGTTTGATCTGGCTGGACTTGTCC
CAGAACCGCCTGCACACCCTCCTGCCCCAAACCCTGCGCAACCTCCCCAAGAGCCTACAGGTGCTGCGTCTCCGTGA
CAATTACCTGGCCTTCTTTAAGTGGTGGAGCCTCCACTTCCTGCCCAAACTGGAAGTCCTCGACCTGGCAGGAAACC
AGCTGAAGGCCCTGACCAATGGCAGCCTGCCTGCTGGCACCCGGCTCCGGAGGCTGGATGTCAGCTGCAACAGCATC
AGCTTCGTGGCCCCCGGCTTCTTTTCCAAGGCCAAGGAGCTGCGAGAGCTCAACCTTAGCGCCAACGCCCTCAAGAC
AGTGGACCACTCCTGGTTTGGGCCCCTGGCGAGTGCCCTGCAAATACTAGATGTAAGCGCCAACCCTCTGCACTGCG
CCTGTGGGGCGGCCTTTATGGACTTCCTGCTGGAGGTGCAGGCTGCCGTGCCCGGTCTGCCCAGCCGGGTGAAGTGT
GGCAGTCCGGGCCAGCTCCAGGGCCTCAGCATCTTTGCACAGGACCTGCGCCTCTGCCTGGATGAGGCCCTCTCCTG
GGACTGTTTCGCCCTCTCGCTGCTGGCTGTGGCTCTGGGCCTGGGTGTGCCCATGCTGCATCACCTCTGTGGCTGGG
ACCTCTGGTACTGCTTCCACCTGTGCCTGGCCTGGCTTCCCTGGCGGGGGCGGCAAAGTGGGCGAGATGAGGATGCC
CTGCCCTACGATGCCTTCGTGGTCTTCGACAAAACGCAGAGCGCAGTGGCAGACTGGGTGTACAACGAGCTTCGGGG
GCAGCTGGAGGAGTGCCGTGGGCGCTGGGCACTCCGCCTGTGCCTGGAGGAACGCGACTGGCTGCCTGGCAAAACCC
TCTTTGAGAACCTGTGGGCCTCGGTCTATGGCAGCCGCAAGACGCTGTTTGTGCTGGCCCACACGGACCGGGTCAGT
GGTCTCTTGCGCGCCAGCTTCCTGCTGGCCCAGCAGCGCCTGCTGGAGGACCGCAAGGACGTCGTGGTGCTGGTGAT
CCTGAGCCCTGACGGCCGCCGCTCCCGCTACGTGCGGCTGCGCCAGCGCCTCTGCCGCCAGAGTGTCCTCCTCTGGC
CCCACCAGCCCAGTGGTCAGCGCAGCTTCTGGGCCCAGCTGGGCATGGCCCTGACCAGGGACAACCACCACTTCTAT
AACCGGAACTTCTGCCAGGGACCCACGGCCGAATAGCC

A disclosed encoded MOL1b protein has 1032 amino acid residues, referred to as the MOL1b protein. The disclosed MOL1b polypeptide sequence is presented in Table 1D using the one-letter amino acid code.

TABLE 1D
Encoded MOL1b protein sequence.

(SEQ ID NO:115)

MGFCRSALHPLSLLVQAIMLAMTLALGTLPAFLPCELQPHGLVNCNWLFLKSVPHFSMAAPRGNVTSLSLSSNRIHH
LHDSDFAHLPSLRHLNLKWNCPPVGLSPMHFPCHMTIEPSTFLAVPTLEELNLSYNNIMTVPALPKSLISLSLSHTN
ILMLDSASLAGLHALRFLFMDGNCYYKNPCRQALEVAPGALLGLGNLTHLSLKYNNLTVVPRNLPSSLEYLLLSYNR
IVKLAPEDLANLTALRVLDVGGNCRRCDHAPNPCMECPRHFPQLHPDTFSHLSRLEGLVLKDSSLSWLNASWFRGLG
NLRVLDLSENFLYKCITKTKAFQGLTQLRKLNLSFNYQKRVSFAHLSLAPSFGSLVALKELDMHGIFFRSLDETTLR
PLARLPMLQTLRLQMNFINQAQLGIFRAFPGLRYVDLSDNRISGASELTATMGEADGGEKVWLQPGDLAPAPVDTPS
SEDFRPNCSTLNFALDLSRNNLVTVQPEMFAQLSHLQCLRLSHNCISQAVNGSQFLPLTGLQVLDLSHNKLDLYHEH
SFTELPRLEALDLSYNSQPFGMQGVGHNFSFVAHLRTLRHLSLAHNNIHSQVSQQLCSTSLRALDFSGNALGHMWAE
GDLYLHFFQGLSGLIWLDLSQNRLHTLLPQTLRNLPKSLQVLRLRDNYLAFFKWWSLHFLPKLEVLDLAGNQLKALT
NGSLPAGTRLRRLDVSCNSISFVAPGFFSKAKELRELNLSANALKTVDHSWFGPLASALQILDVSANPLHCACGAAF
MDFLLEVQAAVPGLPSRVKCGSPGQLQGLSIFAQDLRLCLDEALSWDCFALSLLAVALGLGVPMLHHLCGWDLWYCF
HLCLAWLPWRGRQSGRDEDALPYDAFVVFDKTQSAVADWVYNELRGQLEECRGRWALRLCLEERDWLPGKTLFENLW
ASVYGSRKTLFVLAHTDRVSGLLRASFLLAQQRLLEDRKDVVVLVILSPDGRRSRYVRLRQRLCRQSVLLWPHQPSG
QRSFWAQLGMALTRDNHHFYNRNFCQGPTAE*

MOL1c

A disclosed Toll receptor 9-like nucleic acid of 3034 nucleotides, MOL1c, is shown in Table 1E. The disclosed MOL1c open reading frame (“ORF”) begins at the GGA initiation codon at nucleotides 2-4, shown in bold in Table 1E. The encoded polypeptide is alternatively referred to herein as MOL1c or as 248587042. The disclosed MOL1c ORF terminates at a GAG codon at nucleotides 3032-3034. As shown in Table 1E the start and stop codons are in bold letters. Because MOL1c does not start or stop at traditional initiation and termination codons, MOL1c could be a partial open reading frame which extends further in the 5′ and/or 3′ directions.

TABLE 1E
MOL1c nucleotide sequence.

(SEQ ID NO:116)

TGGATCCCTGGGTACCTTGCCTGCCTTCCTACCCTGTGAGCTCCAGCCCCACGGCCTGGTGAACTGCAACTGGCTGTTCC
TGAAGTCTGTGCCCCACTTCTCCATGGCAGCACCCCGTGGCAATGTCACCAGCCTTTCCTTGTCCTCCAACCGCATCCAC
CACCTCCATGATTCTGACTTTGCCCACCTGCCCAGCCTGCGGCATCTCAACCTCAAGTGGAACTGCCCGCCGGTTGGCCT
CAGCCCCATGCACTTCCCCTGCCACATGACCATCGAGCCCAGCACCTTCTTGGCTGTGCCCACCCTGGAAGAGCTAAACC
TGAGCTACAACAACATCATGACTGTGCCTGCGCTGCCCAAATCCCTCATATCCCTGTCCCTCAGCCATACCAACATCCTG
ATGCTAGACTCTGCCAGCCTCGCCGGCCTGCATGCCCTGCGCTTCCTATTCATGGACGGCAACTGTTATTACAAGAACCC
CTGCAGGCAGGCACTGGAGGTGGCCCCGGGTGCCCTCCTTGGCCTGGGCAACCTCACCCACCTGTCACTCAAGTACAACA
ACCTCACTGTGGTGCCCCGCAACCTGCCTTCCAGCCTGGAGTATCTGCTGTTGTCCTACAACCGCATCGTCAAACTGGCG
CCTGAGGACCTGGCCAATCTGACCGCCCTGCGTGTGCTCGATGTGGGCGGAAATTGCCGCCGCTGCGACCACGCTCCCAA
CCCCTGCATGGAGTGCCCTCGTCACTTCCCCCAGCTACATCCCGATACCTTCAGCCACCTGAGCCGTCTTGAAGGCCTGG
TGTTGAAGGACAGTTCTCTCTCCTGGCTGAATGCCAGTTGGTTCCGTGGGCTGGGAAACCTCCGAGTGCTGGACCTGAGT
GAGAACTTCCTCTACAAATGCATCACTAAAACCAAGGCCTTCCAGGGCCTAACACAGCTGCGCAAGCTTAACCTGTCCTT
CAATTACCAAAAGAGGGTGTCCTTTGCCCACCTGTCTCTGGCCCCTTCCTTCGGGAGCCTGGTCGCCCTGAAGGAGCTGG
ACATGCACGGCATCTTCTTCCGCTCACTCGATGAGACCACGCTCCGGCCACTGGCCCGCCTGCCCATGCTCCAGACTCTG
CGTCTGCAGATGAACTTCATCAACCAGGCCCAGCTCGGCATCTTCAGGGCCTTCCCTGGCCTGCGCTACGTGGACCTGTC
GGACAACCGCATCAGCGGAGCTTCGGAGCTGACAGCCACCATGGGGGAGGCAGATGGAGGGGAGAAGGTCTGGCTGCAGC
CTGGGGACCTTGCTCCGGCCCCAGTGGACACTCCCAGCTCTGAAGACTTCAGGCCCAACTGCAGCACCCTCAACTTCACC
TTGGATCTGTCACGGAACAACCTGGTGACCGTGCAGCCGGAGATGTTTGCCCAGCTCTCGCACCTGCAGTGCCTGCGCCT
GAGCCACAACTGCATCTCGCAGGCAGTCAATGGCTCCCAGTTCCTGCCGCTGACCGGTCTGCAGGTGCTAGACCTGTCCC
ACAATAAGCTGGACCTCTACCACGAGCACTCATTCACGGAGCTACCACGACTGGAGGCCCTGGACCTCAGCTACAACAGC
CAGCCCTTTGGCATGCAGGGCGTGGGCCACAACTTCAGCTTCGTGGCTCACCTGCGCACCCTGCGCCACCTCAGCCTGGC
CCACAACAACATCCACAGCCAAGTGTCCCAGCAGCTCTGCAGTACGTCGCTGCGGGCCCTGGACTTCAGCGGCAATGCAC
TGGGCCATATGTGGGCCGAGGGAGACCTCTATCTGCACTTCTTCCAAGGCCTGAGCGGTTTGATCTGGCTGGACTTGTCC
CAGAACCGCCTGCACACCCTCCTGCCCCAAACCCTGCGCAACCTCCCCAAGAGCCTACAGGTGCTGCGTCTCCGTGACAA
TTACCTGGCCTTCTTTAAGTGGTGGAGCCTCCACTTCCTGCCCAAACTGGAAGTCCTCGACCTGGCAGGAAACCAGCTGA
AGGCCCTGACCAATGGCAGCCTGCCTGCTGGCACCCGGCTCCGGAGGCTGGATGTCAGCTGCAACAGCATCAGCTTCGTG
GCCCCCGGCTTCTTTTCCAAGGCCAAGGAGCTGCGAGAGCTCAACCTTAGCGCCAACGCCCTCAAGACAGTGGACCACTC
CTCGTTTGGGCCCCTGGCGAGTGCCCTGCAAATACTAGATGTAAGCGCCAACCCTCTGCACTGCGCCTGTGGGGCGGCCT
TTATGGACTTCCTGCTGGAGGTGCAGGCTGCCGTGCCCGGTCCGCCCAGCCGGGTGAAGTGTGGCAGTCCGGGCCAGCTC
CAGGGCCTCAGCATCTTTGCACAGGACCTGCGCCTCTGCCTGGATGAGGCCCTCTCCTGGGACTGTTTCGCCCTCTCGCT
GCTGGCTGTGGCTCTGGGCCTGGGTGTGCCCATGCTGCATCACCTCTGTGGCTGGGACCTCTGGTACTGCTTCCACCTGT
GCCTGGCCTGGCTTCCCTGGCGGGGGCGGCAAAGTGGGCGAGATGAGGATGCCCTGCCCTACGATGCCTTCGTGGTCTTC
GACAAAACGCAGAGCGCAGTGGCAGACTGGGTGTACAACGAGCTTCGGGGGCAGCTGGAGGAGTGCCGTGGGCGCTGGGC
ACTCCGCCTGTGCCTGGAGGAACGCGACTGGCTGCCTGGCAAAACCCTCTTTGAGAACCTGTGGGCCTCGGTCTATGGCA
GCCGCAAGACGCTGTTTGTGCTGGCCCACACGGACCGGGTCAGTGGTCTCTTGCGCGCCAGCTTCCTGCTGGCCCAGCAG
CGCCTGCTGGAGGACCGCAAGGACGTCGTGGTGCTGGTGATCCTGAGCCCTGACGGCCGCCGCTCCCGCTACGTGCGGCT
GCGCCAGCGCCTCTGCCGCCAGAGTGTCCTCCTCTGGCCCCACCAGCCCAGTGGTCAGCGCAGCTTCTGGGCCCAGCTGG
GCATGGCCCTGACCAGGGACAACCACCACTTCTATAACCGGAACTTCTGCCAGGGACCCACGGCCGAACTCGAG

A disclosed encoded MOL1c protein has 1011 amino acid residues, referred to as the MOL1c protein. The disclosed MOL1c polypeptide sequence is presented in Table 1F using the one-letter amino acid code.

TABLE 1F
Encoded MOL1c protein sequence.

(SEQ ID NO:117)

GSLGTLPAFLPCELQPHGLVNCNWLFLKSVPHFSMAAPRGNVTSLSLSSNRIHHLHDSDFAHLPSLRHLNLKWNCPP
VGLSPMHFPCHMTIEPSTFLAVPTLEELNLSYNNIMTVPALPKSLISLSLSHTNILMLDSASLAGLHALRFLFMDGN
CYYKNPCRQALEVAPGALLGLGNLTHLSLKYNNLTVVPRNLPSSLEYLLLSYNRIVKLAPEDLANLTALRVLDVGGN
CRRCDHAPNPCMECPRHFPQLHPDTFSHLSRLEGLVLKDSSLSWLNASWFRGLGNLRVLDLSENFLYKCITKTKAFQ
GLTQLRKLNLSFNYQKRVSFAHLSLAPSFGSLVALKELDMHGIFFRSLDETTLRPLARLPMLQTLRLQMNFINQAQL
GIFRAFPGLRYVDLSDNRISGASELTATMGEADGGEKVWLQPGDLAPAPVDTPSSEDFRPNCSTLNFTLDLSRNNLV
TVQPEMFAQLSHLQCLRLSHNCISQAVNGSQFLPLTGLQVLDLSHNKLDLYHEHSFTELPRLEALDLSYNSQPFGMQ
GVGHNFSFVAHLRTLRHLSLAHNNIHSQVSQQLCSTSLRALDFSGNALGHMWAEGDLYLHFFQGLSGLIWLDLSQNR
LHTLLPQTLRNLPKSLQVLRLRDNYLAFFKWWSLHFLPKLEVLDLAGNQLKALTNGSLPAGTRLRRLDVSCNSISFV
APGFFSKAKELRELNLSANALKTVDHSWFGPLASALQILDVSANPLHCACGAAFMDFLLEVQAAVPGPPSRVKCGSP
GQLQGLSIFAQDLRLCLDEALSWDCFALSLLAVALGLGVPMLHHLCGWDLWYCFHLCLAWLPWRGRQSGRDEDALPY
DAFVVFDKTQSAVADWVYNELRGQLEECRGRWALRLCLEERDWLPGKTLFENLWASVYGSRKTLFVLAHTDRVSGLL
RASFLLAQQRLLEDRKDVVVLVILSPDGRRSRYVRLRQRLCRQSVLLWPHQPSGQRSFWAQLGMALTRDNHHFYNRN
FCQGPTAELE*

MOL1d

A disclosed Toll receptor 9-like nucleic acid of 3111 nucleotides, MOL1d, is shown in Table 1G. The disclosed MOL1d open reading frame (“ORF”) begins at the GGA initiation codon at nucleotides 1-3, shown in bold in Table 1G. The encoded polypeptide is alternatively referred to herein as MOL1d or as 248651580. The disclosed MOL1d ORF terminates at a GAG codon at nucleotides 3109-3111. As shown in Table 1G the start and stop codons are in bold letters. Because MOL1d does not start or stop at traditional initiation and termination codons, MOL1d could be a partial open reading frame which extends further in the 5′ and/or 3′ directions.

TABLE 1G
MOL1d nucleotide sequence.

(SEQ ID NO:118)

GGATCCACCATGGGTTTCTGCCGCAGCGCCCTGCACCCGCTGTCTCTCCTGGTGCAGGCCATCATGCTGGCCATGAC
CCTGGCCCTGGGTACCTTGCCTGCCTTCCTACCCTGTGAGCTCCAGCCCCACGGCCTGGTGAACTGCAACTGGCTGT
TCCTGAAGTCTGTGCCCCACTTCTCCATGGCAGCACCCCGTGGCAATGTCACCAGCCTTTCCTTGTCCTCCAACCGC
ATCCACCACCTCCATGATTCTGACTTTGCCCACCTGCCCAGCCTGCGGCATCTCAACCTCAAGTGGAACTGCCCGCC
GGTTGGCCTCAGCCCCATGCACTTCCCCTGCCACATGACCATCGAGCCCAGCACCTTCTTGGCTGTGCCCACCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCGCCGGCCTGCATGCCCTGCGCTTCCTATTCATGGACGGCAA
CTGTTATTACAAGAACCCCTGCAGGCAGGCACTGGAGGTGGCCCCGGGTGCCCTCCTTGGCCTGGGCAACCTCACCC
ACCTGTCACTCAAGTACAACAACCTCACTGTGGTGCCCCGCAACCTGCCTTCCAGCCTGGAGTATCTGCTGTTGTCC
TACAACCGCATCGTCAAACTGGCGCCTGAGGACCTGGCCAATCTGACCGCCCTGCGTGTGCTCGATGTGGGCGGAAA
TTGCCGCCGCTGCGACCACGCTCCCAACCCCTGCATGGAGTGCCCTCGTCACTTCCCCCAGCTACATCCCGATACCT
TCAGCCACCTGAGCCGTCTTGAAGGCCTGGTGTTGAAGGACAGTTCTCTCTCCTGGCTGAATGCCAGTTGGTTCCGT
GGGCTGGGAAACCTCCGAGTGCTGGACCTGAGTGAGAACTTCCTCTACAAATGCATCACTAAAACCAAGGCCTTCCA
GGGCCTAACACAGCTGCGCAAGCTTAACCTGTCCTTCAATTACCAAAAGAGGGTGTCCTTTGCCCACCTGTCTCTGG
CCCCTTCCTTCGGGAGCCTGGTCGCCCTGAAGGAGCTGGACATGCACGGCATCTTCTTCCGCTCACTCGATGAGACC
ACGCTCCGGCCACTGGCCCGCCTGCCCATGCTCCAGACTCTGCGTCTGCAGATGAACTTCATCAACCAGGCCCAGCT
CGGCATCTTCAGGGCCTTCCCTGGCCTGCGCTACGTGGACCTGTCGGACAACCGCATCAGCGGAGCTTCGGAGCTGA
CAGCCACCATGGGGGAGGCAGATGGAGGGGAGAAGGTCTGGCTGCAGCCTGGGGACCTTGCTCCGGCCCCAGTGGAC
ACTCCCAGCTCTGAAGACTTCAGGCCCAACTGCAGCACCCTCAACTTCACCTTGGATCTGTCACGGAACAACCTGGT
GACCGTGCAGCCGGAGATGTTTGCCCAGCTCTCGCACCTGCAGTGCCTGCGCCTGAGCCACAACTGCATCTCGCAGG
CAGTCAATGGCTCCCAGTTCCTGCCGCTGACCTGTCTGCAGGTGCTAGACCTGTCCCACAATAAGCTGGACCTCTAC
CACGAGCACTCATTCACGGAGCTACCACGACTGGAGGCCCTGGACCTCAGCTACAACAGCCAGCCCTTTGGCATGCA
GGGCGTGGGCCACAACTTCAGCTTCGTGGCTCACCTGCGCACCCTGCGCCACCTCAGCCTGGCCCACAACAACATCC
ACAGCCAAGTGTCCCAGCAGCTCTGCAGTACGTCGCTGCGGGCCCTGGACTTCAGCGGCAATGCACTGGGCCATATG
TGGGCCGAGGGAGACCTCTATCTGCACTTCTTCCAAGGCCTGAGCGGTTTGATCTGGCTGGACTTGTCCCAGAACCG
CCTGCACACCCTCCTGCCCCAAACCCTGCGCAACCTCCCCAAGAGCCTACAGGTGCTGCGTCTCCGTGACAATTACC
TGGCCTTCTTTAAGTGGTGGAGCCTCCACTTCCTGCCCAAACTGGAAGTCCTCGACCTGGCAGGAAACCAGCTGAAG
GCCCTGACCAATGGCAGCCTGCCTGCTGGCACCCGGCTCCGGAGGCTGGATGTCAGCTGCAACAGCATCAGCTTCGT
GGCCCCCGGCTTCTTTTCCAAGGCCAAGGAGCTGCGAGAGCTCAACCTTAGCGCCAACGCCCTCAAGACAGTGGACC
ACTCCTGGTTTGGGCCCCTGGCGAGTGCCCTGCAAATACTAGATGTAAGCGCCAACCCTCTGCACTGCGCCTGTGGG
GCGGCCTTTATGGACTTCCTGCTGGAGGTGCAGGCTGCCGTGCCCGGTCTGCCCAGCCGGGTGAAGTGTGGCAGTCC
GGGCCAGCTCCAGGGCCTCAGCATCTTTGCACAGGACCTGCGCCTCTGCCTGGATGAGGCCCTCTCCTGGGACTGTT
TCGCCCTCTCGCTGCTGGCTGTGGCTCTGGGCCTGGGTGTGCCCATGCTGCATCACCTCTGTGGCTGGGACCTCTGG
TACTGCTTCCACCTGTGCCTGGCCTGGCTTCCCTGGCGGGGGCGGCAAAGTGGGCGAGATGAGGATGCCCTGCCCTA
CGATGCCTTCGTGGTCTTCGACAAAACGCAGAGCGCAGTGGCAGACTGGGTGTACAACGAGCTTCGGGGGCAGCTGG
AGGAGTGCCGTGGGCGCTGGGCACTCCGCCTGTGCCTGGAGGAACGCGACTGGCTGCCTGGCAAAACCCTCTTTGAG
AACCTGTGGGCCTCGGTCTATGGCAGCCGCAAGACGCTGTTTGTGCTGGCCCACACGGACCGGGTCAGTGGTCTCTT
GCGCGCCAGCTTCCTGCTGGCCCAGCAGCGCCTGCTGGAGGACCGCAAGGACGTCGTGGTGCTGGTGATCCTGAGCC
CTGACGGCCGCCGCTCCCGCTACGTGCGGCTGCGCCAGCGCCTCTGCCGCCAGAGTGTCCTCCTCTGGCCCCACCAG
CCCAGTGGTCAGCGCAGCTTCTGGGCCCAGCTGGGCATGGCCCTGACCAGGGACAACCACCACTTCTATAACCGGAA
CTTCTGCCAGGGACCCACGGCCGAACTCGAG

A disclosed encoded MOL1d protein has 1037 amino acid residues, referred to as the MOL1d protein. The disclosed MOL1d polypeptide sequence is presented in Table 1H using the one-letter amino acid code.

TABLE 1H
Encoded MOL1d protein sequence.

(SEQ ID NO:119)

GSTMGFCRSALHPLSLLVQAIMLAMTLALGTLPAFLPCELQPHGLVNCNWLFLKSVPHFSMAAPRGNVTSLSLSSNR
IHHLHDSDFAHLPSLRHLNLKWNCPPVGLSPMHFPCHMTIEPSTFLAVPTLEELNLSYNNIMTVPALPKSLISLSLS
HTNILMLDSASLAGLHALRFLFMDGNCYYKNPCRQALEVAPGALLGLGNLTHLSLKYNNLTVVPRNLPSSLEYLLLS
YNRIVKLAPEDLANLTALRVLDVGGNCRRCDHAPNPCMECPRHFPQLHPDTFSHLSRLEGLVLKDSSLSWLNASWFR
GLGNLRVLDLSENFLYKCITKTKAFQGLTQLRKLNLSFNYQKRVSFAHLSLAPSFGSLVALKELDMHGIFFRSLDET
TLRPLARLPMLQTLRLQMNFINQAQLGIFRAFPGLRYVDLSDNRISGASELTATMGEADGGEKVWLQPGDLAPAPVD
TPSSEDFRPNCSTLNFTLDLSRNNLVTVQPEMFAQLSHLQCLRLSHNCISQAVNGSQFLPLTCLQVLDLSHNKLDLY
HEHSFTELPRLEALDLSYNSQPFGMQGVGHNFSFVAHLRTLRHLSLAHNNIHSQVSQQLCSTSLRALDFSGNALGHM
WAEGDLYLHFFQGLSGLIWLDLSQNRLHTLLPQTLRNLPKSLQVLRLRDNYLAFFKWWSLHFLPKLEVLDLAGNQLK
ALTNGSLPAGTRLRRLDVSCNSISFVAPGFFSKAKELRELNLSANALKTVDHSWFGPLASALQILDVSANPLHCACG
AAFMDFLLEVQAAVPGLPSRVKCGSPGQLQGLSIFAQDLRLCLDEALSWDCFALSLLAVALGLGVPMLHHLCGWDLW
YCFHLCLAWLPWRGRQSGRDEDALPYDAFVVFDKTQSAVADWVYNELRGQLEECRGRWALRLCLEERDWLPGKTLFE
NLWASVYGSRKTLFVLAHTDRVSGLLRASFLLAQQRLLEDRKDVVVLVILSPDGRRSRYVRLRQRLCRQSVLLWPHQ
PSGQRSFWAQLGMALTRDNHHFYNRNFCQGPTAELE

MOL1e

A disclosed Toll receptor 9-like nucleic acid of 3111 nucleotides, MOL1e, is shown in Table 1I. The disclosed MOL1e open reading frame (“ORF”) begins at the GGA initiation codon at nucleotides 1-3, shown in bold in Table 1G. The encoded polypeptide is alternatively referred to herein as MOL1e or as 255304731. The disclosed MOL1e ORF terminates at A GAG codon at nucleotides 3109-3111. As shown in Table 1I the start and stop codons are in bold letters. Because MOL1e does not start or stop at traditional initiation and termination codons, MOL1e could be a partial open reading frame which extends further in the 5′ and/or 3′ directions.

TABLE 1I
MOL1e nucleotide sequence.

(SEQ ID NO:120)

GGATCCACCATGGGTTTCTGCCGCAGCGCCCTGCACCCGCTGTCTCTCCTGGTGCAGGCCATCATGCTGGCCATGAC
CCTGGCCCTGGGTACCTTGCCTGCCTTCCTACCCTGTGAGCTCCAGCCCCACGGCCTGGTGAACTGCAACTGGCTGT
TCCTGAAGTCTGTGCCCCACTTCTCCATGGCAGCACCCCGTGGCAATGTCACCAGCCTTTCCTTGTCCTCCAACCGC
ATCCACCACCTCCATGATTCTGACTTTGCCCACCTGCCCAGCCTGCGGCATCTCAACCTCAAGTGGAACTGCCCGCC
GGTTGGCCTCAGCCCCATGCACTTCCCCTGCCACATGACCATCGAGCCCAGCACCTTCTTGGCTGTGCCCACCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCGCCGGCCTGCATGCCCTGCGCTTCCTATTCATGGACGGCAA
CTGTTATTACAAGAACCCCTGCAGGCAGGCACTGGAGGTGGCCCCGGGTGCCCTCCTTGGCCTGGGCAACCTCACCC
ACCTGTCACTCAAGTACAACAACCTCACTGTGGTGCCCCGCAACCTGCCTTCCAGCCTGGAGTATCTGCTGTTGTCC
TACAACCGCATCGTCAAACTGGCGCCTGAGGACCTGGCCAATCTGACCGCCCTGCGTGTGCTCGATGTGGGCGGAAA
TTGCCGCCGCTGCGACCACGCTCCCAACCCCTGCATGGAGTGCCCTCGTCACTTCCCCCAGCTACATCCCGATACCT
TCAGCCACCTGAGCCGTCTTGAAGGCCTGGTGTTGAAGGACAGTTCTCTCTCCTGGCTGAATGCCAGTTGGTTCCGT
GGGCTGGGAAACCTCCGAGTGCTGGACCTGAGTGAGAACTTCCTCTACAAATGCATCACTAAAACCAAGGCCTTCCA
GGGCCTAACACAGCTGCGCAAGCTTAACCTGTCCTTCAATTACCAAAAGAGGGTGTCCTTTGCCCACCTGTCTCTGG
CCCCTTCCTTCGGGAGCCTGGTCGCCCTGAAGGAGCTGGACATGCACGGCATCTTCTTCCGCTCACTCGATGAGACC
ACGCTCCGGCCACTGGCCCGCCTGCCCATGCTCCAGACTCTGCGTCTGCAGATGAACTTCATCAACCAGGCCCAGCT
CGGCATCTTCAGGGCCTTCCCTGGCCTGCGCTACGTGGACCTGTCGGACAACCGCATCAGCGGAGCTTCGGAGCTGA
CAGCCACCATGGGGGAGGCAGATGGAGGGGAGAAGGTCTGGCTGCAGCCTGGGGACCTTGCTCCGGCCCCAGTGGAC
ACTCCCAGCTCTGAAGACTTCAGGCCCAACTGCAGCACCCTCAACTTCACCTTGGATCTGTCACGGAACAACCTGGT
GACCGTGCAGCCGGAGATGTTTGCCCAGCTCTCGCACCTGCAGTGCCTGCGCCTGAGCCACAACTGCATCTCGCAGG
CAGTCAATGGCTCCCAGTTCCTGCCGCTGACCGGTCTGCAGGTGCTAGACCTGTCCCACAATAAGCTGGACCTCTAC
CACGAGCACTCATTCACGGAGCTACCGCGACTGGAGGCCCTGGACCTCAGCTACAACAGCCAGCCCTTTGGCATGCA
GGGCGTGGGCCACAACTTCAGCTTCGTGGCTCACCTGCGCACCCTGCGCCACCTCAGCCTGGCCCACAACAACATCC
ACAGCCAAGTGTCCCAGCAGCTCTGCAGTACGTCGCTGCGGGCCCTGGACTTCAGCGGCAATGCACTGGGCCATATG
TGGGCCGAGGGAGACCTCTATCTGCACTTCTTCCAAGGCCTGAGCGGTTTGATCTGGCTGGACTTGTCCCAGAACCG
CCTGCACACCCTCCTGCCCCAAACCCTGCGCAACCTCCCCAAGAGCCTACAGGTGCTGCGTCTCCGTGACAATTACC
TGGCCTTCTTTAAGTGGTGGAGCCTCCACTTCCTGCCCAAACTGGAAGTCCTCGACCTGGCAGGAAATCAGCTGAAG
GCCCTGACCAATGGCAGCCTGCCTGCTGGCACCCGGCTCCGGAGGCTGGATGTCAGCTGCAACAGCATCAGCTTCGT
GGCCCCCGGCTTCTTTTCCAAGGCCAAGGAGCTGCGAGAGCTCAACCTTAGCGCCAACGCCCTCAAGACAGTGGACC
ACTCCTGGTTTGGGCCCCTGGCGAGTGCCCTGCAAATACTAGATGTAAGCGCCAACCCTCTGCACTGCGCCTGTGGG
GCGGCCTTTATGGACTTCCTGCTGGAGGTGCAGGCTGCCGTGCCCGGTCTGCCCAGCCGGGTGAAGTGTGGCAGTCC
GGGCCAGCTCCAGGGCCTCAGCATCTTTGCACAGGACCTGCGCCTCTGCCTGGATGAGGCCCTCTCCTGGGACTGTT
TCGCCCTCTCGCTGCTGGCTGTGGCTCTGGGCCTGGGTGTGCCCATGCTGCATCACCTCTGTGGCTGGGACCTCTGG
TACTGCTTCCACCTGTGCCTGGCCTGGCTTCCCTGGCGGGGGCGGCAAAGTGGGCGAGATGAGGATGCCCTGCCCTA
CGATGCCTTCGTGGTCTTCGACAAAACGCAGAGCGCAGTGGCAGACTGGGTGTACAACGAGCTTCGGGGGCAGCTGG
AGGAGTGCCGTGGGCGCTGGGCACTCCGCCTGTGCCTGGAGGAACGCGACTGGCTGCCTGGCAAAACCCTCTTTGAG
AACCTGTGGGCCTCGGTCTATGGCAGCCGCAAGACGCTGTTTGTGCTGGCCCACACGGACCGGGTCAGTGGTCTCTT
GCGCGCCAGCTTCCTGCTGGCCCAGCAGCGCCTGCTGGAGGACCGCAAGGACGTCGTGGTGCTGGTGATCCTGAGCC
CTGACGGCCGCCGCTCCCGCTACGTGCGGCTGCGCCAGCGCCTCTGCCGCCAGAGTGTCCTCCTCTGGCCCCACCAG
CCCAGTGGTCAGCGCAGCTTCTGGGCCCAGCTGGGCATGGCCCTGACCAGGGACAACCACCACTTCTATAACCGGAA
CTTCTGCCAGGGACCCACGGCCGAACTCGAG

The reverse complement of MOL1e is shown below in Table 1J.

TABLE 1J
MOL1e nucleotide sequence.

(SEQ ID NO:121)

CTCGAGTTCGGCCGTGGGTCCCTGGCAGAAGTTCCGGTTATAGAAGTGGTGGTTGTCCCTGGTCAGGGCCATGCCCA
GCTGGGCCCAGAAGCTGCGCTGACCACTGGGCTGGTGGGGCCAGAGGAGGACACTCTGGCGGCAGAGGCGCTGGCGC
AGCCGCACGTAGCGGGAGCGGCGGCCGTCAGGGCTCAGGATCACCAGCACCACGACGTCCTTGCGGTCCTCCAGCAG
GCGCTGCTGGGCCAGCAGGAAGCTGGCGCGCAAGAGACCACTGACCCGCTCCGTGTGGGCCAGCACAAACAGCGTCT
TGCGGCTGCCATAGACCGAGGCCCACAGGTTCTCAAAGAGGGTTTTGCCAGGCAGCCAGTCGCGTTCCTCCAGGCAC
AGGCGGAGTGCCCAGCGCCCACGGCACTCCTCCAGCTGCCCCCGAAGCTCGTTGTACACCCAGTCTGCCACTGCGCT
CTGCGTTTTGTCGAAGACCACGAAGGCATCGTAGGGCAGGGCATCCTCATCTCGCCCACTTTGCCGCCCCCGCCAGG
GAAGCCAGGCCAGGCACAGGTGGAAGCAGTACCAGAGGTCCCAGCCACAGAGGTGATGCAGCATGGGCACACCCAGG
CCCAGAGCCACAGCCAGCAGCGAGAGGGCGAAACAGTCCCAGGAGAGGGCCTCATCCAGGCAGAGGCGCAGGTCCTG
TGCAAAGATGCTGAGGCCCTGGAGCTGGCCCGGACTGCCACACTTCACCCGGCTGGGCAGACCGGGCACGGCAGCCT
GCACCTCCAGCAGGAAGTCCATAAAGGCCGCCCCACAGGCGCAGTGCAGAGGGTTGGCGCTTACATCTAGTATTTGC
AGGGCACTCGCCAGGGGCCCAAACCAGGAGTGGTCCACTGTCTTGAGGGCGTTGGCGCTAAGGTTGAGCTCTCGCAG
CTCCTTGGCCTTGGAAAAGAAGCCGGGGGCCACGAAGCTGATGCTGTTGCAGCTGACATCCAGCCTCCGGAGCCGGG
TGCCAGCAGGCAGGCTGCCATTGGTCAGGGCCTTCAGCTGATTTCCTGCCAGGTCGAGGACTTCCAGTTTGGGCAGG
AAGTGGAGGCTCCACCACTTAAAGAAGGCCAGGTAATTGTCACGGAGACGCAGCACCTGTAGGCTCTTGGGGAGGTT
GCGCAGGGTTTGGGGCAGGAGGGTGTGCAGGCGGTTCTGGGACAAGTCCAGCCAGATCAAACCGCTCAGGCCTTGGA
AGAAGTGCAGATAGAGGTCTCCCTCGGCCCACATATGGCCCAGTGCATTGCCGCTGAAGTCCAGGGCCCGCAGCGAC
GTACTGCAGAGCTGCTGGGACACTTGGCTGTGGATGTTGTTGTGGGCCAGGCTGAGGTGGCGCAGGGTGCGCAGGTG
AGCCACGAAGCTGAAGTTGTGGCCCACGCCCTGCATGCCAAAGGGCTGGCTGTTGTAGCTGAGGTCCAGGGCCTCCA
GTCGCGGTAGCTCCGTGAATGAGTGCTCGTGGTAGAGGTCCAGCTTATTGTGGGACAGGTCTAGCACCTGCAGACCG
GTCAGCGGCAGGAACTGGGAGCCATTGACTGCCTGCGAGATGCAGTTGTGGCTCAGGCGCAGGCACTGCAGGTGCGA
GAGCTGGGCAAACATCTCCGGCTGCACGGTCACCAGGTTGTTCCGTGACAGATCCAAGGTGAAGTTGAGGGTGCTGC
AGTTGGGCCTGAAGTCTTCAGAGCTGGGAGTGTCCACTGGGGCCGGAGCAAGGTCCCCAGGCTGCAGCCAGACCTTC
TCCCCTCCATCTGCCTCCCCCATGGTGGCTGTCAGCTCCGAAGCTCCGCTGATGCGGTTGTCCGACAGGTCCACGTA
GCGCAGGCCAGGGAAGGCCCTGAAGATGCCGAGCTGGGCCTGGTTGATGAAGTTCATCTGCAGACGCAGAGTCTGGA
GCATGGGCAGGCGGGCCAGTGGCCGGAGCGTGGTCTCATCGAGTGAGCGGAAGAAGATGCCGTGCATGTCCAGCTCC
TTCAGGGCGACCAGGCTCCCGAAGGAAGGGGCCAGAGACAGGTGGGCAAAGGACACCCTCTTTTGGTAATTGAAGGA
CAGGTTAAGCTTGCGCAGCTGTGTTAGGCCCTGGAAGGCCTTGGTTTTAGTGATGCATTTGTAGAGGAAGTTCTCAC
TCAGGTCCAGCACTCGGAGGTTTCCCAGCCCACGGAACCAACTGGCATTCAGCCAGGAGAGAGAACTGTCCTTCAAC
ACCAGGCCTTCAAGACGGCTCAGGTGGCTGAAGGTATCGGGATGTAGCTGGGGGAAGTGACGAGGGCACTCCATGCA
GGGGTTGGGAGCGTGGTCGCAGCGGCGGCAATTTCCGCCCACATCGAGCACACGCAGGGCGGTCAGATTGGCCAGGT
CCTCAGGCGCCAGTTTGACGATGCGGTTGTAGGACAACAGCAGATACTCCAGGCTGGAAGGCAGGTTGCGGGGCACC
ACAGTGAGGTTGTTGTACTTGAGTGACAGGTGGGTGAGGTTGCCCAGGCCAAGGAGGGCACCCGGGGCCACCTCCAG
TGCCTGCCTGCAGGGGTTCTTGTAATAACAGTTGCCGTCCATGAATAGGAAGCGCAGGGCATGCAGGCCGGCGAGGC
TGGCAGAGTCTAGCATCAGGATGTTGGTATGGCTGAGGGACAGGGATATGAGGGATTTGGGCAGCGCAGGCACAGTC
ATGATGTTGTTGTAGCTCAGGTTTAGCTCTTCCAGGGTGGGCACAGCCAAGAAGGTGCTGGGCTCGATGGTCATGTG
GCAGGGGAAGTGCATGGGGCTGAGGCCAACCGGCGGGCAGTTCCACTTGAGGTTGAGATGCCGCAGGCTGGGCAGGT
GGGCAAAGTCAGAATCATGGAGGTGGTGGATGCGGTTGGAGGACAAGGAAAGGCTGGTGACATTGCCACGGGGTGCT
GCCATGGAGAAGTGGGGCACAGACTTCAGGAACAGCCAGTTGCAGTTCACCAGGCCGTGGGGCTGGAGCTCACAGGG
TAGGAAGGCAGGCAAGGTACCCAGGGCCAGGGTCATGGCCAGCATGATGGCCTGCACCAGGAGAGACAGCGGGTGCA
GGGCGCTGCGGCAGAAACCCATGGTGGATCC

A disclosed encoded MOL1e protein has 1037 amino acid residues, referred to as the MOL1e protein. The disclosed MOL1e polypeptide sequence is presented in Table 1K using the one-letter amino acid code.

TABLE 1K

Encoded MOL1e protein sequence.

(SEQ ID NO:122)

GSTMCFCRSALHPLSLLVQAIMLAMTLALGTLPAFLPCELQPHGLVNCNWLFLKSVPHFSMAAPRGNVTSLSLSSNR
IHHLHDSDFAHLPSLRHLNLKWNCPPVGLSPMHFPCHMTIEPSTFLAVPTLEELNLSYNNTMTVPALPKSLISLSLS
HTNTLMLDSASLAGLHALRFLFMDCNCYYKNPCRQALEVAPGALLGLGNLTHLSLKYNNLTVVPRNLPSSLEYLLLS
YNRIVKLAPEDLANLTALRVLDVGGNCRRCDHAPNPCMECPRHFPQLHPDTFSHLSRLEGLVLKDSSLSWLNASWFR
GLGNLRVLDLSENFLYKCITKTKAFQGLTQLRHLNLSFNYQKRVSFAHLSLAPSFGSLVALKELDMHGTFFRSLDET
TLRPLARLPMLQTLRLQMNFINQAQLGIFRAFPGLRYVDLSDNRISGASELTATMGEADGCEKVWLQPGDLAPAPVD
TPSSEDFRPNCSTLNFTLDLSRNNLVTVQPEMFAQLSHLQCLRLSHNCISQAVNCSQFLPLTGLQVLDLSHNKLDLY
HEHSFTELPRLEALDLSYNSQPFCMQGVCHNFSFVAHLRTLRHLSLAHNNIHSQVSQQLCSTSLRALDFSGNALGHM
WAEGDLYLHFFQGLSGLIWLDLSQNRLHTLLPQTLRNLPKSLQVLRLRDNYLAFFKWWSLHFLPKLEVLDLAGNQLK
ALTNGSLPAGTRLRRLDVSCNSISFVAPGFFSKAKELRELNLSANALKTVDHSWFGPLASALQILDVSANPLHCACG
AAFMDFLLEVQAAVPGLPSRVKCCSPGQLQGLSIFAQDLRLCLDEALSWDCFALSLLAVALGLGVPMLHHLCGWDLW
YCFHLCLAWLPWRGRQSGRDEDALPYDAFVVFDKTQSAVADWVYNELRGQLEECRGRWALRLCLEERDWLPGRTLFE
NLWASVYGSRKTLFVLAHTDRVSGLLRASFLLAQQRLLEDRKDVVVLVILSFDGRRSRYVRLRQRLCRQSVLLWPHQ
PSGQRSFWAQLGMALTRDNHHFYNRNFCQGPTAELE

MOL1f

A disclosed Toll receptor 9-like nucleic acid of 3111 nucleotides, MOL1f, is shown in Table 1L. The disclosed MOL1f open reading frame (“ORF”) begins at the GGA initiation codon at nucleotides 1-3, shown in bold in Table 1G. The encoded polypeptide is alternatively referred to herein as MOL1f or as 255304783. The disclosed MOL1f ORF terminates at a GAG codon at nucleotides 3109-3111. As shown in Table 1L the start and stop codons are in bold letters. Because MOL1f does not start or stop at traditional initiation and termination codons, MOL1f could be a partial open reading frame which extends further in the 5′ and/or 3′ directions.

TABLE 1L

MOL1f nucleotide sequence.

(SEQ ID NO:123)

GGATCCACCATGGGTTTCTGCCCCAGCGCCCTGCACCCGCTGTCTCTCCTGGTGCAGCCCATCATGCTGGCCATGAC
CCTCGCCCTGGGTACCTTGCCTGCCTTCCTACCCTCTGAGCTCCAGCCCCACGGCCTGGTGAACTGCAACTCGCTCT
TCCTGAAGTCTGTGCCCCACTTCTCCATCCCAGCACCCCGTGGCAATGTCACCAGCCTTTCCTTGTCCTCCAACCCC
ATCCACCACCTCCATGATTCTGACTTTGCCCACCTGCCCAGCCTGCGGCATCTCAACCTCAAGTGGAACTGCCCCCC
GGTTGCCCTCAGCCCCATGCACTTCCCCTGCCACATGACCATCGAGCCCAGCACCTTCTTGCCTCTGCCCATCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCGCCGGCCTGCATGCCCTGCGCTTCCTATTCATGGACGGCAA
CTGTTATTACAAGAACCCCTGCACGCACCCACTCGAGGTGGCCCCGGGTGCCCTCCTTGGCCTGGGCAACCTCACCC
ACCTGTCACTCAAGTACAACAACCTCACTGTGGTGCCCCCCAACCTGCCTTCCAGCCTGGAGTATCTGCTGTTGTCC
TACAACCGCATCGTCAAACTCGCGCCTGAGGACCTGGCCAATCTGACCGCCCTGCGTGTGCTCGATGTGGGCCGAAA
TTCCCGCCGCTGCGACCACGCTCCCAACCCCTGCATGCAGTCCCCTCGTCACTTCCCCCAGCTACATCCCGATACCT
TCAGCCACCTGAGCCGTCTTGAAGGCCTGGTCTTGAAGGACACTTCTCTCTCCTGGCTGAATGCCAGTTGGTTCCGT
GGGCTGCCAAACCTCCGAGTGCTGGACCTGAGTGAGAACTTCCTCTACAAATGCATCACTAAAACCAAGGCCTTCCA
GGGCCTAACACAGCTGCGCAAGCTTAACCTGTCCTTCAATTACCAAAAGAGGCTGTCCTTTGCCCACCTCTCTCTGG
CCCCTTCCTTCCGCAGCCTCGTCGCCCTGAAGGACCTGGACATCCACGGCATCTTCTTCCGCTCACTCGATGAGACC
ACGCTCCCCCCACTGGCCCCCCTGCCCATGCTCCAGACTCTGCGTCTGCAGATGAACTTCATCAACCAGGCCCAGCT
CGGCATCTTCAGGCCCTTCCCTGGCCTGCGCTACCTGGACCTGTCGGACAACCGCATCAGCGGAGCTTCGGAGCTGA
CAGCCACCATGGGGGAGGCAGATGGAGGGGAGAACCTCTGGCTCCAGCCTGGGGACCTTGCTCCGGCCCCAGTCGAC
ACTCCCAGCTCTGAAGACTTCAGGCCCAACTGCACCACCCTCAACTTCACCTTGGATCTGTCACGGAACAACCTGGT
GACCGTGCAGCCGGAGATCTTTGCCCAGCTCTCGCACCTGCAGTCCCTCCCCCTCAGCCACAACTGCATCTCGCAGG
CAGTCAATGGCTCCCAGTTCCTCCCCCTCACCGGTCTGCAGGTCCTAGACCTCTCCCACAATAAGCTGGACCTCTAC
CACGAGCACTCATTCACGGACCTACCACGACTGGAGGCCCTGGACCTCACCTACAACAGCCAGCCCTTTGGCATGCA
GGGCGTGGGCCACAACTTCAGCTTCGTGGCTCACCTCCCCACCCTGCGCCACCTCAGCCTGCCCCACAACAACATCC
ACAGCCAAGTGTCCCAGCAGCTCTGCAGTACGTCGCTGCGGGCCCTGGACTTCAGCGGCAATGCACTGCGCCATATG
TGGGCCGAGCGAGACCTCTATCTGCACTTCTTCCAAGGCCTGAGCGGTTTCATCTGGCTGGACTTGTCCCAGAACCG
CCTGCACACCCTCCTCCCCCAAACCCTGCGCAACCTCCCCAAGAGCCTACAGGTGCTGCGTCTCCGTGACAATTACC
TGGCCTTCTTTAAGTGGTGGAGCCTCCACTTCCTGCCCAAACTGGAAGTCCTCCACCTGGCAGGAAACCAGCTGAAG
GCCCTGACCAATGGCAGCCTGCCTGCTCCCACCCGGCTCCGGAGGCTGGATGTCAGCTGCAACAGCATCAGCTTCGT
GGCCCCCGCCTTCTTTTCCAAGGCCAAGCAGCTGCGAGAGCTCAACCTTAGCGCCAACGCCCTCAAGACAGTGGACC
ACTCCTGGTTTGCGCCCCTGGCGAGTGCCCTGCAAATACTAGATGTAAGCGCCCACCCTCTGCACTGCCCCTGTGGG
GCGGCCTTTATGGACTTCCTGCTGGAGGTGCACGCTCCCGTGCCCGGTCCGCCCAGCCGGGTGAAGTGTGGCAGTCC
GGGCCAGCTCCAGGGCCTCAGCATCTTTCCACACGACCTGCGCCTCTGCCTGGATGACGCCCTCTCCTGGGACTGTT
TCGCCCTCTCCCTCCTGGCTGTGGCTCTCGGCCTGGCTGTGCCCATGCTGCATCACCTCTGTGGCTCGCACCTCTGG
TACTGCTTCCACCTCTGCCTGGCCTGGCTTCCCTGGCCGGGGCGGCAAAGTGGGCGACATGACGATCCCCTGCCCTA
CGATCCCTTCGTGGTCTTCCACAAAACCCAGACCGCAGTGGCAGACTGGGTGTACAACGAGCTTCGGGCGCAGCTGG
AGGAGTGCCGTGGGCGCTGGGCACTCCGCCTGTGCCTGGAGGAACGCCACTGGCTGCCTGGCAAAACCCTCTTTGAC
AACCTCTGGGCCTCGGTCTATCGCAGCCGCAACACGCTGTTTCTGCTCGCCCACACGGACCGGGTCAGTGGTCTCTT
GCGCCCCAGCTTCCTGCTCGCCCAGCAGCCCCTGCTGGAGGACCGCAAGGACGTCGTGGTGCTGGTGATCCTGAGCC
CTGACGGCCGCCGCTCCCCCTACGTGCGGCTGCGCCACCGCCTCTGCCGCCAGAGTGTCCTCCTCTGGCCCCACCAG
CCCAGTGGTCAGCGCAGCTTCTGGGCCCAGCTGGGCATGGCCCTGACCAGCGACAACCACCACTTCTATAACCGGAA
CTTCTGCCACGGACCCACGCCCGAACTCGAG

A disclosed encoded MOL1f protein has 1037 amino acid residues, referred to as the MOL1f protein. The disclosed MOL1f polypeptide sequence is presented in Table 1M using the one-letter amino acid code.

TABLE 1M

Encoded MOL1f protein sequence.

(SEQ ID NO:124)

GSTMGFCRSALHPLSLLVQAIMLAMTLALGTLPAFLPCELQPHGLVNCNWLFLKSVPHFSMAAPRGNVTSLSLSSNR
IHHLHDSDFAHLPSLRHLNLKWNCPPVGLSPMHFPCHMTIEPSTELAVPILEELNLSYNNTMTVPALPKSLTSLSLS
HTNILMLDSASLAGLHALRFLFMDGNCYYKNPCRQALEVAPGALLGLGNLTHLSLKYNNLTVVPRNLPSSLEYLLLS
YNRIVKLAPEDLANLTALRVLDVCCNCRRCDHAPNPCMECPRHFFQLHPDTFSHLSRLEGLVLKDSSLSWLNASWFR
GLGNLRVLDLSENFLYKCITKTRAFQGLTQLRKLNLSFNYQKRVSFAHLSLAPSFGSLVALKELDMHGIFFRSLDET
TLRPLARLPMLQTLRLQMNFINQAQLGIFRAFPGLRYVDLSDNRISGASELTATMGEADGGEKVWLQPGDLAPAPVD
TPSSEDFRPNCSTLNPTLDLSRNNLVTVQPEMFAQLSHLQCLRLSHNCISQAVNGSQFLPLTGLQVLDLSHNKLDLY
HEHSFTELPRLEALDLSYNSQPFGMQCVGHNFSFVAHLRTLRHLSLAHNNIHSQVSQQLCSTSLRALDFSGNALGHM
WAEGDLYLHFFQGLSCLIWLDLSQNRLHTLLPQTLRNLPKSLQVLRLRDNYLAFFKWWSLHFLPKLEVLDLAGNQLK
ALTNGSLPAGTRLRRLDVSCNSISFVAPCFFSKAKELRELNLSANALKTVDHSWFCPLASALQILDVSAHPLHCACG
AAFMDFLLEVQAAVPGPPSRVKCGSPGQLQGLSIFAQDLRLCLDEALSWDCFALSLLAVALCLGVPMLHHLCGWDLW
YCFHLCLAWLPWRGRQSCRDEDALPYDAFVVFDKTQSAVADWVYNELRGQLEECRGRWALRLCLEERDWLPCKTLFE
NLWASVYGSRKTLFVLAHTDRVSGLLRASFLLAQQRLLEDRKDVVVLVILSPDGRRSRYVRLRQRLCRQSVLLWPHQ
PSGQRSFWAQLGMALTRDNHHFYNRNFCQGPTAELE

MOL1g

A disclosed Toll receptor 9-like nucleic acid of 3111 nucleotides, MOL1g, is shown in Table 1N. The disclosed MOL1g open reading frame (“ORF”) begins at the GGA initiation codon at nucleotides 1-3, shown in bold in Table 1G. The encoded polypeptide is alternatively referred to herein as MOL1g or as 255341675. The disclosed MOL1g ORF terminates at a GAG codon at nucleotides 3109-3111. As shown in Table 1N the start and stop codons are in bold letters. Because MOL1g does not start or stop at traditional initiation and termination codons, MOL1g could be a partial open reading frame which extends further in the 5′ and/or 3′ directions.

TABLE 1N

MOL1g nucleotide sequence.

(SEQ ID NO:125)

GGATCCACCATGGGTTTCTGCCGCAGCGCCCTGCACCCGCTGTCTCTCCTGGTGCAGGCCATCATCCTGGCCATGAC
CCTGCCCCTGGGTACCTTGCCTGCCTTCCTACCCTCTGAGCTCCAGCCCCACGGCCTGGTGAACTGCAACTGCCTGT
TCCTCAAGTCTGTGCCCCACTTCTCTATGGCAGCACCCCGTGGCAATGTCACCAGCCTTTCCTTGTCCTCCAACCGC
ATCCACCACCTCCATCATTCTGACTTTGCCCACCTGCCCAGCCTGCGCCATCTCAACCTCAAGTGGAACTGCCCGCC
GGTTCGCCTCAGCCCCATGCACTTCCCCTGCCACATGACCATCGAGCCCAGCACCTTCTTGGCTGTGCCCACCCTGG
AAGAGCTAAACCTGAGCTACAACAACATCATGACTGTGCCTGCCCTCCCCAAATCCCTCATATCCCTCTCCCTCAGC
CATACCAACATCCTGATGCTAGACTCTGCCAGCCTCGCCGGCCTGCATGCCCTGCGCTTCCTATTCATGGACGGCAA
CTGTTATTACAAGAACCCCTCCAGGCAGGCACTGGAGGTGGCCCCGGCTGCCCTCCTTGCCCTGGGCAACCTCACCC
ACCTGTCACTCAAGTACAACAACCTCACTGTGGTGCCCCGCAACCTGCCTTCCAGCCTGGAGTATCTGCTCTTGTCC
TACAACCGCATCGTCAAACTGGCCCCTGAGGACCTGGCCAATCTGACCGCCCTGCGTGTGCTCGATGTGGGCGGAAA
TTCCCCCCGCTCCGACCACGCTCCCAACCCCTGCATGGAGTGCCCTCGTCACTTCCCCCAGCTACATCCCGATACCT
TCAGCCACCTGAGCCGTCTTGAAGCCCTCGTGTTCAAGGACAGTTCTCTCTCCTCCCTGAATCCCAGTTGCTTCCGT
GGGCTGGGAAACCTCCGAGTGCTGGACCTGAGTGAGAACTTCCTCTACAAATGCATCACTAAAACCAAGCCCTTCCA
GGGCCTAACACAGCTGCGCAAGCTTAACCTGTCCTTCAATTACCAAAAGAGCGTGTCCTTTGCCCACCTGTCTCTGG
CCCCTTCCTTCGGGAGCCTGGTCGCCCTGAAGCAGCTGGACATGCACGGCATCTTCTTCCGCTCACTCGATGAGACC
ACGCTCCGGCCACTGGCCCGCCTGCCCATGCTCCAGACTCTGCGTCTGCAGATGAACTTCATCAACCAGGCCCAGCT
CGGCATCTTCACGGCCTTCCCTGGCCTGCGCTACGTGCACCTGTCCGACAACCGCATCAGCGGAGCTTCGGAGCTGA
CAGCCACCATGGGGGAGGCAGATGGAGGGGAGAAGGTCTGGCTGCAGCCTCGGGACCTTGCTCCGGCCCCAGTGGAC
ACTCCCAGCTCTGAAGACTTCAGGCCCAACTGCAGCACCCTCAACTTCACCTTGGATCTCTCACGGAACAACCTGGT
GACCGTGCAGCCGGAGATGTTTGCCCAGCTCTCGCACCTGCAGTGCCTGCGCCTGACCCACAACTGCATCTCCCAGC
CAGTCAATGGCTCCCAGTTCCTGCCGCTGACCGGTCTGCAGGTGCTACACCTGTCCCACAATAAGCTGGACCTCTAC
CACGAGCACTCATTCACGGAGCTACCACGACTGGAGGCCCTGGACCTCAGCTACAACAGCCAGCCCTTTGGCATGCA
GGCCGTGGGCCACAACTTCAGCTTCGTGGCTCACCTGCGCACCCTGCGCCACCTCAGCCTGCCCCACAACAACATCC
ACAGCCAAGTCTCCCAGCAGCTCTGCAGTACGTCGCTGCGGGCCCTGGACTTCAGCGGCAATGCACTGGGCCATATG
TCGGCCGAGGGAGACCTCTATCTGCACTTCTTCCAACGCCTGAGCGGTTTGATCTGGCTGGACTTGTCCCAGAACCG
CCTGCACACCCTCCTCCCCCAAACCCTCCGCAACCTCCCCAAGAGCCTACACGTGCTGCGTCTCCGTGACAATTACC
TGGCCTTCTTTAAGTGGTGGAGCCTCCACTTCCTGCCCAAACTGGAAGTCCTCGACCTGGCAGGAAACCAGCTGAAG
GCCCTGACCAATGGCAGCCTGCCTGCTGGCACCCGCCTCCCGAGGCTGCATGTCAGCTGCAACAGCATCAGCTTCGT
GCCCCCCGGCTTCTTTTCCAAGGCCAAGGAGCTGCCAGAGCTCAACCTTAGCGCCAACGCCCTCAAGACAGTGGACC
ACTCCTGGTTTGGGCCCCTGGCGAGTGCCCTGCAAATACTAGATGTAAGCGCCAACCCTCTGCACTGCGCCTGTGGC
GCGGCCTTTATGGACTTCCTGCTGGAGGTGCACGCTGCCGTGCCCGGTCCGCCCAGCCGGGTGAAGTGTGGCAGTCC
GGCCCAGCTCCAGGCCCTCAGCATCTTTGCACAGGACCTGCGCCTCTCCCTGGATGAGGCCCTCTCCTGGGACTGTT
TCGCCCTCTCGCTGCTGGCTGTGGCTCTGGGCCTGGGTGTGCCCATGCTGCATCACCTCTGTGGCTGGGACCTCTGG
TACTCCTTCCACCTGTCCCTGGCCTGGCTTCCCTGGCGGGGGCGGCAAAGTGGGCGAGATGAGGATGCCCTGCCCTA
CGATCCCTTCGTGGTCTTCGACAAAACGCAGACCGCAGTGGCAGACTGGGTGTACAACCAGCTTCGGGGGCAGCTCG
AGGAGTGCCGTGGGCGCTGGGCACTCCGCCTGTCCCTGGACGAACGCGACTGGCTCCCTGGCAAAACCCTCTTTGAG
AACCTGTGGCCCTCGGTCTATCGCAGCCGCAAGACCCTGTTTGTGCTGGCCCACACGCACCGGCTCAGTGGTCTCTT
CCGCGCCAGCTTCCTGCTGGCCCAGCAGCGCCTGCTGGAGGACCGCAAGGACGTCGTCGTGCTGGTGATCCTGAGCC
CTCACGGCCGCCGCTCCCGCTACGTGCGGCTGCCCCACCGCCTCTGCCCCCAGAGTGTCCTCCTCTGGCCCCACCAG
CCCAGTGGTCAGCGCAGCTTCTGCGCCCAGCTGGGCATGGCCCTGACCAGCCACAACCACCACTTCTATAACCGGAA
CTTCTGCCAGCGACCCACCGCCGAACTCGAG

The reverse complement of MOL1g is shown below in Table 1O.

TABLE 10

MOL1g nucleotide sequence.

(SEQ ID NO:126)

CTCGAGTTCGGCCGTGGGTCCCTGGCAGAAGTTCCGGTTATAGAAGTGGTGGTTGTCCCTGGTCAGGGCCATGCCCA
GCTGGGCCCAGAAGCTGCGCTGACCACTGGGCTGGTGGGGCCAGACCAGGACACTCTGGCGGCAGAGGCGCTGGCGC
AGCCCCACGTAGCGGGAGCCGCGGCCGTCAGGGCTCAGGATCACCACCACCACGACGTCCTTGCGGTCCTCCAGCAG
GCGCTGCTGGGCCAGCAGGAAGCTGGCGCGCAAGAGACCACTGACCCGGTCCGTGTGGGCCAGCACAAACAGCCTCT
TGCGGCTGCCATAGACCGAGGCCCACACGTTCTCAAAGAGGGTTTTGCCAGGCAGCCAGTCGCGTTCCTCCAGGCAC
AGGCGGAGTGCCCAGCGCCCACGGCACTCCTCCAGCTGCCCCCCAAGCTCGTTGTACACCCAGTCTGCCACTGCGCT
CTGCGTTTTGTCGAAGACCACGAAGGCATCGTAGGGCAGGGCATCCTCATCTCGCCCACTTTGCCGCCCCCGCCAGG
GAAGCCAGCCCAGGCACAGGTGGAAGCAGTACCAGAGGTCCCACCCACAGAGGTGATCCAGCATGGGCACACCCAGG
CCCAGAGCCACAGCCACCAGCGAGAGGGCGAAACAGTCCCAGGAGAGGGCCTCATCCAGGCAGAGGCGCACGTCCTG
TGCAAAGATGCTGAGGCCCTGGACCTGCCCCGGACTGCCACACTTCACCCGCCTGGGCGGACCGGGCACGGCAGCCT
GCACCTCCACCAGGAAGTCCATAAACGCCGCCCCACAGGCGCAGTGCAGAGGGTTGGCCCTTACATCTAGTATTTGC
ACGGCACTCGCCAGCGGCCCAAACCAGGAGTGGTCCACTCTCTTCAGGGCGTTGGCGCTAAGGTTGACCTCTCCCAG
CTCCTTCGCCTTGGAAAAGAACCCGGGGGCCACGAACCTGATGCTGTTCCAGCTGACATCCAGCCTCCGGAGCCGGG
TGCCACCACGCACGCTGCCATTGGTCAGCGCCTTCAGCTCGTTTCCTGCCAGCTCGAGGACTTCCACTTTGGGCAGG
AACTGGAGGCTCCACCACTTAAAGAACGCCAGGTAATTGTCACGGAGACCCAGCACCTGTAGGCTCTTGGGGAGGTT
GCGCAGGGTTTGGGGCAGGAGCGTGTGCAGCCGGTTCTGGGACAAGTCCAGCCACATCAAACCCCTCAGGCCTTGGA
AGAAGTGCAGATAGAGGTCTCCCTCGGCCCACATATCGCCCAGTGCATTCCCGCTGAAGTCCACGGCCCGCAGCGAC
GTACTGCAGAGCTGCTGGGACACTTGGCTGTGGATGTTGTTGTGGGCCAGGCTGACGTGGCGCAGCGTGCGCAGGTG
AGCCACGAAGCTGAAGTTGTGGCCCACGCCCTGCATGCCAAAGGGCTGGCTGTTGTAGCTGAGGTCCAGGGCCTCCA
GTCGTGGTAGCTCCCTGAATGAGTGCTCGTGGTAGAGGTCCAGCTTATTGTGCGACAGGTCTAGCACCTGCAGACCG
GTCAGCGGCAGGAACTGGCAGCCATTGACTGCCTGCGAGATGCAGTTGTGGCTCAGGCGCAGGCACTCCAGGTGCGA
GAGCTGGGCAAACATCTCCGGCTGCACGGTCACCACGTTGTTCCGTGACAGATCCAAGGTGAACTTGAGGGTGCTGC
AGTTGGGCCTGAAGTCTTCAGAGCTGGGAGTGTCCACTGGGCCCGGAGCAAGGTCCCCAGGCTGCAGCCAGACCTTC
TCCCCTCCATCTGCCTCCCCCATGGTGCCTGTCAGCTCCGAAGCTCCGCTCATGCGGTTGTCCGACAGGTCCACGTA
GCGCAGGCCAGCGAAGGCCCTGAAGATCCCGAGCTGGCCCTCGTTGATGAACTTCATCTGCAGACGCAGAGTCTGGA
GCATCGGCAGGCGGGCCAGTGGCCGGAGCGTGGTCTCATCGACTGAGCGGAAGAAGATGCCCTCCATGTCCAGCTCC
TTCAGGGCGACCAGGCTCCCCAAGGAAGGGGCCAGAGACAGCTGGGCAAAGCACACCCTCTTTTGGTAATTGAAGGA
CAGGTTAAGCTTGCGCAGCTCTGTTAGGCCCTGGAAGGCCTTCGTTTTACTGATGCATTTGTAGAGGAACTTCTCAC
TCAGGTCCAGCACTCGGACGTTTCCCAGCCCACGGAACCAACTCGCATTCACCCAGGAGAGAGAACTGTCCTTCAAC
ACCAGGCCTTCAACACGGCTCAGGTGCCTGAACCTATCGGGATCTAGCTGGGGGAAGTCACGAGGGCACTCCATGCA
GGGGTTGGGAGCGTGGTCGCAGCGGCGGCAATTTCCGCCCACATCGAGCACACGCAGGGCGGTCAGATTGCCCACGT
CCTCACGCCCCAGTTTGACGATGCGGTTCTAGGACAACAGCAGATACTCCACGCTGGAAGGCAGGTTGCGGGGCACC
ACAGTGAGGTTGTTCTACTTGAGTGACACGTGGGTGAGGTTGCCCAGGCCAAGGAGCCCACCCGGGGCCACCTCCAG
TGCCTGCCTGCAGGGGTTCTTCTAATAACAGTTGCCGTCCATGAATAGGAAGCGCAGGGCATGCAGGCCCCCGAGGC
TGGCAGAGTCTAGCATCAGCATGTTGGTATGGCTGAGGGACAGGCATATCACGGATTTGGGCAGCGCAGGCACAGTC
ATGATGTTGTTGTACCTCAGGTTTAGCTCTTCCAGGGTGGGCACAGCCAAGAAGGTGCTGGCCTCGATGGTCATGTG
GCACGGGAAGTGCATGGGGCTGAGGCCAACCGGCGGGCAGTTCCACTTGAGGTTGAGATGCCGCAGGCTCGGCAGGT
GGGCAAAGTCAGAATCATGGAGGTGGTGGATGCGCTTGGAGGACAAGGAAAGGCTGGTCACATTCCCACGGGGTGCT
GCCATAGAGAACTGGGGCACAGACTTCACGAACAGCCAGTTGCACTTCACCAGGCCGTGGGGCTGGAGCTCACAGCG
TAGGAAGCCAGGCAAGGTACCCAGGCCCAGCGTCATGGCCAGCATGATGGCCTGCACCAGGAGAGACAGCGGGTGCA
GGCCGCTGCGGCAGAAACCCATGGTGGATCC

A disclosed encoded MOL1g protein has 1037 amino acid residues, referred to as the MOL1g protein. The disclosed MOL1g polypeptide sequence is presented in Table 1P using the one-letter amino acid code.

TABLE 1P

Encoded MOL1g protein sequence.

(SEQ ID NO:127)

GSTMGFCRSALHPLSLLVQAIMLANTLALGTLPAFLFCELQPHGLVNCNWLFLKSVPHFSMAAPRGNVTSLSLSSNR
IHHLHDSDFAHLPSLRHLNLKWNCPPVCLSPMHFPCHMTIEPSTFLAVPTLEELNLSYNNIMTVPALPKSLTSLSLS
HTNILMLDSASLAGLHALRFLFMDGNCYYKNPCRQALEVAPGALLGLGNLTFLSLKYNNLTVVPRNLPSSLEYLLLS
YNRTVKLAPEDLANLTALRVLDVGGNCRRCDHAPNPCMECPRHFPQLEPDTFSHLSRLEGLVLKDSSLSWLNASWFR
GLGNLRVLDLSENFLYKCITKTKAFQGLTQLRKLNLSFNYQKRVSFAHLSLAPSFGSLVALKELDMHCTFFRSLDET
TLRPLARLPMLQTLRLQMNFINQAQLGTFPAFPGLRYVDLSDNRISGASELTATMGEADGGEKVWLQPGDLAPAPVD
TPSSEDFRPNCSTLNFTLDLSRNNLVTVQPEMFAQLSHLQCLRLSHNCTSQAVNGSQFLPLTGLQVLDLSHNKLDLY
HEHSFTELPRLEALDLSYNSQPFGMQGVGHNFSFVAHLRTLRHLSLAHNNIHSQVSQQLCSTSLRALDFSGNALGHM
WAEGDLYLHFFQGLSGLIWLDLSQNRLHTLLPQTLRNLPKSLQVLRLRDNYLAFFKWWSLHFLPKLEVLDLAGNQLK
ALTNGSLPAGTPIRRLDVSCNSISFVAPCFFSKAKELRELNLSANALKTVDHSWFGPLASALQILDVSANPLHCACG
AAFMDFLLEVQAAVPCPPSRVKCGSPGQLQGLSIFAQDLRLCLDEALSWDCFALSLLAVALGLGVPMLHHLCGWDLW
YCFHLCLAWLPWRGRQSGRDEDALPYDAFVVFDKTQSAVADWVYNELRGQLEECRGRWALRLCLEERDWLPGKTLFE
NLWASVYGSRKTLFVLAHTDRVSCLLRASFLLAQQRLLEDRKDVVVLVILSFDCRRSRYVRLRQRLCRQSVLLWPHQ
PSGQRSFWAQLGMALTRDNHHFYNRNFCQGPTAELE

MOL1h

A disclosed Toll receptor 9-like nucleic acid of 2145 nucleotides, MOL1h, is shown in Table 1Q. The disclosed MOL1h open reading frame (“ORF”) begins at the GGA initiation codon at nucleotides 1-3, shown in bold in Table 1Q. The encoded polypeptide is alternatively referred to herein as MOL1h or as 248210503. The disclosed MOL1h ORF terminates at a GAG codon at nucleotides 2143-2145. As shown in Table 1Q the start and stop codons are in bold letters. Because MOL1h does not start or stop at traditional initiation and termination codons, MOL1h could be a partial open reading frame which extends further in the 5′ and/or 3 ′ directions.

TABLE 1Q

MOL1h nucleotide sequence.

(SEQ ID NO:128)

GGATCCAATGTCACCAGCCTTTTCTTGTCCTCCAACCCCATCCACCACCTCCATGATTCTGACTTTGCCCACCTCCC
CAGCCTGCCGCATCTCAACCTCAAGTGGAACTGCCCGCCGGTTCGCCTCACCCCCATGCACTTCCCCTGCCACATCA
CCATCGAGCCCACCACCTTCTTGGCTGTGCCCACCCTGGAAGAGCTAAACCTGAGCTACAACAACATCATGACTGTG
CCTCCGCTGCCCAAATCCCTCATATCCCTGTCCCTCAGCCATACCAACATCCTGATGCTAGACTCTGCCAGCCTCGC
CGGCCTGCATCCCCTCCCCTTCCTATTCATGCACCGCAACTGTTATTACAAGAACCCCTGCAGGCAGGCACTGGAGC
TGGCCCCGCGTGCCCTCCTTGGCCTGGCCAACCTCACCCACCTGTCACTCAAGTACAACAACCTCACTGTGGTGCCC
CGCAACCTGCCTTCCAGCCTGGAGTATCTCCTGTTGTCCTACAACCGCATCGTCAAACTGCCGCCTGACGACCTGGC
CAATCTGACCGCCCTGCGTGTGCTCGATGTGGGCGGAAATTGCCCCCGCTGCCACCACGCTCCCAACCCCTGCATGG
AGTGCCCTCGTCACTTCCCCCAGCTACATCCCGATACCTTCAGCCACCTGAGCCGTCTTGAAGGCCTGGTGTTGAAG
CACAGTTCTCTCTCCTGGCTGAATGCCAGTTGGTTCCGTGGGCTGGGAAACCTCCGAGTGCTGGACCTGAGTGAGAA
CTTCCTCTACAAATCCATCACTAAAACCAACGCCTTCCAGCGCCTAACACAGCTGCGCAAGCTTAACCTGTCCTTCA
ATTACCAAAAGAGGCTGTCCTTTGCCCACCTGTCTCTGGCCCCTTCCTTCGGCAGCCTGGTCGCCCTCAAGCAGCTG
GACATGCACGGCATCTTCTTCCGCTCACTCGATGAGACCACGCTCCGGCCACTGGCCCGCCTGCCCATGCTCCAGAC
TCTGCGTCTGCACATGAACTTCATCAACCAGGCCCAGCTCGGCATCTTCAGGGCCTTCCCTGGCCTGCGCTACGTGG
ACCTGTCGGACAACCGCATCAGCCGAGCTTCGGAGCTCACAGCCACCATGGGGGAGGCACATCGAGGGCAGAAGGTC
TGGCTGCAGCCTGCGGACCTTGCTCCGGCCCCAGTGGACACTCCCACCTCTGAAGACTTCAGCCCCAACTGCAGCAC
CCTCAACTTCACCTTCCATCTGTCACGGAACAACCTGGTGACCGTGCAGCCCGAGATGTTTGCCCAGCTCTCGCACC
TGCAGTGCCTGCGCCTGAGCCACAACTGCATCTCGCACGCAGTCAATGGCTCCCAGTTCCTGCCCCTGACCGGTCTG
CAGGTGCTAGACCTGTCCCACAATAAGCTGGACCTCTACCACGAGCACTCATTCACGGAGCTACCACCACTGGAGCC
CCTGGACCTCAGCTACAACACCCAGCCCTTTGGCATGCAGGGCGTGGGCCACAACTTCAGCTTCCTGGCTCACCTGC
GCACCCTGCGCCACCTCAGCCTGGCCCACAACAACATCCACAGCCAAGTCTCCCAGCAGCTCTGCAGTACGTCGCTG
CGGGCCCTGGACTTCAGCGCCAATGCACTCGGCCATATGTGGCCCGAGGGAGACCTCTATCTGCACTTCTTCCAAGG
CCTGAGCGGTTTGATCTGGCTGGACTTGTCCCAGAACCGCCTGCACACCCTCCTGCCCCAAACCCTCCGCAACCTCC
CCAAGAGCCTACAGGTGCTCCGTCTCCCTGACAATTACCTGGCCTTCTTTAAGTGGTGGAGCCTCCACTTCCTGCCC
AAACTGGAAGTCCTCGACCTGGCAGGAAACCAGCTGAAGGCCCTCACCAATGGCAGCCTGCCTGCTGGCACCCGGCT
CCGGAGGCTGGATGTCAGCTGCAACAGCATCAGCTTCGTGGCCCCCCCCTTCTTTTCCAAGGCCAAGGAGCTGCGAG
AGCTCAACCTTAGCGCCAACGCCCTCAAGACAGTGGACCACTCCTGGTTTGGGCCCCTGCCGAGTGCCCTGCAAATA
CTACATGTAAGCGCCAACCCTCTCCACTGCCCCTGTGCCGCGGCCTTTATCCACTTCCTGCTCGAG

A disclosed encoded MOL1h protein has 715 amino acid residues, referred to as the MOL1h protein. The disclosed MOL1h polypeptide sequence is presented in Table 1R using the one-letter amino acid code.

TABLE 1R

Encoded MOL1h protein sequence.

(SEQ ID NO:129)

GSNVTSLFLSSNRIHHLHDSDFAHLPSLRHLNLKWNCPPVGLSPMHFPCHMTIEPSTFLAVPTLEELNLSYNNTMTV
PALPKSLISLSLSHTNILMLDSASLAGLHALRFLFMDCNCYYKNPCRQALEVAPGALLGLGNLTHLSLKYNNLTVVP
RNLPSSLEYLLLSYNRIVKLAPEDLANLTALRVLDVGCNCRRCDHAPNPCMECPRHFPQLHPDTFSHLSRLEGLVLK
DSSLSWLNASWFRGLGNLRVLDLSENFLYKCITKTKAFQGLTQLRKLNLSFNYQKRVSFAHLSLAPSFGSLVALKEL
DMHGIFFRSLDETTLRPLARLPMLQTLRLQMNFINQAQLGIFRAFPGLRYVDLSDNRISGASELTATMGEADGGEKV
WLQPCDLAPAPVDTPSSEDFRPNCSTLNFTLDLSRNNLVTVQPEMFAQLSHLQCLRLSHNCISQAVNGSQFLPLTGL
QVLDLSHNKLDLYHEHSFTELPRLEALDLSYNSQPFGMQGVGHNFSFVAHLRTLRHLSLAHNNIHSQVSQQLCSTSL
RALDFSGNALGHMWAEGDLYLHFFQGLSGLIWLDLSQNRLHTLLPQTLRNLPKSLQVLRLRDNYLAFFKWWSLHFLP
KLEVLDLAGNQLKALTNGSLPAGTRLRRLDVSCNSISFVAPGFFSKAKELRELNLSANALKTVDHSWFGPLASALQI
LDVSANPLHCACGAAFMDFLLE

MOL1i

A disclosed Toll receptor 9-like nucleic acid of 2145 nucleotides, MOL1i, is shown in Table 1S. The disclosed MOL1i open reading frame (“ORF”) begins at the GGA initiation codon at nucleotides 1-3, shown in bold in Table 1S. The encoded polypeptide is alternatively referred to herein as MOL1i or as 248210481. The disclosed MOL1i ORF terminates at a GAG codon at nucleotides 2143-2145. As shown in Table 1S the start and stop codons are in bold letters. Because MOL1i does not start or stop at traditional initiation and termination codons, MOL1i could be a partial open reading frame which extends further in the 5′ and/or 3′ directions.

TABLE 1S

MOL1i nucleotide sequence.

(SEQ ID NO:130)

GCATCCAATGTCACCAGCCTTTCCTTGTCCTCCAACCGCATCCACCACCTCCATGATTCTGACTTTGCCCACCTGCC
CAGCCTGCGGCATCTCAACCTCAAGTGCAACTGCCCGCCGCTTGCCCTCACCCCCATGCACTTCCCCTGCCACATGA
CCATCCAGCCCACCACCTTCTTGGCTGTCCCCACCCTGGAAGAGCTAAACCTGAGCTACAACAACATCATGACTGTG
CCTGCCCTGCCCAAATCCCTCATATCCCTGTCCCTCAGCCATACCAACATCCTCATGCTAGACTCTGCCAGCCTCGC
CGGCCTGCATCCCCTGCGCTTCCTATTCATGGACGGCAACTGTTATTACAAGAACCCCTGCAGGCAGGCACTGGAGG
TGGCCCCGGGTGCCCTCCTTGGCCTCGGCAACCTCACCCACCTCTCACTCAACTACAACAACCTCACTGTGGTCCCC
CGCAACCTGCCTTCCAGCCTGGAGTATCTGCTGTTGTCCTACAACCGCATCGTCAAACTGGCGCCTGAGGACCTCGC
CAATCTGACCCCCCTCCGTGTGCTCGATGTGCGCGGAAATTGCCCCCGCTGCCACCACGCTCCCAACCCCTACATGG
AGTGCCCTCGTCACTTCCCCCAGCTACATCCCGATACCTTCAGCCACCTGAGCCGTCTTGAAGGCCTGGTGTTCAAG
GACAGTTCTCTCTCCTGGCTGAATGCCAGTTGGTTCCGTGGGCTGGGAAACCTCCGACTGCTGGACCTGAGTGAGAA
CTTCCTCTACAAATGCATCACTAAAACCAAGGCCTTCCAGGGCCTAACACAGCTGCGCAAGCTTAACCTGTCCTTCA
ATTACCAAAAGAGGGTGTCCTTTGCCCACCTGTCTCTGGCCCCTTCCTTCGGGAGCCTCGTCGCCCTGAAGGAGCTG
GACATGCACGGCATCTTCTTCCGCTCACTCGATGAGACCACGCTCCGGCCACTGGCCCGCCTGCCCATGCTCCAGAC
TCTGCGTCTGCAGATGAACTTCATCAACCAGGCCCAGCTCGGCATCTTCAGGGCCTTCCCTGGCCTGCGCTACGTGG
ACCTGTCGGACAACCACATCACCGGAGCTTCGGAGCTGACAGCCACCATCGGGGAGGCAGATGGAGCGCAGAAGGTC
TCGCTGCAGCCTGGGCACCTTCCTCCGGCCCCAGTGGACACTCCCAGCTCTGAAGACTTCAGGCCCAACTCCAGCAC
CCTCAACTTCACCTTGGATCTGTCACGGAACAACCTGGTGACCGTGCAGCCGGAGATCTTTGCCCAGCTCTCGCACC
TGCAGTGCCTGCGCCTGAGCCACAACTGCATCTCGCAGGCAGTCAATGGCTCCCAGTTCCTGCCGCTGACCGGTCTC
CAGGTGCTAGACCTGTCCCACAATAAGCTCGACCTCTACCACCAGCACTCATTCACGGAGCTACCACGACTGGAACC
CCTGCACCTCAGCTACAACAGCCAGCCCTTTGCCATCCACGGCGTGGGCCACAACTTCAGCTTCGTGGCTCACCTGC
GCACCCTGCGCCACCTCAGCCTGGCCCACAACAACATCCACAGCCAAGTGTCCCAGCAGCTCTGCAGTACGTCGCTG
CGGGCCCTGGACTTCACCGGCAATGCACTGGGCCATATGTGGGCCGAGGGAGACCTCTATCTGCACTTCTTCCAAGG
CCTGAGCGGTTTCATCTGGCTCGACTTGTCCCAGAACCCCCTGCACACCCTCCTGCCCCAAACCCTGCGCAACCTCC
CCAAGAGCCTACAGGTGCTGCGTCTCCGTGACAATTACCTGGCCTTCTTTAAGTGGTGGAGCCTCCACTTCCTGCCC
AAACTGGAAGTCCTCGACCTGGCACGAAACCACCTGAAGGCCCTGACCAATGGCAGCCTCCCTGCTGGCACCCGGCT
CCGGAGGCTGCATGTCAGCTGCAACAGCATCAGCTTCGTGGCCCCCGCCTTCTTTTCCAACCCCAAGGAGCTCCGAG
AGCTCAACCTTAGCGCCAACGCCCTCAAGACAGTGGACCACTCCTCGTTTCGGCCCCTGGCGAGTGCCCTCCAAATA
CTAGATGTAAGCGCCAACCCTCTGCACTGCGCCTGTCGGGCCGCCTTTATGGACTTCCTGCTCCAG

A disclosed encoded MOL1i protein has 715 amino acid residues, referred to as the MOL1i protein. The disclosed MOL1i polypeptide sequence is presented in Table 1T using the one-letter amino acid code.

TABLE 1T

Encoded MOL1i protein sequence.

(SEQ ID NO:131)

GSNVTSLSLSSNRIHHLHDSDFAGLPSLRHLNLKWNCPPVGLSPMHFPCHMTIEPSTFLAVPTLEELNLSYNNIMTV
PALPKSLISLSLSHTNILMLDSASLACLHALRFLFMDGNCYYKNPCRQALEVAPGALLGLGNLTHLSLKYNNLTVVP
RNLPSSLEYLLLSYNRIVKLAPEDLANLTALRVLDVGGNCRRCDHAPNPCMECPRHFPQLHPDTFSHLSRLECLVLK
DSSLSWLNASWFRGLGNLRVLDLSENFLYKCITKTKAFQGLTQLRKLNLSFNYQKRVSFAHLSLAPSFGSLVALKEL
DMHCIFFRSLDETTLRPLARLPMLQTLRLQMNFTNQAQLGIFRAFPGLRYVDLSDNHISGASELTATMGEADCGEKV
WLQPGDLAPAPVDTPSSEDFRPNCSTLNFTLELSRNNLVTVQPEMFAQLSHLQCLRLSHNCISQAVNGSQFLPLTGL
QVLDLSHNKLDLYHEHSFTELPRLEALDLSYNSQPFGMQGVCHNFSFVAHLRTLRHLSLAHNNIHSQVSQQLCSTSL
RALDFSGNALGHMWAEGDLYLHFFQGLSGLIWLDLSQNRLHTLLPQTLRNLPKSLQVLRLRDNYLAFFKWWSLHFLP
KLEVLDLAGNQLKALTNCSLPAGTRLRRLDVSCNSISFVAPCFFSKAKELRELNLSANALKTVDHSWFGPLASALQI
LDVSANPLHCACGAAFMDFLLE

MOL1j

A disclosed Toll receptor 9-like nucleic acid of 2145 nucleotides, MOL1j, is shown in Table 1U. The disclosed MOL1j open reading frame (“ORF”) begins at the GGA initiation codon at nucleotides 1-3, shown in bold in Table 1U. The encoded polypeptide is alternatively referred to herein as MOL1j or as 248210473. The disclosed MOL1j ORF terminates at a GAG codon at nucleotides 2143-2145. As shown in Table 1U the start and stop codons are in bold letters. Because MOL1j does not start or stop at traditional initiation and termination codons, MOL1j could be a partial open reading frame which extends further in the 5′ and/or 3 ′ directions.

TABLE 1U

MOL1j nucleotide sequence.

(SEQ ID NO:132)

GGATCCAATGTCACCAGCCTTTCCTTGTCCTCCAACCGCATCCACCACCTCCATGATTCTGACTTTGCCCACCTGCC
CAGCCTGCGGCATCTCAACCTCAAGTGGAACTGCCCGCCCGTTGGCCTCACCCCCATGCACTTCCCCTGCCACATGA
CCATCGAGCCCAGCACCTTCTTGGCTGTGCCCACCCTGGAAGAGCTAAACCTGAGCTACAACAACATCATGACTGTG
CCTGCGCTCCCCAAATCCCTCATATCCCTCTCCCTCAGCCATACCAACATCCTGATGCTAGACTCTGCCAGCCTCGC
CGGCCTCCATCCCCTGCGCTTCCTATTCATGGACGGCAACTGTTATTACAAGAACCCCTCCAGGCAGGCACTGGAGG
TGGCCCCGGGTGCCCTCCTTGGCCTCGGCAACCTCACCCACCTGTCACTCAAGTACAACAACCTCACTGTGGTGCCC
CGCAACCTGCCTTCCAGCCTCGAGTATCTGCTGTTGTCCTACAACCGCATCGTCAAACTGGCGCCTCAGGACCTGGC
CAATCTGACCGCCCTCCGTGTGCTCGATGTGGGCGGAAATTGCCCCCGCTGCGACCACGCTCCCAACCCCTCCATGG
AGTGCCCTCGTCACTTCCCCCAGCTACATCCCGATACCTTCAGCCACCTGAGCCGTCTTGAAGGCCTGGTGTTGAAG
GACACTTCTCTCTCCTGGCTGAATGCCAGTTGGTTCCGTGGGCTCGGAAACCTCCGAGTGCTGGACCTCAGTGAGAA
CTTCCTCTACAAATGCATCACTAAAACCAAGGCCTTCCAGCGCCTAACACAGCTCCGCAAGCTTAACCTGTCCTTCA
ATTACCAAAAGAGGGTGTCCTTTGCCCACCTGTCTCTGGCCCCTTCCTTCGGGAGCCTGGTCGCCCTGAAGGAGCTG
GACATGCACGGCATCTTCTTCCCCTCACTCGATGAGACCACGCTCCGGCCACTGGCCCGCCTGCCCATGCTCCAGAC
TCTGCGTCTGCAGATGAACTTCATCAACCAGGCCCAGCTCGGCATCTTCAGGGCCTTCCCTGGCCTGCCCTACGTGG
ACCTCTCGGACAACCGCATCAGCGGAGCTTCGGAGCTGACAGCCACCATGCGGCAGGCAGATGGAGGGGAGAACGTC
TGCCTGCAGCCTGCGGACCTTGCTCCGGCCCCAGTGGACACTCCCAGCTCTGAAGACTTCAGGCCCAACTGCAGCAC
CCTCAACTTCACCTTGGATCTGTCACGGAACAACCTGGTGACCGTGCAGCCGGACATGTTTGCCCAGCTCTCGCACC
TGCAGTCCCTGCGCCTGAGCCACAACTGCATCTCGCAGGCAGTCAATCGCTCCCAGTTCCTGCCGCTGACCGGTCTG
CAGGTGCTAGACCTGTCCCACAATAAGCTGCACCTCTACCACGAGCACTCATTCACGGAGCTACCACGACTCGAGGC
CCTCCACCTCAACTACAACAGCCACCCCTTTGGCATGCAGCGCGTGGGCCACAACTTCAGCTTCGTGGCTCACCTGC
GCACCCTGCCCCACCTCAGCCTCGCCCACAACAACATCCACAGCCAAGTGTCCCAGCACCTCTGCAGTACGTCGCTG
CGGGCCCTGGACTTCAGCGCCAATGCACTGGGCCATATGTGGGCCGAGGGAGACCTCTATCTGCACTTCTTCCAAGG
CCTGAGCGGTTTGATCTGGCTGGACTTGTCCCAGAACCGCCTGCACACCCTCCTGCCCCAAACCCTGCGCAACCTCC
CCAAGAGCCTACACGTCCTGCGTCTCCGTGACAATTACCTGGCCTTCTTTAAGTGGTGGAGCCTCCACTTCCTGCCC
AAACTCGAAGTCCTCGACCTGGCAGGAAACCAGCTCAAGGCCCTGACCAATGGCAGCCTGCCTGCTGGCACCCGGCT
CCGGAGGCTGGATGTCAGCTGCAACAGCATCAGCTTCGTCGCCCCCCCCTTCTTTTCCAAGGCCAAGGAGCTGCGAG
AGCTCAACCTTACCCCCAACCCCCTCAAGACAGTGGACCACTCCTGGTTTGGCCCCCTGCCGAGTGCCCTGCAAATA
CTAGATGTAAGCGCCAACCCTCTGCACTGCGCCTGTGCCCCGGCCTTTATGGACTTCCTGCTCGAG

A disclosed encoded MOL1j protein has 715 amino acid residues, referred to as the MOL1j protein. The disclosed MOL1j polypeptide sequence is presented in Table 1V using the one-letter amino acid code.

TABLE 1V

Encoded MOL1j protein sequence.

(SEQ ID NO:133)

GSNVTSLSLSSNRIHHLHDSDFAHLPSLRHLNLKWNCPPVGLSPMHFPCHMTIEPSTFLAVPTLEELNLSYNNIMTV
PALPKSLISLSLSHTNILMLDSASLAGLHALRFLFMDGNCYYKNFCRQALEVAPCALLCLGNLTHLSLKYNNLTVVP
RNLPSSLEYLLLSYNRIVKLAPEDLANLTALRVLDVGGNCRRCDHAPNPCMECPRHFPQLHPDTFSHLSRLEGLVLK
DSSLSWLNASWFRGLGNLRVLDLSENPLYKCITKTKAFQGLTQLRHLNLSFNYQKRVSFAHLSLAPSFGSLVALKEL
DMHGIFFRSLDETTLRPLARLPMLQTLRLQMNFINQAQLGIFRAFPGLRYVDLSDNRISGASELTATMGEADGGEKV
WLQPGDLAPAPVDTPSSEDFRPNCSTLNFTLDLSRNNLVTVQPEMFAQLSHLQCLRLSHNCISQAVNGSQFLPLTGL
QVLELSHNKLDLYHEHSFTELPRLEALDLNYNSQPFGMQGVGHNFSFVAHLRTLRHLSLAHNNIHSQVSQQLCSTSL
RALDFSGNALCHMWAECDLYLHFFQGLSGLIWLDLSQNRLHTLLPQTLRNLPKSLQVLRLRDNYLAFFKWWSLHFLP
KLEVLDLACNQLKALTNGSLPAGTRLRRLDVSCNSISFVAPGFFSKAKELRELNLSANALKTVDHSWFGPLASALQI
LDVSANPLHCACGAAFMDFLLE

MOL1k

A disclosed Toll receptor 9-like nucleic acid of 2145 nucleotides, MOL1k, is shown in Table 1W. The disclosed MOL1k open reading frame (“ORF”) begins at the GGA initiation codon at nucleotides 1-3, shown in bold in Table 1W. The encoded polypeptide is alternatively referred to herein as MOL1k or as 248210474. The disclosed MOL1k ORF terminates at a GAG codon at nucleotides 2143-2145. As shown in Table 1W the start and stop codons are in bold letters. Because MOL1k does not start or stop at traditional initiation and termination codons, MOL1k could be a partial open reading frame which extends further in the 5′ and/or 3′ directions.

TABLE 1W

MOL1k nucleotide sequence.

(SEQ ID NO:134)

GGATCCAATGTCACCACCCTTTCCTTCTCCTCCAACCGCATCCACCACCTCCATGATTCTGACTTTGCCCACCTGCC
CAGCCTGCGGCATCTCAACCTCAAGTCGAACTGCCCGCCGGTTGGCCTCAGCCCCATCCACTTCCCCTGCCACATGA
CCATCGAGCCCAGCACCTTCTTGGCTCTGCCCACCCTGGAAGAGCTAAACCTGAGCTACAACAACATCATGACTGTG
CCTGCGCTGCCCAAATCCCTCATATCCCTGTCCCTCAGCCATACCAACATCCTGATCCTAGACTCTGCCAGCCTCGC
CGGCCTGCATCCCCTGCGCTTCCTATTCATGGACGGCAACTGTTATTACAAGAACCCCTGCAGGCAGGCACTGCAGG
TGGCCCCGGGTGCCCTCCTTGGCCTGGGCAACCTCACCCACCTGTCACTCAAGTACAACAACCTCACTGTGCTGCCC
CGCAACCTGCCTTCCAGCCTGGAGTATCTGCTGTTGTCCTACAACCCCATCGTCAAACTGGCGCCTGAGGACCTGGC
CAATCTGACCGCCCTGCGTGTCCTCGATCTGGGCGGAAATTGCCGCCCCTGCGACCACGCTCCCTACCCCTGCATGG
AGTGCCCTCGTCACTTCCCCCAGCTACATCCCGATACCTTCACCCACCTGAGCCCTCTTGAAGCCCTAGTGTTGAAG
GACAGTTCTCTCTCCTGGCTGAATGCCAGTTGGTTCCGTGCGCTGGGAAACCTCCGAGTGCTGGACCTGAGTGAGAA
CTTCCTCTACAAATGCATCACTAAAACCAAGGCCTTCCAGGGCCTAACACAGCTGCGCAAGCTTAACCTGTCCTTCA
ATTACCAAAAGAGGGTGTCCTTTGCCCACCTGTCTCTGGCCCCTTCCTTCGGGAGCCTGGTCGCCCTGAAGGAGCTG
GACATGCACGGCATCTTCTTCCCCTCACTCGATGACACCACGCTCCGGCCACTGCCCCGCCTGCCCATGCTCCAGAC
TCTGCGTCTCCAGATGAACTTCATCAACCAGCCCCAGCTCGGCATCTTCAGCGCCTTCCCTGGCCTGCGCTACGTGG
ACCTGTCGGACAACCGCATCAGCGGACCTTCGGAGCTGACAGCCACCATCGGGGAGGCAGATGCAGCGGAGAACGTC
TCGCTGCAGCCTGGGGACCTTGCTCCCGCCCCAGTGGACACTCCCAGCTCTGAAGACTTCAGGCCCAACTGCAGCAC
CCTCAACTTCACCTTGGATCTGTCACGGAACAACCTGGTGACCGTGCAGCCGGAGATGTTTGCCCAGCTCTCGCACC
TGCAGTGCCTCCGCCTGAGCCACAACTGCATCTCGCAGGCAGTCAATGGCTCCCAGTTCCTCCCCCTGACCGGTCTG
CAGGTGCTAGACCTGTCCCACAATAAGCTGGACCTCTACCACCAGCACTCATTCACGGAGCTACCACGACTGGAGGC
CCTCGACCTCAGCTACAACAGCCAGCCCTTTGGCATGCAGCGCGTGGGCCACAACTTCAGCTTCGTGGCTCACCTGC
GCACCCTGCGCCACCTCAGCCTGGCCCACAACAACATCCACACCCAAGTGTCCCAGCAGCTCTGCAGTACCTCGCTG
CGGGCCCTGGACTTCAGCGGCAATGCACTGGGCCATATGTGGCCCGAGCGACACCTCTATCTGCACTTCTTCCAAGG
CCTGAGCCCTTTGATCTCGCTGCACTTGTCCCAGAACCGCCTGCACACCCTCCTGCCCCAAACCCTGCGCAACCTCC
CCAAGAGCCTACACGTGCTCCGTCTCCGTAACAATTACCTGGCCTTCTTTAACTGGTGGAGCCTCCACTTCCTGCCC
AAACTGGAAGTCCTCGACCTGGCAGGAAACCAGCTGAAGGCCCTGACCAATGGCAGCCTGCCTGCTGGCACCCGGCT
CCGGAGGCTGCATGTCAGCTGCAACACCATCACCTTCGTGGCCCCCGGCTTCTTTTCCAAGCCCAAGGAGCTGCGAC
AGCTCAACCTTAGCGCCAACGCCCTCAAGACAGTGGACCACTCCTGGTTTGGGCCCCTCGCGAGTCCCCTGCAAATA
CTAGATGTAAGCGCCAACCCTCTCCACTGCGCCTGTCGGGCGGCCTTTATGGACTTCCTGCTCGAG

A disclosed encoded MOL1k protein has 715 amino acid residues, referred to as the MOL1k protein. The disclosed MOL1k polypeptide sequence is presented in Table 1X using the one-letter amino acid code.

TABLE 1X

Encoded MOL1k protein sequence.

(SEQ ID NO:135)

GSNVTSLSLSSNRIHHLHDSDFAHLPSLRHLNLKWNCPPVGLSPMHFPCHMTIEPSTFLAVPTLEELNLSYNNIMTV
PALPKSLISLSLSHTNILMLDSASLACLHALRFLFMDGNCYYKNPCRQALEVAPGALLGLGNLTHLSLKYNNLTVVP
RNLPSSLEYLLLSYNRIVKLAPEDLANLTALRVLDVGCNCRRCDHAPNPCMECPRHFPQLHPDTFSHLSRLECLVLK
DSSLSWLNASWFRGLGNLRVLDLSENFLYKCITKTKAFQCLTQLRKLNLSFNYQKRVSFAHLSLAPSFCSLVALKEL
DMHGIFFRSLDETTLRPLARLPMLQTLRLQMNFINQAQLCTFPAFPGLRYVDLSDNRISGASELTATMCEADGGEKV
WLQPGDLAPAPVDTPSSEDFRPNCSTLNFTLDLSRNNLVTVQPEMFAQLSHLQCLRLSHNCISQAVNGSQFLPLTGL
QVLDLSHNKLDLYHEHSFTELPRLEALDLSYNSQPFGMQGVGHNFSFVAHLRTLRHLSLAHNNTHSQVSQQLCSTSL
RALDFSGNALCHMWAEGDLYLHFFQGLSGLIWLDLSQNRLHTLLPQTLRNLPKSLQVLRLRNNYLAFFKWWSLHFLP
KLEVLDLAGNQLKALTNGSLPAGTRLRRLDVSCNSISFVAPCFFSKAKELRELNLSANALKTVDHSWPGFLASALQI
LDVSANPLHCACGAAFMDFLLE

MOL1l

A disclosed Toll receptor 9-like nucleic acid of 2145 nucleotides, MOL1l, is shown in Table 1Y. The disclosed MOL1l open reading frame (“ORF”) begins at the GGA initiation codon at nucleotides 1-3, shown in bold in Table 1Y. The encoded polypeptide is alternatively referred to herein as MOL1l or as 246484229. The disclosed MOL1l ORF terminates at a GAG codon at nucleotides 2143-2145. As shown in Table 1Y the start and stop codons are in bold letters. Because MOL1l does not start or stop at traditional initiation and termination codons, MOL1l could be a partial open reading frame which extends further in the 5′ and/or 3′ directions.

TABLE 1Y

MOL1l nucleotide sequence.

(SEQ ID NO:136)

GGATCCAATCTCACCAGCCTTTCCTTCTCCTCCAACCGCATCCACCACCTCCATGATTCTGACTTTGCCCACCTGCC
CAGCCTGCGGCATCTCAACCTCAAGTGGAACTGCCCCCCGGTTGGCCTCACCCCCATGCACTTCCCCTGCCACATGA
CCATCGAGCCCAGCACCTTCTTGCCTGTGCCCACCCTCGAAGAGCTAAACCTGAGCTACAACAACATCATGACTGTG
CCTGCGCTGCCCAAATCCCTCATATCCCTGTCCCTCAGCCATACCAACATCCTGATGCTACACTCTGCCAGCCTCGC
CGGCCTGCATGCCCTGCGCTTCCTATTCATCGACGGCAACTGTTATTACAAGAACCCCTGCACGCAGGCACTGGAGG
TGGCCCCGGGTGCCCTCCTTGGCCTGGGCAACCTCACCCACCTGTCACTCAACTACAACAACCTCACTGTGGTGCCC
CGCAACCTCCCTTCCACCCTGGAGTATCTGCTGTTGTCCTACAACCGCATCCTCAAACTGGCGCCTGAGGACCTGGC
CAATCTCACCGCCCTGCGTGTCCTCGATGTGGGCGGAAATTGCCGCCGCTGCGACCACCCTCCCAACCCCTGCATGG
AGTGCCCTCGTCACTTCCCCCAGCTACATCCCGATACCTTCAGCCACCTGACCCGTCTTGAAGGCCTCGTGTTGAAG
GACAGTTCTCTCTCCTGGCTGAATGCCAGTTGGTTCCGTGGGCTCGGAAACCTCCGAGTGCTGGACCTGAGTCAGAA
CTTCCTCTACAAATGCATCACTAAAACCAAGGCCTTCCAGCGCCTAACACAGCTGCGCAAGCTTAACCTGTCCTTCA
ATTACCAAAAGAGGGTGTCCTTTGCCCACCTGTCTCTGGCCCCTTCCTTCGCGAGCCTGGTCGCCCTGAACGAGCTG
GACATGCACGGCATCTTCTTCCCCTCACTCGATGAGACCACGCTCCGGCCACTCGCCCGCCTGCCCATGCTCCAGAC
TCTGCCTCTGCAGATGAACTTCATCAACCAGGCCCAGCTCCGCATCTTCAGGGCCTTCCCTGGCCTGCGCTACCTGG
ACCTGTCGGACAACCGCATCACCGGAGCTTCGGAGCTGACAGCCACCATGGGGGAGGCAGATGGAGCGCACAAGGTC
TGGCTGCAGCCTGGGGACCTTCCTCCGCCCCCAGTGGACACTCCCAGCTCTGAAGACTTCAGGCCCAACTGCAGCAC
CCTCAACTTCACCTTGGATCTGTCACGGAACAACCTGGTCACCGTGCAGCCGGAGATCTTTGCCCAGCTCTCGCACC
TGCAGTGCCTGCGCCTGAGCCACAACTGCATCTCCCAGGCAGTCAATGGCTCCCACTTCCTGCCGCTGACCGGTCTG
CAGGTGCTAGACCTGTCCCACAATAACCTGGACCTCTACCACGAGCACTCATTCACCGAGCTACCACGACTGGAGGC
CCTGGACCTCACCTACAACAGCCAGCCCTTTCGCATGCAGGGCGTGGGCCACAACTTCAGCTTCGTCGCTCACCTGC
GCACCCTGCGCCACCTCAGCCTGCCCCACAACAACATCCACAGCCAAGTGTCCCAGCAGCTCTGCAGTACGTCCCTG
CGGGCCCTGGACTTCAGCGGCAATGCACTGGGCCATATGTGGGCCGAGGGAGACCTCTATCTGCACTTCTTCCAAGG
CCTGAGCGGTTTGATCTGGCTGGACTTGTCCCAGAACCGCCTGCACACCCTCCTGCCCCAAACCCTCCCCAACCTCC
CCAAGAGCCTACAGGTGCTCCGTCTCCGTGACAACTACCTGGCCTTCTTTAAGTGGTCGAGCCTCCACTTCCTGCCC
AAACTCCAAGTCCTCCACCTGGCAGGAAACCAGCTGAAGGCCCTGACCAATGCCAGCCTGCCTGCTGGCACCCGGCT
CCGGAGCCTGGATGTCAGCTGCAACACCATCAGCTTCGTGCCCCCCCCCTTCTTTTCCAAGGCCAAGGAGCTGCGAG
AGCTCAACCTTAGCGCCAACGCCCTCAAGACAGTGGACCACTCCTGCTTTGGGCCCCTGGCGAGTGCCCTGCAAATA
CTAGATGTAAGCGCCAACCCTCTGCACTGCGCCTGTGGGGCGGCCTTTATGGACTTCCTGCTCGAG

A disclosed encoded MOL1l protein has 715 amino acid residues, referred to as the MOL1l protein. The disclosed MOL1l polypeptide sequence is presented in Table 1Z using the one-letter amino acid code.

MOL1m

A disclosed Toll receptor 9-like nucleic acid of 3033 nucleotides, MOL1m, is shown in Table 1AAA. The disclosed MOL1m open reading frame (“ORF”) begins at the GGA initiation codon at nucleotides 1-3, shown in bold in Table 1AAA. The encoded polypeptide is alternatively referred to herein as MOL1m or as 258065840. The disclosed MOL1m ORF terminates at a GAG codon at nucleotides 3031-3033. As shown in Table 1AAA the start and stop codons are in bold letters. Because MOL1m does not start or stop at traditional initiation and termination codons, MOL1m could be a partial open reading frame which extends further in the 5′ and/or 3′ directions.

TABLE 1AAA

MOL1m nucleotide sequence.

(SEQ ID NO:158)

GGATCCCTGGGTACCTTGCCTGCCTTCCTACCCTGTGAGCTCCAGCCCCACGGCCTGGTGAACTGCAACTGCCTGTT
CCTGAAGTCTGTGCCCCACTTCTCCATGGCAGCACCCCGTGGCAATGTCACCAGCCTTTCCTTCTCCTCCAACCGCA
TCCACCACCTCCATCATTCTGACTTTGCCCACCTCCCCAGCCTGCGGCATCTCAACCTCAAGTGGAACTGCCCGCCG
GTTGGCCTCAGCCCCATGCACTTCCCCTGCCACATGACCATCGAGCCCAGCACCTTCTTGGCTGTGCCCACCCTGGA
AGAGCTAAACCTGAGCTACAACAACATCATCACTCTGCCTGCGCTGCCCAAATCCCTCATATCCCTGTCCCTCAGCC
ATACCAACATCCTGATGCTAGACTCTGCCAGCCTCGCCGGCCTGCATCCCCTGCGCTTCCTATTCATGGACGGCAAC
TGTTATTACAAGAACCCCTGCAGCCAGGCACTCGAGGTGGCCCCGGGTGCCCTCCTTGGCCTGGCCAACCTCACCCA
CCTGTCACTCAAGTACAACAACCTCACTGTGGTGCCCCGCAACCTGCCTTCCAGCCTGGAGTATCTGCTGTTGTCCT
ACAACCGCATCGTCAAACTGGCGCCTGACGACCTGGCCAATCTGACCCCCCTGCGTGTGCTCGATGTGGGCGCAAAT
TGCCGCCCCTGCGACCACGCTCCCAACCCCTCCATGGAGTGCCCTCGTCACTTCCCCCAGCTACATCCCGATACCTT
CAGCCACCTGAGCCGTCTTGAAGGCCTGGTGTTGAAGGACAGTTCTCTCTCCTGGCTGAATGCCAGTTCGTTCCGTC
GGCTGGGAACCTCCCACTGCTGGCACCTGACTGAGAACTTCCTCTACAAATGCATCACTAAAACCAAGGCCTTCCAG
GGCCTAACACAGCTGCGCAAGCTTAACCTGTCCTTCAATTACCAAAAGACGGTGTCCTTTGCCCACCTGTCTCTGGC
CCCTTCCTTCGGGAGCCTGGTCGCCCTGAAGGAGCTGGACATGCACGGCATCTTCTTCCCCTCACTCGATGAGACCA
CCCTCCGGCCACTGGCCCGCCTCCCCATCCTCCAGACTCTCCGTCTGCAGATGAACTTCATCAACCAGGCCCAGCTC
GGCATCTTCAGGGCCTTCCCTGCCCTGCGCTACGTGGACCTCTCCGACAACCGCATCAGCGGAGCTTCCGACCTGAC
AGCCACCATGGGCGAGGCAGATCGAGGGGAGAAGGTCTGGCTGCAGCCTGGGCACCTTGCTCCGGCCCCAGTGGACA
CTCCCAGCTCTGAAGACTTCAGGCCCAACTGCAGCACCCTCAACTTCACCTTCGATCTGTCACGCAACAACCTGGTG
ACCGTGCAGCCCCACATCTTTGCCCACCTCTCGCACCTGCAGTGCCTGCGCCTGAGCCACAACTGCATCTCCCAGGC
AGTCAATGGCTCCCAGTTCCTGCCCCTGACCGGTCTGCAGGTGCTAGACCTGTCCCACAATAAGCTGGACCTCTACC
ACGAGCACTCATTCACGGAGCTACCGCGACTGGAGGCCCTGGACCTCACCTACAACAGCCAGCCCTTTGGCATGCAG
GGCGTGGGCCACAACTTCAGCTTCGTGGCTCACCTCCCCACCCTGCGCCACCTCAGCCTCGCCCACAACAACATCCA
CAGCCAAGTGTCCCAGCAGCTCTGCAGTACGTCGCTCCGGGCCCTGGACTTCACCGGCAATGCACTCGGCCATATGT
GGGCCGACGGAGACCTCTATCTGCACTTCTTCCAAGGCCTGAGCGGTTTGATCTGGCTCGACTTGTCCCAGAACCGC
CTGCACACCCTCCTGCCCCAAACCCTGCGCAACCTCCCCAAGACCCTACAGGTGCTGCCTCTCCGTCACAATTACCT
GGCCTTCTTTAAGTGGTGGAGCCTCCACTTCCTGCCCAAACTGGAAGTCCTCCACCTGGCAGCAAACCAGCTGAAGG
CCCTGACCAATGGCAGCCTGCCTGCTGGCACCCGGCTCCGGAGGCTGGATGTCAGCTGCAACAGCATCAGCTTCGTG
GCCCCCGGCTTCTTTTCCAAGGCCAACGAGCTGCGACAGCTCAACCTTAGCGCCAACGCCCTCAAGACAGTGGACCA
CTCCTGGTTTGGCCCCCTCGCGAGTGCCCTGCAAATACTAGATGTAAGCCCCAACCCTCTGCACTGCGCCTGTGGGG
CGGCCTTTATGGACTTCCTGCTGGAGGTGCACGCTGCCGTGCCCGGTCTGCCCAGCCGGGTGAACTGTGGCAGTCCG
CGCCAGCTCCAGCGCCTCAGCATCTTTGCACAGGACCTCCGCCTCTGCCTCGATGAGGCCCTCTCCTGGGACTCTTT
CGCCCTCTCGCTGCTGGCTGTGGCTCTGGGCCTGGGTGTGCCCATGCTGCATCACCTCTGTCCCTCCGACCTCTGGT
ACTGCTTCCACCTGTGCCTGGCCTCGCTTCCCTGGCGGGGGCGGCAAAGTGGGCGACATGAGGATCCCCTGCCCTAC
GATGCCTTCGTGGTCTTCCACAAAACGCAGAGCGCAGTGGCAGACTGGGTCTACAACGAGCTTCGGGGGCAGCTGGA
GGAGTCCCGTGGGCGCTGCGCACTCCGCCTGTGCCTGGAGGAACGCCACTGGCTGCCTGGCAAAACCCTCTTTGAGA
ACCTGTGGGCCTCGCTCTATGGCAGCCGCAAGACGCTGTTTGTCCTGGCCCACACGGACCCCGTCAGTGGTCTCTTG
CGCGCCAGCTTCCTGCTGCCCCAGCAGCGCCTGCTGGAGGACCGCAAGGACGTCGTGGTGCTGCTGATCCTGAGCCC
TGACGCCCGCCGCTCCCGCTACGTCCGGCTGCGCCAGCGCCTCTGCCGCCAGAGTCTCCTCCTCTCGCCCCACCAGC
CCAGTGGTCAGCGCAGCTTCTGGGCCCAGCTCGGCATGCCCCTGACCAGGGACAACCACCACTTCTATAACCGGAAC
TTCTGCCAGGGACCCACGGCCGAACTCGAG

The reverse complement of MOL1m is shown in Table 1AAB.

TABLE 1AAB

MOL1m reverse complement nucleotide sequence.

(SEQ ID NO:178)

CTCGAGTTCGGCCGTGCGTCCCTGGCAGAAGTTCCGGTTATAGAAGTGGTGGTTGTCCCTGGTCAGCGCCATGCCCA
GCTGGGCCCAGAAGCTGCGCTCACCACTGCGCTGGTGGGGCCAGAGCAGGACACTCTGGCGGCAGAGGCGCTGGCCC
AGCCGCACGTAGCGGGAGCGGCGGCCGTCAGGGCTCAGGATCACCAGCACCACGACGTCCTTGCCGTCCTCCAGCAG
GCGCTGCTGGGCCAGCACGAAGCTGGCGCGCAAGAGACCACTGACCCGGTCCGTCTGGGCCAGCACAAACAGCGTCT
TGCCGCTGCCATAGACCGACGCCCACAGGTTCTCAAAGAGCGTTTTGCCAGGCAGCCAGTCGCGTTCCTCCAGGCAC
AGGCGGAGTGCCCAGCGCCCACGGCACTCCTCCAGCTGCCCCCGAAGCTCGTTGTACACCCAGTCTGCCACTGCCCT
CTGCGTTTTGTCGAAGACCACGAAGGCATCGTAGGGCAGGGCATCCTCATCTCCCCCACTTTGCCGCCCCCGCCAGG
GAAGCCAGGCCACGCACAGGTGGAAGCAGTACCAGAGGTCCCAGCCACAGAGGTGATGCAGCATGGGCACACCCAGG
CCCAGAGCCACAGCCAGCAGCGAGAGGCCGAAACAGTCCCACGAGAGCCCCTCATCCACGCAGAGGCGCAGGTCCTG
TGCAAAGATGCTCAGGCCCTGGAGCTGGCCCGGACTGCCACACTTCACCCGGCTCGGCAGACCGGGCACGCCAGCCT
GCACCTCCAGCAGGAAGTCCATAAAGGCCGCCCCACAGGCGCAGTGCAGAGGGTTGGCGCTTACATCTAGTATTTGC
AGCGCACTCGCCACCGGCCCAAACCAGGACTGGTCCACTGTCTTGAGGGCCTTGGCGCTAACCTTGAGCTCTCGCAG
CTCCTTGGCCTTGGAAAAGAAGCCGGGGGCCACGAAGCTGATGCTGTTGCACCTGACATCCAGCCTCCGGAGCCGGG
TGCCAGCAGGCACGCTGCCATTGGTCAGGGCCTTCAGCTGGTTTCCTGCCAGGTCGAGGACTTCCAGTTTGGGCAGG
AAGTGGAQGCTCCACCACTTAAAGAAGGCCAGGTAATTCTCACGCAGACGCAGCACCTGTAGGCTCTTGGGGAGGTT
GCGCAGGGTTTGGGGCAGGAGGCTGTGCAGGCGGTTCTGCGACAACTCCAGCCAGATCAAACCCCTCACCCCTTGGA
AGAAGTGCAGATAGAGGTCTCCCTCGGCCCACATATGGCCCAGTGCATTCCCGCTGAACTCCAGCGCCCCCAGCGAC
GTACTGCAGACCTGCTGGCACACTTGCCTCTGGATGTTCTTGTGGGCCAGGCTGAGCTGGCGCAGGGTCCGCAGGTG
AGCCACGAACCTGAAGTTGTGGCCCACGCCCTGCATGCCAAAGGGCTCCCTGTTGTAGCTGAGGTCCAGGGCCTCCA
GTCGCCGTAGCTCCGTGAATGACTGCTCGTGGTAGAGGTCCAGCTTATTCTGGGACAGGTCTAGCACCTGCAGACCG
GTCAGCGGCAGGAACTGGGAGCCATTGACTGCCTGCGAGATGCAGTTGTCGCTCAGGCGCAGGCACTGCAGGTGCGA
GAGCTGGGCAAACATCTCCCGCTGCACGGTCACCAGGTTGTTCCGTGACAGATCCAAGGTGAAGTTCAGCGTGCTGC
AGTTGGCCCTGAAGTCTTCAGAGCTGGGAGTGTCCACTGCGGCCGCAGCAAGGTCCCCACGCTGCAGCCAGACCTTC
TCCCCTCCATCTGCCTCCCCCATGGTGGCTGTCAGCTCCCAAGCTCCGCTGATGCGGTTCTCCCACACGTCCACGTA
GCGCAGGCCAGGGAAGGCCCTGAAGATGCCGAGCTGGGCCTGGTTGATGAAGTTCATCTCCAGACGCAGAGTCTGCA
GCATCCGCAGGCGGGCCACTGGCCGGAGCGTGGTCTCATCGAGTGAGCGGAAGAAGATCCCGTGCATGTCCAGCTCC
TTCAGGGCGACCAGGCTCCCGAAGGAAGGGGCCAGAGACACGTGGGCAAAGGACACCCTCTTTTGGTAATTGAAGGA
CAGGTTAAGCTTGCGCAGCTGTGTTAGGCCCTGGAAGGCCTTGGTTTTAGTGATGCATTTGTAGAGCAAGTTCTCAC
TCAGCTCCAGCACTCGGAGGTTTCCCAGCCCACGGAACCAACTGGCATTCAGCCAGGAGAGACAACTGTCCTTCAAC
ACCAGGCCTTCAAGACGGCTCACCTGGCTGAACCTATCGGGATGTAGCTGGGGGAAGTGACGACCCCACTCCATGCA
GGGGTTGGGAGCGTGGTCGCAGCGGCGGCAATTTCCGCCCACATCGAGCACACGCAGGGCGGTCAGATTCGCCAGGT
CCTCAGGCCCCACTTTCACGATCCGGTTGTAGGACAACAGCAGATACTCCAGGCTCGAAGGCAGGTTGCGGGGCACC
ACACTGAGGTTGTTGTACTTGAGTGACAGGTGGGTGAGCTTGCCCAGGCCAAGGACGGCACCCCGGGCCACCTCCAG
TGCCTGCCTCCAGGGGTTCTTGTAATAACAGTTGCCGTCCATCAATAGGAAGCGCAGGCCATGCAGGCCGCCGAGCC
TGGCAGAGTCTAGCATCACGATGTTGGTATGGCTGAGGGACACGCATATGAGGGATTTCGGCACCGCAGGCACAGTC
ATGATGTTGTTGTAGCTCAGGTTTAGCTCTTCCAGGGTGGGCACAGCCAAGAAGGTGCTGGGCTCGATGGTCATGTG
GCAGGGCAAGTGCATGGGCCTGAGGCCAACCGGCGGGCAGTTCCACTTGAGCTTGAGATGCCGCAGCCTGGGCAGGT
GGGCAAAGTCAGAATCATGGAGGTGGTGCATGCGGTTGGACGACAAGCAAAGGCTGGTGACATTGCCACCCGGTGCT
GCCATGGAGAAGTGGGGCACACACTTCAGGAACAGCCAGTTGCAGTTCACCAGGCCGTGGGGCTCGAGCTCACAGGG
TAGGAAGGCAGGCAACGTACCCAGGGATCC

A disclosed encoded MOL1m protein has 1011 amino acid residues, referred to as the MOL1m protein. The disclosed MOL1m polypeptide sequence is presented in Table 1AAC using the one-letter amino acid code.

TABLE 1AAC

Encoded MOL1m protein sequence.

(SEQ ID NO:159)

GSLGTLPAFLPCELQPHGLVNCNWLFLKSVPHFSMAAPRGNVTSLSLSSNRIHHLHDSDFAHLPSLRHLNLKWNCPP
VGLSPMHFPCHMTTEPSTFLAVFTLEELNLSYNNIMTVPALPKSLISLSLSHTNILMLDSASLAGLHALRFLFMDGN
CYYKNPCRQALEVAPGALLGLGNLTHLSLKYNNLTVVPRNLPSSLEYLLLSYNRIVKLAPEDLANLTALRVLDVGGN
CRRCDHAPNPCMECPRHFPQLHPDTFSHLSRLEGLVLKDSSLSWLNASWFRGLCNLRVLDLSENFLYKCTTKTKAFQ
GLTQLRKLNLSFNYQKRVSFAHLSLAPSFGSLVALKELDMHGIFFRSLDETTLRPLARLPMLQTLRLQMNPINQAQL
GIFRAFPGLRYVDLSDNRISGASELTATMGEADCCEKVWLQPGDLAPAPVDTPSSEDFRPNCSTLNFTLDLSRNNLV
TVQPEMFAQLSHLQCLRLSHNCISQAVNGSQFLPLTGLQVLDLSHNKLDLYHEHSFTELPRLEALDLSYNSQPFGMQ
GVGHNFSFVAHLRTLRHLSLAHNNTHSQVSQQLCSTSLRALDFSCNALCHMWAEGDLYLHFFQGLSGLTWLDLSQNR
LHTLLPQTLRNLPKSLQVLRLRDNYLAFFKWWSLHFLPKLEVLDLAGNQLKALTNGSLPAGTRLRRLDVSCNSISFV
APGFFSKAKELRELNLSANALKTVDHSWFGPLASALQILDVSANPLHCACGAAFMDFLLEVQAAVPCLPSRVKCGSP
GQLQGLSIFAQDLRLCLDEALSWDCFALSLLAVALGLGVPMLHHLCGWDLWYCFHLCLAWLPWRGRQSGRDEDALPY
DAFVVFDKTQSAVADWVYNELRGQLEECRGRWALRLCLEERDWLPGKTLFENLWASVYGSRKTLFVLAHTDRVSGLL
RASFLLAQQRLLEDRKDVVVLVILSPDGRRSRYVRLRQRLCRQSVLLWPHQPSGQRSFWAQLGMALTRDNHHFYNRN
FCQGPTAELE

MOL1n

A disclosed Toll receptor 9-like nucleic acid of 2389 nucleotides, MOL1n, is shown in Table 1AAD. The disclosed MOL1n open reading frame (“ORF”) begins at the AAC initiation codon at nucleotides 2-4, shown in bold in Table 1AAD. The encoded polypeptide is alternatively referred to herein as MOL1n or as 263483006. The disclosed MOL1n ORF terminates at a GGC codon at nucleotides 2387-2389. As shown in Table 1AAD the start and stop codons are in bold letters. Because MOL1n does not start or stop at traditional initiation and termination codons, MOL1n could be a partial open reading frame which extends further in the 5′ and/or 3′ directions.

TABLE 1AAD

MOL1n nucleotide sequence.

(SEQ ID NO:160)

CACCGGATCCCTGCGTACCTTGCCTGCCTTCCTACCCTGTCAGCTCCAGCCCCACGCCCTCCTGAACTGCAACTCG
CTCTTCCTGAAGTCTGTGCCCCACTTCTCCATGGCACCACCCCGTCCCAATGTCACCAGCCTTTCCTTGTCCTCCA
ACCGCATCCACCACCTCCATGATTCTGACTTTGCCCACCTGCCCAGCCTGCGGCATCTCAACCTCAAGTGGAACTG
CCCGCCGGTTGGCCTCAGCCCCATGCACTTCCCCTGCCACATGACCATCGAGCCCAGCACCTTCTTGGCTATGCCC
ACCCTGGAAGAGCTAAACCTGAGCTACAACAACATCATGACTGTGCCTGCGCTGCCCAAATCCCTCATATCCCTGT
CCCTCAGCCATACCAACATCCTGATGCTAGACTCTGCCAGCCTCGCCGGCCTGCATGCCCTGCGCTTCCTATTCAT
CGACGGCAACTGTTATTACAAGAACCCCTGCAGGCAGCCACTCGAGGTGGCCCCCGGTGCCCTCCTTGGCCTGGGC
AACCTCACCCACCTGTCACTCAAGTACAACAACCTCACTGTCGTGCCCCGCAACCTGCCTTCCAGCCTGGAGTATC
TGCTGTTCTCCTACAACCCCATCGTCAAACTGGCGCCTGAGGACCTCGCCAATCTGACCGCCCTGCGTGTCCTCGA
TGTGCGCGGAAATTGCCGCCGCTGCGACCACGCTCCCAACCCCTCCATCCAGTCCCCTCCTCACTTCCCCCACCTA
CATCCCGATACCTTCAGCCACCTGACCCGTCTTGAAGCCCTGGTCTTGAAGGACAGTTCTCTCTCCTGGCTGAATG
CCAGTTGCTTCCGTGGGCTGGGAAACCTCCCAGTGCTGGACCTGAGTGAGAACTTCCTCTACAAATGCATCACTAA
AACCAAGCCCTTCCAGGGCCTAACACAGCTCCGCAAGCTTAACCTGTCCTTCAATTACCAAAAGAGGGTGTCCTTT
GCCCACCTGTCTCTGGCCCCTTCCTTCGGGAGCCTGGTCGCCCTGAAGCACCTGGACATGCACGGCATCTTCTTCC
GCTCACTCGATGAGACCACGCTCCCGCCACTCGCCCGCCTGCCCATGCTCCAGACTCTGCGTCTGCAGATGAACTT
CATCAACCAGGCCCAGCTCGCCATCTTCAGGGCCTTCCCTGGCCTGCGCTACGTGGACCTGTCGGACAACCGCATC
AGCGGAGCTTCGGAGCTGACAGCCACCATGGGGGAGGCAGATCGAGGGGAGAAGGTCTGGCTCCAGCCTGGGCACC
TTGCTCCGGCCCCAGTGGACACTCCCAGCTCTGAAGACTTCAGGCCCAACTCCAGCACCCTCAACTTCACCTTGGA
TCTGTCACGGAACAACCTGGTGACCGTGCAGCCGGAGATGTTTCCCCAGCTCTCGCACCTGCAGTGCCTGCGCCTG
AGCCACAACTGCATCTCGCAGGCAGTCAATGGCTCCCAGTTCCTGCCCCTGACCGGTCTGCAGGTGCTAGACCTGT
CCCACAATAAGCTGGACCTCTACCACGAGCACTCATTCACGGAGCTACCACCACTGGAGGCCCTGCACCTCAGCTA
CAACACCCACCCCTTTGCCATGCAGGGCGTGGCCCACAACTTCAGCTTCGTGGCTCACCTGCGCACCCTCCGCCAC
CTCAGCCTGGCCCACAACAACATCCACAGCCAAGTGTCCCAGCAGCTCTGCACTACGTCGCTGCGCGCCCTGGACT
TCAGCGGCAATGCACTGGGCCATATGTGGGCCGAGGGAGACCTCTATCTGCACTTCTTCCAAGGCCTGAGCGGTTT
GATCTGGCTGGACTTGTCCCAGAACCGCCTGCACACCCTCCTGCCCCAAACCCTGCGCAACCTCCCCAAGAGCCTA
CAGGTGCTGCGTCTCCGTGACAATTACCTGGCCTTCTTTAACTGCTGGACCCTCCACTTCCTGCCCAAACTGGAAG
TCCTCGACCTGGCAGGAAACCAGCTGAACCCCCTGACCAATGGCAGCCTGCCTGCTGCCACCCGGCTCCGGAGGCT
GGATGTCAGCTGCAACAGCATCAGCTTCGTGGCCCCCGGCTTCTTTTCCAAGGCCAAGGAGCTGCGAGACCTCAAC
CTTAGCGCCAACGCCCTCAAGACAGTGGACCACTCCTGGTTTGGGCCCCTGGCCAGTGCCCTGCAAATACTAGATG
TAAGCGCCAACCCTCTGCACTGCGCCTGTGGGGCGGCCTTTATGGACTTCCTGCTCCAGGTGCAGCCTGCCGTGCC
CGCTCCGCCCAGCCGGGTGAAGTGTCGCAGTCCGGCCCAGCTCCACGGCCTCAGCATCTTTGCACAGCACCTGCGC
CTCTGCCTGCATGAGGCCCTCTCCCTCCACGGC

A disclosed encoded MOL1n protein has 796 amino acid residues, referred to as the MOL1n protein. The disclosed MOL1n polypeptide sequence is presented in Table 1AAE using the one-letter amino acid code.

TABLE 1AAE

Encoded MOL1n protein sequence.

(SEQ ID NO:161)

TGSLGTLPAFLPCELQPHGLVNCNWLFLKSVPHFSMAAPRGNVTSLSLSSNRIHHLHDSDFAHLPSLRHLNLKWNCP
PVGLSPMHFPCHMTIEPSTFLAVPTLEELNLSYNNTMTVPALPKSLISLSLSHTNILMLDSASLAGLHALRFLFMDG
NCYYKNPCRQALEVAPGALLGLGNLTHLSLKYNNLTVVPRNLPSSLEYLLLSYNRIVKLAPEDLANLTALRVLDVGG
NCRRCDHAPNPCMECPRHEPQLHPDTFSHLSRLEGLVLKDSSLSWLNASWFRGLGNLRVLDLSENFLYKCITRTKAF
QGLTQLRKLNLSFNYQKRVSFAHLSLAPSFGSLVALKELDMHGIFFRSLDETTLRPLARLPMLQTLRLQMNFINQAQ
LGIFRAFPGLRYVDLSDNRISGASELTATMGEADGGEKVWLQPGDLAPAPVDTPSSEDFRPNCSTLNFTLDLSRNNL
VTVQPEMFAQLSHLQCLRLSHNCISQAVNGSQFLPLTGLQVLDLSHNKLDLYHEHSFTELPRLEALDLSYNSQPFGM
QGVGHNFSFVAHLRTLRHLSLAHNNIHSQVSQQLCSTSLRALDFSGNALGHMWAEGDLYLHFFQGLSGLIWLDLSQN
RLHTLLPQTLRNLPKSLQVLRLRDNYLAFFKWWSLHFLPKLEVLDLACNQLKALTNGSLPACTRLRRLDVSCNSISF
VAPGFFSKAKELRELNLSANALKTVDHSWFCPLASALQILDVSANPLHCACGAAFMDFLLEVQAAVPGPPSRVKCGS
PGQLQGLSIFAQDLRLCLDEALSLEG

MOL1o

A disclosed Toll receptor 9-like nucleic acid of 3049 nucleotides, MOL1o, is shown in Table 1AAF. The disclosed MOL1o open reading frame (“ORF”) begins at the ATG initiation codon at nucleotides 1-3, shown in bold in Table 1AAF. The encoded polypeptide is alternatively referred to herein as MOL1o or as CG54674-01. The disclosed MOL1o ORF terminates at a TAG codon at nucleotides 3043-3045. As shown in Table 1AAF the start and stop codons are in bold letters. Because MOL1o does not stop at a traditional termination codon, MOL1o could be a partial open reading frame which extends further in the 3′ direction.

TABLE 1AAF
MOL1o nucleotide sequence.

(SEQ ID NO:162)

ATGCTGGCCATGACCCTGGCCCTGGGTACCTTGCCTGCCTTCCTACCCTGTGAGCTCCAGCCCCACGGCCTGGTGAA
CTGCAACTGGCTGTTCCTGAAGTCTGTGCCCCACTTCTCCATGGCAGCACCCCGTGGCAATGTCACCAGCCTTTCCT
TGTCCTCCAACCGCATCCACCACCTCCATGATTCTGACTTTGCCCACCTGCCCAGCCTGCGGCATCTCAACCTCAAG
TGGAACTGCCCGCCGGTTGGCCTCAGCCCCATGCACTTCCCCTGCCACATGACCATCGAGCCCAGCACCTTCTTGGC
TGTGCCCACCCTGGAAGAGCTAAACCTGAGCTACAACAACATCATGACTGTGCCTGCGCTGCCCAAATCCCTCATAT
CCCTGTCCCTCAGCCATACCAACATCCTGATGCTAGACTCTGCCAGCCTCGCCGGCCTGCATGCCCTGCGCTTCCTA
TTCATGGACGGCAACTGTTATTACAAGAACCCCTGCAGGCAGGCACTGGAGGTGGCCCCGGGTGCCCTCCTTGGCCT
GGGCAACCTCACCCACCTGTCACTCAAGTACAACAACCTCACTGTGGTGCCCCGCAACCTGCCTTCCAGCCTGGAGT
ATCTGCTGTTGTCCTACAACCGCATCGTCAAACTGGCGCCTGAGGACCTGGCCAATCTGACCGCCCTGCGTGTGCTC
GATGTGGGCGGAAATTGCCGCCGCTGCGACCACGCTCCCAACCCCTGCATGGAGTGCCCTCGTCACTTCCCCCAGCT
ACATCCCGATACCTTCAGCCACCTGAGCCGTCTTGAAGGCCTGGTGTTGAAGGACAGTTCTCTCTCCTGGCTGAATG
CCAGTTGGTTCCGTGGGCTGGGAAACCTCCGAGTGCTGGACCTGAGTGAGAACTTCCTCTACAAATGCATCACTAAA
ACCAAGGCCTTCCAGGGCCTAACACAGCTGCGCAAGCTTAACCTGTCCTTCAATTACCAAAAGAGGGTGTCCTTTGC
CCACCTGTCTCTGGCCCCTTCCTTCGGGAGCCTGGTCGCCCTGAAGGAGCTGGACATGCACGGCATCTTCTTCCGCT
CACTCGATGAGACCACGCTCCGGCCACTGGCCCGCCTGCCCATGCTCCAGACTCTGCGTCTGCAGATGAACTTCATC
AACCAGGCCCAGCTCGGCATCTTCAGGGCCTTCCCTGGCCTGCGCTACGTGGACCTGTCGGACAACCGCATCAGCGG
AGCTTCGGAGCTGACAGCCACCATGGGGGAGGCAGATGGAGGGGAGAAGGTCTGGCTGCAGCCTGGGGACCTTGCTC
CGGCCCCAGTGGACACTCCCAGCTCTGAAGACTTCAGGCCCAACTGCAGCACCCTCAACTTCACCTTGGATCTGTCA
CGGAACAACCTGGTGACCGTGCAGCCGGAGATGTTTGCCCAGCTCTCGCACCTGCAGTGCCTGCGCCTGAGCCACAA
CTGCATCTCGCAGGCAGTCAATGGCTCCCAGTTCCTGCCGCTGACCGGTCTGCAGGTGCTAGACCTGTCCCACAATA
AGCTGGACCTCTACCACGAGCACTCATTCACGGAGCTACCACGACTGGAGGCCCTGGACCTCAGCTACAACAGCCAG
CCCTTTGGCATGCAGGGCGTGGGCCACAACTTCAGCTTCGTGGCTCACCTGCGCACCCTGCGCCACCTCAGCCTGGC
CCACAACAACATCCACAGCCAAGTGTCCCAGCAGCTCTGCAGTACGTCGCTGCGGGCCCTGGACTTCAGCGGCAATG
CACTGGGCCATATGTGGGCCGAGGGAGACCTCTATCTGCACTTCTTCCAAGGCCTGAGCGGTTTGATCTGGCTGGAC
TTGTCCCAGAACCGCCTGCACACCCTCCTGCCCCAAACCCTGCGCAACCTCCCCAAGAGCCTACAGGTGCTGCGTCT
CCGTGACAATTACCTGGCCTTCTTTAAGTGGTGGAGCCTCCACTTCCTGCCCAAACTGGAAGTCCTCGACCTGGCAG
GAAACCAGCTGAAGGCCCTGACCAATGGCAGCCTGCCTGCTGGCACCCGGCTCCGGAGGCTGGATGTCAGCTGCAAC
AGCATCAGCTTCGTGGCCCCCGGCTTCTTTTCCAAGGCCAAGGAGCTGCGAGAGCTCAACCTTAGCGCCAACGCCCT
CAAGACAGTGGACCACTCCTGGTTTGGGCCCCTGGCGAGTGCCCTGCAAATACTAGATGTAAGCGCCAACCCTCTGC
ACTGCGCCTGTGGGGCGGCCTTTATGGACTTCCTGCTGGAGGTGCAGGCTGCCGTGCCCGGTCTGCCCAGCCGGGTG
AAGTGTGGCAGTCCGGGCCAGCTCCAGGGCCTCAGCATCTTTGCACAGGACCTGCGCCTCTGCCTGGATGAGGCCCT
CTCCTGGGACTGTTTCGCCCTCTCGCTGCTGGCTGTGGCTCTGGGCCTGGGTGTGCCCATGCTGCATCACCTCTGTG
GCTGGGACCTCTGGTACTGCTTCCACCTGTGCCTGGCCTGGCTTCCCTGGCGGGGGCGGCAAAGTGGGCGAGATGAG
GATGCCCTGCCCTACGATGCCTTCGTGGTCTTCGACAAAACGCAGAGCGCAGTGGCAGACTGGGTGTACAACGAGCT
TCGGGGGCAGCTGGAGGAGTGCCGTGGGCGCTGGGCACTCCGCCTGTGCCTGGAGGAACGCGACTGGCTGCCTGGCA
AAACCCTCTTTGAGAACCTGTGGGCCTCGGTCTATGGCAGCCGCAAGACGCTGTTTGTGCTGGCCCACACGGACCGG
GTCAGTGGTCTCTTGCGCGCCAGCTTCCTGCTGGCCCAGCAGCGCCTGCTGGAGGACCGCAAGGACGTCGTGGTGCT
GGTGATCCTGAGCCCTGACGGCCGCCGCTCCCGCTATGTGCGGCTGCGCCAGCGCCTCTGCCGCCAGAGTGTCCTCC
TCTGGCCCCACCAGCCCAGTGGTCAGCGCAGCTTCTGGGCCCAGCTGGGCATGGCCCTGACCAGGGACAACCACCAC
TTCTATAACCGGAACTTCTGCCAGGGACCCACGGCCGAATAGACTG

A disclosed encoded MOL1o protein has 1014 amino acid residues, referred to as the MOL1o protein. The disclosed MOL1o polypeptide sequence is presented in Table 1AAG using the one-letter amino acid code.

TABLE 1AAG
Encoded MOL1o protein sequence.

(SEQ ID NO:163)

MLAMTLALGTLPAFLPCELQPHGLVNCNWLFLKSVPHFSMAAPRGNVTSLSLSSNRIHHLHDSDFAHLPSLRHLNLK
WNCPPVGLSPMHFPCHMTIEPSTFLAVPTLEELNLSYNNIMTVPALPKSLISLSLSHTNILMLDSASLAGLHALRFL
FMDGNCYYKNPCRQALEVAPGALLGLGNLTHLSLKYNNLTVVPRNLPSSLEYLLLSYNRIVKLAPEDLANLTALRVL
DVGGNCRRCDHAPNPCMECPRHFPQLHPDTFSHLSRLEGLVLKDSSLSWLNASWFRGLGNLRVLDLSENFLYKCITK
TKAFQGLTQLRKLNLSFNYQKRVSFAHLSLAPSFGSLVALKELDMHGIFFRSLDETTLRPLARLPMLQTLRLQMNFI
NQAQLGIFRAFPGLRYVDLSDNRISGASELTATMGEADGGEKVWLQPGDLAPAPVDTPSSEDFRPNCSTLNFTLDLS
RNNLVTVQPEMFAQLSHLQCLRLSHNCISQAVNGSQFLPLTGLQVLDLSHNKLDLYHEHSFTELPRLEALDLSYNSQ
PFGMQGVGHNFSFVAHLRTLRHLSLAHNNIHSQVSQQLCSTSLRALDFSGNALGHMWAEGDLYLHFFQGLSGLIWLD
LSQNRLHTLLPQTLRNLPKSLQVLRLRDNYLAFFKWWSLHFLPKLEVLDLAGNQLKALTNGSLPAGTRLRRLDVSCN
SISFVAPGFFSKAKELRELNLSANALKTVDHSWFGPLASALQILDVSANPLHCACGAAFMDFLLEVQAAVPGLPSRV
KCGSPGQLQGLSIFAQDLRLCLDEALSWDCFALSLLAVALGLGVPMLHHLCGWDLWYCFHLCLAWLPWRGRQSGRDE
DALPYDAFVVFDKTQSAVADWVYNELRGQLEECRGRWALRLCLEERDWLPGKTLFENLWASVYGSRKTLFVLAHTDR
VSGLLRASFLLAQQRLLEDRKDVVVLVILSPDGRRSRYVRLRQRLCRQSVLLWPHQPSGQRSFWAQLGMALTRDNHH
FYNRNFCQGPTAE

MOL1p

A disclosed Toll receptor 9-like nucleic acid of 3057 nucleotides, MOL1p, is shown in Table 1AAH. The disclosed MOL1p open reading frame (“ORF”) begins at the GGA initiation codon at nucleotides 1-3, shown in bold in Table 1AAH. The encoded polypeptide is alternatively referred to herein as MOL1p or as 263676346. The disclosed MOL1p ORF terminates at a GAG codon at nucleotides 3055-3057. As shown in Table 1AAH the start and stop codons are in bold letters. Because MOL1p does not start or stop at traditional initiation and termination codons, MOL1p could be a partial open reading frame which extends further in the 5′ and/or 3′ directions.

TABLE 1AAH
MOL1p nucleotide sequence.

(SEQ ID NO:164)

GGATCCACCATGCTGGCCATGACCCTGGCCCTGGGTACCTTGCCTGCCTTCCTACCCTGTGAGCTCCAGCCCCACGG
CCTGGTGAACTGCAACTGGCTGTTCCTGAAGTCTGTGCCCCACTTCTCCATGGCAGCACCCCGTGGCAATGTCACCA
GCCTTTCCTTGTCCTCCAACCGCATCCACCACCTCCATGATTCTGACTTTGCCCACCTGCCCAGCCTGCGGCATCTC
AACCTCAAGTGGAACTGCCCGCCGGTTGGCCTCAGCCCCATGCACTTCCCCTGCCACATGACCATCGAGCCCAGCAC
CTTCTTGGCTGTGCCCACCCTGGAAGAGCTAAACCTGAGCTACAACAACATCATGACTGTGCCTGCGCTGCCCAAAT
CCCTCATATCCCTGTCCCTCAGCCATACCAACATCCTGATGCTAGACTCTGCCAGCCTCGCCGGCCTGCATGCCCTG
CGCTTCCTATTCATGGACGGCAACTGTTATTACAAGAACCCCTGCAGGCAGGCACTGGAGGTGGCCCCGGGTGCCCT
CCTTGGCCTGGGCAACCTCACCCACCTGTCACTCAAGTACAACAACCTCACTGTGGTGCCCCGCAACCTGCCTTCCA
GCCTGGAGTATCTGCTGTTGTCCTACAACCGCATCGTCAAACTGGCGCCTGAGGACCTGGCCAATCTGACCGCCCTG
CGTGTGCTCGATGTGGGCGGAAATTGCCGCCGCTGCGACCACGCTCCCAACCCCTGCATGGAGTGCCCTCGTCACTT
CCCCCAGCTACATCCCGATACCTTCAGCCACCTGAGCCGTCTTGAAGGCCTGGTGTTGAAGGACAGTTCTCTCTCCT
GGCTGAATGCCAGTTGGTTCCGTGGGCTGGGAAACCTCCGAGTGCTGGACCTGAGTGAGAACTTCCTCTACAAATGC
ATCACTAAAACCAAGGCCTTCCAGGGCCTAACACAGCTGCGCAAGCTTAACCTGTCCTTCAATTACCAAAAGAGGGT
GTCCTTTGCCCACCTGTCTCTGGCCCCTTCCTTCGGGAGCCTGGTCGCCCTGAAGGAGCTGGACATGCACGGCATCT
TCTTCCGCTCACTCGATGAGACCACGCTCCGGCCACTGGCCCGCCTGCCCATGCTCCAGACTCTGCGTCTGCAGATG
AACTTCATCAACCAGGCCCAGCTCGGCATCTTCAGGGCCTTCCCTGGCCTGCGCTACGTGGACCTGTCGGACAACCG
CATCAGCGGAGCTTCGGAGCTGACAGCCACCATGGGGGAGGCAGATGGAGGGGAGAAGGTCTGGCTGCAGCCTGGGG
ACCTTGCTCCGGCCCCAGTGGACACTCCCAGCTCTGAAGACTTCAGGCCCAACTGCAGCACCCTCAACTTCACCTTG
GATCTGTCACGGAACAACCTGGTGACCGTGCAGCCGGAGATGTTTGCCCAGCTCTCGCACCTGCAGTGCCTGCGCCT
GAGCCACAACTGCATCTCGCAGGCAGTCAATGGCTCCCAGTTCCTGCCGCTGACCGGTCTGCAGGTGCTAGACCTGT
CCCACAATAAGCTGGACCTCTACCACGAGCACTCATTCACGGAGCTACCACGACTGGAGGCCCTGGACCTCAGCTAC
AACAGCCAGCCCTTTGGCATGCAGGGCGTGGGCCACAACTTCAGCTTCGTGGCCCACCTGCGCACCCTGCGCCACCT
CAGCCTGGCCCACAACAACATCCACAGCCAAGTGTCCCAGCAGCTCTGCAGTACGTCGCTGCGGGCCCTGGACTTCA
GCGGCAATGCACTGGGCCATATGTGGGCCGAGGGAGACCTCTATCTGCACTTCTTCCAAGGCCTGAGCGGTTTGATC
TGGCTGGACTTGTCCCAGAATCGCCTGCACACCCTCCTGCCCCAAACCCTGCGCAACCTCCCCAAGAGCCTACAGGT
GCTGCATCTCCGTGACAATTACCTGGCCTTCTTTAAGTGGTGGAGCCTCCACTTCCTGCCCAAACTGGAAGTCCTCG
ACCTGGCAGGAAACCAGCTGAAGGCCCTGACCAATGGCAGCCTGCCTGCTGGCACCCGGCTCCGGAGGCTGGATGTC
AGCTGCAACAGCATCAGCTTCGTGGCCCCCGGCTTCTTTTCCAAGGCCAAGGAGCTGCGAGAGCTCAACCTTAGCGC
CAACGCCCTCAAGACAGTGGACCACTCCTGGTTTGGGCCCCTGGCGAGTGCCCTGCAAATACTAGATGTAAGCGCCA
ACCCTCTGCACTGCGCCTGTGGGGCGGCCTTTATGGACTTCCTGCTGGAGGTGCAGGCTGCCGTGCCCGGTCTGCCC
AGCCGGGTGAAGTGTGGCAGTCCGGGCCAGCTCCAGGGCCTCAGCATCTTTGCACAGGACCTGCGCCTCTGCCTGGA
TGAGGCCCTCTCCTGGGACTGTTTCGCCCTCTCGCTGCTGGCTGTGGCTCTGGGCCTGGGTGTGCCCATGCTGCATC
ACCTCTGTGGCTGGGACCTCTGGTACTGCTTCCACCTGTGCCTGGCCTGGCTTCCCTGGCGGGGGCGGCAAAGTGGG
CGAGATGAGGATGCCCTGCCCTACGATGCCTTCGTGGTCTTCGACAAAACGCAGAGCGCAGTGGCAGACTGGGTGTA
CAACGAGCTTCGGGGGCAGCTGGAGGAGTGCCGTGGGCGCTGGGCACTCCGCCTGTGCCTGGAGGAACGCGACTGGC
TGCCTGGCAAAACCCTCTTTGAGAACCTGTGGGCCTCGGTCTATGGCAGCCGCAAGACGCTGTTTGTGCTGGCCCAC
ACGGACCGGGTCAGTGGTCTCTTGCGCGCCAGCTTCCTGCTGGCCCAGCAGCGCCTGCTGGAGGACCGCAAGGACGT
CGTGGTGCTGGTGATCCTGAGCCCTGACGGCCGCCGCTCCCGCTACGTGCGGCTGCGCCAGCGCCTCTGCCGCCAGA
GTGTCCTCCTCTGGCCCCACCAGCCCAGTGGTCAGCGCAGCTTCTGGGCCCAGCTGGGCATGGCCCTGACCAGGGAC
AACCACCACTTCTATAACCGGAACTTCTGCCAGGGACCCACGGCCGAACTCGAG

TABLE 1AAI
MOL1p reverse complement nucleotide sequence.

(SEQ ID NO:179)

CTCGAGTTCGGCCGTGGGTCCCTGGCAGAAGTTCCGGTTATAGAAGTGGTGGTTGTCCCTGGTCAGGGCCATGCCCA
GCTGGGCCCAGAAGCTGCGCTGACCACTGGGCTGGTGGGGCCAGAGGAGGACACTCTGGCGGCAGAGGCGCTGGCGC
AGCCGCACGTAGCGGGAGCGGCGGCCGTCAGGGCTCACGATCACCAGCACCACGACGTCCTTGCGGTCCTCCAGCAG
GCGCTGCTGGGCCAGCAGGAAGCTGGCGCGCAAGAGACCACTGACCCGGTCCGTGTGGGCCAGCACAAACAGCGTCT
TGCGGCTGCCATAGACCGAGGCCCACAGGTTCTCAAAGAGGGTTTTGCCAGGCAGCCAGTCGCGTTCCTCCAGGCAC
AGGCGGAGTGCCCAGCGCCCACGGCACTCCTCCAGCTGCCCCCGAAGCTCGTTGTACACCCAGTCTGCCACTGCGCT
CTGCGTTTTGTCGAAGACCACGAAGGCATCGTAGGGCAGGGCATCCTCATCTCGCCCACTTTGCCGCCCCCGCCAGG
GAAGCCAGGCCAGGCACAGGTGGAAGCAGTACCAGAGGTCCCAGCCACAGAGGTGATGCAGCATGGGCACACCCAGG
CCCAGAGCCACAGCCAGCAGCGAGAGGGCGAAACAGTCCCAGGAGAGGGCCTCATCCAGGCAGAGGCGCAGGTCCTG
TGCAAAGATGCTGAGGCCCTGGAGCTGGCCCGGACTGCCACACTTCACCCGGCTGGGCAGACCGGGCACGGCAGCCT
GCACCTCCAGCAGGAAGTCCATAAAGGCCGCCCCACAGGCGCAGTGCAGAGGGTTGGCGCTTACATCTAGTATTTGC
AGGGCACTCGCCAGGGGCCCAAACCAGGAGTGGTCCACTGTCTTGAGGGCGTTGGCGCTAAGGTTGAGCTCTCGCAG
CTCCTTGGCCTTGGAAAAGAAGCCGGGGGCCACGAAGCTGATGCTGTTGCAGCTGACATCCAGCCTCCGGAGCCGGG
TGCCAGCAGGCAGGCTGCCATTGGTCAGGGCCTTCAGCTGGTTTCCTGCCAGGTCGAGGACTTCCAGTTTGGGCAGG
AAGTGGAGGCTCCACCACTTAAAGAAGGCCAGGTAATTGTCACGGAGATGCAGCACCTGTAGGCTCTTGGGGAGGTT
GCGCAGGGTTTGGGGCAGGAGGGTGTGCAGGCGATTCTGGGACAAGTCCAGCCAGATCAAACCGCTCAGGCCTTGGA
AGAAGTGCAGATAGAGGTCTCCCTCGGCCCACATATGGCCCAGTGCATTGCCGCTGAAGTCCAGGGCCCGCAGCGAC
GTACTGCAGAGCTGCTGGGACACTTGGCTGTGGATGTTGTTGTGGGCCAGGCTGAGGTGGCGCAGGGTGCGCAGGTG
GGCCACGAAGCTGAAGTTGTGGCCCACGCCCTGCATGCCAAAGGGCTGGCTGTTGTAGCTGAGGTCCAGGGCCTCCA
GTCGTGGTAGCTCCGTGAATGAGTGCTCGTGGTAGAGGTCCAGCTTATTGTGGGACAGGTCTAGCACCTGCAGACCG
GTCAGCGGCAGGAACTGGGAGCCATTGACTGCCTGCGAGATGCAGTTGTGGCTCAGGCGCAGGCACTGCAGGTGCGA
GAGCTGGGCAAACATCTCCGGCTGCACGGTCACCAGGTTGTTCCGTGACAGATCCAAGGTGAAGTTGAGGGTGCTGC
AGTTGGGCCTGAAGTCTTCAGAGCTGGGAGTGTCCACTGGGGCCGGAGCAAGGTCCCCAGGCTGCAGCCAGACCTTC
TCCCCTCCATCTGCCTCCCCCATGGTGGCTGTCAGCTCCGAAGCTCCGCTGATGCGGTTGTCCGACAGGTCCACGTA
GCGCAGGCCAGGGAAGGCCCTGAAGATGCCGAGCTGGGCCTGGTTGATGAAGTTCATCTGCAGACGCAGAGTCTGGA
GCATGGGCAGGCGGGCCAGTGGCCGGAGCGTGGTCTCATCGAGTGAGCGGAAGAAGATGCCGTGCATGTCCAGCTCC
TTCAGGGCGACCAGGCTCCCGAAGGAAGGGGCCAGAGACAGGTGGGCAAAGGACACCCTCTTTTGGTAATTGAAGGA
CAGGTTAAGCTTGCGCAGCTGTGTTAGGCCCTGGAAGGCCTTGGTTTTAGTGATGCATTTGTAGAGGAAGTTCTCAC
TCAGGTCCAGCACTCGGAGGTTTCCCAGCCCACGGAACCAACTGGCATTCAGCCAGGAGAGAGAACTGTCCTTCAAC
ACCAGGCCTTCAAGACGGCTCAGGTGGCTGAAGGTATCGGGATGTAGCTGGGGGAAGTGACGAGGGCACTCCATGCA
GGGGTTGGGAGCGTGGTCGCAGCGGCGGCAATTTCCGCCCACATCGAGCACACGCAGGGCGGTCAGATTGGCCAGGT
CCTCAGGCGCCAGTTTGACGATGCGGTTGTAGGACAACAGCAGATACTCCAGGCTGGAAGGCAGGTTGCGGGGCACC
ACAGTGAGGTTGTTGTACTTGAGTGACAGGTGGGTGAGGTTGCCCAGGCCAAGGAGGGCACCCGGGGCCACCTCCAG
TGCCTGCCTGCAGGGGTTCTTGTAATAACAGTTGCCGTCCATGAATAGGAAGCGCAGGGCATGCAGGCCGGCGAGGC
TGGCAGAGTCTAGCATCAGGATGTTGGTATGGCTGAGGGACAGGGATATGAGGGATTTGGGCAGCGCAGGCACAGTC
ATGATGTTGTTGTAGCTCAGGTTTAGCTCTTCCAGGGTGGGCACAGCCAAGAAGGTGCTGGGCTCGATGGTCATGTG
GCAGGGGAAGTGCATGGGGCTGAGGCCAACCGGCGGGCAGTTCCACTTGAGGTTGAGATGCCGCAGGCTGGGCAGGT
GGGCAAAGTCAGAATCATGGAGGTGGTGGATGCGGTTGGAGGACAAGGAAAGGCTGGTGACATTGCCACGGGGTGCT
GCCATGGAGAAGTGGGGCACAGACTTCAGGAACAGCCAGTTGCAGTTCACCAGGCCGTGGGGCTGGAGCTCACAGGG
TAGGAAGGCAGGCAAGGTACCCAGGGCCAGGGTCATGGCCAGCATGGTGGATCC

A disclosed encoded MOL1p protein has 1019 amino acid residues, referred to as the MOL1p protein. The disclosed MOL1p polypeptide sequence is presented in Table 1AAJ using the one-letter amino acid code.

TABLE 1AAJ
Encoded MOL1p protein sequence.

(SEQ ID NO:165)

GSTMLAMTLALGTLPAFLPCELQPHGLVNCNWLFLKSVPHFSMAAPRGNVTSLSLSSNRIHHLHDSDFAHLPSLRHL
NLKWNCPPVGLSPMHFPCHMTIEPSTFLAVPTLEELNLSYNNIMTVPALPKSLISLSLSHTNILMLDSASLAGLHAL
RFLFMDGNCYYKNPCRQALEVAPGALLGLGNLTHLSLKYNNLTVVPRNLPSSLEYLLLSYNRIVKLAPEDLANLTAL
RVLDVGGNCRRCDHAPNPCMECPRHFPQLHPDTFSHLSRLEGLVLKDSSLSWLNASWFRGLGNLRVLDLSENFLYKC
ITKTKAFQGLTQLRKLNLSFNYQKRVSFAHLSLAPSFGSLVALKELDMHGIFFRSLDETTLRPLARLPMLQTLRLQM
NFINQAQLGIFRAFPGLRYVDLSDNRISGASELTATMGEADGGEKVWLQPGDLAPAPVDTPSSEDFRPNCSTLNFTL
DLSRNNLVTVQPEMFAQLSHLQCLRLSHNCISQAVNGSQFLPLTGLQVLDLSHNKLDLYHEHSFTELPRLEALDLSY
NSQPFGMQGVGHNFSFVAHLRTLRHLSLAHNNIHSQVSQQLCSTSLRALDFSGNALGHMWAEGDLYLHFFQGLSGLI
WLDLSQNRLHTLLPQTLRNLPKSLQVLHLRDNYLAFFKWWSLHFLPKLEVLDLAGNQLKALTNGSLPAGTRLRRLDV
SCNSISFVAPGFFSKAKELRELNLSANALKTVDHSWFGPLASALQILDVSANPLHCACGAAFMDFLLEVQAAVPGLP
SRVKCGSPGQLQGLSIFAQDLRLCLDEALSWDCFALSLLAVALGLGVPMLHHLCGWDLWYCFHLCLAWLPWRGRQSG
RDEDALPYDAFVVFDKTQSAVADWVYNELRGQLEECRGRWALRLCLEERDWLPGKTLFENLWASVYGSRKTLFVLAH
TDRVSGLLRASFLLAQQRLLEDRKDVVVLVILSPDGRRSRYVRLRQRLCRQSVLLWPHQPSGQRSFWAQLGMALTRD
NHHFYNRNFCQGPTAELE

MOL1q

A disclosed Toll receptor 9-like nucleic acid of 3057 nucleotides, MOL1q, is shown in Table 1AAK. The disclosed MOL1q open reading frame (“ORF”) begins at the GGA initiation codon at nucleotides 1-3, shown in bold in Table 1AAK. The encoded polypeptide is alternatively referred to herein as MOL1q or as 264289162. The disclosed MOL1q ORF terminates at a GAG codon at nucleotides 3055-3057. As shown in Table 1AAK the start and stop codons are in bold letters. Because MOL1q does not start or stop at traditional initiation and termination codons, MOL1q could be a partial open reading frame which extends further in the 5′ and/or 3′ directions.

TABLE 1AAK
MOL1q nucleotide sequence.

(SEQ ID NO:166)

GGATCCACCATGCTGGCCATGACCCTGGCCCTGGGTACCTTGCCTGCCTTCCTACCCTGTGAGCTCCAGCCCCACGG
CCTGGTGAACTGCAACTGGCTGTTCCTGAAGTCTGTGCCCCACTTCTCCATGGCAGCACCCCGTGGCAATGTCACCA
GCCTTTCCTTGTCCTCCAACCGCATCCACCACCTCCATGATTCTGACTTTGCCCACCTGCCCAGCCTGCGGCATCTC
AACCTCAAGTGGAACTGCCCGCCGGTTGGCCTCAGCCCCATGCACTTCCCCTGCCACATGACCATCGAGCCCAGCAC
CTTCTTGGCTGTGCCCACCCTGGAAGAGCTAAACCTGAGCTACAACAACATCATGACTGTGCCTGCGCTGCCCAAAT
CCCTCATATCCCTGTCCCTCAGCCATACCAACATCCTGATGCTAGACTCTGCCAGCCTCGCCGGCCTGCATGCCCTG
CGCTTCCTATTCATGGACGGCAACTGTTATTACAAGAACCCCTGCAGGCAGGCACTGGAGGTGGCCCCGGGTGCCCT
CCTTGGCCTGGGCAACCTCACCCACCTGTCACTCAAGTACAACAACCTCACTGTGGTGCCCCGCAACCTGCCTTCCA
GCCTGGAGTATCTGCTGTTGTCCTACAACCGCATCGTCAAACTGGCGCCTGAGGACCTGGCCAATCTGACCGCCCTG
CGTGTGCTCGATGTGGGCGGAAATTGCCGCCGCTGCGACCACGCTCCCAACCCCTGCATGGAGTCCCCTCGTCACTT
CCCCCAGCTACATCCCGATACCTTCAGCCACCTGAGCCGTCTTGAAGGCCTGGTGTTGAAGGACAGTTCTCTCTCCT
GGCTGAATGCCAGTTGGTTCCGTGGGCTGGGAAACCTCCGAGTGCTGGACCTGAGTGAGAACTTCCTCTACAAATGC
ATCACTAAAACCAAGGCCTTCCAGGGCCTAACACAGCTGCGCAAGCTTAACCTGTCCTTCAATTACCAAAAGAGGGT
GTCCTTTGCCCACCTGTCTCTGGCCCCTTCCTTCGGGAGCCTGGTCGCCCTGAAGGAGCTGGACATGCACGGCATCT
TCTTCCGCTCACTCGATGAGACCACGCTCCGGCCACTGGCCCGCCTGCCCATGCTCCAGACTCTGCGTCTGCAGATG
AACTTCATCAACCAGGCCCAGCTCGGCATCTTCAGGGCCTTCCCTGGCCTGCGCTACGTGGACCTGTCGGACAACCG
CATCAGCGGAGCTTCGGAGCTGACAGCCACCATGGGGGAGGCAGATGGAGGGGAGAAGGTCTGGCTGCAGCCTGGGG
ACCTTGCTCCGGCCCCAGTGGACACTCCCAGCTCTGAAGACTTCAGGCCCAACTGCAGCACCCTCAACTTCACCTTG
GATCTGTCACGGAACAACCTGGTGACCGTGCAGCCGGAGATGTTTGCCCAGCTCTCGCACCTGCAGTGCCTGCGCCT
GAGCCACAACTGCATCTCGCAGGCAGTCAATGGCTCCCAGTTCCTGCCGCTGACCGGTCTGCAGGTGCTAGACCTGT
CCCACAATAAGCTGGACCTCTACCACGAGCACTCATTCACGGAGCTACCACGACTGGAGGCCCTGGACCTCAGCTAC
AACAGCCAGCCCTTTGGCATGCAGGGCGTGGGCCACAACTTCAGCTTCGTGGCTCACCTGCGCACCCTGCGCCACCT
CAGCCTGGCCCACAACAACATCCACAGCCAAGTGTCCCAGCAGCTCTGCAGTACGTCGCTGCGGGCCCTGGACTTCA
GCGGCAATGCACTGGGCCATATGTGGGCCGAGGGAGACCTCTATCTGCACTTCTTCCAAGGCCTGAGCGGTTTGATC
TGGCTGGACTTGTCCCAGAACCGCCTGCACACCCTCCTGCCCCAAACCCTGCGCAACCTCCCCAAGAGCCTACAGGT
GCTGCGTCTCCGTGACAATTACCTGGCCTTCTTTAAGTGGTGGAGCCTCCACTTCCTGCCCAAACTGGAAGTCCTCG
ACCTGGCAGGAAACCAGCTGAAGGCCCTGACCAATGGCAGCCTGCCTGCTGGCACCCGGCTCCGGAGGCTGGATGTC
AGCTGCAACAGCATCAGCTTCGTGGCCCCCGGCTTCTTTTCCAAGGCCAAGGAGCTGCGAGAGCTCAACCTTAGCGC
CAACGCCCTCAAGACAGTGGACCACTCCTGGTTTGGGCCCCTGGCGAGTGCCCTGCAAATACTAGATGTAAGCGCCA
ACCCTCTGCACTGCGCCTGTGGGGCGGCCTTTATGGACTTCCTGCTGGAGGTGCAGGCTGCCGTGCCCGGTCTGCCC
AGCCGGGTGAAGTGTGGCAGTCCGGGCCAGCTCCAGGGCCTCAGCATCTTTGCACAGGACCTGCGCCTCTGCCTGGA
TGAGGCCCTCTCCTGGGACTGTTTCGCCCTCTCGCTGCTGGCTGTGGCTCTGGGCCTGGGTGTGCCCATGCTGCATC
ACCTCTGTGGCTGGGACCTCTGGTACTGCTTCCACCTGTGCCTGGCCTGGCTTCCCTGGCGGGGGCGGCAAAGTGGG
CGAGATGAGGATGCCCTGCCCTACGATGCCTTCGTGGTCTTCGACAAAACGCAGAGCGCAGTGGCAGACTGGGTGTA
CAACGAGCTTCGGGGGCAGCTGGAGGAGTGCCGTGGGCGCTGGGCACTCCGCCTGTGCCTGGAGGAACGCGACTGGC
TGCCTGGCAAAACCCTCTTTGAGAACCTGTGGGCCTCGGTCTATGGCAGCCGCAAGACGCTGTTTGTGCTGGCCCAC
ACGGACCGGGTCAGTGGTCTCTTGCGCGCCAGCTTCCTGCTGGCCCAGCAGCGCCTGCTGGAGGACCGCAAGGACGT
CGTGGTGCTGGTGATCCTGAGCCCTGACGGCCGCCGCTCCCGCTACGTGCGGCTGCGCCAGCGCCTCTGCCGCCAGA
GTGTCCTCCTCTGGCCCCACCAGCCCAGTGGTCAGCGCAGCTTCTGGGCCCAGCTGGGCATGGCCCTGACCAGGGAC
AACCACCACTTCTATAACCGGAACTTCTGCCAGGGACCCACGGCCGAACTCGAG

TABLE 1AAL
MOL1q reverse complement nucleotide sequence.

(SEQ ID NO:180)

CTCGAGTTCGGCCGTGGGTCCCTGGCAGAAGTTCCGGTTATAGAAGTGGTGGTTGTCCCTGGTCAGGGCCATGCCCA
GCTGGGCCCAGAAGCTGCGCTGACCACTGGGCTGGTGGGGCCAGAGGAGGACACTCTGGCGGCAGAGGCGCTGGCGC
AGCCGCACGTAGCGGGAGCGGCGGCCGTCAGGGCTCAGGATCACCAGCACCACGACGTCCTTGCGGTCCTCCAGCAG
GCGCTGCTGGGCCAGCAGGAAGCTGGCGCGCAAGAGACCACTGACCCGGTCCGTGTGGGCCAGCACAAACAGCGTCT
TGCGGCTGCCATAGACCGAGGCCCACAGGTTCTCAAAGAGGGTTTTGCCAGGCAGCCAGTCGCGTTCCTCCAGGCAC
AGGCGGAGTGCCCAGCGCCCACGGCACTCCTCCAGCTGCCCCCGAAGCTCGTTGTACACCCAGTCTGCCACTGCGCT
CTGCGTTTTGTCGAAGACCACGAAGGCATCGTAGGGCAGGGCATCCTCATCTCGCCCACTTTGCCGCCCCCGCCAGG
GAAGCCAGGCCAGGCACAGGTGGAAGCAGTACCAGAGGTCCCAGCCACAGAGGTGATGCAGCATGGGCACACCCAGG
CCCAGAGCCACAGCCAGCAGCGAGAGGGCGAAACAGTCCCAGGAGAGGGCCTCATCCAGGCAGAGGCGCAGGTCCTG
TGCAAAGATGCTGAGGCCCTGGAGCTGGCCCGGACTGCCACACTTCACCCGGCTGGGCAGACCGGGCACGGCAGCCT
GCACCTCCAGCAGGAAGTCCATAAAGGCCGCCCCACAGGCGCAGTGCAGAGGGTTGGCGCTTACATCTAGTATTTGC
AGGGCACTCGCCAGGGGCCCAAACCAGGAGTGGTCCACTGTCTTGAGGGCGTTGGCGCTAAGGTTGAGCTCTCGCAG
CTCCTTGGCCTTGGAAAAGAAGCCGGGGGCCACGAAGCTGATGCTGTTGCAGCTGACATCCAGCCTCCGGAGCCGGG
TGCCAGCAGGCAGGCTGCCATTGGTCAGGGCCTTCAGCTGGTTTCCTGCCAGGTCGAGGACTTCCAGTTTGGGCAGG
AAGTGGAGGCTCCACCACTTAAAGAAGGCCAGGTAATTGTCACGGAGACGCAGCACCTGTAGGCTCTTGGGGAGGTT
GCGCAGGGTTTGGGGCAGGAGGGTGTGCAGGCGGTTCTGGGACAAGTCCAGCCAGATCAAACCGCTCAGGCCTTGGA
AGAAGTGCAGATAGAGGTCTCCCTCGGCCCACATATGGCCCAGTGCATTGCCGCTGAAGTCCAGGGCCCGCAGCGAC
GTACTGCAGAGCTGCTGGGACACTTGGCTGTGGATGTTGTTGTGGGCCAGGCTGAGGTGGCGCAGGGTGCGCAGGTG
AGCCACGAAGCTGAAGTTGTGGCCCACGCCCTGCATGCCAAAGGGCTGGCTGTTGTAGCTGAGGTCCAGGGCCTCCA
GTCGTGGTAGCTCCGTGAATGAGTGCTCGTGGTAGAGGTCCAGCTTATTGTGGGACAGGTCTAGCACCTGCAGACCG
GTCAGCGGCAGGAACTGGGAGCCATTGACTGCCTGCGAGATGCAGTTGTGGCTCAGGCGCAGGCACTGCAGGTGCGA
GAGCTGGGCAAACATCTCCGGCTGCACGGTCACCAGGTTGTTCCGTGACAGATCCAAGGTGAAGTTGAGGGTGCTGC
AGTTGGGCCTGAAGTCTTCAGAGCTGGGAGTGTCCACTGGGGCCGGAGCAAGGTCCCCAGGCTGCAGCCAGACCTTC
TCCCCTCCATCTGCCTCCCCCATGGTGGCTGTCAGCTCCGAAGCTCCGCTGATGCGGTTGTCCGACAGGTCCACGTA
GCGCAGGCCAGGGAAGGCCCTGAAGATGCCGAGCTGGGCCTGGTTGATGAAGTTCATCTGCAGACGCAGAGTCTGGA
GCATGGGCAGGCGGGCCAGTGGCCGGAGCGTGGTCTCATCGAGTGAGCGGAAGAAGATGCCGTGCATGTCCAGCTCC
TTCAGGGCGACCAGGCTCCCGAAGGAAGGGGCCAGAGACAGGTGGGCAAAGGACACCCTCTTTTGGTAATTGAAGGA
CAGGTTAAGCTTGCGCAGCTGTGTTAGGCCCTGGAAGGCCTTGGTTTTAGTGATGCATTTGTAGAGGAAGTTCTCAC
TCAGGTCCAGCACTCGGAGGTTTCCCAGCCCACGGAACCAACTGGCATTCAGCCAGGAGAGAGAACTGTCCTTCAAC
ACCAGGCCTTCAAGACGGCTCAGGTGGCTGAAGGTATCGGGATGTAGCTGGGGGAAGTGACGAGGGCACTCCATGCA
GGGGTTGGGAGCGTGGTCGCAGCGGCGGCAATTTCCGCCCACATCGAGCACACGCAGGGCGGTCAGATTGGCCAGGT
CCTCAGGCGCCAGTTTGACGATGCGGTTGTAGGACAACAGCAGATACTCCAGGCTGGAAGGCAGGTTGCGGGGCACC
ACAGTGAGGTTGTTGTACTTGAGTGACAGGTGGGTGAGGTTGCCCAGGCCAAGGAGGGCACCCGGGGCCACCTCCAG
TGCCTGCCTGCAGGGGTTCTTGTAATAACAGTTGCCGTCCATGAATAGGAAGCGCAGGGCATGCAGGCCGGCGAGGC
TGGCAGAGTCTAGCATCAGGATGTTGGTATGGCTGAGGGACAGGGATATGAGGGATTTGGGCAGCGCAGGCACAGTC
ATGATGTTGTTGTAGCTCAGGTTTAGCTCTTCCAGGGTGGGCACAGCCAAGAAGGTGCTGGGCTCGATGGTCATGTG
GCAGGGGAAGTGCATGGGGCTGAGGCCAACCGGCGGGCAGTTCCACTTGAGGTTGAGATGCCGCAGGCTGGGCAGGT
GGGCAAAGTCAGAATCATGGAGGTGGTGGATGCGGTTGGAGGACAAGGAAAGGCTGGTGACATTGCCACGGGGTGCT
GCCATGGAGAAGTGGGGCACAGACTTCAGGAACAGCCAGTTGCAGTTCACCAGGCCGTGGGGCTGGAGCTCACAGGG
TAGGAAGGCAGGCAAGGTACCCAGGGCCAGGGTCATGGCCAGCATGGTGGATCC

A disclosed encoded MOL1q protein has 1019 amino acid residues, referred to as the MOL1q protein. The disclosed MOL1q polypeptide sequence is presented in Table 1AAM using the one-letter amino acid code.

TABLE 1AAM
Encoded MOL1q protein sequence.

(SEQ ID NO:167)

GSTMLAMTL ALGTLPAFLPCELQPHGLVNCNWLFLKSVPHFSMAAPRGNVTSLSLSSNRIHHLHDSDFAHLPSLRHL
NLKWNCPPVGLSPMHFPCHMTIEPSTFLAVPTLEELNLSYNNIMTVPALPKSLISLSLSHTNILMLDSASLAGLHAL
RFLFMDGNCYYKNPCRQALEVAPGALLGLGNLTHLSLKYNNLTVVPRNLPSSLEYLLLSYNRIVKLAPEDLANLTAL
RVLDVGGNCRRCDHAPNPCMECPRHFPQLHPDTFSHLSRLEGLVLKDSSLSWLNASWFRGLGNLRVLDLSENFLYKC
ITKTKAFQGLTQLRKLNLSFNYQKRVSFAHLSLAPSFGSLVALKELDMHGIFFRSLDETTLRPLARLPMLQTLRLQM
NFINQAQLGIFRAFPGLRYVDLSDNRISGASELTATMGEADGGEKVWLQPGDLAPAPVDTPSSEDFRPNCSTLNFTL
DLSRNNLVTVQPEMFAQLSHLQCLRLSHNCISQAVNGSQFLPLTGLQVLDLSHNKLDLYHEHSFTELPRLEALDLSY
NSQPFGMQGVGHNFSFVAHLRTLRHLSLAHNNIHSQVSQQLCSTSLRALDFSGNALGHMWAEGDLYLHFFQGLSGLI
WLDLSQNRLHTLLPQTLRNLPKSLQVLRLRDNYLAFFKWWSLHFLPKLEVLDLAGNQLKALTNGSLPAGTRLRRLDV
SCNSISFVAPGFFSKAKELRELNLSANALKTVDHSWFGPLASALQILDVSANPLHCACGAAFMDFLLEVQAAVPGLP
SRVKCGSPGQLQGLSIFAQDLRLCLDEALSWDCFALSLLAVALGLGVPMLHHLCGWDLWYCFHLCLAWLPWRGRQSG
RDEDALPYDAFVVFDKTQSAVADWVYNELRGQLEECRGRWALRLCLEERDWLPGKTLFENLWASVYGSRKTLFVLAH
TDRVSGLLRASFLLAQQRLLEDRKDVVVLVILSPDGRRSRYVRLRQRLCRQSVLLWPHQPSGQRSFWAQLGMALTRD
NHHFYNRNFCQGPTAELE

MOL1r

A disclosed Toll receptor 9-like nucleic acid of 3111 nucleotides, MOL1r, is shown in Table 1AAN. The disclosed MOL1r open reading frame (“ORF”) begins at the GGA initiation codon at nucleotides 1-3, shown in bold in Table 1AAN. The encoded polypeptide is alternatively referred to herein as MOL1r or as 248651580. The disclosed MOL1r ORF terminates at a GAG codon at nucleotides 3109-3111. As shown in Table 1AAN the start and stop codons are in bold letters. Because MOL1r does not start or stop at traditional initiation or termination codons, MOL1r could be a partial open reading frame which extends further in the 5′ and/or 3′ directions.

TABLE 1AAN
MOL1r nucleotide sequence.

(SEQ ID NO:168)

GGATCCACCATGGGTTTCTGCCGCAGCGCCCTGCACCCGCTGTCTCTCCTGGTGCAGGCCATCATGCTGGCCATGAC
CCTGGCCCTGGGTACCTTGCCTGCCTTCCTACCCTGTGAGCTCCAGCCCCACGGCCTGGTGAACTGCAACTGGCTGT
TCCTGAAGTCTGTGCCCCACTTCTCCATGGCAGCACCCCGTGGCAATGTCACCAGCCTTTCCTTGTCCTCCAACCGC
ATCCACCACCTCCATGATTCTGACTTTGCCCACCTGCCCAGCCTGCGGCATCTCAACCTCAAGTGGAACTGCCCGCC
GGTTGGCCTCAGCCCCATGCACTTCCCCTGCCACATGACCATCGAGCCCAGCACCTTCTTGGCTGTGCCCACCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCGCCGGCCTGCATGCCCTGCGCTTCCTATTCATGGACGGCAA
CTGTTATTACAAGAACCCCTGCAGGCAGGCACTGGAGGTGGCCCCGGGTGCCCTCCTTGGCCTGGGCAACCTCACCC
ACCTGTCACTCAAGTACAACAACCTCACTGTGGTGCCCCGCAACCTGCCTTCCAGCCTGGAGTATCTGCTGTTGTCC
TACAACCGCATCGTCAAACTGGCGCCTGAGGACCTGGCCAATCTGACCGCCCTGCGTGTGCTCGATGTGGGCGGAAA
TTGCCGCCGCTGCGACCACGCTCCCAACCCCTGCATGGAGTGCCCTCGTCACTTCCCCCAGCTACATCCCGATACCT
TCAGCCACCTGAGCCGTCTTGAAGGCCTGGTGTTGAAGGACAGTTCTCTCTCCTGGCTGAATGCCAGTTGGTTCCGT
GGGCTGGGAAACCTCCGAGTGCTGGACCTGAGTGAGAACTTCCTCTACAAATGCATCACTAAAACCAAGGCCTTCCA
GGGCCTAACACAGCTGCGCAAGCTTAACCTGTCCTTCAATTACCAAAAGAGGGTGTCCTTTGCCCACCTGTCTCTGG
CCCCTTCCTTCGGGAGCCTGGTCGCCCTGAAGGAGCTGGACATGCACGGCATCTTCTTCCGCTCACTCGATGAGACC
ACGCTCCGGCCACTGGCCCGCCTGCCCATGCTCCAGACTCTGCGTCTGCAGATGAACTTCATCAACCAGGCCCAGCT
CGGCATCTTCAGGGCCTTCCCTGGCCTGCGCTACGTGGACCTGTCGGACAACCGCATCAGCGGAGCTTCGGAGCTGA
CAGCCACCATGGGGGAGGCAGATGGAGGGGAGAAGGTCTGGCTGCAGCCTGGGGACCTTGCTCCGGCCCCAGTGGAC
ACTCCCAGCTCTGAAGACTTCAGGCCCAACTGCAGCACCCTCAACTTCACCTTGGATCTGTCACGGAACAACCTGGT
GACCGTGCAGCCGGAGATGTTTGCCCAGCTCTCGCACCTGCAGTGCCTGCGCCTGAGCCACAACTGCATCTCGCAGG
CAGTCAATGGCTCCCAGTTCCTGCCGCTGACCTGTCTGCAGGTGCTAGACCTGTCCCACAATAAGCTGGACCTCTAC
CACGAGCACTCATTCACGGAGCTACCACGACTGGAGGCCCTGGACCTCAGCTACAACAGCCAGCCCTTTGGCATGCA
GGGCGTGGGCCACAACTTCAGCTTCGTGGCTCACCTGCGCACCCTGCGCCACCTCAGCCTGGCCCACAACAACATCC
ACAGCCAAGTGTCCCAGCAGCTCTGCAGTACGTCGCTGCGGGCCCTGGACTTCAGCGGCAATGCACTGGGCCATATG
TGGGCCGAGGGAGACCTCTATCTGCACTTCTTCCAAGGCCTGAGCGGTTTGATCTGGCTGGACTTGTCCCAGAACCG
CCTGCACACCCTCCTGCCCCAAACCCTGCGCAACCTCCCCAAGAGCCTACAGGTGCTGCGTCTCCGTGACAATTACC
TGGCCTTCTTTAAGTGGTGGAGCCTCCACTTCCTGCCCAAACTGGAAGTCCTCGACCTGGCAGGAAACCAGCTGAAG
GCCCTGACCAATGGCAGCCTGCCTGCTGGCACCCGGCTCCGGAGGCTGGATGTCAGCTGCAACAGCATCAGCTTCGT
GGCCCCCGGCTTCTTTTCCAAGGCCAAGGAGCTGCGAGAGCTCAACCTTAGCGCCAACGCCCTCAAGACAGTGGACC
ACTCCTGGTTTGGGCCCCTGGCGAGTGCCCTGCAAATACTAGATGTAAGCGCCAACCCTCTGCACTGCGCCTGTGGG
GCGGCCTTTATGGACTTCCTGCTGGAGGTGCAGGCTGCCGTGCCCGGTCTGCCCAGCCGGGTGAAGTGTGGCAGTCC
GGGCCAGCTCCAGGGCCTCAGCATCTTTGCACAGGACCTGCGCCTCTGCCTGGATGAGGCCCTCTCCTGGGACTGTT
TCGCCCTCTCGCTGCTGGCTGTGGCTCTGGGCCTGGGTGTGCCCATGCTGCATCACCTCTGTGGCTGGGACCTCTGG
TACTGCTTCCACCTGTGCCTGGCCTGGCTTCCCTGGCGGGGGCGGCAAAGTGGGCGAGATGAGGATGCCCTGCCCTA
CGATGCCTTCGTGGTCTTCGACAAAACGCAGAGCGCAGTGGCAGACTGGGTGTACAACGAGCTTCGGGGGCAGCTGG
AGGAGTGCCGTGGGCGCTGGGCACTCCGCCTGTGCCTGGAGGAACGCGACTGGCTGCCTGGCAAAACCCTCTTTGAG
AACCTGTGGGCCTCGGTCTATGGCAGCCGCAAGACGCTGTTTGTGCTGGCCCACACGGACCGGGTCAGTGGTCTCTT
GCGCGCCAGCTTCCTGCTGGCCCAGCAGCGCCTGCTGGAGGACCGCAAGGACGTCGTGGTGCTGGTGATCCTGAGCC
CTGACGGCCGCCGCTCCCGCTACGTGCGGCTGCGCCAGCGCCTCTGCCGCCAGAGTGTCCTCCTCTGGCCCCACCAG
CCCAGTGGTCAGCGCAGCTTCTGGGCCCAGCTGGGCATGGCCCTGACCAGGGACAACCACCACTTCTATAACCGGAA
CTTCTGCCAGGGACCCACGGCCGAACTCGAG

A disclosed encoded MOL1r protein has 1037 amino acid residues, referred to as the MOL1r protein. The disclosed MOL1r polypeptide sequence is presented in Table 1AAO using the one-letter amino acid code.

TABLE 1AAO
Encoded MOL1r protein sequence.

(SEQ ID NO:169)

GSTMGFCRSALHPLSLLVQAIMLAMTLALGTLPAFLPCELQPHGLVNCNWLFLKSVPHFSMAAPRGNVTSLSLSSNR
IHHLHDSDFAHLPSLRHLNLKWNCPPVGLSPMHFPCHMTIEPSTFLAVPTLEELNLSYNNIMTVPALPKSLISLSLS
HTNILMLDSASLAGLHALRFLFMDGNCYYKNPCRQALEVAPGALLGLGNLTHLSLKYNNLTVVPRNLPSSLEYLLLS
YNRIVKLAPEDLANLTALRVLDVGGNCRRCDHAPNPCMECPRHFPQLHPDTFSHLSRLEGLVLKDSSLSWLNASWFR
GLGNLRVLDLSENFLYKCITKTKAFQGLTQLRKLNLSFNYQKRVSFAHLSLAPSFGSLVALKELDMHGIFFRSLDET
TLRPLARLPMLQTLRLQMNFINQAQLGIFRAFPGLRYVDLSDNRISGASELTATMGEADGGEKVWLQPGDLAPAPVD
TPSSEDFRPNCSTLNFTLDLSRNNLVTVQPEMFAQLSHLQCLRLSHNCISQAVNGSQFLPLTCLQVLDLSHNKLDLY
HEHSFTELPRLEALDLSYNSQPFGMQGVGHNFSFVAHLRTLRHLSLAHNNIHSQVSQQLCSTSLRALDFSGNALGHM
WAEGDLYLHFFQGLSGLIWLDLSQNRLHTLLPQTLRNLPKSLQVLRLRDNYLAFFKWWSLHFLPKLEVLDLAGNQLK
ALTNGSLPAGTRLRRLDVSCNSISFVAPGFFSKAKELRELNLSANALKTVDHSWFGPLASALQILDVSANPLHCACG
AAFMDFLLEVQAAVPGLPSRVKCGSPGQLQGLSIFAQDLRLCLDEALSWDCFALSLLAVALGLGVPMLHHLCGWDLW
YCFHLCLAWLPWRGRQSGRDEDALPYDAFVVFDKTQSAVADWVYNELRGQLEECRGRWALRLCLEERDWLPGKTLFE
NLWASVYGSRKTLFVLAHTDRVSGLLRASFLLAQQRLLEDRKDVVVLVILSPDGRRSRYVRLRQRLCRQSVLLWPHQ
PSGQRSFWAQLGMALTRDNHHFYNRNFCQGPTAELE

MOL1s

A disclosed Toll receptor 9-like nucleic acid of 3111 nucleotides, MOL1s, is shown in Table 1AAP. The disclosed MOL1s open reading frame (“ORF”) begins at the GGA initiation codon at nucleotides 1-3, shown in bold in Table 1AAP. The encoded polypeptide is alternatively referred to herein as MOL1s or as 255304731. The disclosed MOL1s ORF terminates at a GAG codon at nucleotides 3109-3111. As shown in Table 1AAP the start and stop codons are in bold letters. Because MOL1s does not start or stop at traditional initiation or termination codons, MOL1s could be a partial open reading frame which extends further in the 5′ and/or 3′ directions.

TABLE 1AAP
MOL1s nucleotide sequence.

(SEQ ID NO:170)

GGATCCACCATGGGTTTCTGCCGCAGCGCCCTGCACCCGCTGTCTCTCCTGGTGCAGGCCATCATGCTGGCCATGAC
CCTGCCCCTGGGTACCTTGCCTCCCTTCCTACCCTGTGACCTCCAGCCCCACGGCCTGGTGAACTGCAACTGCCTCT
TCCTGAAGTCTGTGCCCCACTTCTCCATGGCAGCACCCCGTGGCAATGTCACCAGCCTTTCCTTGTCCTCCAACCGC
ATCCACCACCTCCATGATTCTGACTTTGCCCACCTGCCCAGCCTGCGGCATCTCAACCTCAAGTGGAACTGCCCGCC
GGTTGGCCTCAGCCCCATGCACTTCCCCTGCCACATGACCATCGACCCCACCACCTTCTTCGCTGTGCCCACCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCCCCGCCCTGCATGCCCTGCGCTTCCTATTCATCGACGGCAA
CTGTTATTACAAGAACCCCTGCAGGCAGGCACTGGACGTGGCCCCGGGTGCCCTCCTTGGCCTGGGCAACCTCACCC
ACCTGTCACTCAAGTACAACAACCTCACTGTGGTGCCCCGCAACCTGCCTTCCAGCCTGGACTATCTGCTGTTGTCC
TACAACCCCATCGTCAAACTGGCGCCTGAGGACCTGGCCAATCTGACCCCCCTGCGTCTGCTCCATGTGGGCCGAAA
TTGCCGCCGCTGCGACCACGCTCCCAACCCCTGCATGGAGTGCCCTCGTCACTTCCCCCAGCTACATCCCGATACCT
TCAGCCACCTGAGCCGTCTTGAAGGCCTGGTGTTGAAGGACAGTTCTCTCTCCTCCCTGAATGCCAGTTGGTTCCGT
CCGCTCGGAAACCTCCGAGTGCTCGACCTGAGTCAGAACTTCCTCTACAAATCCATCACTAAAACCAAGGCCTTCCA
GGGCCTAACACAGCTGCGCAAGCTTAACCTGTCCTTCAATTACCAAAAGAGGGTCTCCTTTGCCCACCTGTCTCTGG
CCCCTTCCTTCGGGACCCTCGTCGCCCTGAAGGAGCTGGACATGCACGGCATCTTCTTCCGCTCACTCCATGAGACC
ACGCTCCGGCCACTGGCCCGCCTGCCCATGCTCCAGACTCTGCGTCTGCAGATGAACTTCATCAACCACGCCCAGCT
CGGCATCTTCAGGGCCTTCCCTCGCCTGCGCTACGTCGACCTGTCGGACAACCGCATCAGCGGAGCTTCGGAGCTGA
CACCCACCATGGGGGAGGCAGATGGAGCGGAGAAGGTCTGGCTGCAGCCTGGGCACCTTGCTCCGGCCCCAGTGGAC
ACTCCCAGCTCTCAAGACTTCAGGCCCAACTGCAGCACCCTCAACTTCACCTTGGATCTGTCACGGAACAACCTGGT
GACCGTGCAGCCGGAGATCTTTGCCCACCTCTCGCACCTGCAGTGCCTGCGCCTGAGCCACAACTGCATCTCGCAGG
CAGTCAATGCCTCCCAGTTCCTCCCGCTCACCGGTCTGCAGCTGCTAGACCTCTCCCACAATAAGCTGGACCTCTAC
CACCACCACTCATTCACGGAGCTACCGCGACTGGAGGCCCTGGACCTCAGCTACAACAGCCAGCCCTTTGGCATGCA
GGGCGTGGGCCACAACTTCAGCTTCGTCCCTCACCTGCGCACCCTGCGCCACCTCAGCCTCGCCCACAACAACATCC
ACAGCCAAGTGTCCCAGCAGCTCTGCAGTACGTCGCTGCGGGCCCTGGACTTCACCCGCAATGCACTGGCCCATATG
TCGCCCGAGGGAGACCTCTATCTGCACTTCTTCCAAGGCCTGAGCGGTTTCATCTGGCTGGACTTGTCCCAGAACCG
CCTGCACACCCTCCTGCCCCAAACCCTGCGCAACCTCCCCAACAGCCTACAGGTGCTCCGTCTCCGTGACAATTACC
TGGCCTTCTTTAAGTGGTCGAGCCTCCACTTCCTGCCCAAACTGGAAGTCCTCGACCTGGCACGAAATCAGCTGAAG
GCCCTGACCAATGGCAGCCTGCCTGCTGGCACCCGGCTCCGGAGGCTGGATGTCAGCTGCAACAGCATCAGCTTCGT
GGCCCCCGGCTTCTTTTCCAAGGCCAAGGAGCTGCGACAGCTCAACCTTAGCGCCAACGCCCTCAAGACAGTGGACC
ACTCCTGGTTTGGGCCCCTGGCGACTGCCCTCCAAATACTACATGTAAGCGCCAACCCTCTGCACTGCGCCTGTGCG
GCGGCCTTTATGGACTTCCTGCTCGAGGTGCAGGCTGCCGTCCCCGGTCTGCCCAGCCGGCTGAAGTCTGGCAGTCC
GCGCCAGCTCCAGGGCCTCACCATCTTTGCACAGGACCTGCCCCTCTCCCTGGATCAGGCCCTCTCCTGCCACTGTT
TCGCCCTCTCGCTCCTGGCTGTGGCTCTGGGCCTGGGTGTGCCCATGCTGCATCACCTCTGTGCCTGGGACCTCTGG
TACTGCTTCCACCTGTGCCTGGCCTGGCTTCCCTGCCGGGGGCGGCAAAGTCCGCGAGATGAGGATGCCCTGCCCTA
CGATGCCTTCGTGGTCTTCGACAAAACCCACAGCGCAGTGGCAGACTGGGTGTACAACGAGCTTCGGGGGCAGCTGG
AGGAGTCCCGTGGGCGCTGGGCACTCCGCCTGTGCCTGGAGGAACGCGACTGGCTGCCTGGCAAAACCCTCTTTGAG
AACCTGTGGGCCTCGGTCTATGGCAGCCCCAAGACGCTGTTTGTCCTGGCCCACACCGACCGGGTCAGTGGTCTCTT
GCGCCCCAGCTTCCTGCTCGCCCAGCAGCGCCTGCTGGAGGACCGCAAGGACGTCGTGGTGCTGGTGATCCTGAGCC
CTGACGGCCGCCGCTCCCGCTACGTGCGGCTCCGCCACCGCCTCTCCCCCCAGAGTGTCCTCCTCTGGCCCCACCAG
CCCAGTGGTCAGCGCAGCTTCTGGGCCCAGCTGGGCATGGCCCTGACCAGGGACAACCACCACTTCTATAACCGGAA
CTTCTGCCAGGGACCCACGGCCGAACTCGAG

TABLE 1AAQ
MOL1s reverse complement nucleotide sequence.

(SEQ ID NO:181)

CTCCAGTTCCCCCGTGCGTCCCTGGCAGAAGTTCCGGTTATACAAGTGCTGGTTGTCCCTCGTCAGCCCCATGCCCA
GCTGGGCCCAGAAGCTGCGCTGACCACTGGCCTGGTGGGGCCAGACGACCACACTCTGGCGGCAGAGCCGCTCGCGC
AGCCGCACGTACCCGGAGCGGCCGCCGTCAGGGCTCAGGATCACCAGCACCACGACGTCCTTGCGGTCCTCCAGCAG
GCGCTGCTGGGCCAGCAGGAAGCTGGCGCCCAAGAGACCACTGACCCCGTCCGTGTGCGCCAGCACAAACAGCGTCT
TGCGGCTGCCATAGACCGAGGCCCACAGGTTCTCAAAGACGGTTTTGCCAGGCAGCCAGTCGCGTTCCTCCAGGCAC
AGGCGCAGTGCCCAGCGCCCACGGCACTCCTCCAGCTGCCCCCGAAGCTCGTTGTACACCCAGTCTGCCACTGCGCT
CTGCGTTTTGTCGAAGACCACGAAGGCATCGTACGGCAGGGCATCCTCATCTCCCCCACTTTCCCGCCCCCGCCAGG
GAAGCCACGCCAGCCACAGGTGGAAGCAGTACCAGAGGTCCCAGCCACAGAGGTGATGCAGCATGCCCACACCCAGG
CCCAGAGCCACAGCCAGCAGCGAGAGCCCGAAACACTCCCACCAGAGGGCCTCATCCAGGCAGAGCCCCAGGTCCTG
TGCAAAGATCCTGAGCCCCTGGAGCTGGCCCGGACTGCCACACTTCACCCGGCTGGGCAGACCGGGCACGGCAGCCT
GCACCTCCAGCAGGAAGTCCATAAAGGCCGCCCCACAGGCGCAGTGCAGAGGGTTGGCGCTTACATCTACTATTTGC
AGGGCACTCGCCAGCGGCCCAAACCAGGAGTCGTCCACTGTCTTCAGCCCGTTGCCGCTAAGGTTGAGCTCTCGCAG
CTCCTTGGCCTTGGAAAACAACCCGGGGGCCACGAAGCTGATGCTGTTGCAGCTGACATCCAGCCTCCGGACCCGGG
TGCCAGCAGGCAGGCTCCCATTGGTCAGGGCCTTCAGCTGATTTCCTGCCAGGTCGAGGACTTCCAGTTTGGGCAGG
AAGTGGAGGCTCCACCACTTAAAGAAGGCCAGGTAATTGTCACCGACACGCAGCACCTGTACCCTCTTGGGGAGGTT
GCGCAGCGTTTCGGCCAGCAGGCTCTGCAGGCGGTTCTGGGACAAGTCCAGCCAGATCAAACCGCTCAGGCCTTGGA
AGAAGTGCAGATAGAGGTCTCCCTCGGCCCACATATGGCCCAGTGCATTGCCGCTGAAGTCCAGGCCCCGCACCGAC
GTACTGCAGAGCTGCTGGGACACTTGGCTGTGGATGTTCTTCTGGGCCAGGCTGAGGTGGCGCAGGGTGCGCAGGTG
ACCCACGAAGCTGAACTTGTGGCCCACGCCCTGCATGCCAAAGGGCTGGCTGTTGTAGCTGAGGTCCAGGGCCTCCA
GTCGCGGTAGCTCCGTGAATGAGTGCTCGTGGTAGAGCTCCACCTTATTGTGGGACAGCTCTACCACCTGCAGACCG
GTCAGCGCCAGGAACTGCGACCCATTGACTGCCTGCGAGATGCAGTTGTGGCTCAGGCGCAGGCACTGCAGGTGCGA
CAGCTCGGCAAACATCTCCGCCTGCACGGTCACCAGCTTGTTCCGTCACAGATCCAACGTGAACTTGAGGGTGCTGC
AGTTGGGCCTGAAGTCTTCAGAGCTGGGAGTGTCCACTGGGGCCCCACCAAGGTGGGCAGCCGCAGGCCAGACCTTC
TCCCCTCCATCTCCCTCCCCCATCGTCCCTGTCAGCTCCGAAGCTCCGCTGATGCGGTTGTCCGACAGGTCCACGTA
GCGCAGGCCAGGGAAGCCCCTGAAGATGCCGAGCTCGGCCTGCTTCATCAAGTTCATCTGCAGACGCAGAGTCTGGA
GCATCGGCAGGCGGCCCAGTGGCCGGAGCGTGGTCTCATCGAGTGAGCGCAAGAAGATCCCGTCCATGTCCAGCTCC
TTCAGGGCGACCAGGCTCCCGAAGGAAGGGGCCAGAGACAGGTGCGCAAAGGACACCCTCTTTTGGTAATTGAAGGA
CAGGTTAAGCTTGCGCACCTGTGTTAGGCCCTGGAAGGCCTTGGTTTTAGTGATGCATTTGTAGAGGAAGTTCTGAC
TCAGGTCCAGCACTCGGAGGTTTCCCAGCCCACGCAACCAACTGGCATTCAGCCACGAGAGACAACTGTCCTTCAAC
ACCAGGCCTTCAAGACGGCTCACGTCGCTCAAGGTATCGGGATGTAGCTGGGGGAAGTGACGAGGCCACTCCATGCA
GGGGTTGGGAGCGTGGTCGCAGCGGCGCCAATTTCCGCCCACATCGAGCACACGCAGGGCGGTCAGATTGGCCAGGT
CCTCACGCGCCAGTTTGACGATGCCGTTGTAGGACAACAGCAGATACTCCAGGCTGGAAGGCAGGTTGCGGGGCACC
ACAGTGAGGTTGTTGTACTTGAGTGACAGGTGGGTGACCTTGCCCAGGCCAAGGAGGGCACCCCCGGCCACCTCCAG
TGCCTCCCTGCAGGCGTTCTTGTAATAACAGTTGCCGTCCATGAATAGGAAGCGCAGGCCATGCAGCCCGGCGAGGC
TGGCAGAGTCTAGCATCACGATGTTGGTATGGCTGAGCGACAGGGATATGAGGGATTTGGGCAGCGCAGGCACAGTC
ATGATGTTGTTGTAGCTCAGGTTTAGCTCTTCCAGGGTGGGCACAGCCAAGAACCTGCTGGGCTCGATGGTCATGTG
GCAGGGCAAGTGCATGGGGCTGAGGCCAACCGGCGCGCACTTCCACTTGAGGTTGAGATGCCGCAGGCTGGCCAGGT
GGGCAAAGTCAGAATCATGGACCTGCTCGATGCGGTTGGAGGACAAGGAAAGGCTGGTGACATTGCCACGCGCTGCT
GCCATGGAGAAGTGGGGCACAGACTTCAGGAACACCCAGTTGCAGTTCACCAGCCCGTGGGGCTCGAGCTCACAGGG
TAGGAAGGCAGGCAAGGTACCCAGCGCCAGCGTCATCCCCAGCATGATGCCCTGCACCAGGACAGACAGCGGGTGCA
GGGCGCTCCCCCAGAAACCCATGGTGGATCC

A disclosed encoded MOL1s protein has 1037 amino acid residues, referred to as the MOL1s protein. The disclosed MOL1s polypeptide sequence is presented in Table 1AAR using the one-letter amino acid code.

TABLE 1AAR
Encoded MOL1s protein sequence.

(SEQ ID NO:171)

GSTMCFCRSALHPLSLLVQAIMLAMTLALGTLPAFLPCELQPHGLVNCNWLFLKSVPHFSMAAPRGNVTSLSLSSNR
IHHLHDSDFAHLPSLRHLNLKWNCPPVGLSPMHFPCHMTIEPSTFLAVPTLEELNLSYNNIMTVPALPKSLISLSLS
HTNILMLDSASLAGLHALRFLFMDGNCYYKNFCRQALEVAPGALLGLGNLTHLSLKYNNLTVVPRNLPSSLEYLLLS
YNRIVKLAPEDLANLTALRVLDVGGNCRRCDHAPNPCMECPRHFPQLHPDTFSHLSRLEGLVLKDSSLSWLNASWFR
GLGNLRVLDLSENFLYKCITKTKAFQGLTQLRKLNLSFNYQKRVSFAHLSLAPSFGSLVALKELDMHGIFFRSLDET
TLRPLARLPMLQTLRLQMNFINQAQLGIFRAFPGLRYVDLSDNRTSGASELTATMGEADGGEKVWLQPGDLAPAPVD
TPSSEDFRPNCSTLNFTLDLSRNNLVTVQPEMFAQLSHLQCLRLSHNCISQAVNGSQFLPLTGLQVLDLSHNKLDLY
HEHSFTELPRLEALDLSYNSQPFGMQGVGHNFSFVAHLRTLRHLSLAHNNIHSQVSQQLCSTSLPALDFSGNALGHM
WAEGDLYLHFFQGLSGLIWLDLSQNRLHTLLPQTLRNLPKSLQVLRLRDNYLAFFKWWSLHFLPKLEVLDLAGNQLK
ALTNGSLPAGTRLRRLDVSCNSISFVAPGFFSKAKELRELNLSANALKTVDHSWFGPLASALQILDVSANPLHCACG
AAFMDFLLEVQAAVPGLPSRVKCGSPGQLQGLSIFAQDLRLCLDEALSWDCFALSLLAVALGLGVPMLHHLCGWDLW
YCFHLCLAWLPWRCRQSGRDEDALPYDAFVVFDKTQSAVADWVYNELRGQLEECRGRWALRLCLEERDWLPGKTLFE
NLWASVYGSRKTLFVLAHTDRVSGLLRASFLLAQQRLLEDRKDVVVLVILSPDCRRSRYVRLRQRLCRQSVLLWPHQ
PSGQRSFWAQLGMALTRDNHHFYNRNFCQGPTAELE

MOL1t

A disclosed Toll receptor 9-like nucleic acid of 3111 nucleotides, MOL1t, is shown in Table 1AAS. The disclosed MOL1t open reading frame (“ORF”) begins at the GGA initiation codon at nucleotides 1-3, shown in bold in Table 1AAS. The encoded polypeptide is alternatively referred to herein as MOL1t or as 255304783. The disclosed MOL1t ORF terminates at a GAG codon at nucleotides 3109-3111. As shown in Table 1AAS the start and stop codons are in bold letters. Because MOL1t does not start or stop at traditional initiation or termination codons, MOL1t could be a partial open reading frame which extends further in the 5′ and/or 3′ directions.

TABLE 1AAS
MOL1t nucleotide sequence.

(SEQ ID NO:172)

GGATCCACCATGGGTTTCTGCCGCAGCGCCCTGCACCCCCTGTCTCTCCTGGTGCACGCCATCATGCTGGCCATGAC
CCTGGCCCTGGCTACCTTCCCTGCCTTCCTACCCTGTGACCTCCAGCCCCACGGCCTGCTGAACTGCAACTGGCTGT
TCCTGAAGTCTGTGCCCCACTTCTCCATGGCAGCACCCCCTGGCAATGTCACCAGCCTTTCCTTCTCCTCCAACCGC
ATCCACCACCTCCATGATTCTGACTTTGCCCACCTGCCCAGCCTGCGGCATCTCAACCTCAACTGGAACTGCCCGCC
GGTTGGCCTCAGCCCCATGCACTTCCCCTGCCACATGACCATCGAGCCCAGCACCTTCTTGGCTGTGCCCATCCTGG
AAGAGCTAAACCTGAGCTACAACAACATCATGACTGTGCCTGCGCTGCCCAAATCCCTCATATCCCTGTCCCTCAGC
CATACCAACATCCTGATGCTAGACTCTGCCAGCCTCGCCGGCCTGCATGCCCTGCGCTTCCTATTCATCGACGGCAA
CTGTTATTACAAGAACCCCTGCACGCAGGCACTGGAGGTGGCCCCGCGTGCCCTCCTTGGCCTGGCCAACCTCACCC
ACCTGTCACTCAAGTACAACAACCTCACTGTGGTGCCCCCCAACCTGCCTTCCAGCCTGGAGTATCTGCTGTTGTCC
TACAACCGCATCGTCAAACTGGCGCCTGAGGACCTGCCCAATCTGACCGCCCTCCGTGTGCTCGATCTGGGCGGAAA
TTGCCGCCGCTGCGACCACGCTCCCAACCCCTGCATGGAGTGCCCTCGTCACTTCCCCCAGCTACATCCCGATACCT
TCAGCCACCTGAGCCGTCTTGAACGCCTGGTGTTCAAGGACAGTTCTCTCTCCTGGCTGAATGCCAGTTGGTTCCGT
GGGCTGGGAAACCTCCGAGTGCTGGACCTGAGTGAGAACTTCCTCTACAAATGCATCACTAAAACCAAGGCCTTCCA
GGGCCTAACACAGCTGCGCAAGCTTAACCTGTCCTTCAATTACCAAAAGAGCCTGTCCTTTGCCCACCTGTCTCTGG
CCCCTTCCTTCGGGAGCCTGGTCGCCCTGAAGGAGCTCGACATGCACCGCATCTTCTTCCGCTCACTCGATGACACC
ACGCTCCGGCCACTGCCCCCCCTCCCCATGCTCCAGACTCTCCGTCTGCAGATGAACTTCATCAACCAGGCCCAGCT
CGGCATCTTCAGGGCCTTCCCTGGCCTGCGCTACGTGCACCTCTCGGACAACCGCATCAGCGGAGCTTCGGAGCTGA
CAGCCACCATGCGGGAGGCACATGGAGGGGAGAAGGTCTGGCTGCAGCCTGGGCACCTTGCTCCGGCCCCAGTGGAC
ACTCCCAGCTCTGAAGACTTCAGGCCCAACTGCAGCACCCTCAACTTCACCTTGCATCTGTCACGGAACAACCTGGT
GACCCTGCAGCCGCAGATGTTTGCCCAGCTCTCGCACCTGCAGTCCCTGCGCCTGAGCCACAACTGCATCTCGCAGG
CAGTCAATGGCTCCCAGTTCCTGCCGCTCACCGGTCTCCAGGTGCTAGACCTGTCCCACAATAACCTCGACCTCTAC
CACGAGCACTCATTCACGGAGCTACCACCACTGGAGGCCCTGGACCTCAGCTACAACAGCCAGCCCTTTGGCATGCA
GGGCGTGGGCCACAACTTCAGCTTCGTGGCTCACCTGCGCACCCTGCGCCACCTCAGCCTCCCCCACAACAACATCC
ACAGCCAAGTGTCCCAGCAGCTCTGCAGTACGTCGCTCCGGGCCCTGGACTTCAGCGCCAATGCACTGGGCCATATG
TGGGCCGAGGGAGACCTCTATCTGCACTTCTTCCAAGGCCTGAGCGGTTTGATCTGGCTGGACTTGTCCCAGAACCG
CCTGCACACCCTCCTGCCCCAAACCCTGCGCAACCTCCCCAAGAGCCTACAGCTGCTGCCTCTCCCTGACAATTACC
TGGCCTTCTTTAAGTGGTGGAGCCTCCACTTCCTGCCCAAACTGGAAGTCCTCGACCTGGCAGGAAACCAGCTGAAG
GCCCTGACCAATGGCAGCCTCCCTGCTGGCACCCGGCTCCGGAGGCTGGATGTCAGCTGCAACAGCATCAGCTTCGT
GGCCCCCCGCTTCTTTTCCAAGGCCAAGGAGCTGCGACAGCTCAACCTTAGCGCCAACGCCCTCAAGACAGTGGACC
ACTCCTGGTTTCGGCCCCTGGCGAGTGCCCTGCAAATACTAGATGTAAGCGCCCACCCTCTGCACTCCGCCTGTGGG
GCGGCCTTTATGGACTTCCTGCTCGAGGTGCAGCCTGCCGTGCCCGCTCCGCCCAGCCGGGTGAAGTGTGGCACTCC
GGGCCAGCTCCAGGGCCTCAGCATCTTTGCACAGGACCTGCGCCTCTGCCTGGATGAGGCCCTCTCCTGGGACTCTT
TCGCCCTCTCGCTGCTGGCTGTGGCTCTGGGCCTGGGTGTGCCCATCCTGCATCACCTCTGTGGCTGGGACCTCTGG
TACTGCTTCCACCTGTGCCTGGCCTGGCTTCCCTGCCGGGGGCGGCAAAGTGGGCGACATGAGGATGCCCTGCCCTA
CGATCCCTTCGTCGTCTTCGACAAAACGCAGAGCCCAGTCGCAGACTGGGTGTACAACGAGCTTCGGGGGCAGCTGG
AGGAGTGCCGTGGGCGCTGGGCACTCCGCCTGTGCCTGGAGGAACGCCACTGCCTGCCTGGCAAAACCCTCTTTGAG
AACCTGTGGCCCTCGGTCTATCGCAGCCGCAAGACCCTGTTTGTGCTCGCCCACACGGACCGGGTCAGTGGTCTCTT
GCGCGCCAGCTTCCTGCTGGCCCAGCAGCGCCTGCTGGAGGACCGCAAGGACGTCGTGGTGCTGGTGATCCTGAGCC
CTGACCGCCCCCCCTCCCGCTACCTGCGGCTGCGCCAGCGCCTCTGCCGCCAGAGTCTCCTCCTCTCGCCCCACCAG
CCCAGTGGTCAGCGCAGCTTCTGGGCCCACCTCCGCATGGCCCTGACCAGGGACAACCACCACTTCTATAACCCGAA
CTTCTGCCAGCGACCCACCGCCGAACTCGAG

A disclosed encoded MOL1t protein has 1037 amino acid residues, referred to as the MOL1t protein. The disclosed MOL1t polypeptide sequence is presented in Table 1AAT using the one-letter amino acid code.

TABLE 1AAT
Encoded MOL1t protein sequence.

(SEQ ID NO:173)

GSTMGFCRSALHPLSLLVQAIMLAMTLALGTLPAFLPCELQPHCLVNCNWLFLKSVPHFSMAAPRGNVTSLSLSSNR
IHHLHDSDFAHLPSLRHLNLKWNCPPVCLSPMHFPCHMTIEPSTFLAVPILEELNLSYNNIMTVPALPKSLISLSLS
HTNILMLDSASLAGLHALRFLFMDCNCYYKNPCRQALEVAPGALLGLGNLTHLSLKYNNLTVVPRNLPSSLEYLLLS
YNRIVKLAPEDLANLTALRVLDVGGNCRRCDHAPNPCMECPRHFPQLHPDTFSHLSRLEGLVLKDSSLSWLNASWFR
CLCNLRVLDLSENFLYKCITKTKAFQGLTQLRKLNLSFNYQKRVSFAHLSLAPSFGSLVALKELDMHGIFFRSLDET
TLRPLARLPMLQTLRLQMNFINQAQLGIFRAFFGLRYVDLSDNRISGASELTATMGEADGGEKVWLQPGDLAPAPVD
TPSSEDFRPNCSTLNFTLDLSRNNLVTVQPEMFAQLSHLQCLRLSHNCISQAVNGSQFLPLTGLQVLDLSHNKLDLY
HEHSFTELPRLEALDLSYNSQPFGMQGVGHNFSFVAHLRTLRHLSLAHNNIHSQVSQQLCSTSLRALDFSGNALGHM
WAEGDLYLHFFQCLSGLIWLDLSQNRLHTLLPQTLRNLPKSLQVLRLRDNYLAFFKWWSLHFLPKLEVLDLAGNQLK
ALTNGSLPAGTRLRRLDVSCNSISFVAFGFFSKAKELRELNLSANALKTVDHSWFCPLASALQILDVSAHFLHCACG
AAFMDFLLEVQAAVPGPPSRVKCGSPGQLQGLSIFAQDLRLCLDEALSWDCFALSLLAVALGLGVPMLHHLCGWDLW
YCFHLCLAWLPWRGRQSGRDEDALPYDAFVVFDKTQSAVADWVYNELRGQLEECRGRWALRLCLEERDWLPGKTLFE
NLWASVYGSRKTLFVLAHTDRVSGLLPASFLLAQQRLLEDRKDVVVLVTLSPDGRRSRYVRLRQRLCRQSVLLWPHQ
PSGQRSFWAQLGMALTRIDHHFYNRNFCQGPTAELE

MOL1u

A disclosed Toll receptor 9-like nucleic acid of 3111 nucleotides, MOL1u, is shown in Table 1AAU. The disclosed MOL1u open reading frame (“ORF”) begins at the GGA initiation codon at nucleotides 1-3, shown in bold in Table 1AAU. The encoded polypeptide is alternatively referred to herein as MOL1u or as 255341675. The disclosed MOL1u ORF terminates at a GAG codon at nucleotides 3109-3111. As shown in Table 1AAU the start and stop codons are in bold letters. Because MOL1u does not start or stop at traditional initiation or termination codons, MOL1u could be a partial open reading frame which extends further in the 5′ and/or 3′ directions.

TABLE 1AAU
MOL1u nucleotide sequence.

(SEQ ID NO:174)

GGATCCACCATCGGTTTCTGCCGCAGCGCCCTGCACCCGCTGTCTCTCCTGGTGCAGCCCATCATCCTGGCCATGAC
CCTGCCCCTGGGTACCTTGCCTGCCTTCCTACCCTGTGAGCTCCAGCCCCACCGCCTGGTGAACTGCAACTGGCTGT
TCCTGAAGTCTGTGCCCCACTTCTCTATGCCAGCACCCCGTGGCAATGTCACCAGCCTTTCCTTGTCCTCCAACCGC
ATCCACCACCTCCATGATTCTGACTTTGCCCACCTGCCCAGCCTGCGGCATCTCAACCTCAAGTCGAACTGCCCCCC
GGTTGGCCTCAGCCCCATGCACTTCCCCTGCCACATGACCATCGAGCCCAGCACCTTCTTCCCTGTGCCCACCCTGC
AAGAGCTAAACCTGACCTACAACAACATCATGACTGTGCCTGCGCTGCCCAAATCCCTCATATCCCTGTCCCTCAGC
CATACCAACATCCTGATGCTAGACTCTCCCAGCCTCGCCGGCCTGCATGCCCTGCCCTTCCTATTCATGGACGGCAA
CTGTTATTACAAGAACCCCTGCAGGCAGGCACTCGAGGTGGCCCCGGCTCCCCTCCTTGGCCTGGGCAACCTCACCC
ACCTGTCACTCAAGTACAACAACCTCACTCTGGTGCCCCCCAACCTGCCTTCCAGCCTGGAGTATCTGCTGTTGTCC
TACAACCGCATCGTCAACTGGCGCCTGAGGACCTGGCCAATCTGACCGCCCTGCGTGTCCTCGATGTGGGCGGAAAA
TTGCCCCCGCTGCGACCACGCTCCCAACCCCTGCATGGAGTGCCCTCGTCACTTCCCCCAGCTACATCCCGATACCT
TCAGCCACCTCAGCCGTCTTGAAGGCCTGGTGTTGAAGGACAGTTCTCTCTCCTGGCTCAATGCCAGTTGCTTCCGT
GGCCTGGGAPACCTCCGAGTGCTGGACCTGACTCAGAACTTCCTCTACAAATGCATCACTAAAACCAAGGCCTTCCA
GCCCCTAACACAGCTCCCCAAGCTTAACCTGTCCTTCAATTACCAAAAGAGGGTGTCCTTTGCCCACCTGTCTCTGG
CCCCTTCCTTCGCGACCCTGGTCGCCCTGAAGGAGCTGGACATGCACGGCATCTTCTTCCGCTCACTCGATGAGACC
ACCCTCCGGCCACTGCCCCGCCTGCCCATGCTCCAGACTCTGCGTCTGCAGATGAACTTCATCAACCAGGCCCAGCT
CCGCATCTTCAGGCCCTTCCCTGGCCTGCCCTACGTGGACCTGTCGGACAACCGCATCAGCGGAGCTTCGGAGCTCA
CAGCCACCATGGGGGAGGCAGATGGAGGCCAGAAGGTCTGGCTCCAGCCTGCGGACCTTCCTCCGGCCCCAGTGGAC
ACTCCCAGCTCTGAGACTTCAGGCCCAACTGCAGCACCCTCAACTTCACCTTGGATCTGTCACGGAACAAACCTGGT
GACCCTGCACCCGGAGATGTTTGCCCAGCTCTCGCACCTGCAGTGCCTGCCCCTGACCCACAACTGCATCTCGCAGG
CAGTCAATGGCTCCCAGTTCCTGCCGCTGACCGGTCTGCACGTGCTAGACCTGTCCCACATTAAGCTGGACCTCTAC
CACGAGCACTCATTCACCGAGCTACCACGACTGGAGGCCCTGGACCTCAGCTACCACAGCCAGCCCTTTGGCATGCA
GGGCGTGGGCCACAACTTCAGCTTCGTCGCTCACCTGCCCACCCTCCGCCACCTCAGCCTCGCCCACAACAACATCC
ACACCCAACTCTCCCACCAGCTCTGCAGTACGTCGCTGCCGGCCCTGGACTTCAGCGGCAATGCACTGGGCCATATG
TGGGCCCAGGGAGACCTCTATCTGCACTTCTTCCAAGGCCTGAGCGGTTTGATCTGGCTGCACTTGTCCCAGAACCG
CCTGCACACCCTCCTGCCCCAAACCCTGCCCAACCTCCCCAAGAGCCTACAGGTGCTGCCTCTCCGTGACAATTACC
TGCCCTTCTTTAAGTGGTGGAGCCTCCACTTCCTGCCCAAACTGGAAGTCCTCCACCTGGCAGGAAACCAGCTGAAG
GCCCTGACCAATGCCAGCCTGCCTGCTGGCACCCGGCTCCGGAGGCTGGATGTCAGCTCCAACAGCATCAGCTTCGT
GGCCCCCGGCTTCTTTTCCAAGGCCAAGGAGCTGCGAGAGCTCAACCTTACCGCCAACGCCCTCAAGACAGTGCACC
ACTCCTGGTTTGGGCCCCTGGCCACTGCCCTGCAAGTACTAGATGTAACCGCCAACCCTCTGCACTGCGCCTCTGGG
GCGGCCTTTATCGACTTCCTGCTGGAGGTCCACGCTGCCGTGCCCGGTCCGCCCAGCCGGGTGAAGTGTGCCAGTCC
GCGCCAGCTCCAGGCCCTCAGCATCTTTGCACAGGACCTGCCCCTCTGCCTGGATGAGGCCCTCTCCTGGCACTGTT
TCGCCCTCTCCCTGCTGGCTGTGGCTCTGGGCCTGGCTGTCCCCATGCTGCATCACCTCTGTGGCTGGGACCTCTGG
TACTGCTTCCACCTGTGCCTCGCCTGGCTTCCCTGGCGGGGCCGGCAAAGTGGGCGAGATGAGGATGCCCTCCCCTA
CCATGCCTTCGTCGTCTTCGACAAAACCCAGAGCGCAGTGGCAGACTGGGTGTACAACGAGCTTCGCGGGCACCTCG
AGGAGTGCCGTGCGCGCTGGGCACTCCCCCTGTGCCTGGAGGAACGCCACTGGCTGCCTGGCAAAACCCTCTTTGAG
AACCTGTGGGCCTCGGTCTATGGCAGCCGCAAGACCCTGTTTGTGCTCGCCCACACGGACCGGGTCAGTGGTCTCTT
GCGCGCCAGCTTCCTGCTGGCCCAGCAGCCCCTGCTCCAGGACCGCAAGGACGTCGTGGTGCTGGTGATCCTGAGCC
CTGACGGCCGCCGCTCCCCCTACGTGCGGCTCCGCCAGCGCCTCTGCCGCCAGAGTGTCCTCCTCTGGCCCCACCAG
CCCACTGGTCAGCGCAGCTTCTGGGCCCAGCTGGGCATGGCCCTGACCAGGGACAACCACCACTTCTATAACCGGAA
CTTCTGCCAGGGACCCACCGCCGAACTCGAG

TABLE 1AAV
MOL1u reverse complement nucleotide sequence.

(SEQ ID NO:182)

CTCGAGTTCGGCCGTGGGTCCCTGGCAGAAGTTCCGCTTATAGAAGTGGTCGTTGTCCCTGGTCAGGGCCATGCCCA
GCTCGGCCCAGAAGCTGCGCTGACCACTGGCCTGGTGGGGCCAGAGGAGCACACTCTGGCGGCAGAGGCGCTGGCGC
ACCCGCACGTAGCGGCAGCCGCGGCCGTCAGGGCTCAGGATCACCAGCACCACCACGTCCTTGCGGTCCTCCAGCAG
GCGCTCCTGGGCCAGCAGGAACCTGGCGCGCAAGAGACCACTGACCCGGTCCGTGTGGGCCAGCACAAACAGCGTCT
TGCGGCTGCCATAGACCCAGGCCCACAGGTTCTCAAAGAGGCTTTTGCCAGGCAGCCAGTCGCGTTCCTCCAGGCAC
AGGCGGAGTGCCCAGCGCCCACGGCACTCCTCCAGCTGCCCCCGAAGCTCGTTGTACACCCAGTCTGCCACTGCGCT
CTCCGTTTTGTCCAAGACCACGAACGCATCGTAGGCCAGCGCATCCTCATCTCGCCCACTTTGCCGCCCCCGCCAGG
GAAGCCAGGCCAGGCACAGGTGGAAGCAGTACCAGAGGTCCCAGCCACAGAGGTGATGCAGCATCGCCACACCCACG
CCCAGAGCCACAGCCAGCAGCGAGAGGCCGAAACAGTCCCAGGAGAGGGCCTCATCCAGGCAGAGGCGCAGGTCCTG
TGCAAAGATGCTGAGGCCCTGGAGCTGCCCCGCACTGCCACACTTCACCCGCCTCGCCCGACCGGGCACGGCAGCCT
GCACCTCCAGCAGGAAGTCCATAAAGGCCGCCCCACAGGCGCAGTGCAGAGGGTTGGCGCTTACATCTAGTATTTGC
AGGGCACTCGCCAGGGGCCCAAACCAGGAGTGGTCCACTGTCTTGAGGGCGTTGGCGCTAAGGTTGAGCTCTCGCAG
CTCCTTGCCCTTGGAAAACAAGCCGGCGGCCACGAAGCTGATGCTGTTGCAGCTCACATCCAGCCTCCGGAGCCGGG
TGCCAGCAGGCAGGCTGCCATTGGTCAGGGCCTTCACCTGGTTTCCTGCCAGGTCGAGGACTTCCAGTTTGGGCAGG
AACTCGAGGCTCCACCACTTAAAGAAGGCCAGGTAATTGTCACGGAGACGCAGCACCTGTAGGCTCTTGCGGAGGTT
GCGCAGGCTTTGGGGCACGACGGTGTCCAGCCGGTTCTGGGACAAGTCCAGCCAGATCAAACCGCTCAGGCCTTGGA
AGAAGTGCACATAGAGGTCTCCCTCGGCCCACATATGGCCCAGTGCATTGCCCCTGAACTCCAGGGCCCGCAGCGAC
GTACTGCAGAGCTGCTGCGACACTTCGCTGTGGATGTTGTTGTGGGCCAGGCTGAGGTGGCGCAGGGTGCGCAGGTG
AGCCACGAAGCTGAACTTGTGGCCCACGCCCTGCATGCCAAAGGGCTGGCTGTTGTAGCTGAGGTCCAGGGCCTCCA
GTCGTCGTAGCTCCGTGAATGAGTCCTCGTGGTACAGGTCCACCTTATTGTGGGACAGGTCTAGCACCTCCAGACCG
GTCAGCGGCAGGAACTGGGAGCCATTGACTGCCTGCGAGATGCAGTTGTGGCTCAGGCGCAGGCACTGCAGGTCCGA
GAGCTGGGCAAACATCTCCGCCTGCACGGTCACCACGTTGTTCCGTGACAGATCCAAGGTGAAGTTGAGGGTGCTGC
AGTTGGGCCTGAAGTCTTCACAGCTGCGAGTGTCCACTGGGGCCGGAGCAAGGTCCCCAGGCTGCAGCCAGACCTTC
TCCCCTCCATCTGCCTCCCCCATGGTCGCTGTCAGCTCCGAACCTCCGCTGATCCGGTTGTCCGACACGTCCACGTA
GCGCAGGCCACGGAAGGCCCTGAAGATGCCGAGCTCGGCCTGGTTGATCAAGTTCATCTGCACACGCAGAGTCTGCA
GCATGGGCAGGCGGGCCAGTGGCCGGAGCGTGGTCTCATCGAGTGAGCGGAAGAACATGCCGTGCATGTCCAGCTCC
TTCAGGGCGACCAGGCTCCCGAAGGAAGGGGCCAGAGACAGGTGGGCAAAGGACACCCTCTTTTGGTAATTGAAGGA
CAGGTTAAGCTTCCGCAGCTGTGTTAGGCCCTGCAAGGCCTTGGTTTTAGTGATGCATTTGTAGAGGAAGTTCTCAC
TCAGGTCCAGCACTCGGAGCTTTCCCAGCCCACGGAACCAACTGGCATTCAGCCAGCAGAGAGAACTGTCCTTCAAC
ACCAGGCCTTCAAGACGGCTCACGTCGCTGAAGGTATCCGGATGTACCTGGGGGAAGTGACGAGGGCACTCCATGCA
GGGGTTGGGAGCGTCGTCGCAGCCCCGGCAATTTCCGCCCACATCGAGCACACGCAGGGCGGTCACATTGGCCAGGT
CCTCAGGCGCCAGTTTGACGATCCGGTTGTAGGACAACAGCAGATACTCCAGGCTGGAAGCCAGGTTGCGGCGCACC
ACAGTCACGTTCTTGTACTTGAGTGACAGGTGGGTGAGGTTGCCCAGGCCAAGCAGGGCACCCGGGGCCACCTCCAG
TGCCTGCCTGCAGCGGTTCTTGTAATAACAGTTGCCGTCCATGAATAGGAAGCGCAGGGCATGCAGGCCGGCGAGGC
TGGCAGAGTCTAGCATCAGGATGTTGGTATGGCTGAGGGACAGGGATATGAGGGATTTGGGCAGCGCAGGCACAGTC
ATGATGTTCTTGTAGCTCAGGTTTACCTCTTCCAGGGTGGGCACAGCCAAGAAGGTGCTGGGCTCGATGGTCATGTC
GCACGGGAAGTGCATGCGGCTGAGGCCAACCGCCGGGCAGTTCCACTTGAGGTTGAGATGCCGCAGGCTGGGCAGGT
GGGCAAAGTCAGAATCATGGAGGTCGTGGATGCGGTTGGAGGACAACGAAAGGCTGGTGACATTGCCACGGGGTGCT
GCCATAGAGAAGTGGGGCACAGACTTCAGGAACAGCCAGTTGCAGTTCACCAGGCCGTCCCGCTGGAGCTCACAGGG
TAGGAAGGCAGGCAAGGTACCCAGGGCCAGGGTCATGGCCACCATCATGCCCTGCACCAGGAGAGACAGCGGGTGCA
GGGCGCTGCCGCACAAACCCATCCTCGATCC

A disclosed encoded MOL1u protein has 1037 amino acid residues, referred to as the MOL1u protein. The disclosed MOL1u polypeptide sequence is presented in Table 1AAW using the one-letter amino acid code.

TABLE 1AAW
Encoded MOL1u protein sequence.

(SEQ ID NO:175)

GSTMGFCRSALHPLSLLVQAIMLANTLALGTLPAFLPCELQPHGLVNCNWLFLKSVPHFSMAAPRCNVTSLSLSSNR
IHHLHDSDFAHLPSLRHLNLKWNCPPVCLSPMHFPCHMTIEPSTFLAVPTLEELNLSYNNTMTVPALPKSLISLSLS
HTNILMLDSASLAGLHALRFLFMDGNCYYKNPCRQALEVAPGALLCLGNLTHLSLKYNNLTVVPRNLPSSLEYLLLS
YNRTVKLAPEDLANLTALRVLDVCGNCRRCDHAPNPCMECPRHFPQLHPDTFSHLSRLEGLVLKDSSLSWLNASWFR
GLGNLRVLDLSENFLYKCITKTKAFQGLTQLRKLNLSFNYQKRVSFAHLSLAPSFGSLVALKELDMHGIFFRSLDET
TLRPLARLPMLQTLRLQMNFTNQAQLGIFRAFPGLRYVITSDNRTSGASELTATMGEADGGEKVWLQPGDLAPAPVD
TPSSEDFRFNCSTLNFTLDLSRNNLVTVQPEMFAQLSHLQCLRLSHNCISQAVNGSQFLPLTGLQVLDLSHNKLDLY
HEHSFTELPRLEALDLSYNSQPFGMQGVGHNFSEVAHLRTLRHLSLAHNNIHSQVSQQLCSTSLPALDFSGNALGHM
WAEGDLYLHFFQGLSGLIWLDLSQNRLHTLLPQTLRNLPKSLQVLRLRDNYLAFPKWWSLHFLPKLEVLDLAGNQLK
ALTNGSLPAGTRLRRLDVSCNSISEVAPGFFSKAKELRELNLSANALKTVDHSWFGPLASALQILDVSANPLHCACG
AAFMDFLLEVQAAVPGPPSRVKCGSPGQLQGLSTFAQDLRLCLDEALSWDCFALSLLAVALCLGVPMLHHLCGWDLW
YCFHLCLAWLPWRGRQSGRDEDALPYDAFVTFDKTQSAVADWVYNELRGQLEECRGRWALRLCLEERDWLPGKTLFE
NLWASVYGSRKTLFVLAHTDRVSGLLRASFLLAQQRLLEDRKDVVVLVILSPDGRRSRYVRLRQRLCRQSVLLWPHQ
PSGQRSFWAQLGMALTRDNHHFYNRNFCQGPTAELE

The amino acid sequence of MOL1a also had high homology to other proteins as shown in table 1AA.

TABLE 1AA
BLAST results for MOL1a

| Gene Index/ | Protein/ | Length | Identity | Positives | |
|---|---|---|---|---|---|
| Identifier | Organism | (aa) | (%) | (%) | Expect |
| Patp: W86365 | DNAX toll-like | 336 | 335/336 | 335/336 | 0.0 |
| | receptor DTLR- | | (99%) | (99%) | |
| | 10 [Homo | | | | |
| | sapiens] | | | | |
| gi\|8394456\|ref\|NP_{—} | toll-like | 1032 | 960/1014 | 960/1014 | 0.0 |
| 059138.1\| | receptor 9 | | (94%) | (94%) | |
| | [Homo sapiens] | | | | |
| gi\|13648665\|ref\|XP_{—} | toll-like | 1014 | 960/1014 | 960/1014 | 0.0 |
| 003236.2\| | receptor 9 | | (94%) | (94%) | |
| | [Homo sapiens] | | | | |
| gi\|8099654\|gb\|AAF72190.1\| | toll-like | 975 | 921/975 | 921/975 | 0.0 |
| AF259263_1 | receptor 9 form | | (94%) | (94%) | |
| | B [Homo | | | | |
| | sapiens] | | | | |
| gi\|13507173\|gb\|AAK28488.1\| | toll-like | 1032 | 720/1015 | 799/1015 | 0.0 |
| AF314224_1 | receptor 9 [Mus | | (70%) | (77%) | |
| | musculus] | | | | |

A ClustalW analysis comparing disclosed proteins of the invention with related OR protein sequences is given in Table 1BB, with MOL1a shown on line 1.

In the ClustalW alignment of the MOL1a protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function. Residue differences between any MOLX variant sequences herein are written to show the residue in the “a” variant and the residue position with respect to the “a” variant. MOL residues in all following sequence alignments that differ between the individual MOL variants are highlighted with a box and marked with the (o) symbol above the variant residue in all alignments herein.

[image]

[image]

[image]

The interleukin-1 (IL-1) receptor/Toll-like receptor (TLR) superfamily is a recently defined and expanding group of receptors that participate in host responses to injury and infection. The superfamily is defined by the Toll/IL-1 receptor (TIR) domain, which occurs in the cytosolic region of family members, and is further subdivided into two groups based on homology to either the Type I IL-1 receptor or Drosophila Toll receptor extracellular domain. The former group includes the receptor for the important Th1 cytokine IL-18, and T1/ST2, which may have a role in Th2 cell function. The latter group includes six mammalian TLRs, including TLR2 and TLR4, that largely mediate the host response to gram-positive and gram-negative bacteria, respectively. Whether bacterial products are actual ligands for TLRs, or whether they generate ligands via as yet unidentified pattern recognition receptors, has yet to be determined. Signaling pathways activated via the TIR domain trigger the activation of downstream kinases, and transcription factors such as NF-kappaB, and involve the adaptor protein MyD88, which itself contains a TIR domain.

As our primary interface with the environment, the skin is constantly subjected to injury and invasion by pathogens. The fundamental force driving the evolution of the immune system has been the need to protect the host against overwhelming infection. The ability of T and B cells to recombine antigen receptor genes during development provides an efficient, flexible, and powerful immune system with nearly unlimited specificity for antigen. The capacity to expand subsets of antigen-specific lymphocytes that become activated by environmental antigens (memory response) is termed “acquired” immunity. Immunologic memory, although a fundamental aspect of mammalian biology, is a relatively recent evolutionary event that permits organisms to live for years to decades. “Innate” immunity, mediated by genes that remain in germ line conformation and encode for proteins that recognize conserved structural patterns on microorganisms, is a much more ancient system of host defense. Defensins and other antimicrobial peptides, complement and opsonins, and endocytic receptors are all considered components of the innate immune system. None of these, however, are signal-transducing receptors. Most recently, a large family of cell surface receptors that mediate signaling through the NF-kappaB transcription factor has been identified. This family of proteins shares striking homology with plant and Drosophila genes that mediate innate immunity. In mammals, this family includes the type I interleukin-1 receptor, the interleukin-18 receptor, and a growing family of Toll-like receptors, two of which were recently identified as signal-transducing receptors for bacterial endotoxin. In this review, we discuss how interleukin-1 links the innate and acquired immune systems to provide synergistic host defense activities in skin.

In Drosophila the Toll protein is involved in establishment of dorso-ventral polarity in the embryo. In addition, members of the Toll family play a key role in innate antibacterial and antifungal immunity in insects as well as in mammals. These proteins are type-I transmembrane receptors that share an intracellular 200 residue domain with the interleukin-1 receptor (IL-1R), the Toll/IL-1R homologous region (TIR). The similarity between Toll-like receptors (LTRs) and IL-1R is not restricted to sequence homology since these proteins also share a similar signaling pathway. They both induce the activation of a Rel type transcription factor via an adaptor protein and a protein kinase. Interestingly, MyD88, a cytoplasmic adaptor protein found in mammals, contains a TIR domain associated to a DEATH domain (see IPR000488). Besides the mammalian and Drosophila proteins, a TIR domain is also found in a number of plant proteins implicated in host defense. As MyD88, these proteins are cytoplasmic. Site directed mutagenesis and deletion analysis have shown that the TIR domain is essential for Toll and IL-1R activities. Sequence analysis have revealed the presence of three highly conserved regions among the different members of the family: box 1 (FDAFISY), box 2 (GYKLC-RD-PG), and box 3 (a conserved W surrounded by basic residues). It has been proposed that boxes 1 and 2 are involved in the binding of proteins involved in signaling, whereas box 3 is primarily involved in directing localization of receptor, perhaps through interactions with cytoskeletal elements

Toll is a Drosophila gene essential for ontogenesis and antimicrobial resistance. Several hortologues of Toll have been identified and cloned in vertebrates, namely Toll-like receptors (TLR). Human TLR are a growing family of molecules involved in innate immunity. TLR are structurally characterized by a cytoplasmic Toll/interleukin-1R (TIR) domain and by extracellular leucine-rich repeats. TLR characterized so far activate the MyD88/IRAK signaling cascade, which bifurcates and leads to NF-kappaB and c-Jun/ATF2/TCF activation. Genetic, gene transfer, and dominant-negative approaches have involved TLR family members (TLR2 and TLR4) in lipopolysaccharide recognition and signaling. Accumulating evidence suggests that some TLR molecules are also involved in signaling receptor complexes that recognize components of gram-positive bacteria and mycobacteria. However, the definitive role of other TLR is still lacking. A systematic approach has been used to determine whether different human leukocyte populations selectively or specifically expressed TLR mRNA. Based on expression pattern, TLR can be classified as ubiquitous (TLRl), restricted (TLR2, TLR4, and TLR5), and specific (TLR3). Expression and regulation of distinct though overlapping ligand recognition patterns may underlie the existence of a numerous, seemingly redundant, TLR family. Alternately, the expression of a TLR in a single cell type may indicate a specific role for this molecule in a restricted setting.

The amino acids differences between the three MOL1 proteins are shown in Table 1H. Deletions are marked by a delta (Δ). The differences between the three proteins appear to be localized to a few distinct regions. Thus, these proteins may have similar functions, such as serving as olfactory or chemokine receptors.

Uses of the Compositions of the Invention

The above defined information for this invention suggests that this Toll Receptor-like protein may function as a member of a “Toll Receptor family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in pancreatic cancer, adenoma, and other cancers, Larsen syndrome, tachycardia, erythroderma, night blindness, long QT syndrome, brugada syndrome, heart block, cell-mediated immunity, and applications as a mediator in inflammation and/or other pathologies and disorders. For example, a cDNA encoding the Toll Receptor-like protein may be useful in gene therapy, and the Toll Receptor-like protein may be useful when administered to a subject in need thereof. By way of nonlimiting example, the compositions of the present invention will have efficacy for treatment of patients suffering from pancreatic cancer, adenoma, and other cancers, Larsen syndrome, tachycardia, erythroderma, night blindness, long QT syndrome, brugada syndrome, heart block, cell-mediated immunity, and applications as a mediator in inflammation. The novel nucleic acid encoding Toll Receptor-like protein, and the Toll Receptor-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL2

MOL2a

An additional murine GNC2 eIFK-like protein of the invention, referred to herein as MOL2a, is an Olfactory Receptor (“OR”)-like protein. The novel nucleic acid of 4989 nucleotides, (20466828_EXT1, SEQ ID NO:3) encoding a novel GNC2 eIFK-like protein is shown in Table 2A.

TABLE 2A
MOL2a Nucleotide Sequence

(SEQ ID NO:3)

ATGGCTGGGGGCCGTGGCCCCCCCGGGCCCGCCCCGGACGAGCCTCCGGAGAGCTACCCGCAACGACAGGACCACGA
GCTACAGGCCCTGGAGGCCATCTACGGCGCGGACTTCCAAGACCTGCGGCCGGACGCTTGCGCACCGGTTAAGGTCA
AAGAGCCCCCTCAAATCAATTTAGTTTTGTACCCTCAAGGCCTAACTGGTGAAGAAGTATATGTAAAAGTGGATTTG
AGGGTTAAATGCCCACCTACCTATCCAGATGTAGTTCCTGAAATAGAGTTAAAAAATGCCAAAGGTCTATCAAATGA
AACTGTCAATTTGTTAAAATCTCGCCTAGAAGAACTGCCCAAGAAACACTGTGGGGAGGTAGTGATGATCTTTCAAC
TGGCTTACCACGTCCAGTCATTTCTCACCGAGCATAACAAGCCCCCTCCCAACTCTTTTCATGAAGAAATGCTCCAA
AGGCGGGCTCAAGAGGAGCAACAGAGCCTGTTGGAGCCCCAAGCGGAAAGAAGAGCACAGCAACGTGAAATCCTCCA
TGAGATTCAGAGAAGGAAAGAAGACATAAAAGAAGAGAAAAAAAGGAAAGAAATGGCTAACCAGGAACGTTTGGAAA
TTCCTACTTTGTCAAACCAAGATCATACCTCTAAGAAGGACCCAGGAGGACACAGAACCGCTGCCATTCTACATGGA
GGCTCTCCTAACTTTGTAGGAAATGGTAAACATCGGGCAAACTCCTCAGGAAGGTCTACGTTAACCCGAGAACGTCA
GTATTCTCTATGTAATACTGAAGATTCTCCTGGCTCTTGTGAAATTCTGTATTTCAATATGGGGAGTCCTGATCAGC
TCATCGTCCACAAAGGGAAATGTATTCGCAGTGATCAACAACTTGGAAAATTAGTCTACAATGCTTTGGAAACAGCC
ACTGGTGGCTTTCTCTTGTTGTATGAGTCGGTCCTTCAGTGGCAGAAAAAAATGGGTCCATTCCTTACCACTCAAGA
AAAAGAGAAGATTGATAAGTGCAAAAAGCAGATTCAAGGAACACAAACAGAATTCAACTCACTGGTAAAATTGAGCC
ATCCAAATGTACTACGCTACCTTGCAATGAATTCAAAGAGCAACACGACTCCATTCCTGGTGCACATTTTAGTGCAG
CACATTAGTGGGGTCTCTCTTGCTGCACACCTGAGCCACTCAGGCCCCATCCCTGTCCATCAGCTTCGCAGGTACAC
AGCTCAGCTCCTGTCAGGCCTTGATTATCTGCACAGCAATTCTGTGGTGCATAAGGTCCTGAGTGCATCTAATCTCT
TGGTGGATGCACAAGGCACCCTCAAGATTACGGACTATAGCATTTCTAAGCGCCTCGCAGACATTTGCAAGGAGGAT
GTGTTTGAGCAAACCCGAGTTCGTTTTAGTGACAATCCTCTGCCTTATAAAACGGGGAACAAACCAGATGTTTGCCG
TCTTGGCCTTCTGCTCCTGTCCCTCAGCCAAGGACAGGAATGTGCAGACTACCCTGTGACCATCCCTAGTGACTTAC
CAGCTGACTTTCAAGATTTTCTAAAGAAGAGATGTGTGTGCTTGGATGACAAGGAAAGATGCAGTCCCCACCAGTTG
TTGAAACACAGCTTTATAAATCCCCAGCCAAAAATGCCTCTAGTGCAACAAAGTCCTGAATCTGAAGGACAAGATTA
TGTTGAGACTGTTATTCCTAGCAACCGGCTACCCACTGCTGCCTTCTTTAGTGACACACAGACACAGTTTTCCCGAT
ACTTCATTCAGTTTGAAGAATTACAACTTCTTGGTAAAGGAGCTTTTGGAGCTGTCATCAAGGTGCACAACAAGTTG
GACGCCTGCTGCTACGCAGTGAAGCGCATCCCCATCAACCCGGCCAGCCGGCAGTTCCGCAGCATCAAGGGCGAAGT
GACACTGCTGTCACGGCTGCACCATGAGAACATTGTGCGCTACTACAACGCCTCGATCGAGCGGCACGAGCGCCCGG
CGGGACCGGGGACGCCGCCCCCGGACTCCGGCCCCCTGCCCAAGGATGACCCAGCTGCACGCGGGCAGCCGGCGAGC
GACACAGACGCCCTCGACAGCGTAGAGGCCGCCGCGCCGCCACCCATCCTCAGCAGCTCGGTGGAGTGGAGCACTTC
GGGCGAGCGCTCGGCCAGTGCCCGTTTCCCCGCCACCGGCCCGGGCTCCAGCGATCACGAGGACGACCACGAGGACG
AGCACGGTGGCGTCTTCTCCCAGTCCTTCCTGCCTGCTTCAGATTCTGAAAGTGATATTATCTTTGACAATGAACAT
GAGAACAGTAAAAGTCAGAATCAGGATGAAGATTGCAATGAAAAGAATGGCTGCCATGAAAGTGAGCCATCAGTGAC
GACTGAGGCTGTGCACTACCTATACATCCAGATGGAGTACTGTCAGAAGAGCACTTTACGAGACACCATTGACCAGG
GACTGTATCGAGACACCGTCAGACTCTGGACGCTTTTTCGAGAGATTCTGGATGGATTAGCTTATATCCATGAGAAA
GGAATGATTCACCGGGATTTGAACCCTGTCAACATTTTTTTGGATTCTGATGACCATCTCAAAATAGGTGATTTTGG
TTTGGCGACAGACCATCTAGCCTTTTCTGCTGACAGCAAACAAGACGATCAGACACGAGACTTGATTAAGTCAGACC
CTTCAGGTCACTTAACTGGGATGGTTGCCACTGCTCTCTATGTAAGCCCAGAGGTCCAAGGAAGCACCAAATCTGCA
TACAACCAGAAAGTGCATCTCTTCAGCCTGGGAATTATCTTCTTTGAGATGTCCTATCACCCCATGGTCACCGCTTC
AGAAAGCATCTTTGTTCTCAACCAACTCAGAGATCCCACTTCGCCTAACTTTCCAGAAGACTTTGACGATGGAGAGC
ATGCAAAGCAGAAATCAGTCATCTCCTGGCTGTTGAACCACGATCCAGCAAAACGGCCCACAGCCACAGAACTGCTC
AAGAGTGAGCTGCTGCCCCCACCCCAGATGGAGGACTCAGAGCTCCATGAAGTGCTGCACCACACGCTGACCAACGT
GGATGGGAAGGCCTACCGCACCATGATGGCCCACATCTTCTCGCAGCCCATCTCCCCTGCCATCGATTACACCTATG
ACAGCCACATACTGAAGGGCAACTTCTCATCCGTACAGCCAAGATGCAGCAGCATGTGTGTGAAAACCATCATCCGC
ATCTTTAAAAGACATGGTGCTGTTCAGTTGTGTACTCCACTACTGCTTCCCCGAAACAGACAAATATATGAGCACAA
CGAAGCTGCCCTATTCATGGACCACAGCGGGATGCTGGTCATCCTTCCTTTTGACCTGCGGGTCCCTTTTGCAACAT
ATGTGGCAAGAAATAATATATTGAATTTAAAACGGTACTGCATAGAACGTCTGTTCACGCCCCGCAAGTTAGATCGA
TTTCATCCCAAAGAACTTCTCGAGTGTGCCTTTGATATTGTCACTTCTACCACCAACAGCTTTCTCCCCACTGCTGA
AATTATCTACACTATCTATGAAATCATCCAAGACTTTCCAGCACTTCAACAAAGAAATTACAGTATTTATTTAAACC
ATACCATGTTATTGAAAGCAATACTCTTACACTGTGGGATCCCAGAAGATAAACTCAGTCAAGTCTACATTATTCTG
TATGATGCTGTGACAGAGAAGCTGACGAGGAGAGAAGTGGAAGCTAAATTTTGTAATCTGTCTTTGTCTTCTAATAG
TCTGTGTCGACTCTACAAGTTTATTGAACAGAAGGGAGATTTGCAAGATCTTATGCCAACAATAAATTCATTAATAG
AACAGAAAACAGGTATTGCACAGTTGGTGAAGTATGGCTTAAAAGACCTAGAGGAGGTTGTTGGACTGTTGAAGAAA
CTCGGCATCAAGTTACAGGTTTGGGTCTTGATCAATTTGGGCTTGGTTTACAAGGTGCAGCAGCACAATGGAATCAT
CTTCCAGTTTGTGGCTATCATCAAACGAGACCCAAAGGGCTGTACCTGAATCCTCGCAGCTGGAGGCAGATATGACC
TGCTGATTCCCCAGTTTAGAGCGCCACAAGCTCTGGGCCCAGTTCCCACTGCCATTCGGGTCAGCATAGCTATACAC
AAGATATCTGCTCCTGTCCTCAACATGCAGGAATCTGTAAGTTCTCTTACPATAGCCTCTGGGGACCTCCTGGTTGT
AAGTGTGGGCCAGATGTCTATGTCCAGGGCCATAAACCTAACCCAGAAACTCTGGACAGCAGGCATCACAGCAGAAA
TCATGTACGACTGGTCACAGTTTCAGTCCCAAGAGGAATTACAAGACTACTCCAGACATCATGAAATCACCTATGTG
GCCCTTGTCTCGGATAAAGAAGGAAGCCATGTCAAGGTTAAGTCTTTCGAGAAGGAAAGGCAGACAGAGAAGCGTGT
GCTGCAGACTGAACTTGTGGACCATGTACTGCAGAAACTCAGCACTAAACTCACTGATGAAAGGAATTTTAGAGAAG
CTTCCGATAATCTTGCAGTGCAAAATCTGAAGGGGTCATTTTCTAATGCTTCAGGTTTGTTTTGAATCCATGGAGCA
ACAGTGGTTCCCATTGTCAGTGTGCTAGCCCCGGAGAAGCTCTCAGCCAGCACTAGGAGGCGCTATGAAACTCAGGT
ACAAACTCGACTTCAGACCTCCCTTGCCAACTTACATCAGAAAAGCAGTGAAATTGAAATTCTGGCTGTAGTGGATC
TACCCAAAGAAACAATATTACAGTTTTTATCATTAGAGTGGGATGCTGATGAACAGGCATTTAACACAACTGTGAAG
CAGCTGCTGTCACGCCTGCCAAAGCAAAGATACCTCAAATTAGTCTGTGATGAAATTTATAACATCAAAGTAGAAAA
AAAGGTGTCTGTGCTATTTCTGTACAGCTATAGAGATGACTACTACAGAATCTTATTTTAA

An open reading frame (ORF) for MOL2a was identified from nucleotides 1 to 4986. The disclosed MOL2a polypeptide (SEQ ID NO:4) encoded by SEQ ID NO:3 is 1662 amino acid residues, has a molecular weight of 188250.1 and is presented using the one-letter code in Table 2B. The SignalP, Psort and or Hydropathy profile of MOL2a indicate that this sequence does not have a signal peptide and is likely to be localized to the nucleus. Therefore it is likely that MOL2a is available at the appropriate sub-cellular localization and hence accessible for the therapeutic uses described in this application.

TABLE 2B
Encoded MOL2a protein sequence.

(SEQ ID NO:4)

MAGGRGAPGRGRDEPPESYPQRQDHELQALEAIYCADFQDLRFDACGPVKVKEPPEINLVLYPQCLTGEEVYVKVDL
RVKCFPTYPDVVPEIELKNAKGLSNESVNLLKSRLEELAKKHCGEVVMTFELAYHVQSPLSEHNKPPPKSFHEEMLE
RRAQEEQQRLLEAQAERRAQQREILHETQRRKEEIKEEKKRKEMAKQERLETASLSNQDHTSKKDPGGHRTPAILHG
GSPDFVCNGKHRANSSGRSRLRRERQYSVCNSEDSPOSCEILYFNMGSPDQLMVHKCKCIGSDEQLGKLVYNALETA
TCGFVLLYEWVLQWQKKMGPFLTSQEKEKIDKCKKQIQGTETEFNSLVKLSHPNVVRYLAMNSKSKTTPFVVDILVE
HISCVSLAAHLSHSCPIFVRQLRRYTAQLLSCLDYLHSNSVVHKVLSASNVLVDAEGTVKTTDYSISKRLADICKED
VFEQTRVRFSDNALPYKTGKKGDVWRLCLLLLSLSQGQECGEYPVTIPSDLPADFQDFLKKRCVCLDDKERWSPQQL
LKHSFINPQPKMPLVEQSPESECQDYVETVIPSNRLPSAAFFSETQRQFSRYFTEEEELQLLGKCAFCAVIKVQNKL
DGCCYAVKRIPINPASRQPRRIKGEVTLLSRLHHENIVRYYNAWIERHEPPAGPGTPPPDSCPLAKDDRAARCQPAS
DTDGLDSVEAAAPPPILSSSVEWSTSCERSASARFPATCPGSSDDEDDDEDEHGGVFSQSFLPASDSESDIIFDNED
ENSKSQNQDEDCNEKNCCHESEPSVTTEAVHYLYIQMEYCEKSTLRDTIDQGLYRDTVRLWRLFREILDGLAYIHEK
GMIHRDLKPVNIELDSDDHVKICDFGLATDHLAFSADSKQDDQTGDLIKSDFSGHLTGMVGTALYVSPEVQCSTKSA
YNQKVDLFSLGIIFFEMSYHPMVTASERIFVLNQLRDPTSPKFPEDFDDCEHAKQKSVISWLLNHDPAKRPTATELL
KSELLPPPQMEESELHEVLEHTLTNVDGKAYRTMMAQIFSQRTSPAIDYTYDSDILKGNFSTRTAKMQQHVCETIIR
IFKRHGAVQLCTPLLLFRNRQIYEHNEAALFMDHSCMLVMLPFDLRVPFAAYVARNNTLNLKRYCIERVFTPRKLDR
FHPKELLECAFDIVTSTTNSFLPTAEIIYTIYEIIQEFPALQERNYSIYLNHTMLLKATLLHCCIPEDKLSQVYIIL
YDAVTEKLTRREVEARFCNLSLSSNSLCRLYKFIEQKGDLQDLMPTINSLTEQKTCIAQLVKYGLKDLEEVVGLLKK
LGIKLQVWVLINLGLVYKVQQHNGIIFQFVAIIKRRQRAVPEILAAGGRYDLLIPQFRGPQALGPVPTAIGVSIAID
KISAAVLNMEESVSSVTIGSCDLLVVSVGQMSMSPATNLTQKLWTAGITAEIMYDWSQEQSQEELQEYCRHHEITYV
ALVSDKEGSHVKVKSFEKERQTEKRVLETELVDHVLQKLRTKVTDERNFREASDNLAVQNLKGSESNASOLFEIHGA
TVVPIVSVLAPEKLSASTPRRYETQVQTRLQTSLANLHQKSSEIEILAVVDLPKETILQFLSLEWDADEQAFNTTVK
QLLSRLPKQRYLRLVCDEIYNIKVEKKVSVLFLYSYRDDYYRILF

The MOL2a nucleic acid sequence has 3119 of 3723 bases (83%) identical to a Mus musculus GCN2 EIF2alpha kinase mRNA (GENBANK-ID: MMU243533|acc:AJ243533)

The full amino acid sequence of the protein of the invention was found to have 479 of 1662 bases (88%) amino acid residues (88%) identical to, and 1554 of 1662 residues (93%) similar to, the 1648 amino acid residue CAB58363 GCN2 EIF2alpha kinase protein from Mus musculus (ptnr: TREMBLNEW-ACC: CAB58363).

MOL2b

An additional murine GNC2 eIFK-like nucleic acid of the invention, referred to herein as MOL2b, encodes an Olfactory Receptor (“OR”)-like protein. The novel nucleic acid of 5094 nucleotides is disclosed in Table 2C. The disclosed MOL2b open reading frame (“ORF”) begins at the ATG initiation codon at nucleotides 20-22, shown in bold in Table 2C. The encoded polypeptide is alternatively referred to herein as MOL2b or as CG51424-02. The disclosed MOL2b ORF terminates at a TGA codon at nucleotides 4898-4900. As shown in Table 2C the start and stop codons are in bold letters and the 5′ and 3′ untranslated regions are underlined.

TABLE 2C
MOL2b Nucleotide Sequence

(SEQ ID NO:138)

GTCGAGGCGCAGCGCTGCCATGGCTGGGGGCCGTGGGGCCCCCGGGCGCGGCCGGGACGAGCCTCCGGA
GAGCTACCCGCAACGACAGGACCACGAGCTACAGGCCCTGGAGGCCATCTACGGCGCGGACTTCCAAGA
CCTGCGGCCGGACGCTTGCGGACCGGTCAAAGAGCCCCCTGAAATCAATTTAGTTTTGTACCCTCAAGG
CCTAACTGGTGAAGAAGTATATGTAAAAGTGGATTTGAGGGTTAAATGCCCACCTACCTATCCAGATGT
AGTTCCTGAAATAGAGTTAAAAAATGCCAAAGGTCTATCAAATGAAAGTGTCAATTTGTTAAAATCTCG
CCTAGAAGAACTGGCCAAGAAACACTGTGGGGAGGTGATGATCTTTGAACTGGCTTACCACGTGCAGTC
ATTTCTCAGCGAGCATAACAAGCCCCCTCCCAAGTCTTTTCATGAAGAAATGCTGGAAAGGCGGGCTCA
GGAGGAGCAGCAGAGGCTGTTGGAGGCCAAGCGGAAAGAAGAGCAGGAGCAACGTGAAATCCTGCATGA
GATTCAGAGAAGGAAAGAAGAGATAAAAGAAGAGAAAAAAAGGAAAGAAATGGCTAAGCAGGAACGTTT
GGAAATTGCTAGTTTGTCAAACCAAGATCATACCTCTAAGAAGGACCCAGGAGGACACAGAACGGCTGC
CATTCTACATGGAGGCTCTCCTGACTTTGTAGGAAATGGTAAACATCGGGCAAACTCCTCAGGAAGGTC
TAGGCGAGAACGTCAGTATTCTGTATGTAATAGTGAAGATTCTCCTGGCTCTTGTGAAATTCTGTATTT
CAATATGGGGAGTCCTGATCAGCTCATGGTGCACAAAGGGAAATGTATTGGCAGTGATGAACAACTTGG
AAAATTAGTCTACAATGCTTTGGAAACAGCCACTGGTGGCTTTGTCTTGTTGTATGAGTGGGTCCTTCA
GTGGCAGAAAAAAATGGGTCCATTCCTTACCAGTCAAGAAAAAGAGAAGATTGATAAGTGCAAAAAGCA
GATTCAAGGAACAGAAACAGAATTCAACTCACTGGTAAAATTGAGCCATCCAAATGTAGTACGCTACCT
TGCAATGAATCTCAAAGAGCAAGACGACTCCATCGTGGTGGACATTTTAGTGGAGCACATTAGTGGGGT
CTCTCTTGCTGCACACCTGAGCCACTCAGGCCCCATCCCTGTGCATCAGCTTCGCAGGTACACAGCTCA
GCTCCTGTCAGGCCTTGATTATCTGCACAGCAATTCTGTGGTGCATAAGGTCCTGAGTGCATCTAATGT
CTTGGTGGATGCAGAAGGCACCGTCAAGATTACGGACTATAGCATTTCTAAGCGCCTCGCAGACATTTG
CAAGGAGGATGTGTTTGAGCAAACCCGAGTTCGTTTTAGTGACAATGCTCTGCCTTATAAAACGGGGAA
GAAAGGAGATGTTTGGCGTCTTGGCCTTCTGCTGCTGTCCCTCAGCCAAGGACAGGAATGTGGAGAGTA
CCCTGTGACCATCCCTAGTGACTTACCAGCTGACTTTCAAGATTTTCTAAAGAAATGTGTGTGCTTGGA
TGACAAGGAAAGATGGAGTCCCCAGCAGTTGTTGAAACACAGCTTTATAAATCCCCAGCCAAAAATGCC
TCTAGTGGAACAAAGTCCTGAAGATTCTGGAGGACAAGATTATGTTGAGACTGTTATTCCTAGCAACCG
GCTACCCAGTGCTGCCTTCTTTAGTGAGACACAGAGACAGTTTTCCCGATACTTCATTGAGTTTGAAGA
ATTACAACTTCTTGGTAAAGGAGCTTTTGGAGCTGTCATCAAGGTGCAGAACAAGTTGGACGGCTGCTG
CTACGCAGTGAAGCGCATCCCCATCAACCCGGCCAGCCGGCAGTTCCGCAGGATCAAGGGCGAAGTGAC
ACTGCTGTCACGGCTGCACCATGAGAACATTGTGCGCTACTACAACGCCTGGATCGAGCGGCACGAGCG
GCCGGCGGGACCGGGGACGCCGCCCCCGGACTCCGGGCCCCTGGCCIAGGATGACCGAGCTGCACGCGG
GCAGCCGGCGAGCGACACAGACCGCCTGGACAGCGTAGAGGCCGCCGCGCCGCCACCCATCCTCAGCAG
CTCCGTGGAGTGGAGCACTTCGGGCGAGCGCTCGGCCAGTGCCCGTTTCCCCGCCACCGGCCCGGGCTC
CAGCGATGACGAGGACGACGACGAGGACGAGCACGGTGGCGTCTTCTCCCAGTCCTTCCTGCCTGCTTC
AGATTCTGAAAGTGATATTATCTTTGACAATGAAGATGAGAACAGTAAAAGTCAGAATCAGGATGAAGA
TTGCAATGAAAAGAATGGCTGCCATGAAAGTGAGCCATCAGTGACGACTGAGGCTGTGCACTACCTATA
CATCCAGATGGAGTACTGTGAGAAGAGCACTTTACGAGACACCATTGACCAGGGACTGTATCGAGACAC
CGTCAGACTCTGGAGGCTTTTTCGAGAGATTCTGGATGGATTAGCTTATATCCATGAGAAAGGAATGAT
TCACCGGGATTTGAAGCCTGTCAACATTTTTTTGGATTCTGATGACCATGTGAAAATAGGTGATTTTGG
TTTGGCGACAGACCATCTAGCCTTTTCTGCTGACAGCAAACAAGACGATCAGACAGGAGACTTGATTAA
GTCAGACCCTTCAGGTCACTTAACTGGGATGGTTGGCACTGCTCTCTATGTAAGCCCAGAGGTCCAAGG
AAGCACCAAATCTGCATACAACCAGAAAGTGGATCTCTTCAGCCTGGGAATTATCTTCTTTGAGATGTC
CTATCACCCCATGGTCACGGCTTCAGAAAGGATCTTTGTTCTCAACCAACTCAGAGATCCCACTTCGCC
TAAGTTTCCAGAAGACTTTGACGATGGAGAGCATGCAAAGCAGAAATCAGTCATCTCCTGGCTGTTGAA
CCACGATCCAGCAAAACGGCCCACAGCCACAGAGCTGCTCAAGAGTGAGCTGCTGCCCCCACCCCAGAT
GGAGCAGTCAGAGCTGCATGAAGTGCTGCACCACACGCTGACCAACGTGGATGGGAAGGCCTACCGCAC
CATGATGGCCCAGATCTTCTCGCAGCGCATCTCCCCTGCCATCGATTACACCTATGACAGCGACATACT
GAAGGGCAACTTCTCAATCCGTACAGCCAAGATGCAGCAGCATGTGTGTGAAACCATCATCCGCATCTT
TAAAAGACATGGAGCTGTTCAGTTGTGTACTCCACTACTGCTTCCCCGAAACAGACAAATATATGAGCA
CAACGAAGCTGCCCTATTCATGGACCACAGCGGGATGCTGGTGATGCTTCCTTTTGACCTGCGGATCCC
TTTTGCAAGATATGTGGCAAGAAATAATATATTGAATTTAAAACGATACTGCATAGAACGTGTGTTCAG
GCCGCGCAAGTTAGATCGATTTCATCCCAAAGAACTTCTCGAGTGTGCATTTGATATTGTCACTTCTAC
CACCAACAGCTTTCTGCCCACTGCTGAAATTATCTACACTATCTATGAAATCATCCAAGAGTTTCCAGC
ACTTCAGGAAAGAAATTACAGTATTTATTTGAACCATACCATGTTATTGAAAGCAATACTCTTACACTG
TGGGATCCCAGAAGATAAACTCAGTCAAGTCTACATTATTCTGTATGATGCTGTGACAGAGAAGCTGAC
GAGGAGAGAAGTGGAAGCTAAATTTTGTAATCTGTCTTTGTCTTCTAATAGTCTGTGTCGACTCTACAA
GTTTATTGAACAGAAGGGAGATTTGCAAGATCTTATGCCAACAATAAATTCATTAATAAAACAGAAAAC
AGGTATTGCACAGTTGGTGAAGTATGGCTTAAAAGACCTAGAGGAGGTTGTTGGACTGTTGAAGAAACT
CGGCATCAAGTTACAGGTCTTGATCAATTTGGGCTTGGTTTACAAGGTGCAGCAGCACAATGGAATCAT
CTTCCAGTTTGTGGCTTTCATCAAACGAAGGCAAAGGGCTGTACCTGAAATCCTCGCAGCTGGAGGCAG
ATATGACCTGCTGATTCCCCAGTTTAGAGGGCCACAAGCTCTGGGGCCAGTTCCCACTGCCATTGGGGT
CAGCATAGCTATAGACAAGATATCTGCTGCTGTCCTCAACATGGAGGAATCTGTTACAATAAGCTCTTG
TGACCTCCTGGTTGTAAGTGTTGGTCAGATGTCTATGTCCAGGGCCATCAACCTAACCCAGAAACTCTG
GACAGCAGGCATCACAGCAGAAATCATGTACGACTGGTCACAGTCCCAAGAGGAATTACAAGAGTACTC
CAGACATCATGAAATCACCTATGTGGCCCTTGTCTCGGATAAAGAAGGAAGCCATGTCAAGGTTAAGTC
TTTCGAGAAGGAAAGGCAGACAGAGAAGCGTGTGCTGGAGACTGAACTTGTGGACCATGTACTGCAGAA
ACTGAGGACTAAAGTCACTGATGAAAGGAATGGCAGAGAAGCTTCCGATAATCTTGCAGTGCAAAATCT
GAAGGGGTCATTTTCTAATGCTTCAGGTTTGTTTGAATTCCATGGAGCAACAGTGGTTCCCATTGTGAG
TGTGCTAGCCCCGGAGAAGCTGTCAGCCAGCACTAGGAGGCGCTATGAAACTCTGGTGGATCTACCCAA
AGAAACAATATTACAGTTTTTATCATTAGAGTGGGATGCTGATGAACAGGCATTTAACACAACTGTGAA
GCAGCTGCTGTCACGCCTGCCAAAGCAAAGATACCTCAAATTAGTCTGTGATGAAATTTATAACATCAA
AGTAGAAAAAAAGGTGTCTGTGCTATTTCTGTACAGCTATAGAGATGACTACTACAGAATCTTATTTTA
ACCCTAAAGAACTGTCGTTAACCTCATTCAAACAGACAGAGGCTTATACTGGAATAATGGAATGTTGTA
CATTCATCATAATTTAAAATTAAATTCTAAGAAGAGGCTGGGTGCAGTGGCTCACACCTTTAATCCCAG
CACTTTGGGAAGCCAAGGCAGGAAGACTGCTTGAAACCAGGAGTTTGAGACCAGCCT

The disclosed nucleic acid MOL2b sequence, localized to chromosome 15, has 4678 of 4680 bases (99%) identical to a gb:GENBANK-ID:AX056426|acc:AX056426.1 mRNA from Homo sapiens (Sequence 70 from Patent WO0073469) (E=0.0).

The disclosed MOL2b polypeptide (SEQ ID NO:139) encoded by SEQ ID NO:138 is 1626 amino acid residues and is presented using the one-letter code in Table 2D. The SignalP, Psort and or Hydropathy profile of MOL2b indicate that this sequence does not have a signal peptide In one embodiment, MOL2b is likely to be located to the nucleus with a certainty of 0.9800. In other embodiments, MOL2b could also be located to the microbody (peroxisome), with a certainty of 0.3000, the mitochondrial matrix space with a certainty of 0.1000, or to the lysosome (lumen) with a certainty of 0.1000. Therefore it is likely that MOL2b is available at the appropriate sub-cellular localization and hence accessible for the therapeutic uses described in this application.

TABLE 2D
Encoded MOL2b protein sequence.

(SEQ ID NO:139)

MAGGRGAPGRGRDEPPESYPQRQDHELQALEAIYGADFQDLRPDACGPVKEPPEINLVLYPQGLTGEEV
YVKVDLRVKCPPTYPDVVPEIELKNAKGLSNESVNLLKSRLEELAKKHCGEVMIFELAYHVQSFLSEHN
KPPPKSFHEEMLERRAQEEQQRLLEAKRKEEQEQREILHEIQRRKEEIKEEKKRKEMAKQERLEIASLS
NQDHTSKKDPCGHRTAAILHGGSPDFVGNGKHRANSSGRSRRERQYSVCNSEDSPGSCEILYFNNGSPD
QLMVHKGKCIGSDEQLGKLVYNALETATGGFVLLYEWVLQWQKKMGPFLTSQEKEKIDKCKKQIQGTET
EFNSLVKLSHPNVVRYLAMNLKEQDDSIVVDILVEHISGVSLAAHLSHSGPIPVHQLRRYTAQLLSGLD
YLHSNSVVHKVLSASNVLVDAEGTVKITDYSISKRLADICKEDVFEQTRVRFSDNALPYKTGKKGDVWR
LGLLLLSLSQGQECGEYPVTIPSDLPADFQDFLKKCVCLDDKERWSPQQLLKHSFINPQPKMPLVEQSP
EDSGGQDYVETVIPSNRLPSAAFFSETQRQFSRYFIEFEELQLLGKGAFGAVIKVQNKLDGCCYAVKRI
PINPASRQFRRIKGEVTLLSRLHHENIVRYYNAWIERHERPAGPGTPPPDSGPLAKDDRAARGQPASDT
DGLDSVEAAAPPPILSSSVEWSTSGERSASARFPATGPGSSDDEDDDEDEHGGVFSQSFLPASDSESDI
IFDNEDENSKSQNQDEDCNEKNGCHESEPSVTTEAVHYLYIQMEYCEKSTLRDTIDQGLYRDTVRLWRL
FREILDGLAYIHEKGMIHRDLKPVNIFLDSDDHVKIGDFGLATDHLAFSADSKQDDQTGDLIKSDPSGH
LTGMVCTALYVSPEVQGSTKSAYNQKVDLFSLGIIFFEMSYHPMVTASERIFVLNQLRDPTSPKFPEDF
DDGEHAKQKSVISWLLNHDPAKRPTATELLKSELLPPPQMEESELHEVLHHTLTNVDGKAYRTMMAQIF
SQRISPAIDYTYDSDILKGNFSIRTAKMQQHVCETIIRIFKRHGAVQLCTPLLLPRNRQIYEHNEAALF
MDHSGMLVMLPFDLRIPFARYVARNNILNLKRYCIERVFRPRKLDRFHPKELLECAFDIVTSTTNSFLP
TASIIYTIYEIIQEFPALQERNYSIYLNHTMLLKAILLHCCIPEDKLSQVYIILYDAVTEKLTRREVEA
KFCNLSLSSNSLCRLYKFIEQKGDLQDLMPTINSLIKQKTGIAQLVKYGLKDLEEVVGLLKKLGIKLQV
LINLGLVYKVQQHNGIIFQFVAFIKRRQRAVPEILAAGGRYDLLIPQFRGPQALGPVPTAIGVSIAIDK
ISAAVLNMEESVTISSCDLLVVSVGQMSMSRAINLTQKLWTAGITAEIMYDWSQSQEELQEYCRHHEIT
YVALVSDKEGSHVKVKSFEKERQTEKRVLETELVDHVLQKLRTKVTDERNGPEASDNLAVQNLKCSFSN
ASGLFEFHGATVVPIVSVLAPEKLSASTRRRYETLVDLPKETILQFLSLEWDADEQAFNTTVKQLLSRL
PKQRYLKLVCDEIYNIKVEKKVSVLFLYSYRDDYYRILF

The full amino acid sequence of the protein of the invention was found to have 1448 of 1473 amino acid residues (98%) identical to, and 1448 of 1473 amino acid residues (98%) similar to, the 1495 amino acid residue ptnr:SPTREMBL-ACC:Q9P2K8 protein from Homo sapiens (Human) (KIAA1338 PROTEIN).

MOL2b is expressed in at least Aorta, Brain, Colon, Heart, Kidney, Lung, Prostate, Thyroid, Uterus, Whole embryo, Bladder, Bone, Eye, Liver, Muscle, Pancreas, Salivary Gland, and Skin. Expression information was derived from the tissue sources of the sequences that were included in the derivation of the sequence of CuraGen Acc. No. CG51424-02. The sequence is predicted to be expressed in the following tissues because of the expression pattern of (GENBANK-ID: gb:GENBANK-ID:AX056426|acc:AX056426.1) a closely related Sequence 70 from Patent WO0073469 homolog in species Homo sapiens: Bone, and Prostate.

Other BLAST results including the sequences used for ClustalW analysis are presented in Table 2E.

TABLE 2E
BLAST results for MOL2

| Gene Index/ | Protein/ | Length | Identity | Positives | |
|---|---|---|---|---|---|
| Identifier | Organism | (aa) | (%) | (%) | Expect |
| PatP: B65663 | Protein | 1649 | 1626/1662 | 1632/1662 | 0.0 |
| | kinase [Homo | | (97%) | (98%) | |
| | sapiens] | | | | |
| PatP: B43581 | Cancer | 604 | 592/609 | 594/609 | 6.5e−306 |
| | associated | | (97%) | (97%) | |
| | protein [Homo | | | | |
| | sapiens] | | | | |
| PatP: B42761 | ORF2525 | 619 | 585/624 | 596/624 | 1.3e−300 |
| | polypeptide | | (93%) | (95%) | |
| gi\|10764165\|gb\|AAG22591.1\| | GCN2gamma | 1570 | 1313/1577 | 1375/1577 | 0.0 |
| (AF193344) | [Mus | | (83%) | (86%) | |
| | musculus] | | | | |
| gi\|11360320\|pir\|\|T46924 | probable | 938 | 887/946 | 889/946 | 0.0 |
| | translation | | (93%) | (93%) | |
| | initiation | | | | |
| | factor eIF- | | | | |
| | 2alpha kinase | | | | |
| | (EC 2.7.1.-) | | | | |
| | [similarity] - | | | | |
| | human | | | | |
| | (fragment) | | | | |
| gi\|7305017\|ref\|NP_038747.1\| | GCN2 | 1648 | 1374/1647 | 1442/1647 | 0.0 |
| | eIF2alpha | | (83%) | (87%) | |
| | kinase [Mus | | | | |
| | musculus] | | | | |
| gi\|10764161\|gb\|AAG22589.1\| | GCN2alpha | 1370 | 1189/1381 | 1241/1381 | 0.0 |
| | [Mus | | (86%) | (89%) | |
| | musculus] | | | | |
| gi\|7243057\|dbj\|BAA92576.1\| | KIAA1338 | 1495 | 1377/1460 | 1380/1460 | 0.0 |
| (AB037759) | protein [Homo | | (94%) | (94%) | |
| | sapiens] | | | | |
| gi\|10764163\|gb\|AAG22590.1\| | GCN2beta | 1648 | 1373/1647 | 1440/1647 | 0.0 |
| (AF193343) | [Mus musculus] | | (83%) | (87%) | |
| gi\|6065914\|emb\|CAB58360.1\| | putative eIF2 | 548 | 505/556 | 507/556 | 0.0 |
| (AJ243428) | alpha kinase | | (90%) | (90%) | |
| | [Homo | | | | |
| | sapiens] | | | | |

This information is presented graphically in the multiple sequence alignment given in Table 2F (with MOL2 being shown on line 1) as a ClustalW analysis comparing MOL2 with related protein sequences.

[image]

[image]

[image]

[image]

[image]

[image]

Domain Analysis

The presence of identifiable domains in the protein disclosed herein was determined by searches versus domain databases such as Pfam, PROSITE, ProDom, Blocks or Prints and then identified by the Interpro domain accession number. Significant domains are summarized in Table 2G.

TABLE 2G
Domain Analysis of MOL2b

| MODEL | DESCRIPTION | SCORE | E-VALUE | N |
|---|---|---|---|---|
| pkinase | Protein kinase domain | 282.0 | 7.8e−81 | 4 |
| Ribosomal_L23 | Ribosomal protein L23 | 5.0 | 4.1 | 1 |
| GalP_UDP_transf | Galactose-1-phosphate | 2.7 | 6.5 | 1 |
| | uridyl transfer | | | |
| mRNA_cap_enzyme | mRNA capping | −181.3 | 9.6 | 1 |
| | enzyme | | | |

IPR000719; (Euk_pkinase)

Eukaryotic protein kinase

PROSITE: PS00107 PROTEIN_KINASE_ATP

PROFILE: PS50011 PROTEIN_KINASE_DOM

PFAM: PF00069 pkinase

SMART: SM00221 STYKc

IPR002290 Serine/Threonine protein kinase family active site

IPR001245 Tyrosine kinase catalytic domain

GO Hierarchy:

Molecular Function:

protein kinase (GO:0004672)

ATP binding (GO:0005524)

Biological Process:

protein phosphorylation (GO:0006468)

Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

This indicates that the sequence of the invention has properties similar to those of other proteins known to contain this/these domain(s) and similar to the properties of these domains.

Chromosomal Information

MOL2 belongs to genomic DNA [Acc.NO.: AC025168 from GenbankNEW]. Within this GenbankNew entry was a note showing that the sequence was from Chromosome 15q14. Therefore we assign the chromosomal locus of this invention as Chromosome 15q14.

Tissue Expression

MOL2 is expressed in at least the following tissues: brain and liver (derived from literature sources) and thyroid (derived from 20466828_EXT1).

Based on information available on expression of SWISSPROT-ACC:P29089 TYPE-1B ANGIOTENSIN II RECEPTOR (AT1B) (AT3)—Rattus norvegicus (Rat), the closest G-protein coupled receptor family member it is likely that MOL2 is expressed in cardiac tissue, renal tissue, and vascular tissue as angiotensin is expressed in these tissues. MOL2 has similarity to the murine GNC2 eIFK protein, a possible GNC2 eIFK and other GNC2 eIFKs and their functions as described in but not limited to the references below: In eukaryotic cells, protein synthesis is regulated in response to various environmental stresses by phosphorylating the alpha subunit of the eukaryotic initiation factor 2 (eIF2alpha) (Berlanga, et.al., Eur J Biochem; 265(2):754-62; October, 1999). Three different eIF2alpha kinases have been identified in mammalian cells, the heme-regulated inhibitor (HRI), the interferon-inducible RNA-dependent kinase (PKR) and the endoplasmic reticulum-resident kinase (PERK). A fourth eIF2alpha kinase, termed GCN2, was previously characterized from Saccharomyces cerevisiae, Drosophila melanogaster and Neurospora crassa. Berlanga et al. 1999 describe the cloning of a mouse GCN2 cDNA (MGCN2), which represents the first mammalian GCN2 homolog. MGCN2 has a conserved motif, N-terminal to the kinase subdomain V, and a large insert of 139 amino acids located between subdomains IV and V that are characteristic of the known eIF2alpha kinases. Furthermore, MGCN2 contains a class II aminoacyl-tRNA synthetase domain and a degenerate kinase segment, downstream and upstream of the eIF2alpha kinase domain, respectively, and both are singular features of GCN2 protein kinases. MGCN2 mRNA is expressed as a single message of approximately 5.5 kb in a wide range of different tissues, with the highest levels in the liver and the brain. Specific polyclonal anti-(MGCN2) immunoprecipitated an eIF2alpha kinase activity and recognized a 190 kDa phosphoprotein in Western blots from either mouse liver or MGCN2-transfected 293 cell extracts. Interestingly, serum starvation increased eIF2alpha phosphorylation in MGCN2-transfected human 293T cells. This finding provides evidence that GCN2 is the unique eIF2alpha kinase present in all eukaryotes from yeast to mammals and underscores the role of MGCN2 kinase in translational control and its potential physiological significance.

A family of protein kinases regulates translation in response to different cellular stresses by phosphorylation of the alpha subunit of eukaryotic initiation factor-2 (eIF-2alpha). In yeast, an eIF-2alpha kinase, GCN2, functions in translational control in response to amino acid starvation. It is thought that uncharged tRNA that accumulates during amino acid limitation binds to sequences in GCN2 homologous to histidyl-tRNA synthetase (HisRS) enzymes, leading to enhanced kinase catalytic activity. Given that starvation for amino acids also stimulates phosphorylation of eIF-2alpha in mammalian cells, we searched for and identified a GCN2 homologue in mice. Sood et.al., 2000 cloned three different cDNAs encoding mouse GCN2 isoforms, derived from a single gene, that vary in their amino-terminal sequences. Like their yeast counterpart, the mouse GCN2 isoforms contain HisRS-related sequences juxtaposed to the kinase catalytic domain. While GCN2 mRNA was found in all mouse tissues examined, the isoforms appear to be differentially expressed. Mouse GCN2 expressed in yeast was found to inhibit growth by hyperphosphorylation of eIF-2alpha, requiring both the kinase catalytic domain and the HisRS-related sequences. Additionally, lysates prepared from yeast expressing mGCN2 were found to phosphorylate recombinant eIF-2alpha substrate. Mouse GCN2 activity in both the in vivo and in vitro assays required the presence of serine-51, the known regulatory phosphorylation site in eIF-2alpha. Together, those studies identify a new mammalian eIF-2alpha kinase, GCN2, that can mediate translational control. Phosphorylation of the alpha subunit of eukaryotic initiation factor 2 (eIF-2alpha) is a well-characterized mechanism regulating protein synthesis in response to environmental stresses (Yang et al., Mol Cell Biol; 20(8):2706-17; April, 2000). In the yeast Saccharomyces cerevisiae, starvation for amino acids induces phosphorylation of eIF-2alpha by Gcn2 protein kinase, leading to elevated translation of GCN4, a transcriptional activator of more than 50 genes. Uncharged tRNA that accumulates during amino acid limitation is proposed to activate Gcn2p by associating with Gcn2p sequences homologous to histidyl-tRNA synthetase (HisRS) enzymes. Given that eIF-2alpha phosphorylation in mammals is induced in response to both carbohydrate and amino acid limitations, we addressed whether activation of Gcn2p in yeast is also controlled by different nutrient deprivations. It was found that starvation for glucose induces Gcn2p phosphorylation of eIF-2alpha and stimulates GCN4 translation. Induction of eIF-2alpha phosphorylation by Gcn2p during glucose limitation requires the function of the HisRS-related domain but is largely independent of the ribosome binding sequences of Gcn2p. Furthermore, Gcn20p, a factor required for Gcn2 protein kinase stimulation of GCN4 expression in response to amino acid starvation, is not essential for GCN4 translational control in response to limitation for carbohydrates. These results indicate there are differences between the mechanisms regulating Gcn2p activity in response to amino acid and carbohydrate deficiency. Gcn2p induction of GCN4 translation during carbohydrate limitation enhances storage of amino acids in the vacuoles and facilitates entry into exponential growth during a shift from low-glucose to high-glucose medium. Gcn2p function also contributes to maintenance of glycogen levels during prolonged glucose starvation, suggesting a linkage between amino acid control and glycogen metabolism.

Uses of the Compositions of the Invention

The expression pattern, map location and protein similarity information for MOL2 suggest that it may function like a member of the GCN2 eIFαK family. Therefore, the nucleic acids and proteins of the invention are useful in potential therapeutic applications implicated, for example but not limited to, in various pathologies/disorders as described below:

Hyperthyroidism

Hypothyroidism

Von Hippel-Lindau (VHL) syndrome

Alzheimer's disease

Stroke

Tuberous sclerosis

Hypercalceimia

Parkinson's disease

Huntington's disease

Cerebral palsy

Epilepsy

Lesch-Nyhan syndrome

Multiple sclerosis

Ataxia-telangiectasia

Leukodystrophies

Behavioral disorders

Addiction

Anxiety

Pain

Cirrhosis

Transplantation

and/or other pathologies/disorders.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the GCN2 eIFαK-like protein may be useful in gene therapy, and the GCN2 eIFαK-like protein may be useful when administered to a subject in need thereof. By way of nonlimiting example, the compositions of the present invention will have efficacy for treatment of patients suffering from Hyperthyroidism, Hypothyroidism, Von Hippel-Lindau (VHL) syndrome, Alzheimer's disease, Stroke, Tuberous sclerosis, Hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection Cirrhosis, Transplantation and/or other pathologies/disorders. The novel nucleic acid encoding the GCN2 eIFαK-like protein, and the GCN2 eIFαK-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL3

An additional protein of the invention, referred to herein as MOL3, is a human complement C3-like protein. The novel nucleic acid was identified by TblastN using CuraGen Corporation's sequence file for MOL probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein. The novel nucleic acid of 4894 nucleotides (82254077.0.1, SEQ ID NO:5) encoding a novel olfactory receptor-like protein is shown in Table 3A. An open reading frame (ORF) was identified beginning with an ATG initiation codon at nucleotides 1-3 and ending with a TGA codon at nucleotides 4837-4839. Putative untranslated regions downstream from the termination codon are underlined in Table 3A, and the start and stop codons are in bold letters.

TABLE 3A
MOL3 Nucleotide Sequence

(SEQ ID NO:5)

ATGTCCCCGTCTCTCCCTTTCTCTGCCATCTCCCTATGTCTCTCCCATCTCCCTATCCCTCTCCCATCTCCAAGG
TACATCCTGGTGACCCCCCGAGTTCTGAGGGTTGGCAGTCCGGAGACCATTCACATTCAGGCCCACTCAGACTCC
AGACAGCCCCTCACAAGGACCCTCAAGCTGAACCTCACAGTGTGGCACTTCCCCATGAGGAAGACACTCTTGGCA
AGGAGCCAGCTCATTCTCTCACCACGAAACAACTTTATGGACCAGCCACCTGTGACGGTTCCCGAGAGCCTGATG
TACCTCCCAAAACCAGGCCAGCAATATGTCATCATCCGGGCAACTTGGGCACCCACCTCGGGCTCCTCATTCATG
GAGAAGATGGTGCTGGTGGCTCTTCATGCTGGCTACATCTTTATCCACACCCAGAAGACCATCTACACCCCTTCT
CCCCTAGTTCACTACCGCGTGTTCACTGTGAACCACAAGATGGACCCTGTGACCAGGACATTCACTCTGGACATC
AAGAATCCTGATCGGTCCCCAGCTTCCAGAGTCCTTGTCCACTCCCAAGACCAACCTGGGGTGCTTGCACTGTCT
TGGGGCCCTGAAAAGTCCCTGTGCCTCAGTTTGGGCACCTGGACCATCGAAGCCAGCTACCAAAGTACACCCAAG
CAGAAGTTCGAGGCTGCCTTTGATCTGAACGAATATCTCCTCCCATCTTTTCAGGTCCAGCTGGTCCCAAATAAG
ACTTTCTTTTACCTCAAGGATCAGGCTCTGGGCCTTGACATCCAGGCTCGGTATATATTTAACAAGCCAGTGGAC
GGACATCCTTTGGTCATCTTTGGCGTGAAATTGGACTCCTGCCGGATCCCTATCCAAAGCTCCCTGCAGAGGCTG
GAGGTGACTGAAACAGGGCGTGAGATCGTGCAAGCTCAGACCTCAGGGGTGAAGATCATCCAGAGCCCATACAAC
ATCAAGTTCACCAGCACACCCCAGTATTTCAAGCCAGGAATGCCCTTCCACTTTCGGGTCAGAGTCGTACAAAGC
AGTCCTATTCAGATCATATTCCAGTCTCACCTCTCACACCAGGCCACTGCAGCCTTTTCCTTCACCTTACCCCAG
ATTCCACCTCAGGTCTTCATCTCAAATCCTGATGGGTCCCCAGCTTCCAGAGTCCTTGTCCACTCCCAAGACCAA
AAAGTGTACACCTCAGCTGAGGGGTTGGCCACTCTGACCATCAACACAGATGCAAATCTGGACAAGCTCCCCATC
GAGGTGAAAACTGAGGAATCTCTTCAGCCAGAGGAGCAGGCTTCAGCCAAGATGACAGCTTGCCCTTACTTGACT
CAGGATGGGTCAGCAAACTTCCTACACATCGAAGTAAAGACATTGGGCACAGACGTTGGCAGCAGCATCCAGCTG
AGCCTCAACACAAGGCATCACGACCCTAAAACCAAGGACAAGATTACTCACTTCACCATCCTGGTGGTCACCGAG
GGTAAGGCCCGGCACCTTGGCACGCAGGTGGCGCAGGTGGGGGTACCCTCCTTCCCCATTCTGGCCTTTTATTTA
CTTCCCAGAGCAGCAAGCCAAGACCCTGAGTTGGTGGCTGATTCCATATGGATTGATGTCAATGACACATGCATA
GGGCTGAAAGTTGGCTTGAAGAATGATAGATTCTTCCAGTCTTTGGAGCCCAACAGCCAAGTCGAACTCAAGGTG
ACAGGTGATGCACAAGCCACAGTGGGGCTGGTGGCTGTGGACAAGGCTGTCTATGTCTTGAACAGCAAACACAAG
CTCACTCAGAAGAAGGTATGGAATGTGGTGGAGGAACATGACATTGGCTGCACAGGAGGAAGTGCGAAAGACAGA
TTTGCTGTGTTCAAGGATGCTGGATTGGACCTGAAAATCAGCACAGCAATGGATACCGGCCACCAGCAAAGTCAC
AGCTGCCAGCAGGCTGAGGTGGGAGAATCACTTGAACCCGGGAGGCAGAGGTTGCGGTCAGCCGAGATTGCGCCA
CTGCACTCCACCCTGAACAAGTTTAAGACAGAGCTGGAGCAAAAGTGCTCTCAGGCTGGGCTCCGGGAGAGCCCA
GTGGGGCTGTCTTGTGAGGAGAGGACCTGGCATGTCCGCCATGGTCCAGCCTGTGTGGCTGCTTTCCTGGACTCC
TGCTCACACCTGCTCCCTCCAGCGGATGAAGAAGAGGACTTCGATGACCTCTTCTTGGATGACATGCCTGTGCGG
ACCTTCTTCCCCGAGAGTTGGCTCTGGAACAGCATCTCCCATTACCCCATCTCTGTGAAGGTGCCACATTCCATC
ACCACGTGGCAGTTTGTGGTGGTCAGCCTCAAGGCTGGACAAGGTGGTCTCTGTGTCTCGGACCCCTTTGAGCTG
ACAGTTATGAAATCGTTCTTTGTGGACCTTAAGTTGCCCTCCTCCGTCATCACGAATGAGCACGTCCAGATCCAA
GCCATGTTGTACAATTTCAGCCATCGCCAGGCCAAGGTCCGAGTGGAGTTCCCCCACAACGAGACACTGTGCAGT
CCGTCAAAGCCAGGAGCACCATCCCACCAGGTAGTGGTCGTGCCCCCCACCTCCTCCAAGATAGTACACTTTGTC
CTTCTCCCTCTGGAGACAGGCAAAGTGGACGTGGAGGTCAACGCTGTCGGCTACCGGGTCCAGGACCATGTGAAG
AAGACACTCTTGGTCGAAGGTTGTGGTTATTCAGGTCAGACCCAGACAAAACTGGTGCCAAGACAGGAGTTCTTG
AACATGGTACCCGACACGGAGGCGGAAGTGTTTATCAGTGTTCAAGGTGACATCCTTGCTGAGACAATTGTGCGC
ACCCTCACACCCAGTCAGATTCAGCAGCTGCTGCGCGTCCCCACGCCCTCCCCTGAGCAGACGCTGAGCTCCCTG
ACGCCCGTCATCATCCTGTCCCGCTATTTGGATACCACCGGCCAGTGGGGCAAGGTCCGGGTGGAGCACAGCGAC
CAGGTCATGAAGAATATTGGCTACACTCAGATGCTGACCCACCGGAGTTCAGACCGCACCTACCACACCTCCAAG
GGGAACCCAGGAAGCACTTGGCTCACAACCTATCTGTTCCGCCTCTTTCCCCTCGCCTACTCTATGATGACGACC
CAAGTGCTTAGCCTGTCCTCTCTCTGTGACATGGCCAACTGGATCATCATCGACAGGCAGGCAGAGGATGGGCAC
TTCCTGGAGAAGGGCCCTGTGGTCATGACATCCATGTCCGAGGAGGATGTATCCCTCACAGCTCTTCTCCTAATA
GCCCTCAATGACGGAAAGCAGTTGTGCACACAGAAGGTACGACCCAATTTGATGGCCAGCATCGAGAAGGCCGGA
GGATTGCTTGACCTCAGGAGGTTCAGCCTCCAGCGGAGCTATGCCGTAGCCATACCCTCCTATGCACTGGCCGAC
AAAACCCACTGGCCAGTGGATGAGCAGAATCTGGGCTCCCTGTACACCATTGAGGCCACAGCCTATGGGCTCATG
CAGAACCTGGACCTGGGCCGGTACAATGAGACACACCCCATAGCCAAGTGGCTACTAGACAAGCAGGAGCTGCGA
GGAGGCTTCAGCTCCACCCAGCCAGGCAGGAGCAGTCGCCTTTCCCACCCACAGCGCTGGCCACAGGGCTCCCTG
NTGGCCCTTGAAGCTCTGACCCGCTTCCGCGAAGCTGTCCCCTTCAAGGGCATCCAGGATCTCCACGTCCAGATC
AGAGCCCCCAAGACAGCCCTCAATGTGAATTGCTACATTGATCACAGCAATCCCTACCAACAGCGGTCAGCAAAG
TTCCTTGCCCAGGACGACCTAGAGATCAAAGCCAGTGGCAACGGGAGAGGCACCATCTCGATCCTGACAATCTAT
CACAAGTCCCCAGAGTCCCGGGAGGACAACTGCAACCTGTACCACCTGAATGCGACTCTCCACAGTCCCCTAGAA
GAAAATAAAAAGGGAGGTGACACTTTTCGGCTCCCGATGGAAACAAGGTTCCAGAACAATGGAGAGGCCACAATG
ACTATCATGCAGGTCTCCCTGCTCACGGGCTTCTACCCCAACCAGGATGACCTCAAACACCTCACGAGTGATCTG
GAGAGGTACGCCTTTCAGTACAAAACCAAGACAACTACCAGCGACAGCACTGTTGTCCTCTACCTCGAAAAGCTC
TCCCATGAGAAGAACACGGTGCTGGGCTTTCGGGTTCACAGGATCCTGCAGGCGCAGTTCCTGCAGGCCGCCCTC
GTCACCATCTACGACTACTACGAGCCTTCCCGGAGGTGCAGCACTTTCTACAACCTGCCCACACAGCAATCTTCC
CTGAGAAAGATCTCCCACAAAGACATCTGCAGATGTCCAGACGGACAGTGCCCATCCCTGCAGAAGCCCAGTGGC
CAATTGAGGCAGGAGGAGCTCCAGACAACAGCATGTGAGGCAGGCGTGGATTTTGTCTACAAGACAAAGCTGGAA
TCTGTGGAGGTCTCTGCCTCCAACCCTTACGTCTATTACAACACGCAGCTCCAAGACATCATTAACAGTGGTACG
GACCCTGCCAAACCCCTGGCCATGAAGAAATTCGTCTCCCATGCCACTTCCCATGACTCCCTCGGGTTCCAAGAA
CAGGAATCGTACCTCATCATGCGCCAGACGTCACACCTGTGGAGAATCAAATCTGATTACAGCTATGTTCTGCGC
AAGGAGACGTTCCTCATCCTTTGGCCAGCAGATGGAGATGCCAGCAAGAAAGAATTGCGGGACCAACTGGAGGAA
TTTTTGGAATATATGCGCACCCACGCCTCCCAGTCCTGAGCCTCTTCTCCTTTCACGGAGGTGTCATCAGGGCAC
CTCTGCGCCACGTCGGTTT

The disclosed MOL3 polypeptide (SEQ ID NO:6) encoded by SEQ ID NO:5 is 1612 amino acid residues, and is presented using the one-letter code in Table 3B. The MOL3 protein were analyzed for signal peptide prediction and cellular localization. SignalP results predict that MOL3 is cleaved between position 20 and 21 of SEQ ID NO:6. Psort and Hydropathy profiles also predict that MOL3 contains a signal peptide and is likely to be localized at the endoplasmic reticulum (certainty of 0.5500).

TABLE 3B
Encoded MOL3 protein sequence.

(SEQ ID NO:6)

MSPSLPFSAISLCLSHLPMPLPSPRYILVTPRVLRVGSPESIHIQAHSDSRQPLTRTLKVNLTVWDFPMRKTVLA
RSQLILSPGNNFMDQAPVTVPESLMYLPKPGQQYVIIRATWAPTSGSSFMEKMVLVALHAGYIFIQTEKTIYTPS
PLVHYRVFTVNHKMDPVTRTFTLDIKNPDGSPASRVLVHSQDQPGVLALSWGPEKSLCLSLGTWTIEASYQSTPK
QKFEAAFDVKEYVLPSFEVQLVPNKTFFYLKDEALGVDIQARYIFNKPVDGHALVIFGVKLDSCRIPIQSSLQRV
EVTETGGEMVQAETSGVKIIQSPYNIKFTRTPQYFKPGMPFHFRVRVVQSSPIQIIFQSHLSHQATAGFSFTLPQ
IPPQVFISNPDGSPASRVLVHSQDQKVYTSAEGLATLTINTDANLDKLPIEVKTEESLQPEEQASAKMTAWPYLT
QDGSGNFLHIEVKTLGTEVGSSIQLSLNTRHQDPKTKDKITHFTILVVREGKARQLGRQVAQVGVPSFRILAFYL
LPRGASQDPELVADSIWIDVNDRCIGLKVGLKNDRFFQSLEPNSQVELKVTGDAEATVGLVAVDKAVYVLNSKHK
LTQKKVWNVVEEHDIGCTCGSGKDRFAVFKDAGLDLKISTGMDSCHQQSHSCQEAEVGESLEPGRQRLRSAEIAP
LHSSVNKFKTELEQKCCEAGLRESPVGLSCEERTWHVRHGPACVAAFLDCCSHLLPPADEEEDFDDLFLDDMPVP
TLFPESWLWNSISHYPISVKVPDSITTWQFVVVSLKAGQGGLCVSDPFELTVMKSFFVDLKLPSSVIRNEQVQIQ
AMLYNFRDRQAKVRVEFPHKETLCSASKPGAPSHQVVVVPPTSSKIVHFVLLPLETGKVDVEVKAVGYGVQDHVK
KTLLVEGCGYSGQTQTKLVPRQEFLNMVPDTEAEVFISVQGDILGETIVGSLTPSEIQQLLRVPTGCPEQTLSSL
TPVIILSRYLDTTGQWGKVGVEHRDQVMKNIGYTQMLTHRSSDGTYHTSKGNPGSTWLTSYVFRVFALAYSMMTT
QVLSLSSLCDMANWIIIDRQAEDCHFLEKGPVVMTSMSEEDVSLTALVLIALNEGKELCRQKVGPNLMASIEKAG
GLLELRRLRLQRSYAVAIASYALADKTHWPVDEQNLGSLYTIEATAYGLMQKLELGRYNETHAIAKWLLEKQELG
GGFRSTQPGRSSRLSHPQRWPQGSLXALEALTRFREAVPFKGIQDLHVQIRAPKTALNVNWYIDHSNAYOORSAK
FLAQDDLEIKASGNGRGTISILTMYHKSPESREDNCNLYHLNATLHSALEENKKGGETFRLRMETRFQNNGEATM
TIMEVSLLTCFYPNQDDLKQLTSDVERYAFQYKTKTSTSDSTVVLYLEKLSHEKNTVLGFRVHRMLQAEFLQAAL
VTIYDYYEPSRRCSTFYNLPTEQSSLRKICHKDICRCAEGQCPSLQKPSGQLRQEELQTTACEAGVDFVYKTKLE
SVEVSASNPYVYYNTQLEDIIKSGTDPAKPLAMKKFVSHATCHDSLGLQEQESYLIMGQTSDLWRIKSDYSYVLG
KETFLILWPADGDASKKELRDQLEEFLEYMRTHGCQS

The full amino acid sequence of the protein of the invention was found to have 257 of 734 amino acids (35%) identical and 403 of 734 (54%) homolog to a Cavia porcellus (guinea pig) complement C3 precursor (contains: C3A anaphylatoxin) (ACC:P12387; 1666 aa), and 255 of 717 amino acid residues (35%) identical to, and 401 of 717 residues (55%) similar to, the 1663 amino acid residue complement C3 precursor (contains: C3A anaphylatoxin) from Homo sapiens (human) (ACC:P01024).

The disclosed MOL3 protein (SEQ ID NO:6) also has good identity with a number of complement compontent proteins, as shown in Table 3C.

TABLE 3C
BLAST results for MOL3

| Gene Index/ | Protein/ | Length | Identity | Positives | |
|---|---|---|---|---|---|
| Identifier | Organism | (aa) | (%) | (%) | Expect |
| gi\|4557385\|ref\|NP_{—} | complement | 1663 | 633/1750 | 938/1750 | 0.0 |
| 000055.1\| | component 3 | | (36%) | (53%) | |
| | precursor [Homo | | | | |
| | sapiens] | | | | |
| gi\|11869931\|gb\|AAG40565.1\| | complement | 1661 | 617/1733 | 935/1733 | 0.0 |
| AF154933_1 | component C3 | | (35%) | (53%) | |
| (AF154933) | [Sus scrofa] | | | | |
| gi\|309122\|gb\|AAC42013.1\| | preprocomplement | 1663 | 606/1734 | 915/1734 | 0.0 |
| (K02782) | component C3 | | (34%) | (51%) | |
| | [Mus musculus] | | | | |

This information is presented graphically in the multiple sequence alignment given in Table 3D (with MOL3 being shown on line 1) as a ClustalW analysis comparing MOL3 with related protein sequences.

[image]

[image]

[image]

MOL3 shows significant homologies to human complement C3 proteins, as described in, but not limited to, the references below.

It was found that transforming growth factor-betal acts as a potent inhibitor of complement C3 biosynthesis in human pancreatic cancer cell lines. Andoh et al. determined how transforming growth factor (TGF)-beta1 affects complement C3 secretion in the pancreatic cancer cell lines PANC-1 and BxPC-3. It is suggested that TGF-betal may act as a potent inhibitor of C3 secretion in pancreatic cancer cell lines under inflammatory conditions. This action of TGF-betal did not correlate with NF-kappaB activation, but associated with the translocation of Fos protein into the nucleus.

The cellular localization of complement C3 and C4 transcripts were analyzed in intestinal specimens from patients with Crohn's disease. It has been suggested that the increase in C3 and C4 levels in jejunal perfusates of patients with Crohn's disease results from local intestinal synthesis of complement. Laufer et al. suggest that there is local regulated production of complement in the intestine of patients with CD, and subsequent complement activation may contribute to the inflammatory process.

The generation of complement C3 and expression of cell membrane complement inhibitory proteins by human bronchial epithelium cell line. They found that the interrelationship between human airway epithelium and complement proteins may affect airway defence, airway function, and airway epithelial integrity. Local generation of complement C3 and expression of cell membrane CIP by human bronchial epithelium and its modulation by proinflammatory cytokines might be an additional regulatory mechanism of local airway defence and may affect airway function and epithelial integrity in health and disease.

Janssen et al, Am J Kidney Dis January 2000;35(1):21-8 suggested the activation of the acute phase response and complement C3 in patients with IgA nephropathy. The authors have shown ystemic complement activation in patients with immunoglobulin A (IgA) nephropathy and reported that plasma levels of actC3 can indicate disease activity and renal outcome.

Therapeutic Applications

The expression pattern, and protein similarity information for MOL3 may function as a human complement C3-like protein. Therefore, the nucleic acid and protein of the invention are useful in potential therapeutic applications implicated, for example but not limited to, cancer, lung diseases, including asthma, immundeficiencies, inflammation, Crohn's disease, neurological disorders, nephropathy, and other diseases and disorders. The homology to antigenic secreted and membrane proteins suggests that antibodies directed against the novel genes may be useful in treatment and prevention of cancer, lung diseases, including asthma, immundeficiencies, inflammation, Crohn's disease, neurological disorders, nephropathy, and other diseases and disorders.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

The nucleic acids and proteins of the invention are useful in potential therapeutic applications implicated in cancer, lung diseases, including asthma, immundeficiencies, inflammation, Crohn's disease, neurological disorders, nephropathy, and other diseases and disorders. For example, but not limited to, a cDNA encoding the human complement C3-like protein may be useful in gene therapy, and the human complement C3-like protein may be useful when administered to a subject in need thereof. By way of non-limiting example, the compositions of the present invention will have efficacy for treatment of patients suffering from, for example, but not limited to, cancer, lung diseases, including asthma, immundeficiencies, inflammation, Crohn's disease, neurological disorders, nephropathy, and other diseases and disorders. The novel nucleic acid encoding the human complement C3-like protein, and the human complement C3-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL4

MOL4a

The disclosed Wnt 8-like protein, MOL4a (also referred to herein as AC004826), is encoded by a nucleic acid, 1064 nucleotides long (SEQ ID NO:7). An open reading frame was identified beginning with an ATG initiation codon at nucleotides 4-6 and ending with a TGA codon at nucleotides 1057-1059. Putative untranslated regions upstream from the initiation codon and downstream from the termination codon are underlined in Table 4A, and the start and stop codons are in bold letters. The encoded protein having 351 amino acid residues is presented using the one-letter code in Table 4B (SEQ ID NO:8).

TABLE 4A
MOL4a Nucleotide Sequence.

(SEQ ID NO:7)

GCGATGGGGAACCTGTTTATGCTCTGGGCACCTCTGGGCATATGCTGTGCTGCATTCAGTGCCTCTGCCTGGTCA
GTGAACAATTTCCTGATAACAGGTCCCAACCCCTATCTGACCTACACGACTAGTGTGGCCTTGGGTGCCCAGAGT
GGCATCCAGGAGTGCAACTTCCAGTTTGCTTGGGAACGCTGGAACTGCCCTGAAAATGCTCTTCAGCTCTCCACC
CACAACAGGCTGAGAAGTGCTACCAGAGAGACTTCCTTCATACATGCTATCAGCTCTGCTGGAGTCATGTACATC
ATCACCAAGAACTGTACCATGGGTGACTTCGAAAACTGTGGCTGTGATGGGTCAAACAATGGAAAAATACCAGGC
CATGGCTGGATCTCGGGAGGCTGCACCGACAATGTGGAATTTGGGGAAAGGATCTCCAAACTCTTTGTGCACAGT
TTGGAGAAGCGCAAGGATGCCAGAGCCCTGATGAATCTTCACAACAACAGGGCCGGCAGACTGGCAGTGAGAGCC
ACCATGAAAACGACATCCAAATGTCATGGCATCTCTGGGACCTGCAGCATACAGACATGCTGGCTGCAGCTGGCT
GAATTCCGGGAGATGGGAGACTACCTAAAGGCCAAGTATGACCAGCCGCTGAAAATTCAAATGGATAAGCGGCAG
CTGAGAGCTCGCAACACCGCCCACGGCCACTGCGTGCCCGCTGAGCCCTTCCTTCCTACCGCAGAGGCCGAACTG
ATCTTTTTAGAGGAATCACCACATTACTCTACCTGCAATTCCAGCCTGGGCATCTATGGCACAGAGCGTCGTGAC
TGCCTACAGAACAGCCACAACACATCCAGGTGCGAGCGACGTAGCTGTGGGCGCCTGTCCACTGAGTGTGGGCTC
CAGGTCGAAGACACGAAAACTGAGGTCATAAGCAGCTCTAACTGCAAATTCCAGTGGTCCTGTACGGTCAAGTGT
GACCAGTGTAGGCATGTGGTGAGCAAGTATTACTGCGCACGCTCCCCAGGCAGTGCCCAGTCCCTCGGTAACGGC
AGTGCCTGATAATA

The disclosed nucleic acid MOL4a sequence has 881 of 1050 bases (83%) identical to a Mus musculus Wnt 8 mRNA (GENBANK-ID: MMWNT8DPT|acc:Z68889) and 637 of 955 bases (66%) identical to a Homo sapiens Wnt 8 mRNA (GENBANK-ID: HSWNT8|acc:Y11094).

The MOL4a polypeptide (SEQ ID NO:8) encoded by SEQ ID NO:7 is presented using the one-letter amino acid code in Table 4B. The Psort profile for MOL4a predicts that this sequence has a signal peptide and is likely to be localized outside the cell with a certainty of 0.7700. The most likely cleavage site for a MOL4a peptide is between amino acids 24 and 25 based on the SignalP result.

TABLE 4B
MOL4a protein sequence

(SEQ ID NO:8)

MGNLFMLWAALGICCAAFSASAWSVNNFLITGPKAYLTYTTSVALGAQSGIEECKFQFAWERWNCPENALQLSTHN
RLRSATRETSFIHAISSAGVMYIITKNCSMGDFENCGCDGSNNGKIGGHGWIWGGCSDNVEFGERISKLFVDSLEK
GKDARALMNLHNNRAGRLAVRATMKRTCKCHGISGSCSIQTCWLQLAEFREMGDYLKAKYDQALKIEMDKRQLRAG
NSAEGHWVPAEAFLPSAEAELTFLEESPDYCTCNSSLGIYGTEGRECLONSHNTSRWERRSCGRLCTECGLQVEER
KTEVISSCNCKFQWCCTVKCDQCRHVVSKYYCARSPGSAQSLGKGSA

The full amino acid sequence of the disclosed MOL4a polypeptide has 282 of 349 amino acid residues (80%) identical to, and 306 of 349 residues (87%) positive with, the 354 amino acid residue WNT-8D protein from Mus musculus (ptnr:SPTREMBL-ACC: Q64527), (E=1.5×10⁻¹⁶)

MOL4b

The disclosed Wnt 8-like protein, MOL4b (also referred to herein as CG53138-05), is encoded by a nucleic acid, 1155 nucleotides long (SEQ ID NO:140). An open reading frame was identified beginning with an ATG initiation codon at nucleotides 41-43 and ending with a TGA codon at nucleotides 1148-1150. Putative untranslated regions upstream from the initiation codon and downstream from the termination codon are underlined in Table 4C, and the start and stop codons are in bold letters.

TABLE 4C
MOL4b Nucleotide Sequence.

(SEQ ID NO:140)

CGATGGGGAACCTGTTTATGCTCTGGGCAGCTCTGGGCATATGCTGTGCTGCATTCAGTGCCTCTGCCTGGTAAG
TCCTTTCCCAACCCTCACTCCTTGCCAAGGAGCCCCCCATTGTCTCATCCCCATTCACCTCTCCCTCACTTTTTC
TCTTTTTGCTACGTCAGTGAACAATTTCCTGATAACACGTCCCAAGCCCTATCTGACCTACACGACTAGTGTCGC
CTTGGGTGCCCAGAGTGGCATCGAGGAGTGCAAGTTCCAGTTTGCTTGCCAACGCTGCAACTCCCCTCAAAATGC
TCTTCAGCTCTCCACCCACAACAGGCTGAGAAGTGCTACCAGAGAGACTTCCTTCATACATGCTATCAGCTCTGC
TGGAGTCATGTACATCATCACCAAGAACTCTACCATGGGTGACTTCGAAAACTGTGGCTGTGATGGGTCAAACAA
TGGAAAAACAGGACGCCATGGCTGGATCTGGGGAGCCTGCAGCGACAATGTCGAATTTGGGGAAAGGATCTCCAA
ACTCTTTGTGGACAGTTTGGAGAAGGGGAAGGATGCCAGAGCCCCGATGAATCTTCACAACAACAGGGCCGGCAG
ACTGGCAGTCAGACCCACCATGAAAAGGACATGCAAATGTCATGGCATCTCTGGGAGCTGCAGCATACAGACATG
CTGGCTCCAGCTGGCTGAATTCCGGGACATGCGAGACTACCTAAAGGCCAAGTATGACCAGGCGCTGAAAATTGA
AATCGATAACCGGCAGCTCAGAGCTGGGAACAGCCCCGAGGGCCACTGGGTGCCCGCTGAGGCCTTCCTTCCTAC
CGCAGAGGCGGAACTGATCTTTTTAGAGGAATCACCAGATTACTGTACCTCCAATTCCAGCCTGGCCATCTATGG
CACAGAGGGTCGTGAGTCCCTACAGAACACCCACAACACATCCAGGTGGGAGCGACGTAGCTGTCCGCGCCTGTG
CACTGAGTGTGGGCTGCACGTGGAAGACAGGAAAACTGAGGTCATAAGCAGCTGTAACTGCAAATTCCAGTGGTG
CTGTACGGTCAAGTGTGACCAGTGTAGGCATGTCGTGAGCAAGTATTACTGCGCACGCTCCCCAGCCACTGCCCA
GTCCCTGGGTAAGGGCAGTGCCTGATAATA

The disclosed nucleic acid MOL4b sequence has been localized to chromosome 5 and has 993 of 994 bases (99%) identical to a gb:GENBANK-ID:AB057725|acc:AB057725.1 mRNA from Homo sapiens (Homo sapiens mRNA for WNT8A, complete cds) (E=1.7e⁻²³²).

The MOL4b 369 amino acid polypeptide (SEQ ID NO:141) encoded by SEQ ID NO:140 is presented using the one-letter amino acid code in Table 4D. The Psort profile for MOL4b predicts that this sequence has a signal peptide and is likely to be localized outside the cell with a certainty of 0.4132. In other embodiments, NOV4b could also be localized to the lysosome (lumen) with a certainty of 0.1900, to the endoplasmic reticulum (membrane) with a certainty of 0.1000, or to the endoplasmic reticulum (lumen) with a certainty of 0.1000. The most likely cleavage site for a MOL4b peptide is between amino acids 42 and 43 (GRS-VN) based on the SignalP result.

TABLE 4D
MOL4b protein sequence

(SEQ ID NO:141)

MLCCIQCLCLVSPFPTLTPCQGGPHCLIPIHLCLTFSLFGRSVNNFLITGPKAYLTYTTSVALGAQSGIEECKFQF
AWERWNCPENALQLSTHNRLRSATRETSFIHAISSAGVMYIITKNCSMGDFENCGCDGSNNGKTGGHGWIWGGCSD
NVEFGERISKLFVDSLEKGKDARAPMNLHNNRAGRLAVRATMKRTCKCHGISGSCSIQTCWLQLAEFREMGDYLKA
KYDQALKIEMDKRQLRAGNSAEGHWVPAEAFLPSAEAELIFLEESPDYCTCNSSLGIYGTEGRECLQNSHNTSRWE
RRSCGRLCTECGLQVEERKTEVISSCNCKFQWCCTVKCDQCRHVVSKYYCARSPGSAQSLGKGSA

The full amino acid sequence of the disclosed MOL4b polypeptide has 2330 of 337 amino acid residues (97%) identical to, and 330 of 337 amino acid residues (97%) similar to, the 351 amino acid residue ptnr:TREMBLNEW-ACC:BAB60960 protein from Homo sapiens (Human) (WNT8A) (E=2.0×10⁻¹⁸⁵)

Expression information was derived from the tissue sources of the sequences that were included in the derivation of the sequence of MOL4b and MOL4c. The sequences are predicted to be expressed in the teratocarcinoma cell line NT2 because of the expression pattern of (GENBANK-ID: gb:GENBANK-ID:AB057725|acc:AB057725.1) a closely related Homo sapiens mRNA for WNT8A, complete cds homolog.

MOL4c

The disclosed Wnt 8-like protein, MOL4c (also referred to herein as CG53138-06), is encoded by a nucleic acid, 1175 nucleotides long (SEQ ID NO:142). An open reading frame was identified beginning with an ATG initiation codon at nucleotides 41-43 and ending with a TAG codon at nucleotides 1160-1162. Putative untranslated regions upstream from the initiation codon and downstream from the termination codon are underlined in Table 4E, and the start and stop codons are in bold letters.

TABLE 4E
MOL4c Nucleotide Sequence.

(SEQ ID NO:142)

CGATGGGGAACCTGTTTATGCTCTGGGCAGCTCTGGGCATATGCTGTGCTGCATTCAGTGCCTCTGCCTGGTAAG
TCCTTTCCCAACCCTCACTCCTTGCCAAGGAGCCCCCCATTGTCTCATCCCCATTCACCTCTGCCTCACTTTTTC
TCTTTTTGCTAGGTCAGTGAACAATTTCCTGATAACACGTCCCAACGCCTATCTGACCTACACGACTAGTGTGGC
CTTGGCTGCCCACAGTGGCATCGAGGAGTGCAAGTTCCAGTTTCCTTGGCAACGCTGGAACTGCCCTGAAAATGC
TCTTCAGCTCTCCACCCACAACAGGCTGAGAAGTGCTACCAGAGAGACTTCCTTCATACATGCTATCAGCTCTGC
TGGAGTCATGTACATCATCACCAAGAACTGTAGCATGGGTGACTTCGAAAACTGTCCCTGTGATGGGTCAAACAA
TGGAAAAACAGGAGGCCATGGCTGGATCTGGGGAGGCTGCAGCGACAATGTGGAATTTGGGGAAAGGATCTCCAA
ACTCTTTGTGGACAGTTTGGAGAAGGGGAAGCATGCCACAGCCCCGATGAATCTTCACAACAACAGGGCCGGCAG
ACTGGCACTCAGAGCCACCATGAAAAGGACATGCAAATGTCATGGCATCTCTGGGACCTGCAGCATACAGACATG
CTGGCTGCACCTGGCTGAATTCCCGGACATGGGAGACTACCTAAAGGCCAAGTATGACCAGCCGCTGAAAATTGA
AATGCATAAGCGGCACCTGAGACCTCGCAACAGCGCCGAGGCCCACTGGGTGCCCGCTGACGCCTTCCTTCCTAG
CGCAGAGGCGGAACTGATCTTTTTAGAGGAATCACCAGATTACTGTACCTGCAATTCCAGCCTGGCCATCTATGG
CACAGAGGGTCGTCAGTGCCTACAGAACAGCCACAACACATCCAGCTCGCAGCCACGTAGCTGTGGGCGCCTGTG
CACTGACTGTGCGCTGCAGGTGGAAGAGAGGAAAACTGAGGTCATAAGCAGCTGTAACTGCAAATTCCAGTGGTC
CTGTACGGTCAAGTGTGACCAGTGTAGGCATGTGGTGAGCAAGTATTACTGCGCACGCTCCCCAGGCAGTGCCCA
GTCCCTGGGGAGAGTTTGGTTTGGGGTCTATATCTAGAGGGACCTTCAAA

The MOL4c 373 amino acid polypeptide (SEQ ID NO:143) encoded by SEQ ID NO:142 is presented using the one-letter amino acid code in Table 4F.

TABLE 4F
MOL4c protein sequence

(SEQ ID NO:143)

MLCCIQCLCLVSPFPTLTPCQGGPHCLIPIHLCLTFSLFGRSVNNFLITGPKAYLTYTTSVALGAQSGIEECKFQF
AWERWNCPENALQLSTHNRLRSATRETSFIHAISSAGVMYIITKNCSMGDFENCGCDGSNNGKTGGHGWIWGGCSD
NVEFGERISKLFVDSLEKGKDARAPMNLHNNRAGRLAVRATMKRTCKCHGISGSCSIQTCWLQLAEFREMGDYLKA
KYDQALKIEMDKRQLRACNSAEGHWVPAEAFLPSAEAELIFLEESPDYCTCNSSLGTYGTEGRECLQNSHNTSRWE
RRSCGRLCTECGLQVEERKTEVISSCNCKFQWCCTVKCDQCRHVVSKYYCARSPGSAQSLGRVWFGVYI

Expression information was derived from the tissue sources of the sequences that were included in the derivation of the sequence of MOL4b and MOL4c. The sequences are predicted to be expressed in the teratocarcinoma cell line NT2 because of the expression pattern of (GENBANK-ID: gb:GENBANK-ID:AB057725|acc:AB057725.1) a closely related Homo sapiens mRNA for WNT8A, complete cds homolog.

MOL4d

The disclosed Wnt 8-like protein, MOL4d (also referred to herein as CG53138-04), is encoded by a nucleic acid, 1597 nucleotides long (SEQ ID NO:144). An open reading frame was identified beginning with an ATG initiation codon at nucleotides 41-43 and ending with a TAG codon at nucleotides 1166-1168. Putative untranslated regions upstream from the initiation codon and downstream from the termination codon are underlined in Table 4G, and the start and stop codons are in bold letters.

TABLE 4G
MOL4d Nucleotide Sequence.

(SEQ ID NO:144)

CAGAATTTTCTCACATAAATACTGAGGAAGACCCTGCCCTCTCCTCACTCCTCTGGACTTGGCCCTGAGCTGGAC
CTGGTCCACTGGGGTAGGCAGGGCGATGGGGAACCTGTTTATGCTCTGGGCAGCTCTGGGCATATGCTGTGCTGC
ATTCAGTGCCTCTGCCTGGTCAGTGAACAATTTCCTGATAACAGGTCCCAAGGCCTATCTGACCTACACGACTAG
TGTGGCCTTGGGTGCCCAGAGTGGCATCGAGGAGTGCAAGTTCCAGTTTGCTTGGGAACGCTGGAACTGCCCTGA
AAATGCTCTTCAGCTCTCCACCCACAACAGGCTGAGAAGTGCTACCAGAGAGACTTCCTTCATACATGCTATCAG
CTCTGCTGGAGTCATGTACATCATCACCAAGAACTGTAGCATGGGTGACTTCGAAAACTGTGGCTGTGATGGGTC
AAACAATGGAAAAACAGGAGGCCATGGCTGGATCTGGGGAGGCTGCAGCGACAATGTGGAATTTGGGGAAAGGAT
CTCCAAACTCTTTGTGGACAGTTTGGAGAAGGGGAAGGATGCCAGAGCCCTGATGAATCTTCACAACAACAGGGC
CGGCAGACTGGCAGTGAGAGCCACCATGAAAAGGACATGCAAATGTCATGGCATCTCTGGGAGCTGCAGCATACA
GACATGCTGGCTGCAGCTGGCTGAATTCCGGGAGATGGGAGACTACCTAAAGGCCAAGTATGACCAGGCGCTGAA
AATTGAAATGGATAAGCGGCAGCTGAGAGCTGGGAACAGCGCCGAGGGCCACTGGGTGCCCGCTGAGGCCTTCCT
TCCTAGCGCAGAGGCGGAACTGATCTTTTTAGAGGAATCACCAGATTACTGTACCTGCAATTCCAGCCTGGGCAT
CTATGGCACAGAGGGTCGTGAGTGCCTACAGAACAGCCACAACACATCCAGGTGGGAGCGACGTAGCTGTGGGCG
CCTGTGCACTGAGTGTGGGCTGCAGGTGGAAGAGAGGAAAACTGAGGTCATAAGCAGCTGTAACTGCAAATTCCA
GTGGTGCTGTACGGTCAAGTGTGACCAGTGTAGGCATGTGGTGAGCAAGTATTACTGCGCACGCTCCCCAGGCAG
TGCCCAGTCCCTGGGGAGAGTTTGGTTTGGGGTCTATATCTAGAGGGACCTTCAAAGTATTTGTTCCTTTAAATT
TCAGACCATGTCCAACCCAGCTGTGCTGCTGGGAATCAGGAGAATAGAAGCAAAAAACGAAAGAGTTCTGTTCAG
ACTTCTGAAGAGCAGCCTGTGGCTACAAATCTATGCTGATAAATGAGATTGAGAACTCAACTGTATTTTGCCATA
AATGCTTCTAAGATATATCCAGCTGGGACTTCTATTACTCCCTTTGGAAACCTTAAGATCAAAAAGGGAATAAGA
AACCCTTCTTCTGTATCCCAATAATCCACCAGGATAAAGGAGAAACTAGAAATATGCAACTCCCTTGATTTCAGT
GTTTGGCAGGTAACAAAAAATTGAGACCCAGACACTGGTCAACAGGAAAACAATACAGACTCCCAGAATTAGAAA
GTGTTATTTTAATGCAACCTAG

The MOL4d 355 amino acid polypeptide (SEQ ID NO:145) encoded by SEQ ID NO:144 is presented using the one-letter amino acid code in Table 4H.

TABLE 4H
MOL4d protein sequence

(SEQ ID NO:145)

MGNLFMLWAALGICCAAFSASAWSVNNFLITGPKAYLTYTTSVALGAQSGIEECKFQFAWERWNCPENALQLSTHN
RLRSATRETSFIHAISSAGVMYIITKNCSMGDFENCGCDGSNNGKTGGHGWIWGGCSDNVEFGERISKLFVDSLEK
GKDARALMNLHNNRAGRLAVRATMKRTCKCHGISGSCSIQTCWLQLAEFREMGDYLKAKYDQALKIEMDKRQLRAG
NSAEGHWVPAEAFLPSAEAELIFLEESPDYCTCNSSLGIYGTEGRECLQNSHNTSRWERRSCGRLCTECGLQVEER
KTEVISSCNCKFQWCCTVKCDQCRHVVSKYYCARSPGSAQSLGRVWFGVYI

MOL4e

The disclosed Wnt 8-like protein, MOL4e (also referred to herein as 251445570), is encoded by a nucleic acid, 964 nucleotides long (SEQ ID NO:146). An open reading frame was identified beginning with an ACC initiation codon at nucleotides 2-4 and ending with a GGC codon at nucleotides 962-964. Putative untranslated regions upstream from the initiation codon and downstream from the termination codon are underlined in Table 4I, and the start and stop codons are in bold letters. Because the start and stop codons for MOL4e are not tradistional initiation and termination codons, MOL4e could be a partial reading frame that extends further in the 5′ and/or 3′ directions.

TABLE 4I
MOL4e Nucleotide Sequence.

(SEQ ID NO:146)

CACCGGATCCTCTGCCTGGTCAGTGAACAATTTCCTGATAACAGGTCCCAAGGCCTATCTGACCTACACGACTAG
TGTGGCCTTGGGTGCCCAGAGTGGCATCGAGGAGTGCAAGTTCCAGTTTGCTTGGGAACGCTGGAACTGCCCTGA
AAATGCTCTTCAGCTCTCCACCCACAACAGGCTGAGAAGTGCTACCAGAGAGACTTCCTTCATACATGCTATCAG
CTCTGCTGGAGTCATGTACATCATCACCAAGAACTGTAGCATGGGTGACTTCGAAAACTGTGGCTGTGATGGGTC
AAACAATGGAAAAACAGGAGGCCATGGCTGGATCTGGGGAGGCTGCAGCGACAATGTGGAATTTGGGGAAAGGAT
CTCCAAACTCTTTGTGGACAGTTTGGAGAAGGGGAAGGATGCCAGAGCCCTGATGAATCTTCACAACAACAGGGC
CGGCAGACTGGCAGTGAGAGCCACCATGAAAAGGACATGCAAATGTCATGGCATCTCTGGGAGCTGCAGCATACA
GACATGCTGGCTGCAGCTGGCTGAATTCCGGGAGATGGGAGACTACCTAAAGGCCAAGTATGACCAGGCGCTGAA
AATTGAAATGGATAAGCGGCAGCTGAGAGCTGGGAACAGCGCCGAGGGCCACTGGGTGCCCGCTGAGGCCTTCCT
TCCTAGCGCAGAGGCGGAACTGATCTTTTTAGAGGAATCACCAGATTACTGTACCTGCAATTCCAGCCTGGGCAT
CTATGGCACAGAGGGTCGTGAGTGCCTACAGAACAGCCACAACACATCCAGGTGGGAGCGACGTAGCTGTGGGCG
CCTGTGCACTGAGTGTGGGCTGCAGGTGGAAGAGAGGAAAACTGAGGTCATAAGCAGCTGTAACTGCAAATTCCA
GTGGTGCTGTACGGTCAAGTGTGACCAGTGTAGGCATGTGGTGAGCAAGTATTACCTCGAGGGC

The MOL4e 321 amino acid polypeptide (SEQ ID NO:147) encoded by SEQ ID NO:146 is presented using the one-letter amino acid code in Table 4J.

TABLE 4J
MOL4e protein sequence

(SEQ ID NO:147)

TGSSAWSVNNFLITGPKAYLTYTTSVALGAQSGIEECKFQFAWERWNCPENALQLSTHNRLRSATRETSFIHAISS
AGVMYIITKNCSMGDFENCGCDGSNNGKTGGHGWIWGGCSDNVEFGERISKLFVDSLEKGKDARALMNLHNNRAGR
LAVRATMKRTCKCHGISGSCSIQTCWLQLAEFREMGDYLKAKYDQALKIEMDKRQLRAGNSAEGHWVPAEAFLPSA
EAELIFLEESPDYCTCNSSLGIYGTEGRECLQNSHNTSRWERRSCGRLCTECGLQVEERKTEVISSCNCKFQWCCT
VKCDQCRHVVSKYYLEG

MOL4f

The disclosed Wnt 8-like protein, MOL4f (also referred to herein as 250059708), is encoded by a nucleic acid, 952 nucleotides long (SEQ ID NO:148). An open reading frame was identified beginning with an GGC initiation codon at nucleotides 2-4 and ending with a GGC codon at nucleotides 950-952. Putative untranslated regions upstream from the initiation codon and downstream from the termination codon are underlined in Table 4K, and the start and stop codons are in bold letters. Because the start and stop codons for MOL4f are not tradistional initiation and termination codons, MOL4f could be a partial reading frame that extends further in the 5′ and/or 3′ directions.

TABLE 4K
MOL4f Nucleotide Sequence.

(SEQ ID NO:148)

CACCGGATCCGTGAACAATTTCCTGATAACAGGTCCCAAGGCCTATCTGACCTACACGACTAGTGTGGCCTTGGG
TGCCCAGAGTGGCATCGAGGAGTGCAAGTTCCAGTTTGCTTGGGAACGCTGGAACTGCCCTGAAAATGCTCTTCA
GCTCTCCACCCACAACAGGCTGAGAAGTGCTACCAGAGAGACTTCCTTCATACATGCTATCAGCTCTGCTGGAGT
CATGTACATCATCACCAAGAACTGTAGCATGGGTGACTTCGAAAACTGTGGCTGTGATGGGTCAAACAATGGAAA
AACAGGAGGCCATGGCTGGATCTGGGGAGGCTGCAGCGACAATGTGGAATTTGGGGAAAGGATCTCCAAACTCTT
TGTGGACAGTTTGGAGAAGGGGAAGGATGCCAGAGCCCTGATGAATCTTCACAACAACAGGGCCGGCAGACTGGC
AGTGAGAGCCACCATGAAAAGGACATGCAAATGTCATGGCATCTCTGGGAGCTGCAGCATACAGACATGCTGGCT
GCAGCTGGCTGAATTCCGGGAGATGGGAGACTACCTAAAGGCCAAGTATGACCAGGCGCTGAAAATTGAAATGGA
TAAGCGGCAGCTGAGAGCTGGGAACAGCGCCGAGGGCCACTGGGTGCCCGCTGAGGCCTTCCTTCCTAGCGCAGA
GGCGGAACTGATCTTTTTAGAGGAATCACCAGATTACTGTACCTGCAATTCCAGCCTGGGCATCTATGGCACAGA
GGGTCGTGAGTGCCTACAGAACAGCCACAACACATCCAGGTGGGAGCGACGTAGCTGTGGGCGCCTGTGCACTGA
GTGTGGGCTGCAGGTGGAAGAGAGGAAAACTGAGGTCATAAGCAGCTGTAACTGCAAATTCCAGTGGTGCTGTAC
GGTCAAGTGTGACCAGTGTAGGCATGTGGTGAGCAAGTATTACCTCGAGGGC

The MOL4f 317 amino acid polypeptide (SEQ ID NO:149) encoded by SEQ ID NO:148 is presented using the one-letter amino acid code in Table 4L.

TABLE 4L
MOL4f protein sequence

(SEQ ID NO:149)

TGSVNNFLITGPKAYLTYTTSVALGAQSGIEECKFQFAWERWNCPENALQLSTHNRLRSATRETSFIHAISSAGVM
YIITKNCSMGDFENCGCDGSNNGKTGGHGWIWGGCSDNVEFGERISKLFVDSLEKGKDARALMNLHNNRAGRLAVR
ATMKRTCKCHGISGSCSIQTCWLQLAEFREMGDYLKAKYDQALKIEMDKRQLRAGNSAEGHWVPAEAFLPSAEAEL
IFLEESPDYCTCNSSLGIYGTEGRECLQNSHNTSRWERRSCGRLCTECGLQVEERKTEVISSCNCKFQWCCTVKCD
QCRHVVSKYYLEG

MOL4g

The disclosed Wnt 8-like protein, MOL4g (also referred to herein as 246861879), is encoded by a nucleic acid, 726 nucleotides long (SEQ ID NO:150). An open reading frame was identified beginning with an CGC initiation codon at nucleotides 1-3 and ending with a GCG codon at nucleotides 724-726. Putative untranslated regions upstream from the initiation codon and downstream from the termination codon are underlined in Table 4M, and the start and stop codons are in bold letters. Because the start and stop codons for MOL4g are not tradistional initiation and termination codons, MOL4g could be a partial reading frame that extends further in the 5′ and/or 3′ directions.

TABLE 4M
MOL4g Nucleotide Sequence.

(SEQ ID NO:150)

CGCGGATCCACCATGAAATATGTCTTCTATTTGGGTGTCCTCGCTGGGACATTTTTCTTTGCTGACTCATCTGTT
CAGAAAGAAGACCCTGCTCCCTATTTGGTGTACCTCAAGTCTCACTTCAACCCCTGTGTGGGCGTCCTCATCAAA
CCCAGCTGGGTGCTGGCCCCAGCTCACTGCTATTTACCAAATCTGAAAGTGATGCTGGGAAATTTCAAGAGCAGA
GTCAGAGACGGTACTGAACAGACAATTAACCCCATTCAGATCGTCCGCTACTGGAACTACAGTCATAGCGCCCCA
CAGGATGACCTCATGCTCATCAAGCTGGCTAAGCCTGCCATGCTCAATCCCAAAGTCCAGCCCCTTCCCCTCGCC
ACCACCAATGTCAGGCCAGGCACTGTCTGTCTACTCTCAGGTTTGGACTGGAGCCAAGAAAACAGTGGCCGACAC
CCTGACTTGCGGCAGAACCTGGAGGCCCCCGTGATGTCTGATCGAGAATGCCAAAAAACAGAACAAGGAAAAAGC
CACAGGAATTCCTTATGTGTGAAATTTGTGAAAGTATTCAGCCGAATTTTTGGGGAGGTGGCCGTTGCTACTGTC
ATCTGCAAAGACAAGCTCCAGGGAATCGAGGTGGGGCACTTCATGGGAGGGGACGTCGGCATCTACACCAATGTT
TACAAATATGTATCCTGGATTGAGAACACTGCTAAGGACAAGCTCGAGGCG

The MOL4g 242 amino acid polypeptide (SEQ ID NO:151) encoded by SEQ ID NO:150 is presented using the one-letter amino acid code in Table 4N.

TABLE 4N
MOL4g protein sequence

(SEQ ID NO:151)

RGSTMKYVFYLGVLAGTFFFADSSVQKEDPAPYLVYLKSHFNPCVGVLIKPSWVLAPAHCYLPNLKVMLGNFKSRV
RDGTEQTINPIQIVRYWNYSHSAPQDDLMLIKLAKPAMLNPKVQPLPLATTNVRPGTVCLLSGLDWSQENSGRHPD
LRQNLEAPVMSDRECQKTEQGKSHRNSLCVKFVKVFSRIFGEVAVATVICKDKLQGIEVGHFMGGDVGIYTNVYKY
VSWIENTAKDKLEA

BLASTP (Non-Redundant Composite database) analysis of the best hits for alignments with MOL4a are listed in Table 4C.

TABLE 4C
BLASTP results for MOL4a

| Gene Index/ | | Length | Identity | Positives | |
|---|---|---|---|---|---|
| Identifier | Protein/Organism | (aa) | (%) | (%) | Expect |
| gi\|11693046\|gb\| | WNT8d precursor | 355 | 335/348 | 336/348 | 0.0 |
| AAG38662.1\| | [Homo sapiens] | | (96%) | (96%) | |
| (AY009402) | | | | | |
| gi\|6678169\|ref\| | stimulated by | 354 | 271/349 | 295/349 | 1.0e−148 |
| NP_033316.1\| | retinoic acid | | (77%) | (83%) | |
| | gene 11 [Mus | | | | |
| | musculus] | | | | |
| gi\|104264\|pir\|\| | developmental | 387 | 246/335 | 285/335 | 1.0e−136 |
| S18771 | regulator Xwnt-8 - | | (73%) | (84%) | |
| | African clawed | | | | |
| | frog | | | | |
| gi\|1722844\|sp\|P51030\| | WNT-8C PROTEIN | 357 | 242/337 | 283/337 | 1.0e−134 |
| WN8C_CHICK | PRECURSOR | | (71%) | (83%) | |
| | (CWNT-8) | | | | |

This information is presented graphically in the multiple sequence alignment given in Table 4D (with MOL4 being shown on line 1) as a ClustalW analysis comparing MOL4 with related sequences.

[image]

[image]

WNT genes encode intercellular signaling glycoproteins that play important roles in key processes of embryonic development such as mesoderm induction, specification of the embryonic axis, and patterning of the central nervous system, spinal cord, and limbs. The name WNT denotes the relationship of this family to the Drosophila segment polarity gene ‘wingless,’ and to its vertebrate ortholog Int1, a mouse protooncogene; see WNT1. It was noted that multiple WNT genes are known to exist in several species that have been investigated ranging from Drosophila to man. They have been classified into various groups and subgroups on the basis of high sequence homology and common expression patterns. The vertebrate WNT8 subfamily includes genes from Xenopus, zebrafish, and chicken; The first mammalian WNT8 homolog, a human member of the Wnt8 family that they termed WNT8B was characterized on the basis of the very high sequence similarity (90-91% identity) of the inferred protein to those encoded by the Xenopus and zebrafish Wnt8b genes. The human cDNA encodes a 295-amino acid polypeptide that contains a C2H2 zinc finger-like motif. A predominant 1.9-kb mRNA was detected in a variety of adult and fetal tissues. They used PCR typing of a human monochromosomal hybrid cell panel to map the gene to chromosome 10, and fluorescence in situ hybridization for localization at 10q24.

The full-length cDNA sequence and genomic organization of the human WNT8B gene was presented and reported studies of expression of the gene in human and mouse embryos. The WNT8B gene contains six exons separated by small introns, with the exception of intron 1. The predicted protein has 351 amino acids. The gene is expressed predominantly as a transcript of approximately 2.1 kb. The human and mouse expression patterns appeared to be identical and were restricted to the developing brain, with the great majority of expression being found in the developing forebrain. In the latter case, expression was confined to the germinative neuroepithelium of three sharply delimited regions: the dorsomedial wall of the telencephalic ventricles (which includes the developing hippocampus), a discrete region of the dorsal thalamus, and the mammillary and retromammillary regions of the posterior hypothalamus. Expression in the developing hippocampus may suggest a role for WNT8B in patterning of this region, and subchromosomal localization of the human gene to 10q24 may suggest it as a candidate gene for partial epilepsy (EPT; OMIM-600512) in families in which the disease has been linked to markers in this region.

WNT1 is a member of a family of cysteine-rich, glycosylated signaling proteins that mediate diverse developmental processes such as the control of cell proliferation, adhesion, cell polarity, and the establishment of cell fates. Wnt1 was identified as an oncogene activated by the insertion of mouse mammary tumor virus in virus-induced mammary adenocarcinomas. Although Wnt1 is not expressed in the normal mammary gland, expression of Wnt1 in transgenic mice causes mammary tumors. To identify downstream genes in the WNT signaling pathway that are relevant to the transformed cell phenotype, A PCR-based cDNA subtraction strategy was used, suppression subtractive hybridization. It was reported that the identification of two genes, WISP1 and WISP2, that are upregulated in the mouse mammary epithelial cell line transformed by Wnt 1, but not by Wnt4. Together with a third related gene, WISP3, these proteins define a subfamily of the connective tissue growth factor family. Two distinct systems demonstrated WISP induction to be associated with the expression of WNT1. WISP1 genomic DNA was amplified in colon cancer cell lines and in human colon tumors and its RNA overexpressed in 84% of the tumors examined compared with patient-matched normal mucosa. WISP3 also was overexpressed in 63% of colon tumors analyzed. In contrast, WISP2 showed reduced RNA expression in 79% of the tumors. These results suggested that WISP genes may be downstream of WNT1 signaling and that aberrant levels of WISP expression in colon cancer may play a role in colon tumorigenesis.

It was found that the WISP1 cDNA encodes a 367-amino acid protein. Mouse and human WISP1 proteins are 84% identical; both have hydrophobic N-terminal signal sequences, 38 conserved cysteine residues, and 4 potential N-linked glycosylation sites. Alignment of the three human WISP proteins showed that WISP1 and WISP3 are most similar (42%), whereas WISP2 had 37% identity with WISP1 and 32% identity with WISP3.

Uses of the Compositions of the Invention

The above defined information for this invention suggests that MOL4 may function as a member of the “Wnt 8 family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in neurodegenerative disorders, epilepsy, cancers including but not limited to brain tumor, colon cancer and breast cancer, developmental disorders, neural tube defects, and/or other pathologies and disorders. For example, a cDNA encoding the Wnt 8-like protein may be useful in gene therapy, and the Wnt 8-like protein may be useful when administered to a subject in need thereof. By way of nonlimiting example, the compositions of the present invention will have efficacy for treatment of patients suffering from neurodegenerative disorders, epilepsy, cancers including but not limited to brain tumor, colon cancer and breast cancer, developmental disorders, and neural tube defects,. The novel nucleic acid encoding Wnt 8-like protein, and the Wnt 8-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL5

The disclosed novel Beta Thymosin-like MOL5 nucleic acid of 215 nucleotides (also referred to as AC025535) is shown in Table 5A. An ORF begins with an ATG initiation codon at nucleotides 4-7 and ends with a TGA codon at nucleotides 211-213. A putative untranslated region upstream from the initiation codon and downstream from the termination codon is underlined in Table 5A, and the start and stop codons are in bold letters.

TABLE 5A
MOL5 Nucleotide Sequence

(SEQ ID NO:9)

AGTATGGTCTCAGCCCAGCGTTTCACGAGTCTTCAAGCCTTCAGGCTTTCTTTAATCAAGATGAGTGATAACCCA
AACTTGTCAGAAGTGAAGTTTGACAGGTCAAAATTGAAGAAAACTAACACTGGAGAAAAAAATAGGCTTTCTTCC
AAGGAAACTATCCAGCAGGAGAAATACGGTGTTCAAACATCATATAATGGGGGCTGGGCATGATG

The MOL protein encoded by SEQ ID NO:9 has 69 amino acid residues and is presented using the one-letter code in Table 5B. The Psort profile for MOL5 predicts that this sequence has a signal peptide and is likely to be localized at the mitochondrial intermembrane space with a certainty of 0.8800. Using the SIGNALP analysis, the protein of the invention does not appear to contain a predictable signal peptide.

TABLE 5B
Encoded MOL5 protein sequence

(SEQ ID NO:10)

MVSAQRFTSLQAFRLSLIKMSDNPNLSEVKFDRSKLKKTNTGEKNRLSSKETIQQEKYGVQTSYNGGWA

The disclosed nucleic acid sequence for MOL5 has 167 of 191 bases (87%) identical to a Homo sapiens Beta Thymosin mRNA (GENBANK-ID: D82345|acc:D82345) (E=5.1e⁻²⁶).

The full MOL5 amino acid sequence has 37 of 45 amino acid residues (82%) identical to, and 38 of 45 residues (84%) positive with, the 45 amino acid residue Thymosin beta protein from Homo sapiens (ptnr: PIR-ID:JC5274) (E=1.2e⁻¹¹).

MOL5 also has homology to other proteins as shown in BLAST alignment results in Table 5C.

TABLE 5C
BLAST results for MOL5

| Gene Index/ | | Length | Identity | Positives | |
|---|---|---|---|---|---|
| Identifier | Protein/Organism | (aa) | (%) | (%) | Expect |
| ref\|NP_068832.1\| | thymosin, beta, | 45 | 37/45 | 38/45 | 5e−06 |
| | identified in | | (82%) | (84%) | |
| | neuroblastoma | | | | |
| | cells [Homo | | | | |
| | sapiens] | | | | |
| pir\|\|I52084 | thymosin beta-4 | 56 | 27/39 | 33/39 | 2e−04 |
| | precursor - rat | | (69%) | (84%) | |
| | (fragment) | | | | |
| sp\|P20065\|TYB4_MOUSE | THYMOSIN BETA-4 | 50 | 27/39 | 33/39 | 3e−04 |
| | | | (69%) | (84%) | |
| gb\|AAA36746.1\| | thymosin beta-10 | 49 | 24/40 | 32/40 | 0.002 |
| (M92383) | [Homo sapiens] | | (60%) | (80%) | |
| gb\|AAB37101.1\| | thymosin beta- | 45 | 31/39 | 34/39 | 0.002 |
| (U25684) | like protein | | (79%) | (86%) | |
| | [Rattus | | | | |
| | norvegicus] | | | | |

This information is presented graphically in the multiple sequence alignment given in Table 5D (with MOL5 being shown on line 1) as a ClustalW analysis comparing MOL5 with related protein sequences.

[image]

Thymosin-beta-4 induces the expression of terminal deoxynucleotidyl transferase activity in vivo and in vitro, inhibits the migration of macrophages, and stimulates the secretion of hypothalamic luteinizing hormone-releasing hormone. It was noted that the protein was originally isolated from a partially purified extract of calf thymus, thymosin fraction 5, which induced differentiation of T cells and was partially effective in some immuno-compromised animals. Further studies demonstrated that the molecule is ubiquitous; it had been found in all tissues and cell lines analyzed. It is found in highest concentrations in spleen, thymus, lung, and peritoneal macrophages. It was stated that thymosin-beta-4 is an actin monomer sequestering protein that may have a critical role in modulating the dynamics of actin polymerization and depolymerization in nonmuscle cells. Its regulatory role is consistent with the many examples of transcriptional regulation of T-beta-4 and of tissue-specific expression. Lymphocytes have a unique T-beta-4 transcript relative to the ubiquitous transcript found in many other tissues and cells. It was stated that rat thymosin-beta-4 is synthesized as a 44-amino acid propeptide which is processed into a 43-amino acid peptide by removal of the first methionyl residue. The molecule does not have a signal peptide. Human thymosin-beta-4 has a high degree of homology to rat thymosin-beta-4; the coding regions differ by only 9 nucleotides, and these are all silent base changes.

By differential screening of a cDNA library prepared from leukocytes of an acute lymphocytic leukemia patient, a cDNA encoding thymosin-beta-4 was isolated. Using Northern blot analysis, the expression of the 830-nucleotide thymosin-beta-4 mRNA in various primary myeloid and lymphoid malignant cell lines and in hemopoietic cell lines was studied. It was stated that the pattern of thymosin-beta-4 gene expression suggests that it may be involved in an early phase of the host defense mechanism.

A cDNA clone for the human interferon-inducible gene 6-26 was isolated and showed that its sequence was identical to that for the human thymosin-beta-4 gene. By use of a panel of human rodent somatic cell hybrids, it was shown that the 6-26 cDNA recognized seven genes, members of a multigene family, present on chromosomes 1, 2, 4, 9, 11, 20, and X. These genes are symbolized TMSL1, TMSL2, etc., respectively. Li et al. (1996) established that in the mouse there is a single Tmsb4 gene and that the lymphoid-specific transcript is generated by extending the ubiquitous exon 1 with an alternate downstream splice site. By interspecific backcross mapping, they located the mouse gene, which they symbolized Ptmb4, to the distal region of the mouse X chromosome, linked to Btk and Gja6. Thus, the human gene could be predicted to reside on the X chromosome in the general region of Xq21.3-q22, where BTK is located. By analysis of somatic cell hybrids, the thymosin-beta-4, or TB4X, gene were mapped to the X chromosome. They noted that a homologous gene, TB4Y, is present on the Y chromosome.

It was stated that prostate carcinoma is the most prevalent form of cancer in males and the second leading cause of cancer death among older males. The use of the serum prostate-specific antigen test permits early detection of human prostate cancer; however, early detection has not been accompanied by an improvement in determining which tumors may progress to the metastatic stage. The process of tumor metastasis is a multistage event involving local invasion and destruction of extracellular matrix; intravasation into blood vessels, lymphatics or other channels of transport; survival in the circulation; extravasation out of the vessels into the secondary site; and growth in the new location. Common to many components of the metastatic process is the requirement for tumor cell motility. A well-characterized series of cell lines that showed varying metastatic potential was developed from the Dunning rat prostate carcinoma. A direct correlation between cell motility and metastatic potential in the Dunning cell lines was shown. In studies comparing gene expression in poorly and highly motile metastatic cell lines derived from Dunning rat prostate carcinoma using differential mRNA display, Bao et al. (1996) found a novel member of the thymosin-beta family of actin-binding molecules. The molecule, named thymosin-beta-15 by them, was found to deregulate motility in prostate cells directly. In addition, it was expressed in advanced human prostate cancer specimens, but not in normal human prostate or benign prostatic hyperplasia, suggesting its potential use as a new marker for prostate carcinoma progression. Bao et al. (1996) found that thymosin-beta-15 levels correlated positively with the Gleason tumor grade. Coffey (1996) pointed out that the upregulation of thymosin-beta-15 as a positive motility factor and the down regulation of the motility suppressor KAI1 (OMIM-600623) provide the ‘yin and yang’ for metastasis; he speculated that these pathways may provide a new target for therapy.

Angiogenesis is an essential step in the repair process that occurs after injury. In a study, the angiogenic thymic peptide thymosin beta4 (Tbeta4) enhanced wound healing in a rat full thickness wound model was examined. Addition of Tbeta4 topically or intraperitoneally increased reepithelialization by 42% over saline controls at 4 d and by as much as 61% at 7 d post-wounding. Treated wounds also contracted at least 11% more than controls by day 7. Increased collagen deposition and angiogenesis were observed in the treated wounds. We also found that Tbeta4 stimulated keratinocyte migration in the Boyden chamber assay. After 4-5 h, migration was stimulated 2-3-fold over migration with medium alone when as little as 10 pg of Tbeta4 was added to the assay. These results suggest that Tbeta4 is a potent wound healing factor with multiple activities that may be useful in the clinic.

Uses of the Compositions of the Invention

The above defined information for this invention suggests that MOL5 may function as a member of a “Beta Thymosin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in cancer including but not limited to prostate cancer, immunological and autoimmune disorders (i.e., hyperthyroidism), angiogenesis and wound healing, modulation of apoptosis, neurodegenerative and neuropsychiatric disorders, age-related disorders, and other pathological disorders involving spleen, thymus, lung, and peritoneal macrophages and/or other pathologies and disorders. For example, a cDNA encoding the Beta Thymosin-like protein may be useful in gene therapy, and the Beta Thymosin-like protein may be useful when administered to a subject in need thereof. By way of nonlimiting example, the compositions of the present invention will have efficacy for treatment of patients suffering from cancer including but not limited to prostate cancer, immunological and autoimmune disorders (i.e., hyperthyroidism), angiogenesis and wound healing, modulation of apoptosis, neurodegenerative and neuropsychiatric disorders, age-related disorders, and other pathological disorders involving spleen, thymus, lung, and peritoneal macrophages. The novel nucleic acid encoding Beta Thymosin-like protein, and the Beta Thymosin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL6

MOL6a

The disclosed novel Trypsin-like MOL6a nucleic acid of 730 nucleotides (also referred to as GM_{—}87760758_A) is shown in Table 6A. An open reading begins with an ATG initiation codon at nucleotides 8-10 and ends with a TGA codon at nucleotides 713-715. A putative untranslated region upstream from the initiation codon and downstream from the termination codon are underlined in Table 6A, and the start and stop codons are in bold letters.

TABLE 6A
MOL6a Nucleotide Sequence

(SEQ ID NO:11)

GATCACCATGAAATATGTCTTCTATTTGCGTGTCCTCCCTGGGACATTTTTCTTTCCTGACTCATCTGTTCAGAA
AGAAGACCCTGCTCCCTATTTGGTGTACCTCAAGTCTCACTTCAACCCCTCTGTGGCCGTCCTCATCAAACCCAG
CTGGGTCCTGGCCCCAGCTCACTGCTATTTACCAAATCTGAAAGTCATGCTCGGAAATTTCAAGACCAGAGTCAG
AGACGGTACTGAACAGACAATTAACCCCATTCAGATCGTCCGCTACTGGAACTACAGTCATAGCGCCCCACAGGA
TGACCTCATGCTCATCAAGCTGGCTAAGCCTGCCATGCTCAATCCCAAAGTCCAGCCCCTTCCCCTCCCCACCAC
CAATGTCAGCCCAGGCACTGTCTGTCTACTCTCAGGTTTGGACTGGAGCCAAGAAAACAGTGGCCGACACCCTGA
CTTGCGGCAGAACCTGGAGGCCCCCGTGATGTCTGATCGAGAATGCCAAAAAACAGAACAAGGAAAAAGCCACAG
GAATTCCTTATGTGTGAAATTTGTGAAAGTATTCAGCCGAATTTTTGGGGAGGTGGCCGTTGCTACTGTCATCTG
CAAAGACAAGCTCCAGGGAATCGAGGTGGGGCACTTCATGGGAGGGGACGTCGGCATCTACACCAATGTTTACAA
ATATGTATCCTGGATTGAGAACACTGCTAAGGACAAGTAGGACCCTACTTCTCCC

The disclosed nucleic acid sequence has 354 of 581 bases (60%) identical to a Mus musculus prepro-Trypsininogen mRNA (GENBANK-ID: MMTRYAR|acc:X04574) (E value=9.9e−²⁴).

The MOL6a protein encoded by SEQ ID NO:11 has 235 amino acid residues, and is presented using the one-letter code in Table 6B (SEQ ID NO:12). The Psort profile for MOL6a predicts that this sequence has a signal peptide and is likely to be localized on the outside with a certainty of 0.3700. The most likely cleavage site for a peptide is between amino acids 19 and 20, ADS-SV based on the SignalP result.

TABLE 6B
Encoded MOL6a protein sequence.

(SEQ ID NO:12)

MKYVFYLCVLAGTFFFADSSVQKEDPAPYLVYLKSHFNPCVGVLIKPSWVLAPAHCYLPNLKVMLGNFKSRVRDGT
EQTINPIQIVRYWNYSHSAPQDDLMLIKLAKPAMLNPKVQPLPLATTNVRPGTVCLLSGLDWSQENSGRHPDLRQN
LEAPVMSDRECQKTEQGKSHRNSLCVKFVKVFSRIFGEVAVATVICKDKLQGIEVGHFMGGDVGIYTNVYKYVSWI
ENTAKDK

The full amino acid sequence of MOL6a was found to have 79 of 208 amino acid residues (37%) identical to, and 118 of 208 residues (56%) positive with, the 248 amino acid residue TRYPSINOGEN I-P1 PRECURSOR (EC 3.4.21.4) protein from Gallus gallus (ptnr: SWISSNEW—ACC: Q90627) (E value=1.1e−³³).

MOL6b

The disclosed novel Trypsin-like MOL6b nucleic acid of 720 nucleotides (also referred to as CG54548-03) is shown in Table 6C. An open reading begins with an ATG initiation codon at nucleotides 5-7 and ends with a TGA codon at nucleotides 710-712. A putative untranslated region upstream from the initiation codon and downstream from the termination codon are underlined in Table 6C, and the start and stop codons are in bold letters.

TABLE 6C
MOL6b Nucleotide Sequence

(SEQ ID NO:152)

TACCATGAATATCTCTTCTATTTGGCTGTCCTCGCTGGGACATTTTTCTTTGCTGACTCATCTGTTCAGJGA
AGACCCTGCTCCCTATTTCCTGTACCTCAAGTCTCACTTCAACCCCTGTCTCCGCGTCCTCATCAAACCCAGCTG
GGTGCTGGCCCCAGCTCACTGCTATTTACCAAATCTGAAAGTGATGCTGGGAAATTTCAAGAGCAGAGTCAGAGA
CGGTACTGAACAGACAATTAACCCCATTCAGATCGTCCGCTACTGGAACTACAGTCATAGCCCCCCACAGGATGA
CCTCATGCTCATCAAGCTGGCTAACCCTGCCATGCTCAATCCCAAAGTCCAGCCCCTTCCCCTCGCCACCACCAA
TGTCACGCCAGGCACTGTCTGTCTACTCTCAGGTTTGGACTGGACCCAAGAAAACAGTGGCCGACACCCTGACTT
GCGGCAGAACCTCCAGGCCCCCCTGATGTCTGATCGAGAATGCCAAAAAACAGAACAAGGAAAAAGCCACAGGAA
TTCCTTATCTGTGAAATTTGTCAAAGTATTCAGCCGAATTTTTGCCGAGGTGGCCGTTGCTACTGTCATCTGCAA
AGACAAGCTCCAGGGAATCCACGTGGGGCACTTCATGGCACGCGACCTCGGCATCTACACCAATGTTTACAAATA
TGTATCCTCGATTGAGAACACTGCTAAGGACAAGTGAGACCGTAA

The MOL6b protein encoded by SEQ ID NO:152 has 235 amino acid residues, and is presented using the one-letter code in Table 6D (SEQ ID NO:153).

TABLE 6D
Encoded MOL6b protein sequence.

(SEQ ID NO:153)

MKYVFYLGVLAGTFFFADSSVQKEDPAPYLVYLKSHFNPCVGVLIKPSWVLAPAHCYLPNLKVMLGNFKSRVRDGT
EQTINPTQIVRYWNYSHSAPQDDLMLIKLAKPAMLNPKVQPLPLATTNVRPGTVCLLSGLDWSQENSGRHPDLRQN
LEAPVMSDRECQKTEQGKSHRNSLCVKFVKVFSRIFGEVAVATVICKDKLQGIEVGHFMGGDVGIYTNVYKYVSWI
ENTAKDK

MOL6c

The disclosed novel Trypsin-like MOL6c nucleic acid of 516 nucleotides (also referred to as 246861979) is shown in Table 6E. An open reading begins with an CGC initiation codon at nucleotides 1-3 and ends with a GCGcodon at nucleotides 514-516. A putative untranslated region upstream from the initiation codon and downstream from the termination codon are underlined in Table 6E, and the start and stop codons are in bold letters. Because the start and stop codons of MOL6c are not traditioonal initiation and termination codons, MOL6c could be a partial reading frame extending further in the 5′ and/or 3′ directions.

TABLE 6E
MOL6c Nucleotide Sequence

(SEQ ID NO:154)

CGCGCATCCACCATGAAATATGTCTTCTATTTGGGTGTCCTCGCTGCGACATTTTTCTTTGCTGACTCATCTGTT
CAGAAACAAGACCCTGCTCCCTATTTGGTGTACCTCAAGTCTCACTTCAACCCCTGTGTGGGCGTCCTCATCAAA
CCCAGCTGGGTGCTGCCCCCAGCTCACTGCTATTTACCAAATCTGAAAGTCATGCTGGGAAATTTCAAGAGCAGA
GTCAGAGACGGTACTCAACACACAATTAACCCCATTCAGATCGTCCCCTACTCCAACTACACTCATAGCGCCCCA
CAGGATGACCTCATGCTCATCAAGCTCGCTAAGCCTGCCATGCTCAATCCCAAAGTCCAGCCCCTTCCCCTCGCC
ACCACCAATCTCAGGCCAGCCACTGTCTGTCTACTCTCAGGTTTGGACTGCAGCCAAGAAAACAGTGGCCGACAC
CCTGACTTGCGGCAGAACCTGGAGGCCCCCGTGATGTCTGATCGAGAATGCCAAAAACTCGAGGCG

The MOL6c protein encoded by SEQ ID NO:154 has 172 amino acid residues, and is presented using the one-letter code in Table 6F (SEQ ID NO:155).

TABLE 6F
Encoded MOL6c protein sequence.

(SEQ ID NO:155)

RGSTMKYVFYLGVLAGTFFFADSSVQKEDPAPYLVYLKSHFNPCVGVLIKPSWVLAPAHCYLPNLKVMLGNFKSRV
RDGTEQTINPIQIVRYWNYSHSAPQDDLMLIKLAKPAMLNPKVQPLPLATTNVRPGTVCLLSGLDWSQENSGRHPD
LRQNLEAPVMSDRECQKLEA

MOL6d

The disclosed novel Trypsin-like MOL6d nucleic acid of 600 nucleotides (also referred to as 246862070) is shown in Table 6G. An open reading begins with an CGC initiation codon at nucleotides 1-3 and ends with a GCG codon at nucleotides 598-600. A putative untranslated region upstream from the initiation codon and downstream from the termination codon are underlined in Table 6G, and the start and stop codons are in bold letters. Because the start and stop codons of MOL6d are not traditioonal initiation and termination codons, MOL6d could be a partial reading frame extending further in the 5′ and/or 3′ directions.

TABLE 6G
MOL6d Nucleotide Sequence

(SEQ ID NO:156)

CGCGCATCCGGCGTCCTCATCAACCCAGCTGGGTGCTCGCCCCAGCTCACTGCTATTTACCAAATCTGAAAGTG
ATCCTGGCAAATTTCAAGAGCAGAGTCAGAGACGGTACTGAACAGACAATTAACCCCATTCAGATCGTCCGCTAC
TGGAACTACAGTCATAGCGCCCCACAGGATGACCTCATGCTCATCAAGCTGGCTAAGCCTGCCATGCTCAATCCC
AAAGTCCAGCCCCTTCCCCTCGCCACCACCAATGTCAGGCCAGGCACTGTCTGTCTACTCTCAGGTTTGGACTGC
AGCCAAGAAAACAGTGGCCGACACCCTGACTTGCGGCAGAACCTGGAGGCCCCCGTGATGTCTGATCGAGAATGC
CAAAAAACAGAACAAGGAAAAAGCCACAGGAATTCCTTATGTGTGAAATTTGTGAAAGTATTCAGCCGAATTTTT
GGGGAGGTGGCCGTTGCTACTGTCATCTGCAAAGACAAGCTCCAGGGAATCGAGGTGGGGCACTTCATGGGAGGG
GACGTCGGCATCTACACCAATGTTTACAAATATGTATCCTGGATTGAGAACACTGCTAAGGACAAGCTCGAGGCG

The MOL6d protein encoded by SEQ ID NO:156 has 200 amino acid residues, and is presented using the one-letter code in Table 6H (SEQ ID NO:157).

TABLE 6H
Encoded MOL6d protein sequence.

(SEQ ID NO:157)

RGSGVLIKPSWVLAPAHCYLPNLKVMLGNFKSRVRDGTEQTINPIQIVRYWNYSHSAPQDDLMLIKLAKPPAMLNPK
VQPLPLATTNVRPGTVCLLSGLDWSQENSGRHPDLRQNLEAPVMSDRECKQKTEQGKSHRNSLCVKFVKVFSRIFGE
VAVATVICKDKLQGIEVGHFMGGDVGIYTNVYKYVSWIENTAKDKLEA

MOL6a also has high homology to the proteins shown in the BLAST data in Table 6I.

SNP analysis of MOL6a is described in Example 2.

TABLE 6I
BLAST results for MOL6a

| Gene Index/ | | Length | Identity | Positives | |
|---|---|---|---|---|---|
| Identifier | Protein/Organism | (aa) | (%) | (%) | Expect |
| gi\|2499862\|sp\|Q90627\| | TRYPSIN I-P1 | 248 | 79/208 | 118/208 | 2e−31 |
| TRY1_CHICK | PRECURSOR | | (37%) | (55%) | |
| gi\|2118087\|pir\|\|S55067 | trypsin (EC | 248 | 78/208 | 117/208 | 5e−31 |
| | 3.4.21.4) I | | (37%) | (55%) | |
| | precursor, | | | | |
| | pancreatic - | | | | |
| | chicken | | | | |
| gi\|6678439\|ref\|NP_033456.1\| | trypsin 2 [Mus | 246 | 77/215 | 115/215 | 8e−29 |
| | musculus] | | (35%) | (52%) | |
| gi\|1633123\|pdb\|1SLW\|B | Chain B, Rat | 223 | 74/212 | 116/212 | 1e−28 |
| | Anionic N143h, | | (34%) | (53%) | |
| | E151h Trypsin | | | | |
| | Complexed To | | | | |
| | A86h Ecotin | | | | |

MOL6e

In the present invention, the target sequence identified previously, MOL6a, Accession Number GM_{—}87760758_A, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on library containing a wide range of cDNA species. The resulting amplicon was gel purified, cloned and sequenced to high redundancy to provide the sequence reported below, which is designated MOL6e, Accession Number GM_{—}87760758_A_da

The disclosed novel Trypsin-like MOL6e nucleic acid of 730 nucleotides (also referred to as GM_{—}87760758_A_da) is shown in Table 6J. An open reading frame begins with an ATG initiation codon at nucleotides 8-10 and ends with a TGA codon at nucleotides 713-715. A putative untranslated region upstream from the initiation codon and downstream from the termination codon are underlined in Table 6J, and the start and stop codons are in bold letters.

TABLE 6J
MOL6e Nucleotide Sequence

(SEQ ID NO:13)

GATCACCATGAAATATGTCTTCTATTTGGGTGTCCTCGCTGGGACATTTTTCTTTGCTGACTCATCTGTCCAGAA
AGAAGACCCTGCTCCCTATTTGGTGTACCTCAAGTCTCACTTCAACCCCTGTGTGGGCGTCCTCATCAAACCCAG
CTGGGTGCTGGCCCCAGCTCACTGCTATTTACCAAATCTGAAAGTGATGCTGGGAAATTTCAAGAGCAGAGTCAG
AGACGGTACTGAACAGACAATTAACCCCATTCAGATCGTCCGCTACTGGAACTACAGTCATACCGCCCCACAGCA
TGACCTCATGCTCATCAAGCTGGCTAAGCCTGCCATGCTCAATCCCAAACTCCAGCCCCTTCCCCTCCCCACCAC
CAATGTCAGGCCAGGCACTGTCTGTCTACTCTCAGGTTTGGACTGGAGCCAAGAAAACACTGGCCGACACCCTGA
CTTGCGGCAGAACCTGGAGGCCCCCGTGATGTCTGATCGAGAATGCCAAAAAACAGAACAAGGAAAAAGCCACAG
GAATTCCTTATGTGTGAAATTTGTGAAAGTATTCAGCCGAATTTTTGGGGAGGTGGCCGTTGCTACTGTCATCTG
CAAAGACAAGCTCCAGGGAATCGAGGTGGGGCACTTCATGGGAGGGGACGTCGGCATCTACACCAATGTTTACAA
ATATGTATCCTGGATTGAGAACACTGCTAAGGACAAGTGAGACCCTACTTCTCCC

The MOL6e protein encoded by SEQ ID NO:13 has 235 amino acid residues, and is presented using the one-letter code in Table 6K (SEQ ID NO:14). The Psort profile for MOL6a predicts that this sequence has a signal peptide and is likely to be localized on the outside with a certainty of 0.3700. The most likely cleavage site for a peptide is between amino acids 19 and 20 based on the SignalP result.

TABLE 6K
Encoded MOL6e protein sequence.

(SEQ ID NO:14)

MKYVFYLGVLAGTFFFADSSVQKEDPAPYLVYLKSHFNPCVGVLIKPSWVLAPAHCYLPNLKVMLGNFKSRVRDGT
EQTINPIQIVRYWNYSHSAPQDDLMLIKLAKPAMLNPKVQPLPLATTNVRPGTVCLLSGLDWSQENSGRHPDLRON
LEAPVMSDRECQKTEQGKSHRNSLCVKFVKVFSRIFGEVAVATVICKDKLQGIEVGHFMGGDVGIYTNVYKYVSWI
ENTAKDK

The full amino acid sequence of MOL6e was found to have homology with several proteins including those disclosed in the BLASTP data in Table 6L.

TABLE 6L
BLAST results for MOL6e

| | | | Identity | Positives | |
|---|---|---|---|---|---|
| Gene Index/Identifier | Protein/Organism | Length (aa) | (%) | (%) | Expect |
| ACC: Q90627 | TRYPSIN I-P1 | 248 | 79/208 | 118/208 | 3.8e−34 |
| | PRECURSOR (EC | | (37%) | (56%) | |
| | 3.4.21.4) - | | | | |
| | Gallus gallus | | | | |
| | (Chicken) | | | | |
| PIR-ID: S55067 | trypsin (EC | 248 | 78/208 | 117/208 | 7.9e−34 |
| | 3.4.21.4) I | | (37%) | (55%) | |
| | precursor, | | | | |
| | pancreatic - | | | | |
| | chicken | | | | |
| ACC: Q90628 | TRYPSIN I-P38 | 248 | 78/208 | 117/208 | 1.0e−33 |
| | PRECURSOR (EC | | (37%) | (56%) | |
| | 3.4.21.4) - | | | | |
| | Gallus gallus | | | | |
| | (Chicken) | | | | |
| ACC: P07477 | TRYPSIN I | 247 | 76/212 | 112/212 | 1.3e−31 |
| | PRECURSOR (EC | | (35%) | (52%) | |
| | 3.4.21.4) | | | | |
| | (CATIONIC | | | | |
| | TRYPSINOGEN) - | | | | |
| | Homo sapiens | | | | |
| | (Human) | | | | |

MOL6e also has high homology to the proteins shown in the BLASTX alignment data in Table 6M.

TABLE 6M
BLASTX results for MOL6e

| | | | Smallest | |
|---|---|---|---|---|
| | | | Sum | |
| | Reading | High | Prob | |
| Sequences producing High-scoring Segment Pairs: | Frame | Score | P(N) | N |
| ptnr:SWISSPROT-ACC:Q90627 TRYPSIN I-P1 PRECURSOR (EC 3. | +2 | 372 | 2.3e−33 | 1 |
| ptnr:PIR-ID:S55067 trypsin (EC 3.4.21.4) I precursor, | +2 | 369 | 4.7e−33 | 1 |
| ptnr:SWISSPROT-ACC:Q90628 TRYPSIN I-P38 PRECURSOR (EC. | +2 | 368 | 6.0e−33 | 1 |
| ptnr:SWISSPROT-ACC:P07146 TRYPSIN II, ANIONIC PRECURSO | +2 | 350 | 4.9e−31 | 1 |
| ptnr:SWISSPROT-ACC:P07477 TRYPSIN I PRECURSOR (EC 3.4.. | +2 | 348 | 7.9e−31 | 1 |
| ptnr:SPTREMBL-ACC:Q9R0T7 PANCREATIC TRYPSIN - Mus musc. | +2 | 348 | 7.9e−31 | 1 |
| ptnr:SWISSPROT-ACC:P15951 TRYPSIN III PRECURSOR (EC 3.. | +2 | 347 | 1.0e−30 | 1 |

MOL6f

The disclosed novel Trypsin-like MOL6f nucleic acid of 390 nucleotides (also referred to as 256801454) is shown in Table 6BBA. An open reading begins with an CGC initiation codon at nucleotides 1-3 and ends with a GCG codon at nucleotides 388-390. Because the start and stop codons of MOL6f are not traditioonal initiation and termination codons, MOL6f could be a partial reading frame extending further in the 5′ and/or 3′ directions.

TABLE 6BBA
MOL6f Nucleotide Sequence

(SEQ ID NO:176)

CGCGGATCCGGCGTCCTCATCAAACCCAGCTGGGTGCTGGCCCCAGCTCACTGCTATTTACCAAATCTGAAAGTG
ATGCTCGGAAATTTCAAGAGCAGAGTCAGAGACGGTACTGAACAGACAATTAACCCCATTCAGATCGTCCGCTAC
TGGAACTACAGTCATAGCGCCCCACAGGATGACCTCATGCTCATCAAGCTGGCTAAGCCTGCCATGCTCAATCCC
AAAGTCCAGCCCCTTCCCCTCGCCACCACCAATGTCAGGCCAGGCACTGTCTGTCTACTCTCAGGTTTGGACTGG
AGCCAAGAAAACAGTGGCCGACACCCTGACTTGCGGCAGAACCTGGAGGCCCCCGTGATGTCTGATCGAGAATGC
CAAAAACTCGAGGCG

The MOL6f protein encoded by SEQ ID NO:156 has 130 amino acid residues, and is presented using the one-letter code in Table 6BBB (SEQ ID NO:157).

TABLE 6BBB
Encoded MOL6f protein sequence.

(SEQ ID NO:177)

RGSGVLIKPSWVLAPAHCYLPNLKVMLGNFKSRVRDGTEQTINPIQIVRYWNYSHSAPQDDLMLIKLAKPAMLNPK
VQPLPLATTNVRPGTVCLLSGLDWSQENSGRHPDLRQNLEAPVMSDRECQKLEA

This information is presented graphically in the multiple sequence alignment given in Table 6N (with MOL6a being shown on line 1 and MOL6e being shown on line 2) as a ClustalW analysis comparing MOL6 with related protein sequences.

[image]

[image]

MOL6e also contained several single polynucleotide polymorphisms described in Table 6O.

TABLE 6O
SNP for MOL6e

| Position | Nucleotide Change | Number of Occurrences |
|---|---|---|
| 70 | C > A | 2 |
| 70 | C > G | 6 |
| 261 | T > C | 2 |
| 406 | A > C | 2 |
| 573 | G > T | 2 |
| 585 | C > T | 2 |
| 737 | A > G | 4 |

Trypsin (EC 3.4.21.4), like elastase, is a member of the pancreatic family of serine proteases. The gene encoding trypsin-1 (TRY 1) is also referred to as serine protease-1 (PRSS1). MacDonald et al. (1982) reported nucleotide sequences of cDNAs representing 2 pancreatic rat trypsinogens. Using a rat cDNA probe, Honey et al. (1984, 1984) found that a 3.8-kb DNA fragment containing human trypsin-1 gene sequences cosegregated with chromosome 7, and assigned the gene further to 7q22-7qter by study of hybrids with a deletion of this segment. The trypsin gene is on mouse chromosome 6 (Honey et al., 1984). Carboxypeptidase A and trypsin are a syntenic pair conserved in mouse and man. Emi et al. (1986) isolated cDNA clones for two major human trypsinogen isozymes from a pancreatic cDNA library. The deduced amino acid sequences had 89% homology and the same number of amino acids (247), including a 15-amino acid signal peptide and an 8-amino acid activation peptide. Southern blot analysis of human genomic DNA with the cloned cDNA as a probe showed that the human trypsinogen genes constitute a family of more than ten, some of which may be pseudogenes or may be expressed in other stages of development.

Rowen et al. (1996) found that there are eight trypsinogen genes embedded in the beta T cell receptor locus or cluster of genes (TCRB; OMIM-186930), which maps to 7q35. In the 685-kb DNA segment that they sequenced they found five tandemly arrayed 10-kb locus-specific repeats (homology units) at the 3-prime end of the locus. These repeats exhibited 90 to 91% overall nucleotide similarity, and embedded within each is a trypsinogen gene. Alignment of pancreatic trypsinogen cDNAs with the germline sequences showed that these trypsinogen genes contain five exons that span approximately 3.6 kb. Further analyses revealed 2 trypsinogen pseudogenes and one relic trypsinogen gene at the 5-prime end of the sequence, all in inverted transcriptional orientation. They denoted eight trypsinogen genes T1 through T8 from 5-prime to 3-prime. Rowen et al. (1996) found that only two of three pancreatically expressed trypsinogen cDNAs correspond to trypsinogen genes in the TCRB locus; T4 was denoted trypsinogen 1 and T8 was denoted trypsinogen 2 (OMIM-601564). The third pancreatic cDNA, identified independently as trypsinogen 3 (Tani et al., 1990) and 4 (Wiegand et al., 1993), is distinct from the third apparently functional trypsinogen gene (T6) in the TCRB locus but related to the other pancreatic trypsinogens. Rowen et al. (1996) stated that the T6 gene is deleted in a common insertion-deletion polymorphism; if it is functional, its function is apparently not essential. Some of the trypsinogen genes are expressed in nonpancreatic tissues where their function is unknown. Rowen et al. (1996) noted that the intercalation of the trypsinogen genes in the TCRB locus is conserved in mouse and chicken, suggesting shared functional or regulatory constraints, as has been postulated for genes in the major histocompatibility complex (such as class I, II, and III genes) that share similar long-term organizational relationships.

Rowen et al. (1996) mapped the gene corresponding to the third pancreatic trypsinogen cDNA by fluorescence in situ hybridization. They used a cosmid clone containing 3 trypsinogen genes. Strong hybridization to chromosome 7 and weaker hybridization to chromosome 9 were observed. They isolated and partially sequenced 4 cosmid clones from the chromosome 9 region. They found that the region represents a duplication and translocation of a DNA segment from the 3-prime end of the TCRB locus that includes at least seven V(beta) elements and a functional trypsinogen gene denoted T9. The assignment of the PRSS1 gene to 7q35 is established by the demonstration of its sequence within the sequence of the ‘locus’ (OMIM-186930) for the T-cell receptor beta-chain (Rowen et al., 1996). It is further supported by the linkage between microsatellite markers in the 7q35 region and hereditary pancreatitis (OMIM-167800) and the demonstration of mutations in the PRSS1 gene in hereditary pancreatitis.

Whitcomb et al. (1996) stated that the high degree of DNA sequence homology (more than 91%) present among this cluster of five trypsinogen genes identified by Rowen et al. (1996) demanded that highly specific sequence analysis strategies be developed for mutational screening in families with hereditary pancreatitis. This was necessary to ensure that each sequencing run contained only the two alleles corresponding to a single gene, thereby permitting detection of heterozygotes in this autosomal dominant disorder, and not a dozen or more alleles from multiple related trypsinogen-like genes, which would make detection of heterozygotes nearly impossible. In a family with hereditary pancreatitis, Whitcomb et al. (1996) found that affected individuals had a single G-to-A transition mutation in the third exon of cationic trypsinogen (276000.0001). This mutation was predicted to result in an arg105-to-his substitution in the trypsin gene (residue number 117 in the more common chymotrypsin number system). Subsequently, the same mutation was found in a total of five different hereditary pancreatitis kindreds (four from the U.S. and one from Italy) containing a total of 20 affected individuals and six obligate carriers. The mutation was found in none of the obligate unaffected members (individuals who married into the family). Subsequent haplotyping revealed that all four of the American families displayed the same high risk haplotype over a 4-cM region encompassing seven STR markers, confirming the likelihood that these kindreds shared a common ancestor, although no link could be found through eight generations. A fifth family from Italy displayed a unique haplotype indicating that the same mutation had occurred on at least 2 occasions. The G-to-A mutation at codon 117 created a novel enzyme recognition site for AflIII which provided a facile means to screen for the mutation. As with the obligate unaffected members of the pancreatitis kindreds, none of 140 controls possessed the G-to-A mutation as assayed by the lack of AflIII digestion of the amplified exonic DNA.

Failure to thrive, nutritional edema, and hypoproteinemia with normal sweat electrolytes were features of 2 affected male infants reported by Townes (1965) and Townes et al. (1967). A protein hydrolysate diet was beneficial. A male sib of the first patient reported by Townes (1965) had died, apparently of the same condition. Morris and Fisher (1967) reported an affected female who also had imperforate anus. The clinical picture in enterokinase deficiency (OMIM-226200) is closely similar; however, the defect is not in the synthesis of trypsinogen but in the synthesis of the enterokinase which activates proteolytic enzymes produced by the pancreas. Oral pancreatin represents a therapeutically successful form of enzyme replacement (Townes, 1972). Since hereditary pancreatitis has been mapped rather precisely to 7q35 and since a defect in the trypsinogen gene has been identified in hereditary pancreatitis, the assignment of the trypsinogen gene can be refined from 7q32-qter to 7q35.

Ferec et al. (1999) studied 14 families with hereditary pancreatitis and found mutations in the PRSS1 gene in 8 families. In 4 of these families, the mutation (R117H; 276000.0001) had been described by Whitcomb et al. (1996). Three novel mutations were described in 4 other families.

Sahin-Toth et al. (1999) studied the roles of the R117H and N21I (276000.0002) mutations in hereditary pancreatitis. They stated that the R117H mutation is believed to cause pancreatitis by eliminating an essential autolytic cleavage site in trypsin, thereby rendering the protease resistant to inactivation through autolysis. Sahin-Toth et al. (1999) demonstrated that the R117H mutation also significantly inhibited autocatalytic trypsinogen breakdown under Ca(2+)-free conditions and stabilized the zymogen form of rat trypsin. Taken together with findings demonstrating that the N21I mutation stabilized rat trypsinogen against autoactivation and consequent autocatalytic degradation, the observations suggested a unifying molecular pathomechanism for hereditary pancreatitis in which zymogen stabilization plays a central role.

Uses of the Compositions of the Invention

The above defined information for this invention suggests that this Trypsin-like protein may function as a member of a “Trypsin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in failure to thrive, nutritional edema, and hypoproteinemia, trypsinogen deficiency disease, chronic and heriditary pancreatitis, enterkinase defieciency, cancer and/or related pathologies and disorders and/or other pathologies and disorders. For example, a cDNA encoding the Trypsin-like protein may be useful in gene therapy, and the Trypsin-like protein may be useful when administered to a subject in need thereof. By way of nonlimiting example, the compositions of the present invention will have efficacy for treatment of patients suffering from failure to thrive, nutritional edema, and hypoproteinemia, trypsinogen deficiency disease, chronic and heriditary pancreatitis, enterkinase defieciency, cancer. The novel nucleic acid encoding Trypsin-like protein, and the Trypsin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL7

A novel nucleic acid encoding a Kallikrein-like-protein MOL7 was identified by TblastN using CuraGen Corporation's sequence file for MOL7 probes or homologs, and run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein. The disclosed novel MOL7 nucleic acid of 1811 nucleotides (also referred to as 30675745.0.499) is shown in Table 7A. An open reading frame begins with an ATG initiation codon at nucleotides 368-370 and ends with a TAG codon at nucleotides 1553-1555. A putative untranslated region upstream from the initiation codon and downstream from the termination codon are underlined in Table 7A, and the start and stop codons are in bold letters.

TABLE 7A
MOL7 Nucleotide Sequence

(SEQ ID NO:15)

ACAAATCCTTCTGTTGAACTCTACTGTGTCAGGCCAGCCTGAGTTCATTTCTCCTTGAGCAGGAACAGTT
CATGGACGAACTCTGAGGACCATTCTGAGGACAAGAGGCATCCAGTGTCATGAGTGGAACATGCAGCATT
TTATGGCTACAGAGTTAAGGCAAGGGTTGAATTCCACGAGTCAAAAAGCAGCCCTTTTCAGAGACCCAAC
TCTCTGGGGTGCTCAGGGGCTTGGGCTGGATTGAGAAGAAAACTGACAAGAGTAAGCTGCCCTCTCTTCT
CTGGCCATCTCACAAACCACAGTGCGGGCCAACTGGTCCTGCCTCTTTACCACACAGAACCAAGCACTAG
GGATAAGACAGCTGCCCATGGTGTCCGCGGCGGGTCTCTCTGGGGATGGCAAGATGCGAGGGGTGCTCCT
GGTGCTGCTCGGCCTTCTCTATTCTTCCACCAGTTGTGGCGTCCAGAAAGCTTCCGTTTTCTACGGTCCT
GACCCCAAGGAGGGCTTGGTCAGCAGCATGGAGTTCCCGTGGGTGGTGTCGCTGCAGGACTCCCAGTACA
CACACCTGGCTTTCGGCTGCATCCTGAGCGAGTTCTGGGTCCTCAGCATCGCATCCGCCATTCAGAACAG
GAAGGACATTGTCGTTATAGTGGGTATAAGTAACATGGATCCTAGCAAGATTGCTCACACAGAGTATCCA
GTCAATACCATCATCATCCATGAGGACTTTGATAACAACTCCATGAGCAACAACATAGCCCTCCTCAAGA
CAGACACAGCGATGCATTTTGGCAACCTGGTCCAGTCCATCTGCTTCCTCGGCAGAATGCTGCATACACC
ACCAGTCTTGCAGAACTGCTGGGTGTCAGGATGGAATCCCACATCTGCAACAGGAAATCACATGACGATG
AGTGTCCTGAGGAAAATCTTCGTGAAAGATCTTGACATGTGTCCCCTATACAAACTCCAGAAGACAGAAT
GCGGCAGCCACACGAAAGAGGAAACCAAGACTGCCTGCTTGGGGGACCCAGGAAGCCCAATGATGTGCCA
GCTACAGCAGTTCGATCTGTGGGTTCTGACAGGAATCCTGAACTTCGGTGGTGAGACGTGCCCTGGCCTG
TTTCTGTACACCAAGGTGGAAGACTACAGCAAATGGATCACATCCAAGGCTGAGAGGGCCGGCCCTCCCC
TGTCCTCACTCCACCACTGGGAAAAGTTGATTTCTTTCTCCCACCATGGACCAAATGCCGCCATGACACA
GAAGACATATTCTGATTCTGAACTGGGCCATGTTGGATCATACTTGCAGGGACAAAGAAGGACCATCACG
CATTCACGACTAGGAAACAGCTCTAGAGATAGTCTAGATGTTAGGGAGAAGGATGTAAAGGAATCAGGCA
GGTCTCCTGAGGCGTCTGTACAACCCTTATACTATGACTATTACGGTGGGGAGGTGGGGGAAGGTAGGAT
TTTTGCAGGTCAGAACAGGTTGTATCAGCCCGAAGAAATCATCTTGGTTTCCTTCGTGCTTGTTTTCTTT
TGCAGCAGTATCTAGTCCAGGAGCTACCCCACCAAACTGAAGAGTAAACTGAGAATGCTGAGTGCCAGGC
ATTCACCATGCTGTTTTGATGTCTGTTTTTGATAGTTGCACACTGGGGCTGCCACGGATAAGCCCATGGC
ATACACTGGGCTGGCTCTCCCTCCTCTATCCCTCTCCCAGGTGTGGGAAGGTCACTTTCACTATGCTTGT
GAACTAAATGCTGGCTAACAAGTGTCAAAAAAAAAAAAAAAAAAAAAAAAAAAAAAAAAAA

The MOL7 protein encoded by SEQ ID NO:15 has 395 amino acid residues, and is presented using the one-letter code in Table 7B (SEQ ID NO:16). The SignalP, Psort and/or Hydropathy profile for MOL7 predict that MOL7 has a signal peptide and is likely to be localized at the plasma membrane with a certainty of 0.9190. The SignalP shows a signal sequence is coded for in the first 44 amino acids with the most likely cleavage site being between amino acids 30 and 31. This is typical of this type of membrane protein. The molecular weight of MOL7 is 43815.7 Daltons.

TABLE 7B
Encoded MOL7 protein sequence.
(SEQ ID NO:16)

MVSAAGLSGDGKMRGVLLVLLGLLYSSTSCGVQKASVFYGPDPKEGLVSSMEFPWVVSLQDSQYTHLAFGCILSE
FWVLSIASAIQNRKDIVVIVGISNMDPSKIAHTEYPVNTIIIHEDFDNNSMSNNIALLKTDTAMHFGNLVQSICF
LGRMLHTPPVLQNCWVSGWNPTSATGNHMTMSVLRKIFVKDLDMCPLYKLQKTECGSHTKEETKTACLGDPGSPM
MCQLQQFDLWVLRGILNFGGETCPGLFLYTKVEDYSKWITSKAERAGPPLSSLHHWEKLISFSHHGPNAAMTQKT
YSDSELGHVGSYLQGQRRTITHSRLGNSSRDSLDVREKDVKESGRSPEASVQPLYYDYYGGEVGEGRIFAGQNRL
YQPEEIILVSFVLVFFCSSI

MOL7 was found to have 290 of 290 amino acid residues (100%) identical to the 290 amino acid residue hypothetical 32.6 kD protein from Homo sapiens (human) (ACC:CAB70765). This protein has similarity to kallikrein.

MOL7 shows significant homologies to human hypothetical 32.6 kD protein (a protein with similiarities to Kallikrein), as described in, but not limited to, the references below.

Kallikreins are a subgroup of serine proteases and these proteolytic enzymes have diverse physiological functions in many tissues. Growing evidence suggests that many kallikreins are implicated in carcinogenesis. The human kallikrein gene family is localized on chromosome 19q13.3-q13.4 and currently includes three members: KLK1 or pancreatic/renal kallikrein, KLK2 or human glandular kallikrein and KLK3 or prostate-specific antigen (PSA). The latter two genes are almost prostate-specific and they are used for diagnosis and monitoring of prostate cancer and more recently, in breast cancer applications (Yousef et al., Anticancer Res July-August 1999;19(4B):2843-52). These new genes, like the already known kallikreins, may have utility for diagnosis, monitoring and therapeutics of various cancers including those of the breast, prostate and testis.

Monsees et al., Immunopharmacology December 1999;45(1-3):107-14, found that elements of the kallikrein-kinin system are present in rat seminiferous epithelium. Peptide hormones are involved in the paracrine regulation of several physiological processes. The paracrine peptide system may play a role in the regulation of Sertoli cell function or in the Sertoli cell-germ cell crosstalk, and therefore, be involved in mammalian reproduction, especially spermatogenesis.

Chen et al., J Biol Chem Nov. 1, 1996;271(44):27590-4, found that the kallikrein-kinin system participates in blood pressure regulation. One of the kallikrein-kinin system components, kallikrein-binding protein, binds to tissue kallikrein and inhibits its activity in vitro.

The glandular kallikreins are a distinct group of serine proteases with a molecular weight of 25,000-40,000 and an ability to release vasoactive peptides from kininogen in vitro, although the kininogenase activity of different kallikreins is highly variable. The true physiologic role of specific kallikreins is often unrelated to the kininogenase activity. In the mouse a major site of kallikrein synthesis is the male submaxillary gland. Glandular kallikreins are also synthesized in the pancreas and kidney. The several kallikreins found in this tissue include epidermal growth factor binding protein (EGF-BP) and the gamma subunit of nerve growth factor (NGFG; 162040) which are responsible for the processing of EGF (131530) and NGF (162030), respectively. Although EGF-BP and NGFG exhibit strict substrate specificity, they share extensive amino acid sequence homology and immunologic crossreactivity. Mason et al. (1983) concluded that the glandular kallikrein gene family comprises 25-30 highly homologous genes that encode specific proteases involved in the processing of biologically active peptides. All are closely linked on mouse chromosome 7 (assignment by Chinese hamster-mouse hybrid cell studies). Several human kallikrein genes have been isolated.

Schedlich et al. (1987) described a human glandular preprokallikrein gene, hGK-1, isolated from a human genomic library. The 5.2-kb gene encodes a prepropeptide of 261 amino acids. The mature protein is 237 amino acids long and has 66% homology with the sequence predicted for the human kallikrein synthesized in pancreas, kidney, and salivary gland (KLK1; 147910). Seventy-three percent homology with human prostate-specific antigen (APS; 176820) was observed. Expression of the glandular kallikrein gene, like that of the APS gene, seems to be restricted to the prostate. Riegman et al. (1989) found that the glandular kallikrein gene and that for prostate-specific antigen are aligned in a head-to-tail orientation and are separated by about 12 kb. Southern blot analysis of DNA from a panel of human-hamster hybrid cells showed that the genes are situated on chromosome 19. Since the KLK1 gene is also on chromosome 19, these 3 genes probably represent a cluster. From in situ hybridization studies, Qin et al. (1991) concluded that the glandular kallikrein gene and probably other kallikrein genes are located in q13.3 and q13.4 bands of chromosome 19 and are probably near the border of these two bands.

Therapeutic Applications

The expression pattern, and protein similarity information for MOL7 suggest that it may function as human Kallikrein-like protein. Therefore, the nucleic acid and protein of the invention are useful in potential therapeutic applications implicated, for example but not limited to, various cancers including those of the testis, prostate, and breast; mammalian reproduction, especially spermatogenesis; blood pressure regulation; and other diseases and disorders. The homology to antigenic secreted and membrane proteins suggests that antibodies directed against the novel genes may be useful in treatment and prevention of various cancers including those of the testis, prostate, and breast; mammalian reproduction, especially spermatogenesis; blood pressure regulation; and other diseases and disorders.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

The nucleic acids and proteins of the invention are useful in potential therapeutic applications implicated in various cancers including those of the testis, prostate, and breast; mammalian reproduction, especially spermatogenesis; blood pressure regulation; and other diseases and disorders. For example, but not limited to, a cDNA encoding the novel human plasma membrane protein may be useful in gene therapy, and the novel human plasma membrane protein may be useful when administered to a subject in need thereof. By way of non-limiting example, the compositions of the present invention will have efficacy for treatment of patients suffering from, for example, but not limited to, various cancers including those of the testis, prostate, and breast; mammalian reproduction, especially spermatogenesis; blood pressure regulation; and other diseases and disorders. The novel nucleic acid encoding the novel human plasma membrane protein, and the novel human plasma membrane protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

MOL8

MOL8a

A novel human Acetyl LDL Receptor-like nucleic acid was identified by TblastN using CuraGen Corporation's sequence file for MOL probes or homologs and run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein. The disclosed novel MOL8a nucleic acid of 980 nucleotides (also referred to as 11800699-0-16) is shown in Table 8B. An open reading frame begins with an ATG initiation codon at nucleotides 1-3 and ends with a TGA codon at nucleotides 2803-2805. A putative untranslated region downstream from the termination codon are underlined in Table 8A, and the start and stop codons are in bold letters.

TABLE 8A
MOL8a Nucleotide Sequence

(SEQ ID NO:17)

ATGGAGGGCGCAGGGCCCCGGGGGCCGGGCCGGCGCGGCGCCGGGGAGCCGGGGGGCCGCCGTCACCGCTGCTGC
CGTCGCTGCTGCTGCTGCTGCTGCTCTGGATGCTGCCGGACACCGTGGCGCCTCAGGAACTGAACCCTCGCGGCCG
CAACGTGTGCCGTGCTCCCGGCTCCCAGGTGCCCACGTGCTGCGCTGGCTGGAGGCAGCAAGGGGACGAGTGTGGG
ATTGCGGTGTGCGAAGGCAACTCCACGTGCTCAGAGAACGAGGTGTGCGTGAGGCCTGGCGAGTGCCGCTGCCGCC
ACGGCTACTTCGGTGCCAACTGCGACACCAGTGAGCGTGGGGTCGGGCCGGTATTGGTCGGTGGGGCGGAATCCTG
GAGAGATGGGGCGGGGTCCAAGGTGGGGCGGGGTCGGATCCGCCTTCGGGGCGGGTCCCCAGAGGTGGCGGCTGGA
GTGCGGGACGCGGGCAGGTTCCGGCTGGCTGGGGGCACCTACTCAAGCACCGGGGCCTTCCACCCCCTCCGCTCCT
CCCCTGCAGAGTGCCCGCGCCAGTTCTGGGGCCCCGACTGCAAGGAGCTGTGTAGCTGCCACCCACACGGGCAGTG
CGAGGACGTGACAGGCCAGTGTACTTGTCACGCGCGGCGCTGGGGCGCGCGCTGCGAGCATGCGTGCCAGTGCCAG
CACGGCACGTGCCACCCCCGGAGCGGCGCGTGCCGCTGTGAGTCCGGCTGGTGGGGCGCGCAGTGCGCCAGCGCGT
GCTACTGCAGCGCCACGTCGCGCTGCGACCCACAGACCGGCGCCTGCCTGTGCCACGCAGGCTGGTGGGGCCGCAG
CTGCAACAACCAGTGCGCCTGCAACTCGTCTCCCTGCGAGCAGCAGAGCGGCCGCTGTCAGTGCCGCGAGCGTACG
TTCGGCGCGCGCTGCGATCGCTACTGCCAGTGCTTCCGCGGCCGCTGCCACCCTGTGGACGGCACGTGTGCCTGCG
AGCCGGGCTACCGCGGCAAGTACTGTCGCGAGCCGTGCCCCGCCGGCTTCTACGGCTTGGGCTGTCGCCGCCGGTG
TGGCCAGTGCAAGGGCCAGCAGCCGTGCACGGTGGCCGAGGGCCGCTGCTTGACGTGCGAGCCCGGCTGGAACGGA
ACCAAGTGCGACCAGCCTTGCGCCACCGGTTTCTATGGCGAGGGCTGCAGCCACCGCTGTCCGCCATGCCGCGACG
GGCATGCCTGTAACCATGTCACCGGCAAGTGTACGCGCTGCAACGCGGGCTGGATCGGCGACCGGTGCGAGACCAA
GTGTAGCAATGGCACTTACGGCGAGGACTGCGCCTTCGTGTGCGCCGACTGCGGCAGCGGACACTGCGACTTCCAG
TCGGGGCGCTGCCTGTGCAGCCCTGGCGTCCACGGGCCCCACTGTAACGTGACGTGCCCGCCCGGACTCCACGGCG
CGGACTGTGCTCAGGCCTGCAGCTGCCACGAGGACACGTGCGACCCGGTCACTGGTGCCTGCCACCTAGAAACCAA
CCAGCGCAAGGGCGTGATGGGCGCGGGCGCGCTGCTCGTCCTGCTCGTCTGCCTGCTGCTCTCGCTGCTCGGCTGC
TGCTGCGCTTGCCGCGGCAAGGACCCTACGCGCCGGGAGCTTTCGCTTGGGAGGAAGAAGGCGCCGCACCGCACTAT
GCGGGCGCTTCAGTCGCATCAGCATGAAGCTGCCCCGGATCCCGCTCCGGAGGCAGAAACTACCCAAAGTCGTAGT
GGCCCACCACGACCTGGATAACACACTCAACTGCAGCTTCCTGGAGCCACCCTCAGGGCTGGAGCAGCCCTCACCA
TCCTGGTCCTCTCGGGCCTCCTTCTCCTCGTTTGACACCACTGATGAAGGCCCTGTGTACTGTGTACCCCATGAGG
AGGCACCAGCGGAGAGCCGGGACCCCGAAGTCCCCACTGTCCCTGCCGAGGCGCCGGCGCCGTCCCCTGTCCCCTT
GACCACGCCAGCCTCCGCCGAGGAGGCGATACCCCTCCCCGCGTCCTCCGACAGCGAGCGGTCGGCGTCCAGCGTG
GAGGGGCCCGGAGGGGCTCTGTACGCGCGCGTGGCCCGACGCGAGGCCCGGCCGGCCCGGGCCCGGGGCGAGATTG
GGGGCCTGTCGCTGTCGCCATCGCCCGAGCGCAGGAAACCGCCGCCACCTGACCCCGCCACCAAGCCTAAGGTGTC
CTGGATCCACGGCAAGCACAGCGCCGCTGCAGCTGGCCGTGCGCCCTCACCACCGCCGCCAGGCTCCGAGGCCGCG
CCCAGCCCCAGCAAGAGGAAACGGACGCCCAGCGACAAATCGGCGCATACGGTCGAACACGGCAGCCCCCGGACCC
GCGACCCAACGCCGCGCCCCCCGGGGCTGCCCGAGGAGGCGACAGCCCTCGCTGCGCCCTCGCCGCCCAGGGCCCG
AGCGCGCGCCGCGCCCCGGCCTCTTGGAGCCCACGGACGCCGGCGGTCCCCCGCGAAGCGCGCCGAGGCTGCCTCC
ATGTTGGCCGCTGACGTGCGCGGCAAGACTCGCAGCCTGGGCCGCGCCGAGGTGGCCCTGGGCGCGCAGGGCCCCA
GGGAAAAGCCGGCGCCCCCACAGAAAGCCAAGCGCTCCGTGCCGCCAGCCTCGCCCGCCCGCGCGCCCCCAGCGAC
CGAAACCCCGGGGCCTGAGAAGGCGGCGACCGACTTGCCCGCGCCTGAGACCCCCCGGAAGAAGACCCCCATCCAG
AAGCCGCCGCGCAAGAAGAGCCGGAGGCGGCCGGGCGAGCTGGGCAGGGCGGGCGCACCCACCCTGTAG

The MOL8a protein encoded by SEQ ID NO:16 has 324 amino acid residues, and is presented using the one-letter code in Table 8B (SEQ ID NO:18). The SignalP, Psort and/or Hydropathy profile for MOL8a predict that MOL8a has a signal peptide and is likely to be localized at the plasma membrane with a certainty,of 0.6000. The SignalP shows a signal sequence with a cleavage site between amino acids 43 and 44. This is typical of this type of membrane protein. Therefore it is likely that this novel human plasma membrane protein is available at the appropriate sub-cellular localization and hence accessible for the therapeutic uses described in this application.

TABLE 8B
Encoded MOL8a protein sequence.

(SEQ ID NO:18)

MEGAGPRGAGPARRRGAGGPPSPLLPSLLLLLLLWMLPDTVAPQELNPRGRNVCRAPGSQVPTCCAGWRQQGDECG
IAVCEGNSTCSENEVCVRPGECRCRHGYFGANCDTSERGVGPVLVGGAESWRDGAGSKVGRGRIRLRGGSPEVAAG
VRDAGRFRLAGGTYSSTGAFHPLRSSPAECPRQFWGPDCKELCSCHPHGQCEDVTGQCTCHARRWGARCEHACQCQ
HGTCHPRSGACRCESGWWGAQCASACYCSATSRCDPQTGACLCHAGWWGRSCNNQCACNSSPCEQQSGRCQCRERT
FGARCDRYCQCFRGRCHPVDGTCACEPGYRGKYCREPCPAGFYGLGCRRRCGQCKGQQPCTVAEGRCLTCEPGWNG
TKCDQPCATGFYGEGCSHRCPPCRDGHACNHVTGKCTRCNAGWIGDRCETKCSNGTYGEDCAFVCADCGSGHCDFQ
SGRCLCSPGVHGPHCNVTCPPGLHGADCAQACSCHEDTCDPVTGACHLETNQRKGVMGAGALLVLLVCLLLSLLGC
CCACRGKDPTRRELSLGRKKAPHRLCGRFSRISMKLPRIPLRRQKLPKVVVAHHDLDNTLNCSFLEPPSGLEQPSP
SWSSRASFSSFDTTDEGPVYCVFHEEAPAESRDPEVPTVPAEAPAPSPVPLTTPASAEEAIPLPASSDSERSASSV
EGPGGALYARVARREARPARARGEIGGLSLSPSPERRKPPPPDPATKPKVSWIHGKHSAAAAGRAPSPPPPGSEAA
PSPSKRKRTPSDKSAHTVEHGSPRTRDPTPRPPGLPEEATALAAPSPPRARARAAPRPLGAHGRRRSPAKPAEAAS
MLAADVRGKTRSLGRAEVALGAQGPREKPAPPQKAKRSVPPASPARAPPATETPGPEKAATDLPAPETPRKKTPIQ
KPPRKKSREAAGELGRAGAPTL

The full amino acid sequence of the protein of the invention was found to have 576/729 (79%) identical and 596/729 (8 1%) similarity to a murine nurse cell receptor amino acid sequence (PatP Accession No. Y85616). The full amino acid sequence of the protein of the invention was also found to have 296 of 741 amino acid residues (39%) identical and 383 of 741 amino acid residues (51%) homolog to the 830 amino acid residue acetyl LDL receptor precursor from Homo sapiens (human) (ACC:O43701).

MOL8a is expressed in the following tissues: fetal thymus, mammary gland, fetal thymus, pool of ten tissues (adrenal, mammary, prostate, testis, uterus, bone marrow*, melanoma*, pituitary*, thyroid*, spleen) (*from mRNA rather than from total RNA).

MOL8b

A novel nucleic acid was identified by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof. These were further modified by means of similarities using BLAST searches. The sequences were then manually corrected for apparent inconsistencies, thereby obtaining the sequences encoding the full-length protein. The disclosed novel MOL8b nucleic acid of 2598 nucleotides (also referred to as CG50889-02) is shown in Table 8C. An open reading frame begins with an ATG initiation codon at nucleotides 1-3 and ends with a TAG codon at nucleotides 2596-2598. A putative untranslated region upstream from the initiation codon and downstream from the termination codon are underlined in Table 8D, and the start and stop codons are in bold letters.

TABLE 8D
MOL8b Nucleotide Sequence

(SEQ ID NO:19)

ATGGAGGGCGCAGGGCCCCGGGGGGCCGGGCCGGCGCGGCGCCGGGGAGCCGGGGGGCCGCCGTCACCGCTGCTGC
CGTCGCTGCTCCTGCTGCTGCTGCTCTGGATGCTGCCGGACACCGTGGCGCCTCAGGAACTGAACCCTCGCGGCCG
CAACGTGTGCCGTGCTCCCGGCTCCCAGGTGCCCACGTGCTGCGCTGGCTGGAGGCAGCAAGGGGACGAGTGTGGG
ATTGCGGTGTGCGAAGGCAACTCCACGTGCTCAGAGAACGAGGTGTGCGTGAGGCCTGGCGAGTGCCGCTGCCGCC
ACGGCTACTTCGGTGCCAACTGCCACACCAAGTGCCCGCGCCAGTTCTGGGGCCCCGACTGCAAGGAGCTGTGTAG
CTGCCACCCACACGGGCAGTGCGAGGACGTGACAGGCCAGTGTACTTGTCACGCGCGGCGCTGGGGCGCGCGCTGC
GAGCATGCGTGCCAGTGCCAGCACGGCACGTGCCACCCGCGGAGCGGCGCGTGCCGCTGTGAGCCCGGCTGGTGGG
GCGCGCAGTGCGCCAGCGCGTGCTACTGCAGCGCCACGTCGCGCTGCGACCCACAGACCGGCGCCTGCCTGTCCCA
CGCAGGCTGGTGGGGCCGCAGCTGCAACAACCAGTGCGCCTGCAACTCGTCTCCCTGCGAGCAGCAGAGCGGCCGC
TGTCAGTGCCGCGAGCGTACGTTCGGCGCGCGCTGCGATCGCTACTGCCAGTGCTTCCGCGGCCGCTGCCACCCTG
TGGACGGCACGTGTGCCTGCGAGCCGGGCTACCGCGGCAAGTACTGTCGCGAGCCGTGCCCCGCCGGCTTCTACGG
CTTGGGCTGTCGCCGCCGGTGTGGCCAGTGCAAGGGCCAGCAGCCGTGCACGGTGGCCGAGGGCCGCTGCTTGACG
TGCGAGCCCGGCTGGAACGGAACCAAGTGCGACCAGCCTTGCGCCACCGGTTTCTATGGCGAGGGCTGCAGCCACC
GCTGTCCGCCATGCCGCGACGGGCATGCCTGTAACCATGTCACCGGCAAGTGTACGCGCTGCAACGCGGGCTGGAT
CGGCGACCGGTGCGAGACCAAGTGTAGCAATGGCACTTACGGCGAGGACTGCGCCTTCGTGTGCGCCGACTGCGGC
AGCGGACACTGCGACTTCCAGTCGGGGCGCTGCCTGTGCAGCCCTGGCGTCCACGGGCCCCACTGTAACGTGACGT
GCCCGCCCGGACTCCACGGCGCGGACTGTGCTCAGGCCTGCAGCTGCCACGAGGACACGTGCGACCCGGTCACTGG
TGCCTGCCACCTACAAACCAACCAGCGCAAGGGCGTGATGGGCGCGGGCGCGCTGCTCGTCCTGCTCGTCTGCCTG
CTGCTCTCGCTGCTCGGCTGCTGCTGCGCTTGCCGCGGCAAGGACCCTACGCGCCGGGAGCTTTCGCTTGGGAGGA
AGAAGGCGCCGCACCGACTATGCGCGGGCTTCAGTCGCATCAGCATGAAGCTGCCCCGGATCCCGCTCCGGAGGCA
GAAACTACCCAAAGTCGTAGTGGCCCACCACGACCTGGATAACACACTCAACTGCAGCTTCCTGGAGCCACCCTCA
GGGCTGGAGCAGCCCTCACCATCCTGGTCCTCTCGGGCCTCCTTCTCCTCGTTTGACACCACTGATGAAGGCCCTG
TGTACTGTGTACCCCATGAGGAGGCACCAGCGGAGAGCCGGGACCCCGAAGTCCCCACTGTCCCTGCCGAGGCGCC
GGCGCCGTCCCCTGTGCCCTTGACCACGCCAGCCTCCGCCGAGGACGCGATACCCCTCCCCGCGTCCTCCGACAGC
GAGCGGTCGGCGTCCAGCGTGGAGGGGCCCGGAGGGGCTCTGTACGCGCGCGTGGCCCGACGCGAGGCCCGGCCGG
CCCGGGCCCGGGGCGAGATTGGGGGCCTGTCGCTGTCGCCATCGCCCGAGCGCAGGAAACCGCCGCCACCTCACCC
CGCCACCAAGCCTAAGGTCTCCTGGATCCACGGCAAGCACAGCGCCGCTGCAGCTGGCCGTGCGCCCTCACCACCG
CCGCCAGGCTCCGAGGCCGCCCAGAGCCCCAGCAAGAGGAAACGGACGCCCAGCGACAAATCGGCGCATACGGTCG
AACACGGCAGCCCCCGGACCCGCGACCCAACGCCGCGCCCCCCGGGGCTGCCCGAGGAGGCGACAGCCCTCGCTGC
GCCCTCGCCGCCCAGGGCCCGAGCGCGCGCCGCGCCCGGCCTCTTGGAGCCCACGGACGCCGGCGGTCCCCCCGCG
AAGCGCGCCGAGGCTGCCTCCATGTTGGCCGCTGACGTGCGCGGCAAGACTCGCAGCCTGGGCCGCGCCGAGGTGG
CCCTGGGCGCGCAGGGCCCCAGGGAAAAGCCGGCGCCCCCACAGAAAGCCAAGCGCTCCGTGCCGCCAGCCTCGCC
CGCCCGCGCGCCCCCAGCGACCGAAACCCCGGGGCCTGAGAAGGCGGCGACCGACTTGCCCGCGCCTCAGACCCCC
CGGAAGAAGACCCCCATCCAGAAGCCGCCGCGCAAGAAGAGCCGGGAGGCGGCGGGCGAGCTGGGCAGGGCGGGCG
CACCCACCCTGTAG

The disclosed nucleic acid sequence has 1311 of 2041 bases (64%) identical to a gb:GENBANK-ID:D86864|acc:D86864.1 mRNA from Homo sapiens (Homo sapiens mRNA for acetyl LDL receptor, complete cds) (E value=3.2e−⁹⁸)

The MOL8b protein encoded by SEQ ID NO:17 has 865 amino acid residues, and is presented using the one-letter code in Table 8E (SEQ ID NO:20). The SignalP, Psort and/or Hydropathy profile for MOL8a predict that MOL8a has a signal peptide and is likely to be localized at the plasma membrane with a certainty of 0.6000. The SignalP shows a signal sequence with a cleavage site between amino acids 43 and 44. This is typical of this type of membrane protein. Therefore it is likely that this novel human plasma membrane protein is available at the appropriate sub-cellular localization and hence accessible for the therapeutic uses described in this application.

TABLE 8E
Encoded MOL8b protein sequence.

(SEQ ID NO:20)

MEGAGPRGAGPARRRGAGGPPSPLLPSLLLLLLLWMLPDTVAPQELNPRGRNVCRAPGSQVPTCCAGWRQQGDECG
IAVCEGNSTCSENEVCVRPGECRCRHGYFGANCDTKCPRQFWGPDCKELCSCHPHGQCEDVTGQCTCHARRWGARC
EHACQCQHGTCHPRSGACRCEPGWWGAQCASACYCSATSRCDPQTGACLCHAGWWGRSCNNQCACNSSPCEQQSGR
CQCRERTFGARCDRYCQCFRGRCHPVDGTCACEPGYRGKYCREPCPAGFYGLGCRRRCGQCKGQQPCTVAEGRCLT
CEPGWNGTKCDQPCATGFYGEGCSHRCPPCRDGHACNHVTGKCTRCNAGWIGDRCETKCSNGTYGEDCAFVCADCG
SGHCDFQSGRCLCSPGVHGPHCNVTCPPGLHGADCAQACSCHEDTCDPVTGACHLETNQRKGVMGAGALLVLLVCL
LLSLLGCCCACRGKDPTRRELSLGRKKAPHRLCGRFSRISMKLPRIPLRRQKLPKVVVAHHDLDNTLNCSFLEPPS
GLEQPSPSWSSRASFSSFDTTDEGPVYCVPHEEAPAESRDPEVPTVPAEAPAPSPVPLTTPASAEEAIPLPASSDS
ERSASSVEGPGGALYARVARREARPAPARGEIGGLSLSPSPERRKPPPPDPATKPKVSWIHGKHSAAAAGRAPSPP
PPGSEAAPSPSKRKRTPSDKSAHTVEHGSPRTRDPTPRPPGLPEEATALAAPSPPRARARAAPRPLGAHGRRRSPA
KRAEAASMLAADVRGKTRSLGRAEVALGAQGPREKPAPPQKAKRSVPPASPARAPPATETPGPEKAATDLPAPETP
RKKTPIQKPPRKKSREAAGELGRAGAPTL

The full amino acid sequence of the protein of the invention was found to have 340 of 823 amino acid residues (41%) identical to, and 443 of 823 amino acid residues (53%) similar to, the 830 amino acid residue ptnr:SPTREMBL-ACC:O43701 protein from Homo sapiens (Human) (ACETYL LDL RECEPTOR PRECURSOR) (E value=9.0e−¹⁶⁴).

Homology between MOL8a, 8b and the human acetyl LDL receptor are presented graphically in the multiple sequence alignment given in Table 8F (with MOL8a being shown on line 1, and MOL8b being shown on line 2) as a ClustalW analysis comparing MOL8 with related protein sequences.

[image]

[image]

Chromosomal Information:

MOL8 maps to chromosome 22q11. This assignment was made using mapping information associated with genomic clones, public genes and ESTs sharing sequence identity with the disclosed sequence and CuraGen Corporation's Electronic Northern bioinformatic tool.

Tissue Expression

MOL8 is expressed in at least the following tissues: kidney, senescent fibroblasts, lymphocyte, B cell, and germ cell tumors. Expression information was derived from the tissue sources of the sequences that were included in the derivation of the sequence of CuraGen Acc. No. CG50889-02.

MOL8 shows significant homologies to human LDL Receptor-like protein, as described in, but not limited to, the references below. Hypercholesterolemia is an autosomal dominant disorder characterized by elevation of serum cholesterol bound to low density lipoprotein (LDL). Mutations in the LDL receptor (LDLR) gene on chromosome 19 cause this disorder. Familial hypercholesterolemia is characterized by elevation of serum cholesterol bound to low density lipoprotein (LDL) and is, hence, one of the conditions producing the hyperlipoproteinemia II phenotype (see OMIM 144400). Heterozygotes develop tendinous xanthomas, corneal arcus, and coronary artery disease; the last usually becomes evident in the fourth or fifth decade. Homozygotes develop these features at a accelerated rate in addition to planar xanthomas, which may be evident at birth in the web between the first two digits.

Hepatitis C virus (HCV), the principal viral cause of chronic hepatitis, is not readily replicated in cell culture systems, making it difficult to ascertain information on cell receptors for the virus. However, several observations from studies on the role of HCV in mixed cryoglobulinemia provided some insight into HCV entry into cells. Evidence indicated that HCV and other viruses enter cells through the mediation of LDL receptors: by the demonstration that endocytosis of these viruses correlates with LDL receptor activity, by complete inhibition of detectable endocytosis by anti-LDL receptor antibody, by inhibition with anti-apolipoprotein E and anti-apolipoprotein B antibodies, by chemical methods abrogating lipoprotein/LDL receptor interactions, and by inhibition with the endocytosis inhibitor phenylarsine oxide. Agnello et al. (1999) provided confirmatory evidence by the lack of detectable LDL receptor on cells known to be resistant to infection by one of these viruses, bovine viral diarrheal virus (BVDV). Endocytosis via the LDL receptor was shown to be mediated by complexing of the virus to very low density lipoprotein (VLDL) or LDL, but not high density lipoprotein (HDL). Studies using LDL receptor-deficient cells or a cytolytic BVDV system indicated that the LDL receptor may be the main but not exclusive means of cell entry of these viruses.

Therapeutic Uses of the Composition

The expression pattern, and protein similarity information for MOL8 that it may function as human LDL Receptor-like protein. Therefore, the nucleic acid and protein of the invention are useful in potential therapeutic applications implicated, for example but not limited to, metabolic disorders, e.g. Hypercholesterolemia, viral diseases, and other diseases and disorders. The homology to antigenic secreted and membrane proteins suggests that antibodies directed against the novel genes may be useful in treatment and prevention of metabolic disorders, e.g. Hypercholesterolemia, viral diseases, and other diseases and disorders.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

The nucleic acids and proteins of the invention are useful in potential therapeutic applications implicated in various cancers including those metabolic disorders, e.g. Hypercholesterolemia, viral diseases, and other diseases and disorders. For example, but not limited to, a cDNA encoding the novel human plasma membrane protein may be useful in gene therapy, and the novel human plasma membrane protein may be useful when administered to a subject in need thereof. By way of non-limiting example, the compositions of the present invention will have efficacy for treatment of patients suffering from, for example, but not limited to, various cancers including those of the metabolic disorders, e.g. Hypercholesterolemia, viral diseases, and other diseases and disorders. The novel nucleic acid encoding the novel human plasma membrane protein, and the novel human plasma membrane protein of the invention, or fragments thereof,may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

These materials are further useful in the generation of antibodies that bind immuno-specifically to the novel MOL8 substances for use in therapeutic or diagnostic methods. These antibodies may be generated according to methods known in the art, using prediction from hydrophobicity charts, as described in the “Anti-MOLX Antibodies” section below. For example the disclosed MOL8 protein has multiple hydrophilic regions, each of which can be used as an immunogen. In one embodiment, a contemplated MOL8 epitope is from about amino acids 1 to 10. In another embodiment, a MOL8 epitope is from about amino acids 50 to 200. In further embodiment, a MOL8 epitope contains amino acids 210-400, 475-600, or 625-850. These novel proteins can also be used to develop assay system for functional analysis.

MOL9

MOL9a

A novel nucleic acid encoding a neurolysin-like protein was identified by TblastN using CuraGen Corporation's sequence file for MOL9 probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein. The disclosed novel MOL9a nucleic acid of 2355 nucleotides (also referred to as 19506719_B_EXT) is shown in Table 9A. An open reading frame begins with an ATG initiation codon at nucleotides 1-3 and ends with a TGA codon at nucleotides 1915-1917. A putative untranslated region upstream from the initiation codon and downstream from the termination codon are underlined in Table 9A, and the start and stop codons are in bold letters.

The nucleic acid sequence has 1307 of 1428 bases (91%) identical to a pig neurolysin mRNA (GENBANK-ID: AB000170) (Expect=0.0).

TABLE 9A
MOL9a Nucleotide Sequence

(SEQ ID NO:21)

ATGTTGACTTTGCACCAACAGAAATCCCTAATTCTTATTCTTTTTCTGATTCTTTTTAGAGTTGGTGGTTC
CAGGATTTTACTCAGAATGACGTTAGGAAGAGAAGTGATGTCTCCTCTTCAGGCAATGTCTTCCTATACTG
TGGCTGGCAGAAATGTTTTAAGATGGGATCTTTCACCAGAGCAAATTAAAACAAGAACTGAGGAGCTCATT
GTGCAGACCAAACAGGTGTACGATGCTGTTGGAATGCTCGGTATTGAGGAAGTAACTTACGAGAACTGTCT
GCAGGCACTGGCAGTGGAAAGGACCATGCTAGACTTTCCCCAGCATGTATCCTCTGACAAAGAAGTACGAG
CAGCAAGTACAGAAGCAGACAAAAGACTTTCTCGTTTTGATATTGAGATGAGCATGAGAGGAGATATATTT
GAGAGAATTGTTCATTTACAGCAGGAAACCTGTGATCTGGGGAAGATAAAACCTGAGGCCAGACGATACTT
GGAAAAGTCAATTAAAATGGGGAAAAGAAATGGGCTCCATCTTCCTGAACAAGTACAGAATGAAATCAAAT
CAATGAAGAAAAGAATGAGTGAGCTATGTATTGATTTTAACAAAAACCTCAATGAGGATGATACCTTCCTT
GTATTTTCCAAGGCTGAACTTGGTGCTCTTCCTGATGATTTCATTGACAGTTTAGAAAAGACAGATGATGA
CAAGTATAAAATTACCTTAAAATATCCACACTATTTCCCTCTCATGAAGAAATGTTGTATCCCTGAAACCA
GAAGAAGGATGGAAATGGCTTTTAATACAAGGTGCAAAGAGGAAAACACCATAATTTTGCAGCAGCTACTC
CCACTGCGAACCAAGGTGGCCAAACTACTCGGTTATAGCACACATGCTGACTTCGTCCTTGAAATGAACAC
TGCAAAGAGCACAAGCCGCCTAACAGCCTTTCTAGATGATTTAAGCCACAAGTTAAAACCCTTGGGTGAAG
CAGAACGAGAGTTTATTTTGAATTTGAAGAAAAAGGAATGCAAAGACAGGGGTTTTGAATATGATGGGAAA
ATCAATGCCTGGGATCTATATTACTACATGACTCAGACAGAGGAACTCAAGTATTCCATAGACCAAGAGTT
CCTCAAGGAATACTTCCCAATTGAGGTGGTCACTGAAGGCTTGCTGAACACCTACCAGGAGTTGTTGGGAC
TTTCATTTGAACAAATGACAGATGCTCATGTTTGGAACAAGAGTGTTACACTTTATACTCTGAAGGATAAA
GCTACAGCAGAAGTATTGGGACAGTTCTATTTGGACCTCTATCCAAGGCCTAGGGAAGGAAAATACAATCA
TGCGGCCTGCTTCGGTCTCCAGCCTGGCTGCCTTCTGCCTGATCGAAGCCGGATGATGGCAGTGGCTGCCC
TCGTGGTGAACTTCTCACAGCCAGTGGCAGGTCGTCCCTCTCTCCTGAGACACGACGAGGTGAGGACTTAC
TTTCATGAGTTTGGTCACGTGATGCATCAGATTTGTGCACAGACTGATTTTGCACGATTTAGCGGAACAAA
TGTGGAAACTGACTTTGTAGAGGTGCCATCGCAAATGCTTGAAAATTGGGTGTGGGACGTCGATTCCCTCC
GAAGATTGTCAAAACATTATAAAGATGGAAGCCCTATTGCAGACGATCTGCTTGAAAAACTTGTTGCTTCT
AGGCTGGTCAACACAGGTATGGGTTATGTTATTAGTAATATATATTTTCTAGATATGTTTTCATTTCAGTG
CATCCAAGTTGGAATGAAATACAGAAACCTAATCCTGAAACCTGGGGGATCTCTGGACGGCATGGACATGC
TCCACAATTTCTTGAAACGTGAGCCAAACCAAAAAGCGTTCCTAATGAGTAGAGGCCTGCATGCTCCGTGA
ACTGGGGATCTTTGGTAGCCGTCCATGTCTGGAGGACAAGTCGACATCACCATGTGTTACTGGCCTGGAAA
CTGAAGGGAGTTTTGCAAGTGAAAATTTAGATTTCTATTGACATCCTTTTGTTTTCTAATTTTAAAAATTA
TAAAGATGTAAATGGAATTATAAATACTGTGACCTAAGAAAAGACCCACTAGAAAGTAATTGTACTATAAA
ATTTCATAAAACTGGATTTGATTTCTTTTTATGAAAGTTTCATATGAATGTAACTTGATTTTTTACTATTA
TAATCTAGATAATATGATATAAGAGGGCTAAGAATTTTTAAATTGAATCATATATATGATATAATTTGATC
CTTCTTGTATCTTGAAGTTTTGTACTTGGGATTTCTGGACTCGTAAATGAATCATCACATTCTTCTGGTAA
ATATTTTCTTGG

The MOL9a protein encoded by SEQ ID NO:21 and has 638 amino acid residues, and is presented using the one-letter code in Table 9B (SEQ ID NO:22). The SignalP, Psort and or Hydropathy profile indicate that this sequence has a signal peptide between positions 23 and 24 and is likely to be localized at endoplasmic reticulum and plasma membrane (Certainty=0.8200).

TABLE 9B
Encoded MOL9a protein sequence

(SEQ ID NO:22)

MLTLDQQKSLILILFLILFRVGGSRILLRMTLGREVMSPLQAMSSYTVAGRNVLRWDLSPEQIKTRTEELI
VQTKQVYDAVGMLGIEEVTYENCLQALAVERTMLDFPQHVSSDKEVRAASTEADKRLSRFDIEMSMRGDIF
ERIVHLQQETCDLGKIKPEARRYLEKSIKMGKRNGLHLPEQVQNEIKSMKKRMSELCIDFNKNLNEDDTFL
VFSKAELGALPDDFIDSLEKTDDDKYKITLKYPHYFPVMKKCCIPETRRRMEMAFNTRCKEENTIILQQLL
PLRTKVAKLLGYSTHADFVLEMNTAKSTSRVTAFLDDLSQKLKPLGEAEREFILNLKKKECKDRGFEYDGK
INAWDLYYYMTQTEELKYSIDQEFLKEYFPIEVVTEGLLNTYQELLGLSFEQMTDAHVWNKSVTLYTVKDK
ATGEVLGQFYLDLYPRPREGKYNHAACFGLQPGCLLPDGSRMMAVAALVVNFSQPVAGRPSLLRHDEVRTY
FHEFGHVMHQICAQTDFARFSGTNVETDFVEVPSQMLENWVWDVDSLRRLSKHYKDGSPIADDLLEKLVAS
RLVNTGMGYVISNIYFLDMFSFQCIQVGMKYRNLILKPGGSLDGMDMLHNFLKREPNQKAFLMSRGLHAP

The full amino acid sequence of the protein of the invention was found to have 483/564 (85%) identity and 508/564 (90%) similarity to a rabbit endopeptidase (PatP Accession No. R26114), and have 571 of 632 amino acid residues (90%) identical to, and 592 of 632 residues (93.6%) similar to, the 704 amino acid residue neurolysin protein from pig (ptnr:SPTREMBL-ACC: Q02038) (E value=1.1e−³⁰²).

MOL9a also has homology to the proteins shown in the BLAST alignments in Table 9C.

TABLE 9C
BLASTX results for MOL9a

| | | Smallest | |
|---|---|---|---|
| | | Sum | |
| | High | Prob | |
| Sequences producing High-scoring Segment Pairs: | Score | P(N) | N |
| ptnr:SWISSNEW-ACC:Q02038 NEUROLYSIN PRECURSOR (EC 3.4.24.... | 2685 | 1.1e−302 | 2 |
| ptnr:SWTSSPROT-ACC:P42675 NEUROLYSIN PRECURSOR (EC 3.4.24... | 2697 | 2.2e−302 | 2 |
| ptnr:SPTREMBL-ACC:P79433 ENDOPEPTIDASE 24.16 (EC 3.4.-.-)... | 2677 | 7.6e−302 | 2 |
| ptnr:SWISSPROT-ACC:P42676 NEUROLYSIN PRECURSOR (EC 3.4.24... | 2601 | 4.7e−291 | 2 |
| ptnr:SWISSPROT-ACC:P47788 THIMET OLIGOPEPTIDASE (EC 3.4.2... | 1792 | 8.4e−197 | 2 |

Chromosomal Information

MOL9a maps to the Unigene entry Hs. 22151 which maps to chromosome 5 between markers D5S427-D5S647 (69.6-74.7 cM).

Tissue Expression

MOL9a is expressed in at least the following tissues: fetal lung, testis, B-cell, aorta, brain, colon, foreskin, germ cell, heart, kidney, pancreas, stomach, uterus, whole embryo and cancer cell lines MDA-MB-231 and MCF-7.

These materials are further useful in the generation of antibodies that bind immuno-specifically to the novel MOL9a substances for use in therapeutic or diagnostic methods. These antibodies may be generated according to methods known in the art, using prediction from hydrophobicity charts, as described in the “Anti-MOLX Antibodies” section below. For example the disclosed MOL9a protein has multiple hydrophilic regions, each of which can be used as an immunogen. In one embodiment, a contemplated MOL9a epitope is from about amino acids 50 to 75. In another embodiment, a MOL9a epitope is from about amino acids 100 to 150. In further embodiments, MOL9a epitopes are found in amino acids 175-200, 225-300, 325-375, 425-450, 500-550, and 600-625. These novel proteins can also be used to develop assay system for functional analysis.

MOL9b

The cloned open reading frame, codes for a 687 amino acid long protein with an overall 95% amino acid identity, to the mature form of the pig neurolysin precursor (SWISSPROT-ACC:Q02038). Oligonucleotide primers were designed to PCR amplify a DNA segment, representing an ORF, coding for the mature form of 19506719_B EXT. The forward primer includes an, in frame, BamHI restriction site. The reverse primer contains an, in frame, XhoI restriction site. The sequences of the PCR primers are the following:

| | | |
|---|---|---|
| 19506719_B-EXT Mat-Forw: | GGATCCTCCAGGATTTTACTCAGAATGACGTTAGG | (SEQ ID NO:58) |
| 19506719_B-EXT FL-Rev: | CTCGAGCGGAGCATGCAGGCCTCTACTCATTAGGAACG | (SEQ ID NO:59) |

PCR reactions were set up using a total of 5 ng cDNA, consisting equal amounts of cDNA derived from human fetal brain, testis, skeletal muscle and mammary, template, 1 microM of each of the 19506719_B-EXT Mat-Forw and 19506719_B-EXT FL-Rev primers, 5 micromoles dNTP (Clontech Laboratories, Palo Alto Calif.) and 1 microliter of 50xAdvantage-HF 2 polymerase (Clontech Laboratories, Palo Alto Calif.) in 50 microliter volume. The following reaction conditions were used:

| | | |
|---|---|---|
| a) | 96° C. | 3 minutes |
| b) | 96° C. | 30 seconds denaturation |
| c) | 60° C. | 30 seconds annealing |
| d) | 72° C. | 3 minute extension. |
| | | Repeat steps b-d 35 times |
| e) | 72° C. | 10 minutes seconds final extension |

A single, 2.1 kb large, PCR product, was isolated from agarose gel and ligated to pCR2.1 vector (Invitrogen, Carlsbad, Calif.). The cloned insert was sequenced, using vector specific, M13 Forward(−40) and M13 Reverse primers as well as the gene specific primers:

| | |
|---|---|
| GGACCATGCTAGACTTTCC, 19506719_B-EXT | (SEQ ID NO:60) |
| S1; | |
| GGAAAGTCTAGCATGGTCC, 19506719_B-EXT | (SEQ ID NO:61) |
| S2; | |
| GGCTGAACTTGGTGCTCTTCC, 19506719_B- | (SEQ ID NO:62) |
| EXT S3; | |
| GGAAGAGCACCAAGTTCAGCC, 19506719_B- | (SEQ ID NO:63) |
| EXT S4; | |
| GGCTTGCTGAACACCTACC, 19506719_B-EXT | (SEQ ID NO:64) |
| S7; | |
| GGTAGGTGTTCAGCAAGCC, 19506719_B-EXT | (SEQ ID NO:65) |
| S8; | |
| GCACAGACTGATTTTGCACG, 19506719_B-EXT | (SEQ ID NO:66) |
| S9; and | |
| CGTGCAAAATCAGTCTGTGC, 19506719_B-EXT | (SEQ ID NO:67) |
| S10. | |

The disclosed novel MOL9b nucleic acid of 2061 nucleotides (also referred to as MOL9b) is shown in Table 9D. It is thought that MOL9b is an internal fragment of an open reading frame. Therefore its 5′ and 3′ termini may be extended.

TABLE 9D
MOL9b Nucleotide Sequence

(SEQ ID NO:23)

TCCAGGATTTTACTCAGAATGACGTTAGGAAGAGAAGTGATGTCTCCTCTTCAGGCAATGTCTTCCTATAC
TGTGGCTGGCAGAAATGTTTTAAGATGGGATCTTTCACCAGAGCAAATTAAAACAAGAACTGAGGAGCTCA
TTGTGCAGACCAAACAGGTGTACGATGCTGTTCGAATGCTCGGTATTGAGGAAGTAACTTACGAGAACTGT
CTGCAGGCACTGGCAGATGTAGAAGTAAAGTATATAGTGGAAAGGACCATGCTAGACTTTCCCCAGCATGT
ATCCTCTGACAAAGAAGTACGAGCAGCAAGTACAGAAGCAGACAAAAGACTTTCTCGTTTTGATATTGAGA
TGAGCATGAGAGGAGATATATTTGAGAGAATTGTTCATTTACAGGAAACCTGTGATCTGGGGAAGATAAAA
CCTGAGGCCAGACGATACTTGGAAAAGTCAATTAAAATGGGGAAAAGAAATGGGCTCCATCTTCCTGAACA
AGTACAGAATGAAATCAAATCAATGAAGAAAAGAATGAGTGAGCTATGTATTGATTTTAACAAAAACCTCA
ATGAGGATGATACCTTCCTTGTATTTTCCAAGGCTGAACTTGGTGCTCTTCCTGATGATTTCATTGACAGT
TTAGAAAAGACAGATGATCACAAGTATAAAATTACCTTAAAATATCCACACTATTTCCCTGTCATGAAGAA
ATGTTGTATCCCTGAAACCAGAAGAAGGATGGAAATGGCTTTTAATACAAGGTGCAAAGAGGAAAACACCA
TAATTTTGCAGCAGCTACTCCCACTGCGAACCAAGGTGGCCAAACTACTCGGTTATAGCACACATGCTGAC
TTCGTCCTTGAAATGAACACTGCAAAGAGCACAAGCCGCGTAACAGCCTTTCTAGATGATTTAAGCCAGAA
GTTAAAACCCTTGGGTGAAGCAGAACGAGAGTTTATTTTGAATTTGAAGAAAAAGGAATGCAAAGACAGGG
GTTTTGAATATGATGGGAAAATCAATGCCTGGGATCTATATTACTACATGACTCAGACAGAGGAACTCAAG
TATTCCATAGACCAAGAGTTCCTCAAGGAATACTTCCCAATTGAGGTGGTCACTGAAGGCTTGCTGAACAC
CTACCAGGAGTTGTTGGGACTTTCATTTGAACAAATGACAGATGCTCATGTTTGGAACAAGAGTGTTACAC
TTTATACTGTGAAGGATAAAGCTACAGGAGAAGTATTGGGACAGTTCTATTTGGACCTCTATCCAAGGGAA
GGAAAATACAATCATGCGGCCTGCTTCGGTCTCCAGCCTGGCTGCCTTCTGCCTGATGGAAGCCGGATGAT
GGCAGTGGCTGCCCTCGTGGTGAACTTCTCACAGCCAGTGGCAGGTCGTCCCTCTCTCCTGAGACACGACG
AGGTGAGGACTTACTTTCATGAGTTTGGTCACGTGATGCATCAGATTTGTGCACAGACTGATTTTGCACGA
TTTAGCGGAACAAATGTGGAAACTGACTTTGTAGAGGTGCCATCGCAAATGCTTGAAAATTGGGTGTGGGA
CGTCGATTCCCTCCGAAGATTGTCAAAACATTATAAAGATGGAAGCCCTATTGCAGACGATCTGCTTGAAA
AACTTGTTGCTTCTAGGCTGGTCAACACAGGTCTTCTCACCCTGCGCCAGATTGTTTTGAGCAAAGTTGAT
CAGTCTCTTCATACCAACACATCGCTGGATGCTGCAAGTGAATATGCCAAATACTGCTCAGAAATATTAGG
AGTTGCAGCTACTCCAGGCACAAATATGCCAGCTACCTTTGGACATTTGGCAGGGGGATACGATGGCCAAT
ATTATGGATATCTTTGGAGTGAAGTATTTTCCATGGATATGTTTTACAGCTGTTTTAAAAAAGAAGGGATA
ATGAATCCGGAGGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTC
CG

The MOL9b protein encoded by SEQ ID NO:21 has 687 amino acid residues, and is presented using the one-letter code in Table 9E (SEQ ID NO:24).

TABLE 9E
Encoded MOL9b protein sequence

(SEQ ID NO:24)

SRILLRMTLGREVMSPLQAMSSYTVAGRNVLRWDLSPEQIKTRTEELIVQTKQVYDAVGMLGIEEVTYENC
LQALADVEVKYIVERTMLDFPQHVSSDKEVRAASTEADKRLSRFDIEMSMRGDIFERIVHLQETCDLGKIK
PEARRYLEKSIKMGKRNGLHLPEQVQNEIKSMKKRMSELCIDFNKNLNEDDTFLVFSKAELGALPDDFIDS
LEKTDDDKYKITLKYPHYFPVMKKCCIPETRRRMEMAFNTRCKEENTIILQQLLPLRTKVAKLLGYSTHAD
FVLEMNTAKSTSRVTAFLDDLSQKLKPLGEAEREFILNLKKKECKDRGFEYDGKINAWDLYYYMTQTEELK
YSIDQEFLKEYFPIEVVTEGLLNTYQELLGLSFEQMTDAHVWNKSVTLYTVKDKATGEVLGQFYLDLYPRE
GKYNHAACFGLQPGCLLPDGSRMMAVAALVVNFSQPVAGRPSLLRHDEVRTYFHEFGHVMHQICAQTDFAR
FSGTNVETDFVEVPSQMLENWVWDVDSLRRLSKHYKDGSPIADDLLEKLVASRLVNTGLLTLRQIVLSKVD
QSLHTNTSLDAASEYAKYCSEILGVAATPGTNMPATFGHLAGGYDGQYYGYLWSEVFSMDMFYSCFKKEGI
MNPEVGMKYRNLILKPGGSLDGMDMLHNFLKREPNQKAFLMSRGLHAP

MOL9c

In the present invention, the target sequence identified previously, Accession Number 19506719_B_EXT, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG56222-01

The disclosed novel MOL9c nucleic acid of 2167 nucleotides (also referred to as CG56222-01) is shown in Table 9F An open reading frame begins with an ATG initiation codon at nucleotides 16-18 and ends with a TGA codon at nucleotides 2128-2130. A putative untranslated region upstream from the initiation codon and downstream from the termination codon are underlined in Table 9F, and the start and stop codons are in bold letters.

In a search of sequence databases, it was found, for example, that the nucleic acid sequence has 2000 of 2167 bases (92%) identical to a gb:GENBANK-ID:AB000170|acc:AB000170.1 mRNA from Sus scrofa (Porcine mRNA for endopeptidase 24.16, complete cds) (Expect=0.0).

TABLE 9F
MOL9c Nucleotide Sequence

(SEQ ID NO:25)

CCTCTCAGCGCTCCCATGATCGCCCGGTGCCTTTTGGCTGTGCGAAGCCTCCGCAGAGTTGGTGGTTCCAG
GATTTTACTCAGAATGACGTTAGGAAGAGAAGTGATGTCTCCTCTTCAGGCAATGTCTTCCTATACTGTGG
CTGGCAGAAATGTTTTAAGATGGGATCTTTCACCAGAGCAAATTAAAACAAGAACTGAGGAGCTCATTGTG
CAGACCAAACAGGTGTACGATGCTGTTGGAATGCTCGGTATTGAGGAAGTAACTTACGAGAACTGTCTGCA
GGCACTGGCAGATGTAGAAGTAAAGTATATAGTGGAAAGGACCATGCTAGACTTTCCCCAGCATGTATCCT
CTGACAAAGAAGTACGAGCAGCAAGTACAGAAGCAGACAAAAGACTTTCTCGTTTTGATATTGAGATGAGC
ATGAGAGGAGATATATTTGAGAGAATTGTTCATTTACAGGAAACCTGTGATCTGGGGAAGATAAAACCTGA
GGCCAGACGATACTTGGAAAAGTCAATTAAAATGGGGAAAAGAAATGGGCTCCATCTTCCTGAACAAGTAC
AGAATGAAATCAAATCAATGAAGAAAAGAATGAGTGAGCTATGTATTGATTTTAACAAAAACCTCAATGAG
GATGATACCTTCCTTGTATTTTCCAAGGCTGAACTTGGTGCTCTTCCTGATGATTTCATTGACAGTTTAGA
AAAGACAGATGATGACAAGTATAAAATTACCTTAAAATATCCACACTATTTCCCTGTCATGAAGAAATGTT
GTATCCCTGAAACCAGAAGAAGGATGGAAATGGCTTTTAATACAAGGTGCAAAGAGGAAAACACCATAATT
TTGCAGCAGCTACTCCCACTGCGAACCAAGGTGGCCAAACTACTCGGTTATAGCACACATGCTGACTTCGT
CCTTGAAATGAACACTGCAAAGAGCACAAGCCGCGTAACAGCCTTTCTAGATGATTTAAGCCAGAAGTTAA
AACCCTTGGGTGAAGCAGAACGAGAGTTTATTTTGAATTTGAAGAAAAAGGAATGCAAAGACAGGGGTTTT
GAATATGATGGGAAAATCAATGCCTGGGATCTATATTACTACATGACTCAGACAGAGGAACTCAAGTATTC
CATAGACCAAGAGTTCCTCAAGGAATACTTCCCAATTGAGGTGGTCACTGAAGGCTTGCTGAACACCTACC
AGGAGTTGTTGGGACTTTCATTTGAACAAATGACAGATGCTCATGTTTGGAACAAGAGTGTTACACTTTAT
ACTGTGAAGGATAAAGCTACAGGAGAAGTATTGGGACAGTTCTATTTGGACCTCTATCCAAGGGAAGGAAA
ATACAATCATGCGGCCTGCTTCGGTCTCCAGCCTGGCTGCCTTCTGCCTGATGGAAGCCGGATGATGGCAG
TGGCTGCCCTCGTGGTGAACTTCTCACAGCCAGTGGCAGGTCGTCCCTCTCTCCTGAGACACGACGAGGTG
AGGACTTACTTTCATGAGTTTGGTCACGTGATGCATCAGATTTGTGCACAGACTGATTTTGCACGATTTAG
CGGAACAAATGTCGAAACTGACTTTGTAGAGGTGCCATCGCAAATGCTTGAAAATTGGGTGTGGGACGTCG
ATTCCCTCCGAAGATTGTCAAAACATTATAAAGATGGAAGCCCTATTGCAGACGATCTGCTTGAAAAACTT
GTTGCTTCTAGGCTGGTCAACACAGGTCTTCTGACCCTGCGCCAGATTGTTTTGAGCAAAGTTGATCAGTC
TCTTCATACCAACACATCGCTGGATGCTGCAAGTGAATATGCCAAATACTGCTCAGAAATATTAGGAGTTG
CAGCTACTCCAGGCACAAATATGCCAGCTACCTTTGGACATTTGGCAGGGGGATACGATGGCCAATATTAT
GGATATCTTTGGAGTGAAGTATTTTCCATGGATATGTTTTACAGCTGTTTTAAAAAAGAAGGGATAATGAA
TCCGGAGGTTGGAATGAAATACAGAAACCTAATCCTGAAACCTGGGGGATCTCTGGACGGCATGGACATGC
TCCACAATTTCTTGAAACGTGAGCCAAACCAAAAAGCGTTCCTAATGAGTAGAGGCCTGCATGCTCCGTGA
ACTGGGGATCTTTGGTAGCCGTCCATGTCTGGAGGAC

The MOL9c protein encoded by SEQ ID NO:25 has 703 amino acid residues, and is presented using the one-letter code in Table 9G (SEQ ID NO:26). The SignalP, Psort and/or Hydropathy profile for MOL9c predict that MOL9c has a signal peptide and is likely to be localized at the cytoplasm with a certainty of 0.9200. The SignalP predicts a cleavage site at the sequence between amino acids 17 and 18.

TABLE 9G
Encoded MOL9c protein sequence

(SEQ ID NO:26)

MIARCLLAVRSLRRVGGSRILLRMTLGREVMSPLQAMSSYTVAGRNVLRWDLSPEQIKTRTEELIVQTKQV
YDAVGMLGIEEVTYENCLQALADVEVKYIVERTMLDFPQHVSSDKEVRAASTEADKRLSRFDIEMSMRGDI
FERIVHLQETCDLGKIKPEARRYLEKSIKMGKRNGLHLPEQVQNEIKSMKKRMSELCIDFNKNLNEDDTFL
VFSKAELGALPDDFIDSLEKTDDDKYKITLKYPHYFPVMKKCCIPETRRRMEMAFNTRCKEENTIILQQLL
PLRTKVAKLLGYSTHADFVLEMNTAKSTSRVTAFLDDLSQKLKPLGEAEREFILNLKKKECKDRGFEYDGK
INAWDLYYYMTQTEELKYSIDQEFLKEYFPIEVVTEGLLNTYQELLGLSFEQMTDAHVWNKSVTLYTVKDK
ATGEVLGQFYLDLYPREGKYNHAACFGLQPGCLLPDGSRMMAVAALVVNFSQPVAGRPSLLRHDEVRTYFH
EFGHVMHQICAQTDFARFSGTNVETDFVEVPSQMLENWVWDVDSLRRLSKHYKDGSPIADDLLEKLVASRL
VNTGLLTLRQIVLSKVDQSLHTNTSLDAASEYAKYCSEILGVAATPGTNMPATFGHLAGGYDGQYYGYLWS
EVFSMDMFYSCFKKEGIMNPEVGMKYRNLILKPGGSLDGMDMLHNFLKREPNQKAFLMSRGLHAP

The full amino acid sequence of MOL9c was found to have 657 of 704 amino acid residues (93%) identical to, and 687 of 704 amino acid residues (97%) similar to, the 704 amino acid residue ptnr:SWISSPROT-ACC:P42675 protein from Oryctolagus cuniculus (Rabbit) (NEUROLYSIN PRECURSOR (EC 3.4.24.16) (NEUROTENSIN ENDOPEPTIDASE) (MITOCHONDRIAL OLIGOPEPTIDASE M) (MICROSOMAL ENDOPEPTIDASE) (MEP) (E value=0.0)

MOL9c is expressed in at least the following tissues: Artery, Brain, Bronchus, Cartilage, Cervix, Colon, Coronary Artery, Dermis, Epidermis, Foreskin, Heart, Kidney, Liver, Ovary, Pancreas, Pituitary Gland, Placenta, Prostate, Salivary Glands, Synovium/Synovial membrane, Thalamus, Umbilical Vein, Uterus. This information was derived by determined the tissue sources of the sequences that were included in the invention including but not limited to SeqCalling sources, Public EST sources, Literature sources, and/or RACE sources.

Possible SNPs found for MOL9c are listed in Table 9H.

TABLE 9H
SNPs

| Consensus | | Base |
|---|---|---|
| Position | Depth | Change |
| 399 | 99 | A > G |
| 858 | 51 | C > A |
| 863 | 50 | T > A |
| 1242 | 48 | T > C |
| 1810 | 141 | G > A |
| 1824 | 143 | T > C |
| 1892 | 144 | T > C |

These materials are further useful in the generation of antibodies that bind immuno-specifically to the novel MOL9c substances for use in therapeutic or diagnostic methods. These antibodies may be generated according to methods known in the art, using prediction from hydrophobicity charts, as described in the “Anti-MOLX Antibodies” section below. For example the disclosed MOL9c protein has multiple hydrophilic regions, each of which can be used as an immunogen. In one embodiment, a contemplated MOL9c epitope is from about amino acids 25 to 75. In another embodiment, a MOL9c epitope is from about amino acids 100 to 200. In further embodiments, MOL9a epitopes are found in amino acids 250-400, 450-550, and 650-700. These novel proteins can also be used to develop assay system for functional analysis.

Homology between the MOL9 isoforms and other homologous proteins is presented graphically in the multiple sequence alignment given in Table 9I (with MOL9a being shown on line 1, MOL9b on line 2, and MOL9c on line 3) as a ClustalW analysis comparing MOL9 with related protein sequences.

[image]

[image]

[image]

Endopeptidase 24.16 or mitochondrial oligopeptidase, abbreviated here as EP 24.16 (MOP), is a thiol- and metal-dependent oligopeptidase that is found in multiple intracellular compartments in mammalian cells. From an analysis of the corresponding gene, we found that the distribution of the enzyme to appropriate subcellular locations is achieved by the use of alternative sites for the initiation of transcription. The pig EP 24.16 (MOP) gene spans over 100 kilobases and is organized into 16 exons. The core protein sequence is encoded by exons 5-16 which match perfectly with exons 2-13 of the gene for endopeptidase 24.15, another member of the thimet oligopeptidase family. These two sets of 11 exons share the same splice sites, suggesting a common ancestor. Multiple species of mRNA for EP 24.16 (MOP) were detected by the 5′-rapid amplification of cDNA ends and they were shown to have been generated from a single gene by alternative choices of sites for the initiation of transcription and splicing. Two types of transcript were prepared, corresponding to transcription from distal and proximal sites. Their expression in vitro in COS-1 cells indicated that they encoded two isoforms (long and short) which differed only at their amino termini: the long form contained a cleavable mitochondrial targeting sequence and was directed to mitochondria; the short form, lacking such a signal sequence, remained in the cytosol. The complex structure of the EP 24.16 (MOP) gene thus allows, by alternative promoter usage, a fine transcriptional regulation of coordinate expression, in the different subcellular compartments, of the two isoforms arising from a single gene. PMID: 9182559, UI: 97326108 We have isolated a metallopeptidase from rat liver. The peptidase is primarily located in the mitochondrial intermembrane space, where it interacts non-covalently with the inner membrane. The enzyme hydrolyzes oligopeptides, the largest substrate molecule found being dynorphin A1-17; it has no action on proteins, and does not interact with alpha 2-macroglobulin, and can therefore be classified as an oligopeptidase. We term the enzyme oligopeptidase M. Oligopeptidase M acts similarly to thimet oligopeptidase (EC 3.4.24.15) on bradykinin and several other peptides, but hydrolyzes neurotensin exclusively at the -Pro+Tyr- bond (the symbol + is used to indicate a scissile peptide bond) rather than the -Arg+Arg- bond. The enzyme is inhibited by chelating agents and some thiol-blocking compounds, but differs from thimet oligopeptidase in not being activated by thiol compounds. The peptidase is inhibited by Pro-Ile, unlike thimet oligopeptidase, and the two enzymes are separable in chromatography on hydroxyapatite. The N-terminal amino acid sequence of rat mitochondrial oligopeptidase M contains 19 out of 20 residues identical with a segment of rabbit microsomal endopeptidase and 17 matching the corresponding segment of pig-soluble angiotensin II-binding protein. Moreover, the rat protein is recognized by a monoclonal antibody against rabbit soluble angiotensin II-binding protein, all of which is consistent with these proteins being species variants of a single protein that is a homologue of thimet oligopeptidase. The biochemical properties of the mitochondrial oligopeptidase leave us in no doubt that it is neurolysin (EC 3.4.24.16), for which no sequence has previously been reported, and which has not been thought to be mitochondrial. PMID: 7836437, UI: 95138171 We have isolated by immunological screening of a lambda ZAPII cDNA library constructed from rat brain mRNAs a cDNA clone encoding endopeptidase 3.4.24.16. The longest open reading frame encodes a 704-amino acid protein with a theoretical molecular mass of 80,202 daltons and bears the consensus sequence of the zinc metalloprotease family. The sequence exhibits a 60.2% homology with those of another zinc metallopeptidase, endopeptidase 3.4.24.15. Northern blot analysis reveals two mRNA species of about 3 and 5 kilobases in rat brain, ileum, kidney, and testis. We have transiently transfected COS-7 cells with pcDNA3 containing the cloned cDNA and established the overexpression of a 70-75-kDa immunoreactive protein. This protein hydrolyzes QFS, a quenched fluorimetric substrate of endopeptidase 3.4.24.16, and cleaves neurotensin at a single peptide bond, leading to the formation of neurotensin (1-10) and neurotensin (11-13). QFS and neurotensin hydrolysis are potently inhibited by the selective endopeptidase 3.4.24.16 dipeptide blocker Pro-Ile and by dithiothreitol, while the enzymatic activity remains unaffected by phosphoramidon and captopril, the specific inhibitors of endopeptidase 3.4.24.11 and angiotensin-converting enzyme, respectively. Altogether, these physicochemical, biochemical, and immunological properties unambiguously identify endopeptidase 3.4.24.16 as the protein encoded by the isolated cDNA clone. PMID: 7592986, UI: 96070836 A human genomic clone encompassing exons 1-3 of the neurotensin/neuromedin N gene was identified using a canine neurotensin complementary DNA probe. Sequence comparisons revealed that the 120-amino acid portion of the precursor sequence encoded by exons 1-3 is 89% identical to previously determined cow and dog sequences and that the proximal 250 bp of 5′ flanking sequences are strikingly conserved between rat and human. The 5′ flanking sequence contains cis-regulatory sites required for the induction of neurotensin/neuromedin N gene expression in PC12 cells, including AP1 sites and two cyclic adenosine-5′-monophosphate response elements. Oligonucleotide probes based on the human sequence were used to examine the distribution of neurotensin/neuromedin N messenger RNA in the ventral mesencephalon of schizophrenics and age- and sex-matched controls. Neurotensin/neuromedin N messenger RNA was observed in ventral mesencephalic cells some of which also contained melanin pigment or tyrosine hydroxylase messenger RNA. Neurons expressing neurotensin/neuromedin N messenger RNA were observed in the ventral mesencephalon of both schizophrenic and non-schizophrenic humans. PMID: 1436492, UI: 93063858 Neurotensin is a small neuropeptide of 13 amino acids that may function as a neurotransmitter or neuromodulator in the central nervous system. In the CNS, neurotensin is localized to the catecholamine-containing neurons. A catecholamine-producing cell line can also produce NT. Lithium salts, widely used in the treatment of manic-depressive patients, dramatically potentiate NT gene expression in this cell line. Gerhard et al. (1989) used a canine cDNA as a probe on a somatic cell hybrid panel to determine that the human gene is located on chromosome 12. The tridecapeptide neurotensin (162650) is widely distributed in various regions of the brain and in peripheral tissues. In the brain, neurotensin acts as a neuromodulator, in particular of dopamine transmission in the nigrostriatal and mesocorticolimbic systems, suggesting its possible implication in dopamine-associated behavioral neurodegenerative and neuropsychiatric disorders. Its various effects are mediated by specific membrane receptors. Vita et al. (1993) isolated a cDNA encoding the human neurotensin receptor and showed that it predicts a 418-amino acid protein that shares 84% homology with the rat protein. Le et al. (1997) also cloned the human neurotensin receptor (NTR) cDNA and its genomic DNA. The gene is encoded by 4 exons spanning more than 10 kb. The authors identified a highly polymorphic tetranucleotide repeat approximately 3 kb from the gene. Southern blot analysis revealed that the NTR gene is present in the human genome as a single-copy gene. Le et al. (1997) stated that the neurotensin receptor has 7 transmembrane spanning regions and high homology to other receptors that couple to G proteins

Neurolysin is expressed ubiquitously in the rat brain (Massarelli et al. Brain Res Dec. 18, 1999; 851(1-2):261-5; Dauch et al. J Neurochem November 1992; 59(5): 1862-7). It has been suggested that this enzyme plays a role in the regulation of neurologically active peptides (Vincent et al: Br J Pharmacol June 1997; 121(4): 705-10) and activity differs depending on the cellular source of this enzyme whether it is expressed in primary cultured neurons and astrocytes (Vincent et al. J Neurosci Aug. 15, 1996; 16(16): 5049-59). This might play a role in nociception and signal transduction in the brain as well as central nervous system. Related endopeptidases have been shown to play a role in processing angiotensin and important regulator of blood pressure.

Uses of the Compositions of the Invention

The expression pattern, map location and protein similarity information for MOL9 suggest that it may function as neurolysin family. Therefore, the nucleic acids and proteins of the invention are useful in potential therapeutic applications implicated, for example but not limited to, in various pathologies/disorders as described below and/or other pathologies/disorders. Potential therapeutic uses for the invention(s) are, for example but not limited to, the following: (i) Protein therapeutic, (ii) small molecule drug target, (iii) antibody target (therapeutic,,diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues). These may also function in extracellular matrix remodeling in tissues described above.

The nucleic acids and proteins of the invention are useful in potential therapeutic applications implicated in various diseases and disorders described below and/or other pathologies and disorders. For example, but not limited to, a cDNA encoding the neurolysin-like protein may be useful in gene therapy, and the neurolysin-like protein may be useful when administered to a subject in need thereof. By way of nonlimiting example, the compositions of the present invention will have efficacy for treatment of patients suffering from Cancer, Trauma, Viral/bacterial/parasitic infections, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Atherosclerosis, Aneurysms, Hypertension, Fibromuscular dysplasia, Stroke, Scleroderma, Fertility, Diabetes, Von Hippel-Lindau (VHL) syndrome , Pancreatitis, Hirschsprung's disease , Crohn's Disease, Appendicitis, Alzheimer's disease, Stroke, Hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Systemic lupus erythematosus , Autoimmune disease, Asthma, Emphysema, Scleroderma, Autoimmune disease, Renal artery stenosis, Interstitial nephritis, Glomerulonephritis, Polycystic kidney disease, Renal tubular acidosis, IgA nephropathy, Hypercalceimia, Lesch-Nyhan syndrome. The novel nucleic acid encoding the neurolysin-like protein, and the neurolysin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods

MOL10

MOL10a

A novel nucleic acid encoding a protein bearing sequence similarity to Cyclic-Nucleotide-Gated Olfactory Channel-like protein was identified by TblastN using CuraGen Corporation's sequence file for MOL10 probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein. The disclosed novel MOL10a nucleic acid of 1835 nucleotides (also referred to as GM98960647_A) is shown in Table 10A. An open reading frame begins with an ATG initiation codon at nucleotides 54-56 and ends with a TGA codon at nucleotides 1788-1790. A putative untranslated region upstream from the initiation codon and downstream from the termination codon are underlined in Table 10A, and the start and stop codons are in bold letters.

The nucleic acid sequence has 1536 of 1733 bases (88%) identical to a Rattus norvegicus Cyclic-Nucleotide-Gated Olfactory Channel ocnc2 mRNA (GENBANK-ID: U12623) (Expect=5.2e⁻¹⁰⁸).

TABLE 10A
MOL10a Nucleotide Sequence

(SEQ ID NO:27)

TACAGGCAGAGAGGGTGTGGACATCTCACACCCCAGCACCAGACCACAGAACCATGAGCCAGGACACCAAA
GTGAAGACAACAGAGTCCAGTCCCCCAGCCCCATCCAAGGCCAGGAGGAAGTTGCTGCCTGTCCTGGACCC
ATCTGGGGATTACTACTACTGGTGGCTGAACACAATGGTCTTCCCAGTCATGTATAACCTCATCATCCTCG
TGTGCAGGGCCTGCTTCCCCGACTTGCAGCACGGTTATCTGGTGGCCTGGTTGGTGCTGGACTACACGAGT
GACCTGCTATACCTACTAGACATGGTGGTGCGCTTCCACACAGGTGGATTCTTGGAACAGGGCATCCTGGT
GGTGGACAAGGGTAGGATCTCGAGTCGCTACGTTCGCACCTGGAGTTTCTTCTTGGACCTGGCTTCCCTGA
TGCCCACAGATGTGGTCTACGTGCGGCTGGGCCCGCACACACCCACCCTGAGGCTGAACCGCTTTCTCCGC
GCGCCCCGCCTCTTCGAGGCCTTCGACCGCACAGAGACCCGCACAGCTTACCCAAATGCCTTTCGCATTGC
CAAGCTGATGCTTTACATTTTTGTCGTCATCCATTGGAACAGCTGCCTATACTTTGCCCTATCCCGGTACC
TGGGCTTCGGGCGTGACGCATGGGTGTACCCGGACCCCGCGCAGCCTGGCTTTGAGCGCCTGCGGCGCCAG
TACCTCTATAGCTTTTACTTCTCCACGCTGATACTGACTACAGTGGGCGATACACCGCCGCCAGCCAGGGA
AGAAGAGTACCTCTTCATGGTGGGCGACTTCCTGCTGGCCGTCATGGGTTTCGCCACCATCATGGGTAGCA
TGAGCTCTGTCATCTACAACATGAACACTGCAGATGCCGCTTTCTACCCAGATCATGCACTGGTGAAGAAG
TACATGAAGCTGCAGCACGTCAACCGCAAGCTGGAGCGGCGAGTTATTGACTGGTATCAGCACCTGCAGAT
CAACAAGAAGATGACCAACGAGGTAGCCATCTTACAGCACTTGCCTGAGCGGCTGCGGGCAGAAGTGGCTG
TGTCTGTGCACCTGTCCACTCTGAGCCGGGTGCAGATCTTTCAGAACTGTGAGGCCAGCCTGCTGGAGGAG
CTGGTGCTGAAGCTGCAGCCCCAGACCTACTCACCAGGTGAATATGTATGCCGCAAAGGAGACATTGGCCA
AGAGATGTACATCATCCGACAGGGTCAACTGGCCGTGGTGGCAGATGATGGTATCACACAGTATGCTGTGC
TCGGTGCAGGGCTCTACTTTGGGGAGATCAGCATCATCAACATCAAAGGTGGGAACATGTCTGGGAACCGC
CGCACAGCCAACATCAAGAGCCTAGGTTATTCAGACCTATTCTGCCTGAGCAAGGAGGACCTGCAGGAGGT
GCTGAGCGAGTATCCACAAGCACAGACCATCATGGAGGAGAAAGGACGTGAGATCCTGCTGAAAATGAACA
AGTTGGACGTGAATGCTGAGGCAGCTGAGATCGCCCTGCAGGAGGCCACAGAGTCCCGGCTACGAGGCCTA
GACCAGCAGCTGGATGATCTACAGACCAAGTTTGCTCGCCTCCTGGCTGAGCTGGAGTCCAGCGCACTTAA
GATTGCTTACCGCATTGAACGGCTGGAGTGGCAGACTCGAGAGTGGCCAATGCCCGAGGACCTGGCTGAGG
CTGATGACGAGGGTGAGCCTGAGGAGGGAACTTCCAAAGATGAAGAGGGCAGGGCCAGCCAGGAGGGACCC
CCAGGTCCAGAGTGACCCCATCCCCATCCCCAGGATTCCCACCTCCTAGTCAATCCAGAG

The MOL10a protein encoded by SEQ ID NO:27 has 638 amino acid residues, and is presented using the one-letter code in Table 10B (SEQ ID NO:28). PSORT analysis predicts the protein of the invention to be localized in the plasma membrane with a certainty of 0.6000. Using the SIGNALP analysis, it is predicted that the protein of the invention has a signal peptide with most likely cleavage site between positions 57 and 58.

TABLE 10B
Encoded MOL10a protein sequence

(SEQ ID NO:28)

MSQDTKVKTTESSPPAPSKARRKLLPVLDPSGDYYYWWLNTMVFPVMYNLIILVCRACFPDLQHGYLVAWL
VLDYTSDLLYLLDMVVRFHTGGFLEQGILVVDKGRISSRYVRTWSFFLDLASLMPTDVVYVRLGPHTPTLR
LNRFLRAPRLFEAFDRTETRTAYPNAFRIAKLMLYIFVVIHWNSCLYFALSRYLGFGRDAWVYPDPAQPGF
ERLRRQYLYSFYFSTLILTTVGDTPPPAREEEYLFMVGDFLLAVMGFATIMGSMSSVIYNMNTADAAFYPD
HALVKKYMKLQHVNRKLERRVIDWYQHLQINKKMTNEVAILQHLPERLRAEVAVSVHLSTLSRVQIFQNCE
ASLLEELVLKLQPQTYSPGEYVCRKGDIGQEMYIIREGQLAVVADDGITQYAVLGAGLYFGEISIINIKGG
NMSGNRRTANIKSLGYSDLFCLSKEDLREVLSEYPQAQTIMEEKGREILLKMNKLDVNAEAAEIALQEATE
SRLRGLDQQLDDLQTKFARLLAELESSALKIAYRIERLEWQTREWPMPEDLAEADDEGEPEEGTSKDEEGR
ASQEGPPGPE

The full amino acid sequence of the protein of the invention was found to have 1068 of 1649 amino acid residues (64%) identical to, and 1068 of 1649 residues (64%) positive with, the 575 amino acid residue Cyclic-Nucleotide-Gated Olfactory Channel ocnc2 subunit protein from Rattus norvegicus (ptnr:SPTREMBL-ACC: Q64359) (E value=5.5e−⁵⁴), and 0.292 of 556 amino acid residues (52%) identical to, and 404 of 556 residues (72%) positive with, the 694 amino acid residue Cone Photoreceptor cGMP-Gated Channel Alpha Subunit Homo sapiens (Human) (ptnr: TREMBLNEW-ACC: AAC17440) (E value=5.8e−¹⁵⁷)

MOL10b

In the present invention, the target sequence identified previously, MOL10a Accession Number GM98960647_A (also known as CG54557-01), was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated MOL10b (Accession Number CG54557-02). This differs from the previously identified sequence [GM98960647^{—}A (also known as CG54557-01)] at aminoacid position 159 T->A and has deletions at positions 22 R, 93 G and 426 G.

The disclosed novel MOL10b nucleic acid of 2551 nucleotides (also referred to as CG54557-02) is shown in Table 9D. An open reading frame begins with an ATG initiation codon at nucleotides 779-781 and ends with a TGA codon at nucleotides 2504-2506. A putative untranslated region upstream from the initiation codon and downstream from the termination codon are underlined in Table 10A, and the start and stop codons are in bold letters.

The nucleic acid sequence has 11625 of 1857 bases (87%) identical to a gb:GENBANK-ID:RNU12425|acc:U12425.1 mRNA from Rattus norvegicus (Rattus norvegicus olfactory cyclic nucleotide-gated channel mRNA, complete cds) (Expect=4.0e⁻³¹⁶).

TABLE 10D
MOL10b Nucleotide Sequence

(SEQ ID NO:29)

GTTTTTGTTCTTTTGATATAGGAGATATTGAAGCAGGTTCACAAAAAGACAAAAGTTGAAAGATTGGGGAC
CATAAAACACATGGAATGGTTGGTAGGATCAGGCACTAGAAGTCACAAGAAGGATATGAGGACAAAAGCAC
CATAGGATGGCCCCTATCACACTACCTATGAGAATGGTGTGATGGGGGAACGCGTATGTGGAGCTAGATAA
GGGTAGGAAGTACCTTACAAAAATAGAGCTCACTTCTCATGTGAGAGGCATCTCTTTGTCCCTGGAGAATA
GTTTAGCACCTGACATAGATAAGCCATTCAGTAATAGTTGTTAAATAAATAAATAGTGACGCCCAAATAGA
ATTTGCAAAGATAAAACAGAGTCTTTGATCCTACACTAAAACTGACCTCTTCTGACCCAGAGGACACCTAT
GTAGCTCAGTTGCTCTGGAAAGAGGGGAGGAGGAAAACAGAGACAACACTCACGCTTCCCTCTGAGGCATG
CACCCCCACCTTCTCCAGGGATCTCATTACAGGTGTTTAGCTGCGCAGCTGTAAGCCCAGGCCCTGGGAGA
CAGGGCAGAGTGCTAGAGCTACACTGTCTCCACCCCTTCAGTAGCGCTAGCTCTGGTTGTGTTGCTAAGAG
CCCCAAACACAAAGAAGTCACAGCAGAAGCCCAACACCAGCCTCCTTCAGCCAGTCAGCCACTAGTGCCCA
ACTCCAGAAGTCCCCTACAGGCAGAGAGGGTGTGGACATCTCACACCCCAGCACCAGACCACAGPACCATG
AGCCAGGACACCAAAGTGAAGACAACAGAGTCCAGTCCCCCAGCCCCATCCAAGGCCAGCAAGTTGCTGCC
TGTCCTGGACCCATCTGGGGATTACTACTACTGGTGGCTGAACACAATGGTCTTCCCAGTCATGTATAACC
TCATCATCCTCGTGTGCAGAGCCTCCTTCCCCGACTTGCAGCACCGTTATCTGGTGGCCTGGTTGGTGCTG
GACTACACGAGTGACCTGCTATACCTACTAGACATGGTGGTGCGCTTCCACACAGGATTCTTGGAACAGGG
CATCCTCGTGGTGGACAAGGCTAGGATCTCGAGTCGCTACGTTCGCACCTGGAGTTTCTTCTTGGACCTGG
CTTCCCTGATGCCCACAGATCTGGTCTACGTGCGGCTGGGCCCGCACACACCCACCCTGAGGCTAAACCGC
TTTCTCCGCGCCCCCCGCCTCTTCGAGCCCTTCGACCGCGCAGAGACCCGCACAGCTTACCCAAATGCCTT
TCGCATTGCCAAGCTGATGCTTTACATTTTTGTCGTCATCCATTCGAACAGCTGCCTATACTTTGCCCTAT
CCCCGTACCTCCCCTTCGGGCGTGACGCATGGGTGTACCCGGACCCCGCGCACCCTGGCTTTGAGCGCCTG
CGGCGCCAGTACCTCTATAGCTTTTACTTCTCCACGCTGATACTGACTACAGTGGGCCATACACCGCCGCC
AGCCAGGGAAGAAGAGTACCTCTTCATGGTGCCCGACTTCCTGCTGGCCGTCATCGGTTTCGCCACCATCA
TGGGTAGCATGAGCTCTGTCATCTACAACATGAACACTGCACATGCGGCTTTCTACCCAGATCATGCACTG
GTGAAGAAGTACATGAAGCTGCAGCACGTCAACCGCAAGCTGGACCCGCGAGTTATTGAcTGGTATCAGCA
CCTGCAGATCAACAACAAGATGACCAACGAGGTAGCCATCTTACAGCACTTGCCTGAGCGGCTGCGCGCAG
AAGTGGCTGTCTCTGTGCACCTGTCCACTCTGAGCCGGGTGCAGATCTTTCAGAACTGTGACGCCAGCCTG
CTGGAGGAGCTCGTGCTGAGCCTGCAGCCCCACACCTACTCACCAGGTCAATATGTATGCCGCAAAGGAGA
CATTGGCCAAGAGATGTACATCATCCGAGAGGGTCAACTGGCCGTGCTGGCAGATGATGGTATCACACAGT
ATGCTGTGCTCGGTGCAGGGCTCTACTTTGGGGAGATCAGCATCATCAACATCAAAGGGAACATGTCTGGG
AACCGCCGCACAGCCAACATCAAGAGCCTAGGTTATTCAGACCTATTCTGCCTGACCAAGGACGACCTGCG
GGAGGTGCTGAGCGACTATCCACAAGCACAGACCATCATGGAGGAGAAAGGACGTGAGATCCTGCTGAAAA
TGAACAAGTTGGACGTGAATGCTGAGGCAGCTGACATCGCCCTGCAGGAGGCCACAGAGTCCCGCCTACGA
GCCCTAGACCAGCAGCTGGATGATCTACAGACCAAGTTTGCTCGCCTCCTGGCTGAGCTGGAGTCCAGCGC
ACTTAAGATTGCTTACCGCATTGAACGCCTGGAGTGGCAGACTCGACAGTGGCCAATGCCCGAGGACCTGG
CTGAGGCTGATGACGAGGGTGACCCTGAGGAGGGAACTTCCAAAGATGAAGAGGGCAGGGCCAGCCAGGAG
GGACCCCCACGTCCAGAGTGACCCCATCCCCATCCCCAGGATTCCCACCTCCTAGTGAATCCAGAG

The MOL9a protein encoded by SEQ ID NO:29 has 575 amino acid residues, and is presented using the one-letter code in Table 10B (SEQ ID NO:30). PSORT analysis predicts the protein of the invention to be localized in the plasma membrane with a certainty of 0.6000. Using the SIGNALP analysis, it is predicted that the protein of the invention has a signal peptide with most likely cleavage site between positions 56 and 57 (CRA-CF)

TABLE 10E
Encoded MOL10b protein sequence

(SEQ ID NO:30)

MSQDTKVKTTESSPFAPSKARKLLPVLDPSGDYYYWWLNTMVFFVMYNLTTLVCRACFPDLQHGYLVAWLV
LDYTSDLLYLLDMVVRFHTGFLEQGILVVDRCRTSSRYVRTWSFFLELASLMPTDVVYVRLGPHTPTLRLN
RFLRAPRLFEAFDRAETRTAYPNAFRIAKLMLYIFVVIHWNSCLYPALSRYLGFGRDAWVYPDPAQPGFER
LRRQYLYSFYFSTLILTTVGDTPPPAREEEYLFMVGDFLLAVMGFATIMGSMSSVTYNMNTADAAFYPDHA
LVKKYMKLQHVNRKLERRVIDWYQHLQINKKMTNEVAILQHLPERLRAEVAVSVHLSTLSRVQIFQNCEAS
LLEELVLKLQPQTYSPGEYVCRKCDTGQEMYIIREGQLAVVADDGITQYAVLGAGLYFGEISIINIKGNMS
GNRRTANTKSLGYSDLFCLSKEDLREVLSEYPQAQTIMEEKGREILLKMNKLDVNAEAAEIALQEATESRL
RGLDQQLDDLQTKFARLLAELESSALKTAYRIERLEWQTREWPMPEDLAEADDEGEPEEGTSKDEEGRASQ
EGPPGPE

The full amino acid sequence of the protein of the invention was found to have 536 of 575 amino acid residues (93%) identical to, and 552 of 575 amino acid residues (96%) similar to, the 575 amino acid residue ptnr:SWISSPROT-ACC:Q64359 protein from Rattus norvegicus (Rat) (CYCLIC-NUCLEOTIDE-GATED OLFACTORY CHANNEL OCNC2 SUBUNIT) (E value=4.2e−²⁸⁷)

Chromosomal Information:

The Cyclic-nucleotide gated olfactory channel ocnc2 disclosed in this invention maps to chromosome 11. This information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

Tissue Expression

The Cyclic-nucleotide gated olfactory channel ocnc2 disclosed in this invention is expressed in at least the following tissues: Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

In addition, the sequence is predicted to be expressed in the following tissues because of the expression pattern of (GENBANK-ID: gb:GENBANK-ID:RNU12425|acc:U12425.1) a closely related Rattus norvegicus olfactory cyclic nucleotide-gated channel mRNA, complete cds homolog in species Rattus norvegicus: olfactory neuroepithelium.

These materials are further useful in the generation of antibodies that bind immuno-specifically to the novel MOL10b substances for use in therapeutic or diagnostic methods. These antibodies may be generated according to methods known in the art, using prediction from hydrophobicity charts, as described in the “Anti-MOLX Antibodies” section below. For example the disclosed MOL10b protein has multiple hydrophilic regions, each of which can be used as an immunogen. In one embodiment, a contemplated MOL10b epitope is from about amino acids 25 to 75. In another embodiment, a MOL10b epitope is from about amino acids 1 to 30. In further embodiments, MOL10b epitopes are found in amino acids 150-250, 275-350, 375-400. and 425-560. These novel proteins can also be used to develop assay system for functional analysis.

Homology between the MOL10 isoforms and other homologous proteins is presented graphically in the multiple sequence alignment given in Table 91 (with MOL10a being shown on line 1 and MOL10b on line 2) as a ClustalW analysis comparing MOL10 with related protein sequences.

[image]

[image]

Cyclic nucleotide-gated (CNG) channels play central roles in visual and olfactory signal transduction. In the retina, rod photoreceptors express the subunits CNCalpha1 and CNCbeta1a. In cone photoreceptors, only CNCalpha2 expression has been demonstrated so far. Rat olfactory sensory neurons (OSNs) express two homologous subunits, here designated CNCalpha3 and CNCalpha4. This paper describes the characterization of CNCbeta1b, a third subunit expressed in OSNs and establishes it as a component of the native channel. CNCbeta1b is an alternate splice form of the rod photoreceptor CNCbeta1a subunit. Analysis of mRNA and protein expression together suggest co-expression of all three subunits in sensory cilia of OSNs. From single-channel analyses of native rat olfactory channels and of channels expressed heterologously from all possible combinations of the CNCalpha3, -alpha4, and -beta1b subunits, we conclude that the native CNG channel in OSNs is composed of all three subunits. Thus, CNG channels in both rod photoreceptors and olfactory sensory neurons result from coassembly of specific alpha subunits with various forms of an alternatively spliced beta subunit.

Phototransduction is mediated by an enzymatic cascade that ultimately leads to the hydrolysis of cGMP. The photoreceptor cells, rods and cones, integrate and respond to cGMP hydrolysis via a cGMP-gated cation channel in the plasma membrane of the outer segment. Kaupp et al. (1989) cloned this channel from bovine retina. Dhallan et al. (1991) used the bovine sequence to isolate cDNA and genomic DNA encompassing the entire protein coding region of the human homolog. Assignment to chromosome 4 was achieved by study of somatic cell hybrids. Pittler et al. (1992) determined the primary structures of the human and mouse retinal rod cGMP-gated cation channel by analysis of cDNA clones and amplified DNA. The open reading frames predicted polypeptides of 690 and 683 residues, respectively, exhibiting 88% sequence similarity. Significant sequence similarity (59%) of the visual cGMP-gated channel to the olfactory cAMP-gated channel was pointed out. The RNA transcript was found to be 3.2 kb long in human, mouse, and dog. By PCR used in connection with somatic cell hybrid DNAs, Pittler et al. (1992) mapped the CNCG gene to 4p14-q13 near the centromere. By interspecific backcross haplotype analysis, the corresponding gene in the mouse, Cncg, was mapped to a site 0.9 cM proximal to the Kit locus on chromosome 5. Griffin et al. (1993) mapped the CNCG1 gene to 4p12-cen by fluorescence in situ hybridization. It is noteworthy that the rod cGMP PDE beta polypeptide (PDEB; 180072) also maps to 4p, at 4p16.3.

Uses of the Compositions of the Invention

The protein similarity information, expression pattern, and map location for MOL10 suggest that it may have important structural and/or physiological functions characteristic of the Cyclic-nucleotide gated channel family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from:

color blindness, CNS developmental disorders and other diseases, disorders and conditions of the like.

These materials are further useful in the generation of antibodies that bind immunospecifically to the novel substances of the invention for use in therapeutic or diagnostic methods.

A summary of the MOLX nucleic acids and proteins of the invention is provided in Table 11.

TABLE 11
Summary Of Nucleic Acids And Proteins Of The Invention

| | | | | Ami- |
|---|---|---|---|---|
| | | | | no |
| | | | Nucleic | Acid |
| | | | Acid | SEQ |
| | | Clone; Description | SEQ ID | ID |
| Name | Tables | of Homolog | NO | NO |
| MOL1a | 1A, 1B, | MOL1a: GM_79960178 | 1 | 2 |
| MOL1b | 1C, 1D | MOL1b: CG54674-02 | 114 | 115 |
| MOL1c | 1E, 1F | MOL1c: 248587042 | 116 | 117 |
| MOL1d | 1G, 1H | MOL1d: 248651580 | 118 | 119 |
| MOL1e | 1I, 1K | MOL1e: 255304731 | 120 | 122 |
| MOL1f | 1L, 1M | MOL1f: 255304783 | 123 | 124 |
| MOL1g | 1N, 1P | MOL1g: 255341675 | 125 | 127 |
| MOL1h | 1Q, 1R | MOL1h: 248210503 | 128 | 129 |
| MOL1i | 1S, 1T | MOL1i: 248210481 | 130 | 131 |
| MOL1j | 1U, 1V | MOL1j: 248210473 | 132 | 133 |
| MOL1k | 1W, 1X | MOL1k: 248210474 | 134 | 135 |
| MOL1l | 1Y, 1Z | MOL1l: 246484229 | 136 | 137 |
| MOL1m | 1AAA, 1AAC | MOL1m: 258065840 | 158 | 159 |
| MOL1n | 1AAD, 1AAE | MOL1n: 263483006 | 160 | 161 |
| MOL1o | 1AAF, 1AAG | MOL1o: CG54674-01 | 162 | 163 |
| MOL1p | 1AAH, 1AAJ | MOL1p: 263676346 | 164 | 165 |
| MOL1q | 1AAK, 1AAM | MOL1q: 264289162 | 166 | 167 |
| MOL1r | 1AAN, 1AAO | MOL1r: 248651580 | 168 | 169 |
| MOL1s | 1AAP, 1AAR | MOL1s: 255304731 | 170 | 171 |
| MOL1t | 1AAS, 1AAT | MOL1t: 255304783 | 172 | 173 |
| MOL1u | 1AAU, 1AAW | MOL1u: 255341675 | 174 | 175 |
| MOL2a | 2A, 2B | MOL2a: | 3 | 4 |
| | | 20466828_EXT1 | | |
| MOL2b | 2C, 2D | MOL2b: CG51424-02 | 138 | 139 |
| MOL3 | 3A, 3B | MOL3: 82254077.0.1 | 5 | 6 |
| MOL4a | 4A, 4B | MOL4a: AC004826 | 7 | 8 |
| MOL4b | 4C, 4D | MOL4b: CG53138-05 | 140 | 141 |
| MOL4c | 4E, 4F | MOL4c: CG53138-06 | 142 | 143 |
| MOL4d | 4G, 4H | MOL4d: CG53138-04 | 144 | 145 |
| MOL4e | 4I, 4J | MOL4e: 251445570 | 146 | 147 |
| MOL4f | 4K, 4L | MOL4f: 250059708 | 148 | 149 |
| MOL4g | 4M, 4N | MOL4g: 246861879 | 150 | 151 |
| MOL5 | 5A, 5B, | MOL5: AC025535 | 9 | 10 |
| MOL6 | 6A, 6B | MOL6a: | 11 | 12 |
| | | GM_87760758_A | | |
| | 6C, 6D | NOV6b: CG54548-03 | 152 | 153 |
| | 6E, 6F | MOL6c: 246861979 | 154 | 155 |
| | 6G, 6H | MOL6d: 246862070 | 156 | 157 |
| | 6J, 6K | MOL6e: | 13 | 14 |
| | | GM_87760758_A_da | | |
| | 6BBA, 6BBB | MOL6f: 256801454 | 176 | 177 |
| MOL7 | 7A, 7B | MOL7: 30675745.0.499 | 15 | 16 |
| MOL8 | 8A, 8B | MOL8a: 11800699-0-16 | 17 | 18 |
| | 8D, 8E | MOL8b: CG56222-01 | 19 | 20 |
| MOL9 | 9A, 9B | MOL9a: | 21 | 22 |
| | | 19506719_B_EXT | | |
| | 9D, 9E | MOL9b: | 23 | 24 |
| | | 19506719_B_EXT-S773 | | |
| | 9F, 9G | MOL9c: CG56222-01 | 25 | 26 |
| MOL10 | 10A, 10B | MOL10a | 27 | 28 |
| | | GM98960647_A | | |
| | 10D, 10E | MOL10b CG54557-02 | 29 | 30 |

MOLX Nucleic Acids and Polypeptides

One aspect of the invention pertains to isolated nucleic acid molecules that encode MOLX polypeptides or biologically active portions thereof. Also included in the invention are nucleic acid fragments sufficient for use as hybridization probes to identify MOLX-encoding nucleic acids (e.g, MOLX mRNAs) and fragments for use as PCR primers for the amplification and/or mutation of MOLX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof The nucleic acid molecule may be single-stranded or double-stranded, but preferably is comprised double-stranded DNA.

An MOLX nucleic acid can encode a mature MOLX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated MOLX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 as a hybridization probe, MOLX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to MOLX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or a portion of this nucleotide sequence (e.g., a fragment that can be used as a probe or primer or a fragment encoding a biologically-active portion of an MOLX polypeptide). A nucleic acid molecule that is complementary to the nucleotide sequence shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 is one that is sufficiently complementary to the nucleotide sequence shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 that it can hydrogen bond with little or no mismatches to the nucleotide sequence shown SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of MOLX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an MOLX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human MOLX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, as well as a polypeptide possessing MOLX biological activity. Various biological activities of the MOLX proteins are described below.

An MOLX polypeptide is encoded by the open reading frame (“ORF”) of an MOLX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human MOLX genes allows for the generation of probes and primers designed for use in identifying and/or cloning MOLX homologues in other cell types, e.g. from other tissues, as well as MOLX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176; or an anti-sense strand nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176; or of a naturally occurring mutant of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176.

Probes based on the human MOLX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an MOLX protein, such as by measuring a level of an MOLX-encoding nucleic acid in a sample of cells from a subject e.g., detecting MOLX mRNA levels or determining whether a genomic MOLX gene has been mutated or deleted.

“A polypeptide having a biologically-active portion of an MOLX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of MOLX” can be prepared by isolating a portion SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 that encodes a polypeptide having an MOLX biological activity (the biological activities of the MOLX proteins are described below), expressing the encoded portion of MOLX protein (e.g., by recombinant expression in vitro) and assessing the activity of the encoded portion of MOLX.

MOLX Nucleic Acid and Polypeptide Variants

The invention further encompasses nucleic acid molecules that differ from the nucleotide sequences shown SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 due to degeneracy of the genetic code and thus encode the same MOLX proteins as that encoded by the nucleotide sequences shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177.

In addition to the human MOLX nucleotide sequences shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 it will be appreciated by those skilled in the art that DNA sequence polymorphisms that lead to changes in the amino acid sequences of the MOLX polypeptides may exist within a population (e.g., the human population). Such genetic polymorphism in the MOLX genes may exist among individuals within a population due to natural allelic variation. As used herein, the terms “gene” and “recombinant gene” refer to nucleic acid molecules comprising an open reading frame (ORF) encoding an MOLX protein, preferably a vertebrate MOLX protein. Such natural allelic variations can typically result in 1-5% variance in the nucleotide sequence of the MOLX genes. Any and all such nucleotide variations and resulting amino acid polymorphisms in the MOLX polypeptides, which are the result of natural allelic variation and that do not alter the functional activity of the MOLX polypeptides, are intended to be within the scope of the invention.

Moreover, nucleic acid molecules encoding MOLX proteins from other species, and thus that have a nucleotide sequence that differs from the human sequence SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 are intended to be within the scope of the invention. Nucleic acid molecules corresponding to natural allelic variants and homologues of the MOLX cDNAs of the invention can be isolated based on their homology to the human MOLX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding MOLX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, NY.

In a third embodiment, a nucleic acid that is hybridizable to the nucleic acid molecule comprising the nucleotide sequences of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of MOLX sequences that may exist in the population, the skilled artisan will further appreciate that changes can be introduced by mutation into the nucleotide sequences of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 thereby leading to changes in the amino acid sequences of the encoded MOLX proteins, without altering the functional ability of said MOLX proteins. For example, nucleotide substitutions leading to amino acid substitutions at “non-essential” amino acid residues can be made in the sequence of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177. A “non-essential” amino acid residue is a residue that can be altered from the wild-type sequences of the MOLX proteins without altering their biological activity, whereas an “essential” amino acid residue is required for such biological activity. For example, amino acid residues that are conserved among the MOLX proteins of the invention are predicted to be particularly non-amenable to alteration. Amino acids for which conservative substitutions can be made are well-known within the art.

Another aspect of the invention pertains to nucleic acid molecules encoding MOLX proteins that contain changes in amino acid residues that are not essential for activity. Such MOLX proteins differ in amino acid sequence from SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177. Preferably, the protein encoded by the nucleic acid molecule is at least about 60% homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177; more preferably at least about 70% homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177; still more preferably at least about 80% homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177; even morepreferably at least about 90% homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177; and most preferably at least about 95% homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177.

An isolated nucleic acid molecule encoding an MOLX protein homologous to the protein of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 such that one or more amino acid substitutions, additions or deletions are introduced into the encoded protein.

Mutations can be introduced into SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the MOLX protein is replaced with another amino acid residue from the same side chain family. Alternatively, in another embodiment, mutations can be introduced randomly along all or part of an MOLX coding sequence, such as by saturation mutagenesis, and the resultant mutants can be screened for MOLX biological activity to identify mutants that retain activity. Following mutagenesis of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant MOLX protein can be assayed for (i) the ability to form protein:protein interactions with other MOLX proteins, other cell-surface proteins, or biologically-active portions thereof, (ii) complex formation between a mutant MOLX protein and an MOLX ligand; or (iii) the ability of a mutant MOLX protein to bind to an intracellular target protein or biologically-active portion thereof, (e.g avidin proteins).

In yet another embodiment, a mutant MOLX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire MOLX coding strand, or to only a portion thereof. Nucleic acid molecules encoding fragments, homologs, derivatives and analogs of an MOLX protein of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, orantisense nucleic acids complementary to an MOLX nucleic acid sequence of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, are additionally provided.

In one embodiment, an antisense nucleic acid molecule is antisense to a “coding region” of the coding strand of a nucleotide sequence encoding an MOLX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the MOLX protein. The term “noncoding region” refers to 5′ and 3′ sequences which flank the coding region that are not translated into amino acids (i.e., also referred to as 5′ and 3′ untranslated regions).

Given the coding strand sequences encoding the MOLX protein disclosed herein, antisense nucleic acids of the invention can be designed according to the rules of Watson and Crick or Hoogsteen base pairing. The antisense nucleic acid molecule can be complementary to the entire coding region of MOLX mRNA, but more preferably is an oligonucleotide that is antisense to only a portion of the coding or noncoding region of MOLX mRNA. For example, the antisense oligonucleotide can be complementary to the region surrounding the translation start site of MOLX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an MOLX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave MOLX mRNA transcripts to thereby inhibit translation of MOLX mRNA. A ribozyme having specificity for an MOLX-encoding nucleic acid can be designed based upon the nucleotide sequence of an MOLX cDNA disclosed herein (i.e., SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176). For example, a derivative of a Tetrahymena L-19 IVS RNA can be constructed in which the nucleotide sequence of the active site is complementary to the nucleotide sequence to be cleaved in an MOLX-encoding mRNA. See, e.g., U.S. Pat. No. 4,987,071 to Cech, et al. and U.S. Pat. No. 5,116,742 to Cech, et al. MOLX mRNA can also be used to select a catalytic RNA having a specific ribonuclease activity from a pool of RNA molecules. See, e.g., Bartel et al., (1993) Science 261:1411-1418.

Alternatively, MOLX gene expression can be inhibited by targeting nucleotide sequences complementary to the regulatory region of the MOLX nucleic acid (e.g., the MOLX promoter and/or enhancers) to form triple helical structures that prevent transcription of the MOLX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the MOLX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of MOLX can be used in therapeutic and diagnostic applications. For example, PNAs can be used as antisense or antigene agents for sequence-specific modulation of gene expression by, e.g., inducing transcription or translation arrest or inhibiting replication. PNAs of MOLX can also be used, for example, in the analysis of single base pair mutations in a gene (e.g., PNA directed PCR clamping; as artificial restriction enzymes when used in combination with other enzymes, e.g, S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of MOLX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of MOLX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

MOLX Polypeptides

A polypeptide according to the invention includes a polypeptide including the amino acid sequence of MOLX polypeptides whose sequences are provided in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177 while still encoding a protein that maintains its MOLX activities and physiological functions, or a functional fragment thereof.

In general, an MOLX variant that preserves MOLX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated MOLX proteins, and biologically-active portions thereof, or derivatives, fragments, analogs or homologs thereof. Also provided are polypeptide fragments suitable for use as immunogens to raise anti-MOLX antibodies. In one embodiment, native MOLX proteins can be isolated from cells or tissue sources by an appropriate purification scheme using standard protein purification techniques. In another embodiment, MOLX proteins are produced by recombinant DNA techniques. Alternative to recombinant expression, an MOLX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the MOLX protein is derived, or substantially free from chemical precursors or other chemicals when chemically synthesized. The language “substantially free of cellular material” includes preparations of MOLX proteins in which the protein is separated from cellular components of the cells from which it is isolated or recombinantly-produced. In one embodiment, the language “substantially free of cellular material” includes preparations of MOLX proteins having less than about 30% (by dry weight) of non-MOLX proteins (also referred to herein as a “contaminating protein”), more preferably less than about 20% of non-MOLX proteins, still more preferably less than about 10% of non-MOLX proteins, and most preferably less than about 5% of non-MOLX proteins. When the MOLX protein or biologically-active portion thereof is recombinantly-produced, it is also preferably substantially free of culture medium, i.e., culture medium represents less than about 20%, more preferably less than about 10%, and most preferably less than about 5% of the volume of the MOLX protein preparation.

The language “substantially free of chemical precursors or other chemicals” includes preparations of MOLX proteins in which the protein is separated from chemical precursors or other chemicals that are involved in the synthesis of the protein. In one embodiment, the language “substantially free of chemical precursors or other chemicals” includes preparations of MOLX proteins having less than about 30% (by dry weight) of chemical precursors or non-MOLX chemicals, more preferably less than about 20% chemical precursors or non-MOLX chemicals, still more preferably less than about 10% chemical precursors or non-MOLX chemicals, and most preferably less than about 5% chemical precursors or non-MOLX chemicals.

Biologically-active portions of MOLX proteins include peptides comprising amino acid sequences sufficiently homologous to or derived from the amino acid sequences of the MOLX proteins (e.g., the amino acid sequence shown in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177) that include fewer amino acids than the full-length MOLX proteins, and exhibit at least one activity of an MOLX protein. Typically, biologically-active portions comprise a domain or motif with at least one activity of the MOLX protein. A biologically-active portion of an MOLX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native MOLX protein.

In an embodiment, the MOLX protein has an amino acid sequence shown in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177. In other embodiments, the MOLX protein is substantially homologous to SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, and retains the functional activity of the protein of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, yet differs in amino acid sequence due to natural allelic variation or mutagenesis, as described in detail, below. Accordingly, in another embodiment, the MOLX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, and retains the functional activity of the MOLX proteins of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides MOLX chimeric or fusion proteins. As used herein, an MOLX “chimeric protein” or “fusion protein” comprises an MOLX polypeptide operatively-linked to a non-MOLX polypeptide. An “MOLX polypeptide” refers to a polypeptide having an amino acid sequence corresponding to an MOLX protein (SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155,157, 159, 161, 163, 165, 167, 169, 171, 173, 175, and 177), whereas a “non-MOLX polypeptide” refers to a polypeptide having an amino acid sequence corresponding to a protein that is not substantially homologous to the MOLX protein, e.g., a protein that is different from the MOLX protein and that is derived from the same or a different organism. Within an MOLX fusion protein the MOLX polypeptide can correspond to all or a portion of an MOLX protein. In one embodiment, an MOLX fusion protein comprises at least one biologically-active portion of an MOLX protein. In another embodiment, an MOLX fusion protein comprises at least two biologically-active portions of an MOLX protein. In yet another embodiment, an MOLX fusion protein comprises at least three biologically-active portions of an MOLX protein. Within the fusion protein, the term “operatively-linked” is intended to indicate that the MOLX polypeptide and the non-MOLX polypeptide are fused in-frame with one another. The non-MOLX polypeptide can be fused to the N-terminus or C-terminus of the MOLX polypeptide.

In one embodiment, the fusion protein is a GST-MOLX fusion protein in which the MOLX sequences are fused to the C-terminus of the GST (glutathione S-transferase) sequences. Such fusion proteins can facilitate the purification of recombinant MOLX polypeptides.

In another embodiment, the fusion protein is an MOLX protein containing a heterologous signal sequence at its N-terminus. In certain host cells (e.g., mammalian host cells), expression and/or secretion of MOLX can be increased through use of a heterologous signal sequence.

In yet another embodiment, the fusion protein is an MOLX-immunoglobulin fusion protein in which the MOLX sequences are fused to sequences derived from a member of the immunoglobulin protein family. The MOLX-immunoglobulin fusion proteins of the invention can be incorporated into pharmaceutical compositions and administered to a subject to inhibit an interaction between an MOLX ligand and an MOLX protein on the surface of a cell, to thereby suppress MOLX-mediated signal transduction in vivo. The MOLX-immunoglobulin fusion proteins can be used to affect the bioavailability of an MOLX cognate ligand. Inhibition of the MOLX ligand/MOLX interaction may be useful therapeutically for both the treatment of proliferative and differentiative disorders, as well as modulating (e.g. promoting or inhibiting) cell survival. Moreover, the MOLX-immunoglobulin fusion proteins of the invention can be used as immunogens to produce anti-MOLX antibodies in a subject, to purify MOLX ligands, and in screening assays to identify molecules that inhibit the interaction of MOLX with an MOLX ligand.

An MOLX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). An MOLX-encoding nucleic acid can be cloned into such an expression vector such that the fusion moiety is linked in-frame to the MOLX protein.

MOLX Agonists and Antagonists

The invention also pertains to variants of the MOLX proteins that function as either MOLX agonists (i.e., mimetics) or as MOLX antagonists. Variants of the MOLX protein can be generated by mutagenesis (e.g., discrete point mutation or truncation of the MOLX protein). An agonist of the MOLX protein can retain substantially the same, or a subset of, the biological activities of the naturally occurring form of the MOLX protein. An antagonist of the MOLX protein can inhibit one or more of the activities of the naturally occurring form of the MOLX protein by, for example, competitively binding to a downstream or upstream member of a cellular signaling cascade which includes the MOLX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the MOLX proteins.

Variants of the MOLX proteins that function as either MOLX agonists (i.e., mimetics) or as MOLX antagonists can be identified by screening combinatorial libraries of mutants (e.g., truncation mutants) of the MOLX proteins for MOLX protein agonist or antagonist activity. In one embodiment, a variegated library of MOLX variants is generated by combinatorial mutagenesis at the nucleic acid level and is encoded by a variegated gene library. A variegated library of MOLX variants can be produced by, for example, enzymatically ligating a mixture of synthetic oligonucleotides into gene sequences such that a degenerate set of potential MOLX sequences is expressible as individual polypeptides, or alternatively, as a set of larger fusion proteins (e.g., for phage display) containing the set of MOLX sequences therein. There are a variety of methods which can be used to produce libraries of potential MOLX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MOLX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the MOLX protein coding sequences can be used to generate a variegated population of MOLX fragments for screening and subsequent selection of variants of an MOLX protein. In one embodiment, a library of coding sequence fragments can be generated by treating a double stranded PCR fragment of an MOLX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the MOLX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MOLX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify MOLX variants. See, e.g., Arkin and Yourvan, 1992. Proc. Natl. Acad. Sci. USA 89: 7811-7815; Delgrave, et al., 1993. Protein Engineering 6:327-331.

Anti-MOLX Antibodies

The invention encompasses antibodies and antibody fragments, such as F_{ab} or (F_{ab})₂, that bind immunospecifically to any of the MOLX polypeptides of said invention.

An isolated MOLX protein, or a portion or fragment thereof, can be used as an immunogen to generate antibodies that bind to MOLX polypeptides using standard techniques for polyclonal and monoclonal antibody preparation. The full-length MOLX proteins can be used or, alternatively, the invention provides antigenic peptide fragments of MOLX proteins for use as immunogens. The antigenic MOLX peptides comprises at least 4 amino acid residues of the amino acid sequence shown in SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177 and encompasses an epitope of MOLX such that an antibody raised against the peptide forms a specific immune complex with MOLX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of MOLX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nal. Acad. Sci. USA 78: 3824-3828; Kyte and Doolittle, 1982. J. Mol. Biol. 157: 105-142, each incorporated herein by reference in their entirety).

As disclosed herein, MOLX protein sequences of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 1 45, 147, 149, 151, 153, 155, 157, 159, 161, 163, 165, 167, 169, 171, 173, 175, and 177, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as MOLX. Such antibodies include, but are not limited to, polyclonal, monoclonal, chimeric, single chain, F_{ab} and F_{(ab′)2} fragments, and an F_{ab} expression library. In a specific embodiment, antibodies to human MOLX proteins are disclosed. Various procedures known within the art may be used for the production of polyclonal or monoclonal antibodies to an MOLX protein sequence of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed MOLX protein or a chemically-synthesized MOLX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmetle-Guerin and Corynebacterium parvum, or similar immunostimulatory agents. If desired, the antibody molecules directed against MOLX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of MOLX. A monoclonal antibody composition thus typically displays a single binding affinity for a particular MOLX protein with which it immunoreacts. For preparation of monoclonal antibodies directed towards a particular MOLX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: Monoclonal Antibodies and Cancer Therapy, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: Monoclonal Antibodies and Cancer Therapy, Alan R. Liss, Inc., pp. 77-96). Each of the above citations is incorporated herein by reference in their entirety.

According to the invention, techniques can be adapted for the production of single-chain antibodies specific to an MOLX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an MOLX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an MOLX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-MOLX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an MOLX protein is facilitated by generation of hybridomas that bind to the fragment of an MOLX protein possessing such a domain. Thus, antibodies that are specific for a desired domain within an MOLX protein, or derivatives, fragments, analogs or homologs thereof, are also provided herein.

Anti-MOLX antibodies may be used in methods known within the art relating to the localization and/or quantitation of an MOLX protein (e.g., for use in measuring levels of the MOLX protein within appropriate physiological samples, for use in diagnostic methods, for use in imaging the protein, and the like). In a given embodiment, antibodies for MOLX proteins, or derivatives, fragments, analogs or homologs thereof, that contain the antibody derived binding domain, are utilized as pharmacologically-active compounds (hereinafter “Therapeutics”).

An anti-MOLX antibody (e.g., monoclonal antibody) can be used to isolate an MOLX polypeptide by standard techniques, such as affinity chromatography or immunoprecipitation. An anti-MOLX antibody can facilitate the purification of natural MOLX polypeptide from cells and of recombinantly-produced MOLX polypeptide expressed in host cells. Moreover, an anti-MOLX antibody can be used to detect MOLX protein (e.g., in a cellular lysate or cell supernatant) in order to evaluate the abundance and pattern of expression of the MOLX protein. Anti-MOLX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

MOLX Recombinant Expression Vectors and Host Cells

Another aspect of the invention pertains to vectors, preferably expression vectors, containing a nucleic acid encoding an MOLX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., MOLX proteins, mutant forms of MOLX proteins, fusion proteins, etc.).

The recombinant expression vectors of the invention can be designed for expression of MOLX proteins in prokaryotic or eukaryotic cells. For example, MOLX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the MOLX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, MOLX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to MOLX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, MOLX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding MOLX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) MOLX protein. Accordingly, the invention further provides methods for producing MOLX protein using the host cells of the invention. In one embodiment, the method comprises culturing the host cell of invention (into which a recombinant expression vector encoding MOLX protein has been introduced) in a suitable medium such that MOLX protein is produced. In another embodiment, the method further comprises isolating MOLX protein from the medium or the host cell.

Transgenic MOLX Animals

The host cells of the invention can also be used to produce nonhuman transgenic animals. For example, in one embodiment, a host cell of the invention is a fertilized oocyte or an embryonic stem cell into which MOLX protein-coding sequences have been introduced. Such host cells can then be used to create non-human transgenic animals in which exogenous MOLX sequences have been introduced into their genome or homologous recombinant animals in which endogenous MOLX sequences have been altered. Such animals are useful for studying the function and/or activity of MOLX protein and for identifying and/or evaluating modulators of MOLX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that. remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous MOLX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing MOLX-encoding nucleic acid into the male pronuclei of a fertilized oocyte (e.g., by microinjection, retroviral infection) and allowing the oocyte to develop in a pseudopregnant female foster animal. The human MOLX cDNA sequences of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 can be introduced as a transgene into the genome of a non-human animal. Alternatively, a non-human homologue of the human MOLX gene, such as a mouse MOLX gene, can be isolated based on hybridization to the human MOLX cDNA (described further supra) and used as a transgene. Intronic sequences and polyadenylation signals can also be included in the transgene to increase the efficiency of expression of the transgene. A tissue-specific regulatory sequence(s) can be operably-linked to the MOLX transgene to direct expression of MOLX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating The Mouse Embryo, Cold Spring Harbor Laboratory Press, Cold Spring Harbor, N.Y. Similar methods are used for production of other transgenic animals. A transgenic founder animal can be identified based upon the presence of the MOLX transgene in its genome and/or expression of MOLX mRNA in tissues or cells of the animals. A transgenic founder animal can then be used to breed additional animals carrying the transgene. Moreover, transgenic animals carrying a transgene-encoding MOLX protein can further be bred to other transgenic animals carrying other transgenes.

To create a homologous recombinant animal, a vector is prepared which contains at least a portion of an MOLX gene into which a deletion, addition or substitution has been introduced to thereby alter, e.g., functionally disrupt, the MOLX gene. The MOLX gene can be a human gene (e.g., the cDNA of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176), but more preferably, is a non-human homologue of a human MOLX gene. For example, a mouse homologue of human MOLX gene of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 can be used to construct a homologous recombination vector suitable for altering an endogenous MOLX gene in the mouse genome. In one embodiment, the vector is designed such that, upon homologous recombination, the endogenous MOLX gene is functionally disrupted (i.e., no longer encodes a functional protein; also referred to as a “knock out” vector).

Alternatively, the vector can be designed such that, upon homologous recombination, the endogenous MOLX gene is mutated or otherwise altered but still encodes functional protein (e.g., the upstream regulatory region can be altered to thereby alter the expression of the endogenous MOLX protein). In the homologous recombination vector, the altered portion of the MOLX gene is flanked at its 5′- and 3′-termini by additional nucleic acid of the MOLX gene to allow for homologous recombination to occur between the exogenous MOLX gene carried by the vector and an endogenous MOLX gene in an embryonic stem cell. The additional flanking MOLX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced MOLX gene has homologously-recombined with the endogenous MOLX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The MOLX nucleic acid molecules, MOLX proteins, and anti-MOLX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an MOLX protein or anti-MOLX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express MOLX protein (e.g., via a recombinant expression vector in a host cell in gene therapy applications), to detect MOLX mRNA (e.g., in a biological sample) or a genetic lesion in an MOLX gene, and to modulate MOLX activity, as described further, below. In addition, the MOLX proteins can be used to screen drugs or compounds that modulate the MOLX protein activity or expression as well as to treat disorders characterized by insufficient or excessive production of MOLX protein or production of MOLX protein forms that have decreased or aberrant activity compared to MOLX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-MOLX antibodies of the invention can be used to detect and isolate MOLX proteins and modulate MOLX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to MOLX proteins or have a stimulatory or inhibitory effect on, e.g., MOLX protein expression or MOLX protein activity. The invention also includes compounds identified in the screening assays described herein.

In one embodiment, the invention provides assays for screening candidate or test compounds which bind to or modulate the activity of the membrane-bound form of an MOLX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of MOLX protein, or a biologically-active portion thereof, on the cell surface is contacted with a test compound and the ability of the test compound to bind to an MOLX protein determined. The cell, for example, can of mammalian origin or a yeast cell. Determining the ability of the test compound to bind to the MOLX protein can be accomplished, for example, by coupling the test compound with a radioisotope or enzymatic label such that binding of the test compound to the MOLX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of MOLX protein, or a biologically-active portion thereof, on the cell surface with a known compound which binds MOLX to form an assay mixture, contacting the assay mixture with a test compound, and determining the ability of the test compound to interact with an MOLX protein, wherein determining the ability of the test compound to interact with an MOLX protein comprises determining the ability of the test compound to preferentially bind to MOLX protein or a biologically-active portion thereof as compared to the known compound.

In another embodiment, an assay is a cell-based assay comprising contacting a cell expressing a membrane-bound form of MOLX protein, or a biologically-active portion thereof, on the cell surface with a test compound and determining the ability of the test compound to modulate (e.g., stimulate or inhibit) the activity of the MOLX protein or biologically-active portion thereof. Determining the ability of the test compound to modulate the activity of MOLX or a biologically-active portion thereof can be accomplished, for example, by determining the ability of the MOLX protein to bind to or interact with an MOLX target molecule. As used herein, a “target molecule” is a molecule with which an MOLX protein binds or interacts in nature, for example, a molecule on the surface of a cell which expresses an MOLX interacting protein, a molecule on the surface of a second cell, a molecule in the extracellular milieu, a molecule associated with the internal surface of a cell membrane or a cytoplasmic molecule. An MOLX target molecule can be a non-MOLX molecule or an MOLX protein or polypeptide of the invention. In one embodiment, an MOLX target molecule is a component of a signal transduction pathway that facilitates transduction of an extracellular signal (e.g. a signal generated by binding of a compound to a membrane-bound MOLX molecule) through the cell membrane and into the cell. The target, for example, can be a second intercellular protein that has catalytic activity or a protein that facilitates the association of downstream signaling molecules with MOLX.

Determining the ability of the MOLX protein to bind to or interact with an MOLX target molecule can be accomplished by one of the methods described above for determining direct binding. In one embodiment, determining the ability of the MOLX protein to bind to or interact with an MOLX target molecule can be accomplished by determining the activity of the target molecule. For example, thee activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an MOLX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an MOLX protein or biologically-active portion thereof with a test compound and determining the ability of the test compound to bind to the MOLX protein or biologically-active portion thereof. Binding of the test compound to the MOLX protein can be determined either directly or indirectly as described above. In one such embodiment, the assay comprises contacting the MOLX protein or biologically-active portion thereof with a known compound which binds MOLX to form an assay mixture, contacting the assay mixture with a test compound, and determining the ability of the test compound to interact with an MOLX protein, wherein determining the ability of the test compound to interact with an MOLX protein comprises determining the ability of the test compound to preferentially bind to MOLX or biologically-active portion thereof as compared to the known compound.

In still another embodiment, an assay is a cell-free assay comprising contacting MOLX protein or biologically-active portion thereof with a test compound and determining the ability of the test compound to modulate (e.g. stimulate or inhibit) the activity of the MOLX protein or biologically-active portion thereof. Determining the ability of the test compound to modulate the activity of MOLX can be accomplished, for example, by determining the ability of the MOLX protein to bind to an MOLX target molecule by one of the methods described above for determining direct binding. In an alternative embodiment, determining the ability of the test compound to modulate the activity of MOLX protein can be accomplished by determining the ability of the MOLX protein further modulate an MOLX target molecule. For example, the catalytic/enzymatic activity of the target molecule on an appropriate substrate can be determined as described, supra.

In yet another embodiment, the cell-free assay comprises contacting the MOLX protein or biologically-active portion thereof with a known compound which binds MOLX protein to form an assay mixture, contacting the assay mixture with a test compound, and determining the ability of the test compound to interact with an MOLX protein, wherein determining the ability of the test compound to interact with an MOLX protein comprises determining the ability of the MOLX protein to preferentially bind to or modulate the activity of an MOLX target molecule.

The cell-free assays of the invention are amenable to use of both the soluble form or the membrane-bound form of MOLX protein. In the case of cell-free assays comprising the membrane-bound form of MOLX protein, it may be desirable to utilize a solubilizing agent such that the membrane-bound form of MOLX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either MOLX protein or its target molecule to facilitate separation of complexed from uncomplexed forms of one or both of the proteins, as well as to accommodate automation of the assay. Binding of a test compound to MOLX protein, or interaction of MOLX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-MOLX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or MOLX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of MOLX protein binding or activity determined using standard techniques.

Other techniques for immobilizing proteins on matrices can also be used in the screening assays of the invention. For example, either the MOLX protein or its target molecule can be immobilized utilizing conjugation of biotin and streptavidin. Biotinylated MOLX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with MOLX protein or target molecules, but which do not interfere with binding of the MOLX protein to its target molecule, can be derivatized to the wells of the plate, and unbound target or MOLX protein trapped in the wells by antibody conjugation. Methods for detecting such complexes, in addition to those described above for the GST-immobilized complexes, include immunodetection of complexes using antibodies reactive with the MOLX protein or target molecule, as well as enzyme-linked assays that rely on detecting an enzymatic activity associated with the MOLX protein or target molecule.

In another embodiment, modulators of MOLX protein expression are identified in a method wherein a cell is contacted with a candidate compound and the expression of MOLX mRNA-or protein in the cell is determined. The level of expression of MOLX mRNA or protein in the presence of the candidate compound is compared to the level of expression of MOLX mRNA or protein in the absence of the candidate compound. The candidate compound can then be identified as a modulator of MOLX mRNA or protein expression based upon this comparison. For example, when expression of MOLX mRNA or protein is greater (i.e., statistically significantly greater) in the presence of the candidate compound than in its absence, the candidate compound is identified as a stimulator of MOLX mRNA or protein expression. Alternatively, when expression of MOLX mRNA or protein is less (statistically significantly less) in the presence of the candidate compound than in its absence, the candidate compound is identified as an inhibitor of MOLX mRNA or protein expression. The level of MOLX mRNA or protein expression in the cells can be determined by methods described herein for detecting MOLX mRNA or protein.

In yet another aspect of the invention, the MOLX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with MOLX (“MOLX-binding proteins” or “MOLX-bp”) and modulate MOLX activity. Such MOLX-binding proteins are also likely to be involved in the propagation of signals by the MOLX proteins as, for example, upstream or downstream elements of the MOLX pathway.

The two-hybrid system is based on the modular nature of most transcription factors, which consist of separable DNA-binding and activation domains. Briefly, the assay utilizes two different DNA constructs. In one construct, the gene that codes for MOLX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an MOLX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with MOLX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the MOLX sequences, SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or fragments or derivatives thereof, can be used to map the location of the MOLX genes, respectively, on a chromosome. The mapping of the MOLX sequences to chromosomes is an important first step in correlating these sequences with genes associated with disease.

Briefly, MOLX genes can be mapped to chromosomes by preparing PCR primers (preferably 15-25 bp in length) from the MOLX sequences. Computer analysis of the MOLX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the MOLX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the MOLX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosones: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data.

Such data are found, eg, in McKusick, Mandelian Inheritance in Man, available 20 on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the MOLX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The MOLX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the MOLX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The MOLX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining MOLX protein and/or nucleic acid expression as well as MOLX activity, in the context of a biological sample (e.g., blood, serum, cells, tissue) to thereby determine whether an individual is afflicted with a disease or disorder, or is at risk of developing a disorder, associated with aberrant MOLX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with MOLX protein, nucleic acid expression or activity. For example, mutations in an MOLX gene can be assayed in a biological sample. Such assays can be used for prognostic or predictive purpose to thereby prophylactically treat an individual prior to the onset of a disorder characterized by or associated with MOLX protein, nucleic acid expression, or biological activity.

Another aspect of the invention provides methods for determining MOLX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of MOLX in clinical trials.

These and other agents are described in further detail in the following sections.

Diagnostic Assays

An exemplary method for detecting the presence or absence of MOLX in a biological sample involves obtaining a biological sample from a test subject and contacting the biological sample with a compound or an agent capable of detecting MOLX protein or nucleic acid (e.g., mRNA, genomic DNA) that encodes MOLX protein such that the presence of MOLX is detected in the biological sample. An agent for detecting MOLX mRNA or genomic DNA is a labeled nucleic acid probe capable of hybridizing to MOLX mRNA or genomic DNA. The nucleic acid probe can be, for example, a full-length MOLX nucleic acid, such as the nucleic acid of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or a portion thereof, such as an oligonucleotide of at least 15, 30, 50, 100, 250 or 500 nucleotides in length and sufficient to specifically hybridize under stringent conditions to MOLX mRNA or genomic DNA. Other suitable probes for use in the diagnostic assays of the invention are described herein.

An agent for detecting MOLX protein is an antibody capable of binding to MOLX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect MOLX mRNA, protein, or genomic DNA in a biological sample in vitro as well as in vivo. For example, in vitro techniques for detection of MOLX mRNA include Northern hybridizations and in situ hybridizations. In vitro techniques for detection of MOLX protein include enzyme linked immunosorbent assays (ELISAs), Western blots, immunoprecipitations, and immunofluorescence. In vitro techniques for detection of MOLX genomic DNA include Southern hybridizations. Furthermore, in vivo techniques for detection of MOLX protein include introducing into a subject a labeled anti-MOLX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting MOLX protein, mRNA, or genomic DNA, such that the presence of MOLX protein, mRNA or genomic DNA is detected in the biological sample, and comparing the presence of MOLX protein, mRNA or genomic DNA in the control sample with the presence of MOLX protein, mRNA or genomic DNA in the test sample.

The invention also encompasses kits for detecting the presence of MOLX in a biological sample. For example, the kit can comprise: a labeled compound or agent capable of detecting MOLX protein or mRNA in a biological sample; means for determining the amount of MOLX in the sample; and means for comparing the amount of MOLX in the sample with a standard. The compound or agent can be packaged in a suitable container. The kit can further comprise instructions for using the kit to detect MOLX protein or nucleic acid.

Prognostic Assays

The diagnostic methods described herein can furthermore be utilized to identify subjects having or at risk of developing a disease or disorder associated with aberrant MOLX expression or activity. For example, the assays described herein, such as the preceding diagnostic assays or the following assays, can be utilized to identify a subject having or at risk of developing a disorder associated with MOLX protein, nucleic acid expression or activity. Alternatively, the prognostic assays can be utilized to identify a subject having or at risk for developing a disease or disorder. Thus, the invention provides a method for identifying a disease or disorder associated with aberrant MOLX expression or activity in which a test sample is obtained from a subject and MOLX protein or nucleic acid (e.g., mRNA, genomic DNA) is detected, wherein the presence of MOLX protein or nucleic acid is diagnostic for a subject having or at risk of developing a disease or disorder associated with aberrant MOLX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant MOLX expression or activity. For example, such methods can be used to determine whether a subject can be effectively treated with an agent for a disorder. Thus, the invention provides methods for determining whether a subject can be effectively treated with an agent for a disorder associated with aberrant MOLX expression or activity in which a test sample is obtained and MOLX protein or nucleic acid is detected (e.g., wherein the presence of MOLX protein or nucleic acid is diagnostic for a subject that can be administered the agent to treat a disorder associated with aberrant MOLX expression or activity).

The methods of the invention can also be used to detect genetic lesions in an MOLX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an MOLX-protein, or the misexpression of the MOLX gene. For example, such genetic lesions can be detected by ascertaining the existence of at least one of: (i) a deletion of one or more nucleotides from an MOLX gene; (ii) an addition of one or more nucleotides to an MOLX gene; (iii) a substitution of one or more nucleotides of an MOLX gene, (iv) a chromosomal rearrangement of an MOLX gene; (v) an alteration in the level of a messenger RNA transcript of an MOLX gene, (vi) aberrant modification of an MOLX gene, such as of the methylation pattern of the genomic DNA, (vii) the presence of a non-wild-type splicing pattern of a messenger RNA transcript of an MOLX gene, (viii) a non-wild-type level of an MOLX protein, (ix) allelic loss of an MOLX gene, and (x) inappropriate post-translational modification of an MOLX protein. As described herein, there are a large number of assay techniques known in the art which can be used for detecting lesions in an MOLX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the MOLX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an MOLX gene under conditions such that hybridization and amplification of the MOLX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an MOLX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in MOLX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in MOLX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MOLX gene and detect mutations by comparing the sequence of the sample MOLX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the MOLX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type MOLX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MOLX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an MOLX sequence, e.g., a wild-type MOLX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in MOLX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control MOLX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an MOLX gene.

Furthermore, any cell type or tissue, preferably peripheral blood leukocytes, in which MOLX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on MOLX activity (e.g., MOLX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of MOLX protein, expression of MOLX nucleic acid, or mutation content of MOLX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g, Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of MOLX protein, expression of MOLX nucleic acid, or mutation content of MOLX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an MOLX modulator, such as a modulator identified by one of the exemplary screening assays described herein.

Monitoring of Effects During Clinical Trials

Monitoring the influence of agents (e.g., drugs, compounds) on the expression or activity of MOLX (e.g., the ability to modulate aberrant cell proliferation and/or differentiation) can be applied not only in basic drug screening, but also in clinical trials. For example, the effectiveness of an agent determined by a screening assay as described herein to increase MOLX gene expression, protein levels, or upregulate MOLX activity, can be monitored in clinical trails of subjects exhibiting decreased MOLX gene expression, protein levels, or downregulated MOLX activity. Alternatively, the effectiveness of an agent determined by a screening assay to decrease MOLX gene expression, protein levels, or downregulate MOLX activity, can be monitored in clinical trails of subjects exhibiting increased MOLX gene expression, protein levels, or upregulated MOLX activity. In such clinical trials, the expression or activity of MOLX and, preferably, other genes that have been implicated in, for example, a cellular proliferation or immune disorder can be used as a “read out” or markers of the immune responsiveness of a particular cell.

By way of example, and not of limitation, genes, including MOLX, that are modulated in cells by treatment with an agent (eg., compound, drug or small molecule) that modulates MOLX activity (e.g., identified in a screening assay as described herein) can be identified. Thus, to study the effect of agents on cellular proliferation disorders, for example, in a clinical trial, cells can be isolated and RNA prepared and analyzed for the levels of expression of MOLX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of MOLX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an MOLX protein, mRNA, or genomic DNA in the preadministration sample; (iii) obtaining one or more post-administration samples from the subject; (iv) detecting the level of expression or activity of the MOLX protein, mRNA, or genomic DNA in the post-administration samples; (v) comparing the level of expression or activity of the MOLX protein, mRNA, or genomic DNA in the pre-administration sample with the MOLX protein, mRNA, or genomic DNA in the post administration sample or samples; and (vi) altering the administration of the agent to the subject accordingly. For example, increased administration of the agent may be desirable to increase the expression or activity of MOLX to higher levels than detected, i.e., to increase the effectiveness of the agent. Alternatively, decreased administration of the agent may be desirable to decrease expression or activity of MOLX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant MOLX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant MOLX expression or activity, by administering to the subject an agent that modulates MOLX expression or at least one MOLX activity. Subjects at risk for a disease that is caused or contributed to by aberrant MOLX expression or activity can be identified by, for example, any or a combination of diagnostic or prognostic assays as described herein. Administration of a prophylactic agent can occur prior to the manifestation of symptoms characteristic of the MOLX aberrancy, such that a disease or disorder is prevented or, alternatively, delayed in its progression. Depending upon the type of MOLX aberrancy, for example, an MOLX agonist or MOLX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating MOLX expression or activity for therapeutic purposes. The modulatory method of the invention involves contacting a cell with an agent that modulates one or more of the activities of MOLX protein activity associated with the cell. An agent that modulates MOLX protein activity can be an agent as described herein, such as a nucleic acid or a protein, a naturally-occurring cognate ligand of an MOLX protein, a peptide, an MOLX peptidomimetic, or other small molecule. In one embodiment, the agent stimulates one or more MOLX protein activity. Examples of such stimulatory agents include active MOLX protein and a nucleic acid molecule encoding MOLX that has been introduced into the cell. In another embodiment, the agent inhibits one or more MOLX protein activity. Examples of such inhibitory agents include antisense MOLX nucleic acid molecules and anti-MOLX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an MOLX protein or nucleic acid molecule. In one embodiment, the method involves administering an agent (e.g., an agent identified by a screening assay described herein), or combination of agents that modulates (e.g., up-regulates or down-regulates) MOLX expression or activity. In another embodiment, the method involves administering an MOLX protein or nucleic acid molecule as therapy to compensate for reduced or aberrant MOLX expression or activity.

Stimulation of MOLX activity is desirable in situations in which MOLX is abnormally downregulated and/or in which increased MOLX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The MOLX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the MOLX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the MOLX protein, and the MOLX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

### Example 1

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at 80 degrees C.

MOL1

Expression of gene GM_{—}79960178 was assessed using the primer-probe set Ag1605, described in Table 12. Results of the RTQ-PCR runs are shown in Tables 13 and 14.

TABLE 12

Probe Name: Ag1605

| | | | | | |
|---|---|---|---|---|---|
| | | | | Start | SEQ ID |
| Primers | Sequences | Tm | Length | Position | NO: |

| | | | | | |
|---|---|---|---|---|---|
| Forward | 5′-CCTGAGCTACAACAACATCATG-3′ | 58.3 | 22 | 333 | 76 |
| Probe | FAM-5′-CCTCATATCCCTGTCCCTCAGCCATA-3′-TAMRA | 69 | 26 | 378 | 77 |
| Reverse | 5′-GCAGAGTCTAGCATCAGGATGT-3′ | 58.6 | 22 | 407 | 78 |

TABLE 13
Panels 1.3D and 2D

| PANEL 1.3D | | | PANEL 2D | | |
|---|---|---|---|---|---|
| | | Rel. Expr., % | | Rel. | Rel. Expr., % |
| | Rel. Expr., % | 1.3dx4tm5402f_{—} | | Expr., % | 2dx4tm4732f_{—} |
| Tissue | 1.3dtm2653f_{—} | ag1605_{—} | | 2Dtm2728f_{—} | ag1605_{—} |
| Name | ag1605 | b1 | Tissue Name | ag1605 | a2 |
| Liver | 0 | 1.9 | Normal Colon GENPAK 061003 | 70.2 | 70.3 |
| adenocarcinoma | | | | | |
| Pancreas | 0.2 | 0 | 83219 CC Well to Mod Diff | 8.5 | 6.3 |
| | | | (ODO3866) | | |
| Pancreatic | 0 | 17.6 | 83220 CC NAT (ODO3866) | 8.1 | 9.4 |
| ca. | | | | | |
| CAPAN 2 | | | | | |
| Adrenal | 2.2 | 1.3 | 83221 CC Gr.2 rectosigmoid | 4.5 | 12.1 |
| gland | | | (ODO3868) | | |
| Thyroid | 2 | 5.6 | 83222 CC NAT (ODO3868) | 1.6 | 6.3 |
| Salivary | 4 | 8.9 | 83235 CC Mod Diff (ODO3920) | 17.9 | 19.7 |
| gland | | | | | |
| Pituitary | 1.2 | 1.7 | 83236 CC NAT (ODO3920) | 21.5 | 11.6 |
| gland | | | | | |
| Brain (fetal) | 4.4 | 21.2 | 83237 CC Gr.2 ascend colon | 18 | 34.4 |
| | | | (ODO3921) | | |
| Brain | 3.1 | 30.5 | 83238 CC NAT (ODO3921) | 8.9 | 9.8 |
| (whole) | | | | | |
| Brain | 1.8 | 6.6 | 83241 CC from Partial | 7.1 | 4.9 |
| (amygdala) | | | Hepatectomy (ODO4309) | | |
| Brain | 1.9 | 14.6 | 83242 Liver NAT (ODO4309) | 17.1 | 6.9 |
| (cerebellum) | | | | | |
| Brain | 5.8 | 8.7 | 87472 Colon mets to lung | 6.7 | 11.4 |
| (hippocampus) | | | (OD04451-01) | | |
| Brain | 0.8 | 9.3 | 87473 Lung NAT (OD04451-02) | 0.8 | 2.9 |
| (substantia | | | | | |
| nigra) | | | | | |
| Brain | 1.4 | 4.8 | Normal Prostate Clontech A+ | 17.8 | 52.9 |
| (thalamus) | | | 6546-1 | | |
| Cerebral | 3.8 | 2.6 | 84140 Prostate Cancer | 18.6 | 23 |
| Cortex | | | (OD04410) | | |
| Spinal cord | 2.6 | 15.7 | 84141 Prostate NAT (OD04410) | 9.1 | 22 |
| CNS ca. | 0 | 0 | 87073 Prostate Cancer (OD04720- | 20 | 14.5 |
| (glio/astro) | | | 01) | | |
| U87-MG | | | | | |
| CNS ca. | 0.2 | 1.5 | 87074 Prostate NAT (OD04720- | 21.3 | 38.1 |
| (glio/astro) | | | 02) | | |
| U-118-MG | | | | | |
| CNS ca. | 0.8 | 0 | Normal Lung GENPAK 061010 | 69.7 | 85.9 |
| (astro) | | | | | |
| SW1783 | | | | | |
| CNS ca.* | 2.8 | 1.6 | 83239 Lung Met to Muscle | 11.1 | 6.3 |
| (neuro; met) | | | (ODO4286) | | |
| SK-N-AS | | | | | |
| CNS ca. | 0.6 | 0 | 83240 Muscle NAT (ODO4286) | 15.1 | 23.3 |
| (astro) | | | | | |
| SF-539 | | | | | |
| CNS ca. | 0.2 | 0 | 84136 Lung Malignant Cancer | 30.6 | 30.8 |
| (astro) | | | (OD03126) | | |
| SNB-75 | | | | | |
| CNS ca. | 0.3 | 9.7 | 84137 Lung NAT (OD03126) | 8 | 16.3 |
| (glio) | | | | | |
| SNB-19 | | | | | |
| CNS ca. | 0.9 | 4.8 | 84871 Lung Cancer (OD04404) | 41.8 | 45.2 |
| (glio) | | | | | |
| U251 | | | | | |
| CNS ca. | 100 | 7.7 | 84872 Lung NAT (OD04404) | 15.4 | 27.5 |
| (glio) | | | | | |
| SF-295 | | | | | |
| Heart (fetal) | 3.7 | 0.9 | 84875 Lung Cancer (OD04565) | 7.4 | 8.5 |
| Heart | 0.4 | 6.1 | 84876 Lung NAT (OD04565) | 5.3 | 1.2 |
| Fetal | 45.1 | 3.5 | 85950 Lung Cancer (OD04237- | 77.9 | 72.9 |
| Skeletal | | | 01) | | |
| Skeletal | 5.8 | 100 | 85970 Lung NAT (OD04237-02) | 14.3 | 12.9 |
| muscle | | | | | |
| Bone | 2.9 | 22.8 | 83255 Ocular Mel Met to Liver | 2.7 | 6 |
| marrow | | | (ODO4310) | | |
| Thymus | 6.3 | 12.2 | 83256 Liver NAT (ODO4310) | 2.3 | 5.1 |
| Spleen | 7.9 | 14.6 | 84139 Melanoma Mets to Lung | 1.3 | 6.2 |
| | | | (OD04321) | | |
| Lymph node | 16.7 | 57.9 | 84138 Lung NAT (OD04321) | 36.9 | 22.4 |
| Colorectal | 4.3 | 3.4 | Normal Kidney GENPAK 061008 | 9.2 | 19.2 |
| Stomach | 7.5 | 20.7 | 83786 Kidney Ca, Nuclear grade 2 | 4.2 | 6.9 |
| | | | (OD04338) | | |
| Small | 3.4 | 33.5 | 83787 Kidney NAT (OD04338) | 7 | 8.3 |
| intestine | | | | | |
| Colon ca. | 0.5 | 0.6 | 83788 Kidney Ca Nuclear grade | 18.7 | 11.3 |
| SW480 | | | 1/2 (OD04339) | | |
| Colon ca.* | 0 | 0 | 83789 Kidney NAT (OD04339) | 4.9 | 8.5 |
| (SW480 | | | | | |
| met)SW620 | | | | | |
| Colon ca. | 0 | 0 | 83790 Kidney Ca, Clear cell type | 6.3 | 14.8 |
| HT29 | | | (OD04340) | | |
| Colon ca. | 0 | 0 | 83791 Kidney NAT (OD04340) | 10.7 | 14.6 |
| HCT-116 | | | | | |
| Colon ca. | 0.2 | 2.2 | 83792 Kidney Ca, Nuclear grade 3 | 6 | 7.5 |
| CaCo-2 | | | (OD04348) | | |
| 83219 CC | 2 | 0 | 83793 Kidney NAT (OD04348) | 14.5 | 16.5 |
| Well to Mod | | | | | |
| Diff | | | | | |
| (ODO3866) | | | | | |
| Colon ca. | 0.4 | 0 | 87474 Kidney Cancer (OD04622- | 15.4 | 25 |
| HCC-2998 | | | 01) | | |
| Gastric ca.* | 2.3 | 7.2 | 87475 Kidney NAT (OD04622- | 2.3 | 2.8 |
| (liver met) | | | 03) | | |
| NCI-N87 | | | | | |
| Bladder | 0.4 | 2.5 | 85973 Kidney Cancer (OD04450- | 3.6 | 7.3 |
| | | | 01) | | |
| Trachea | 2.8 | 4.9 | 85974 Kidney NAT (OD04450- | 1.3 | 13.3 |
| | | | 03) | | |
| Kidney | 0 | 0 | Kidney Cancer Clontech 8120607 | 7.3 | 5.2 |
| Kidney | 1.4 | 7 | Kidney NAT Clontech 8120608 | 1.1 | 3.6 |
| (fetal) | | | | | |
| Renal ca. | 0 | 0 | Kidney Cancer Clontech 8120613 | 2.8 | 7.5 |
| 786-0 | | | | | |
| Renal ca. | 0.2 | 1.5 | Kidney NAT Clontech 8120614 | 3.7 | 8 |
| A498 | | | | | |
| Renal ca. | 0 | 3 | Kidney Cancer Clontech 9010320 | 17.4 | 14.1 |
| RXF 393 | | | | | |
| Renal ca. | 0.6 | 1.1 | Kidney NAT Clontech 9010321 | 16.4 | 11.3 |
| ACHN | | | | | |
| Renal ca. | 0 | 0 | Normal Uterus GENPAK 061018 | 4.6 | 5.2 |
| UO-31 | | | | | |
| Renal ca. | 0.2 | 1.2 | Uterus Cancer GENPAK 064011 | 17.2 | 32.4 |
| TK-10 | | | | | |
| Liver | 0 | 0.6 | Normal Thyroid Clontech A+ | 17.3 | 18.1 |
| | | | 6570-1 | | |
| Liver (fetal) | 1.4 | 8.8 | Thyroid Cancer GENPAK 064010 | 6.5 | 1.1 |
| Liver ca. | 0.7 | 24.6 | Thyroid Cancer INVITROGEN | 1.7 | 1.6 |
| (hepatoblast) | | | A302152 | | |
| HepG2 | | | | | |
| Lung | 2.2 | 1.3 | Thyroid NAT INVITROGEN | 9.2 | 17.7 |
| | | | A302153 | | |
| Lung (fetal) | 5.1 | 15.8 | Normal Breast GENPAK 061019 | 21.3 | 30.1 |
| Lung ca. | 1.3 | 1.5 | 84877 Breast Cancer (OD04566) | 1.4 | 1.5 |
| (small cell) | | | | | |
| LX-1 | | | | | |
| Lung ca. | 3.3 | 4.2 | 85975 Breast Cancer (OD04590- | 12.9 | 10.1 |
| (small cell) | | | 01) | | |
| NCI-H69 | | | | | |
| Lung ca. | 0.2 | 1.4 | 85976 Breast Cancer Mets | 100 | 88 |
| (s.cell var.) | | | (OD04590-03) | | |
| SHP-77 | | | | | |
| Lung ca. | 0 | 0 | 87070 Breast Cancer Metastasis | 32.8 | 0 |
| (large | | | (OD04655-05) | | |
| cell)NCI- | | | | | |
| H460 | | | | | |
| Lung ca. | 0.8 | 2.9 | GENPAK Breast Cancer 064006 | 25 | 28.5 |
| (non-sm. | | | | | |
| cell) A549 | | | | | |
| Lung ca. | 4 | 9.5 | Breast Cancer Res. Gen. 1024 | 51.8 | 66.3 |
| (non-s.cell) | | | | | |
| NCI-H23 | | | | | |
| Lung ca | 1.8 | 3.4 | Breast Cancer Clontech 9100266 | 25 | 25.1 |
| (non-s.cell) | | | | | |
| HOP-62 | | | | | |
| Lung ca. | 0.3 | 0 | Breast NAT Clontech 9100265 | 18.2 | 31.7 |
| (non-s.cl) | | | | | |
| NCI-H522 | | | | | |
| Lung ca. | 0.5 | 4.7 | Breast Cancer INVITROGEN | 15 | 21 |
| (squam.) | | | A209073 | | |
| SW 900 | | | | | |
| Lung ca. | 0 | 0 | Breast NAT INVITROGEN | 16.2 | 12.3 |
| (squam.) | | | A2090734 | | |
| NCI-H596 | | | | | |
| Mammary | 4.8 | 0 | Normal Liver GENPAK 061009 | 5.6 | 4.9 |
| gland | | | | | |
| Breast ca.* | 0.4 | 0.7 | Liver Cancer GENPAK 064003 | 5.4 | 11.8 |
| (pl. | | | | | |
| effusion) | | | | | |
| MCF-7 | | | | | |
| Breast ca.* | 0.7 | 6.1 | Liver Cancer Research Genetics | 3.7 | 3.6 |
| (pl.ef) | | | RNA 1025 | | |
| MDA-MB- | | | | | |
| 231 | | | | | |
| Breast ca.* | 0 | 5.1 | Liver Cancer Research Genetics | 4.8 | 5.5 |
| (pl. | | | RNA 1026 | | |
| effusion) | | | | | |
| T47D | | | | | |
| Breast ca. | 0.4 | 2.8 | Paired Liver Cancer Tissue | 10.6 | 7 |
| BT-549 | | | Research Genetics RNA 6004-T | | |
| Breast ca. | 0 | 0 | Paired Liver Tissue Research | 19.3 | 28.4 |
| MDA-N | | | Genetics RNA 6004-N | | |
| Ovary | 5.9 | 2.7 | Paired Liver Cancer Tissue | 4.6 | 6 |
| | | | Research Genetics RNA 6005-T | | |
| Ovarian ca. | 0 | 3.2 | Paired Liver Tissue Research | 5 | 0 |
| OVCAR-3 | | | Genetics RNA 6005-N | | |
| Ovarian ca. | 1.4 | 3.7 | Normal Bladder GENPAK 061001 | 18.3 | 11.5 |
| OVCAR-4 | | | | | |
| Ovarian ca. | 0.5 | 2.5 | Bladder Cancer Research Genetics | 7.9 | 21.4 |
| OVCAR-5 | | | RNA 1023 | | |
| Ovarian ca. | 6.2 | 1.7 | Bladder Cancer INVITROGEN | 33 | 18.9 |
| OVCAR-8 | | | A302173 | | |
| Ovarian ca. | 0 | 0 | 87071 Bladder Cancer (OD04718- | 11 | 18.5 |
| IGROV-1 | | | 01) | | |
| Ovarian ca.* | 0 | 1.8 | 87072 Bladder Normal Adjacent | 9.4 | 9.7 |
| (ascites) | | | (OD04718-03) | | |
| SK-OV-3 | | | | | |
| Uterus | 1.8 | 24.6 | Normal Ovary Res. Gen. | 11.6 | 11.3 |
| Placenta | 3.3 | 2.8 | Ovarian Cancer GENPAK 064008 | 23.3 | 22.9 |
| Prostate | 1.6 | 2.5 | 87492 Ovary Cancer (OD04768- | 80.1 | 100 |
| | | | 07) | | |
| Prostate ca.* | 10.4 | 26.5 | 87493 Ovary NAT (OD04768-08) | 3.8 | 6.4 |
| (bone | | | | | |
| met)PC-3 | | | | | |
| Testis | 1.1 | 0 | Normal Stomach GENPAK | 15.9 | 17.1 |
| | | | 061017 | | |
| Melanoma | 0.2 | 0 | Gastric Cancer Clontech 9060358 | 36.3 | 45.8 |
| Hs688(A).T | | | | | |
| Melanoma* | 1.6 | 0 | NAT Stomach Clontech 9060359 | 44.4 | 60.8 |
| (met) | | | | | |
| Hs688(B).T | | | | | |
| Melanoma | 0.3 | 1.2 | Gastric Cancer Clontech 9060395 | 13.8 | 20.4 |
| UACC-62 | | | | | |
| Melanoma | 0 | 0 | NAT Stomach Clontech 9060394 | 78.5 | 93 |
| M14 | | | | | |
| Melanoma | 0 | 0.6 | Gastric Cancer Clontech 9060397 | 5.8 | 11.5 |
| LOX IMVI | | | | | |
| Melanoma* | 0.5 | 4.1 | NAT Stomach Clontech 9060396 | 25.5 | 30.1 |
| (met) SK- | | | | | |
| MEL-5 | | | | | |
| Adipose | 0.8 | 2 | Gastric Cancer GENPAK 064005 | 35.1 | 27.6 |

TABLE 14
Panel 3D

| | Rel. Expr., % |
|---|---|
| Tissue Name | 3dtm5227f_ag1605 |
| 94905_Daoy_Medulloblastoma/Cerebellum_sscDNA | 0.0 |
| 94906_TE671_Medulloblastom/Cerebellum_sscDNA | 0.4 |
| 94907_D283 Med_Medulloblastoma/Cerebellum_sscDNA | 0.0 |
| 94908_PFSK-1_Primitive | 0.0 |
| Neuroectodermal/Cerebellum_sscDNA | |
| 94909_XF-498_CNS_sscDNA | 0.0 |
| 94910_SNB-78_CNS/glioma_sscDNA | 0.0 |
| 94911_SF-268_CNS/glioblastoma_sscDNA | 0.0 |
| 94912_T98G_Glioblastoma_sscDNA | 0.0 |
| 96776_SK-N-SH_Neuroblastoma (metastasis)_sscDNA | 0.0 |
| 94913_SF-295_CNS/glioblastoma_sscDNA | 0.2 |
| 94914_Cerebellum_sscDNA | 1.9 |
| 96777_Cerebellum_sscDNA | 3.4 |
| 94916_NCI-H292_Mucoepidermoid lung carcinoma_sscDNA | 0.4 |
| 94917_DMS-114_Small cell lung cancer_sscDNA | 0.0 |
| 94918_DMS-79_Small cell lung | 3.8 |
| cancer/neuroendocrine_sscDNA | |
| 94919_NCI-H146_Small cell lung | 0.0 |
| cancer/neuroendocrine_sscDNA | |
| 94920_NCI-H526_Small cell lung | 14.6 |
| cancer/neuroendocrine_sscDNA | |
| 94921_NCI-N417_Small cell lung | 0.0 |
| cancer/neuroendocrine_sscDNA | |
| 94923_NCI-H82_Small cell lung | 0.2 |
| cancer/neuroendocrine_sscDNA | |
| 94924_NCI-H157_Squamous cell lung cancer | 0.0 |
| (metastasis)_sscDNA | |
| 94925_NCI-H1155_Large cell lung | 0.0 |
| cancer/neuroendocrine_sscDNA | |
| 94926_NCI-H1299_Large cell lung | 0.0 |
| cancer/neuroendocrine_sscDNA | |
| 94927_NCI-H727_Lung carcinoid_sscDNA | 0.5 |
| 94928_NCI-UMC-11_Lung carcinoid_sscDNA | 0.6 |
| 94929_LX-1_Small cell lung cancer_sscDNA | 0.2 |
| 94930_Colo-205_Colon cancer_sscDNA | 0.0 |
| 94931_KM12_Colon cancer_sscDNA | 0.0 |
| 94932_KM20L2_Colon cancer_sscDNA | 0.0 |
| 94933_NCI-H716_Colon cancer_sscDNA | 0.0 |
| 94935_SW-48_Colon adenocarcinoma_sscDNA | 0.1 |
| 94936_SW1116_Colon adenocarcinoma_sscDNA | 0.0 |
| 94937_LS 174T_Colon adenocarcinoma_sscDNA | 0.0 |
| 94938_SW-948_Colon adenocarcinoma_sscDNA | 0.0 |
| 94939_SW-480_Colon adenocarcinoma_sscDNA | 0.0 |
| 94940_NCI-SNU-5_Gastric carcinoma_sscDNA | 0.2 |
| 94941_KATO III_Gastric carcinoma_sscDNA | 0.4 |
| 94943_NCI-SNU-16_Gastric carcinoma_sscDNA | 0.0 |
| 94944_NCI-SNU-1_Gastric carcinoma_sscDNA | 0.0 |
| 94946_RF-1_Gastric adenocarcinoma_sscDNA | 15.9 |
| 94947_RF-48_Gastric adenocarcinoma_sscDNA | 18.7 |
| 96778_MKN-45_Gastric carcinoma_sscDNA | 0.0 |
| 94949_NCI-N87_Gastric carcinoma_sscDNA | 0.0 |
| 94951_OVCAR-5_Ovarian carcinoma_sscDNA | 0.0 |
| 94952_RL95-2_Uterine carcinoma_sscDNA | 0.0 |
| 94953_HelaS3_Cervical adenocarcinoma_sscDNA | 0.0 |
| 94954_Ca Ski_Cervical epidermoid carcinoma | 0.2 |
| (metastasis)_sscDNA | |
| 94955_ES-2_Ovarian clear cell carcinoma_sscDNA | 0.0 |
| 94957_Ramos/6h stim_”; Stimulated with PMA/ionomycin | 14.7 |
| 6h_sscDNA | |
| 94958_Ramos/14h stim_”; Stimulated with PMA/ionomycin | 22.7 |
| 14h_sscDNA | |
| 94962_MEG-01_Chronic myelogenous leukemia | 0.5 |
| (megokaryoblast)_sscDNA | |
| 94963_Raji_Burkitt's lymphoma_sscDNA | 32.3 |
| 94964_Daudi_Burkitt's lymphoma_sscDNA | 100.0 |
| 94965_U266_B-cell plasmacytoma/myeloma_sscDNA | 0.3 |
| 94968_CA46_Burkitt's lymphoma_sscDNA | 22.5 |
| 94970_RL_non-Hodgkin's B-cell lymphoma_sscDNA | 29.5 |
| 94972_JM1_pre-B-cell lymphoma/leukemia_sscDNA | 23.5 |
| 94973_Jurkat_T cell leukemia_sscDNA | 2.2 |
| 94974_TF-1_Erythroleukemia_sscDNA | 0.3 |
| 94975_HUT 78_T-cell lymphoma_sscDNA | 1.6 |
| 94977_U937_Histiocytic lymphoma_sscDNA | 7.3 |
| 94980_KU-812_Myelogenous leukemia_sscDNA | 0.1 |
| 94981_769-P_Clear cell renal carcinoma_sscDNA | 0.2 |
| 94983_Caki-2_Clear cell renal carcinoma_sscDNA | 0.1 |
| 94984_SW 839_Clear cell renal carcinoma_sscDNA | 1.0 |
| 94986_G401_Wilms' tumor_sscDNA | 0.0 |
| 94987_Hs766T_Pancreatic carcinoma (LN metastasis)_sscDNA | 0.1 |
| 94988_CAPAN-1_Pancreatic adenocarcinoma (liver | 0.0 |
| metastasis)_sscDNA | |
| 94989_SU86.86_Pancreatic carcinoma (liver | 0.0 |
| metastasis)_sscDNA | |
| 94990_BxPC-3_Pancreatic adenocarcinoma_sscDNA | 0.1 |
| 94991_HPAC_Pancreatic adenocarcinoma_sscDNA | 0.1 |
| 94992_MIA PaCa-2_Pancreatic carcinoma_sscDNA | 0.0 |
| 94993_CFPAC-1_Pancreatic ductal adenocarcinoma_sscDNA | 0.2 |
| 94994_PANC-1_Pancreatic epithelioid ductal | 0.0 |
| carcinoma_sscDNA | |
| 94996_T24_Bladder carcinma (transitional cell)_sscDNA | 0.0 |
| 94997_5637_Bladder carcinoma_sscDNA | 0.0 |
| 94998_HT-1197_Bladder carcinoma_sscDNA | 0.0 |
| 94999_UM-UC-3_Bladder carcinma (transitional cell)_sscDNA | 0.0 |
| 95000_A204_Rhabdomyosarcoma_sscDNA | 0.0 |
| 95001_HT-1080_Fibrosarcoma_sscDNA | 0.0 |
| 95002_MG-63_Osteosarcoma (bone)_sscDNA | 0.1 |
| 95003_SK-LMS-1_Leiomyosarcoma (vulva)_sscDNA | 0.2 |
| 95004_SJRH30_Rhabdomyosarcoma (met to bone | 0.0 |
| marrow)_sscDNA | |
| 95005_A431_Epidermoid carcinoma_sscDNA | 0.0 |
| 95007_WM266-4_Melanoma_sscDNA | 0.0 |
| 95010_DU 145_Prostate carcinoma (brain metastasis)_sscDNA | 0.0 |
| 95012_MDA-MB-468_Breast adenocarcinoma_sscDNA | 0.0 |
| 95013_SCC-4_Squamous cell carcinoma of tongue_sscDNA | 0.0 |
| 95014_SCC-9_Squamous cell carcinoma of tongue_sscDNA | 0.3 |
| 95015_SCC-15_Squamous cell carcinoma of tongue_sscDNA | 0.0 |
| 95017_CAL 27_Squamous cell carcinoma of tongue_sscDNA | 0.0 |

TABLE 14A
Panel 2D

| | Rel. Expr., % | Rel. Expr., % |
|---|---|---|
| | 2Dtm2728f_{—} | 2dx4tm4732f_{—} |
| Tissue Name | ag1605 | ag1605_a2 |
| Normal Colon GENPAK 061003 | 70.2 | 70.3 |
| 83219 CC Well to Mod Diff (ODO3866) | 8.5 | 6.3 |
| 83220 CC NAT (ODO3866) | 8.1 | 9.4 |
| 83221 CC Gr.2 rectosigmoid (ODO3868) | 4.5 | 12.1 |
| 83222 CC NAT (ODO3868) | 1.6 | 6.3 |
| 83235 CC Mod Diff (ODO3920) | 17.9 | 19.7 |
| 83236 CC NAT (ODO3920) | 21.5 | 11.6 |
| 83237 CC Gr.2 ascend colon (ODO3921) | 18 | 34.4 |
| 83238 CC NAT (ODO3921) | 8.9 | 9.8 |
| 83241 CC from Partial Hepatectomy (ODO4309) | 7.1 | 4.9 |
| 83242 Liver NAT (ODO4309) | 17.1 | 6.9 |
| 87472 Colon mets to lung (OD04451-01) | 6.7 | 11.4 |
| 87473 Lung NAT (OD04451-02) | 0.8 | 2.9 |
| Normal Prostate Clontech A+ 6546-1 | 17.8 | 52.9 |
| 84140 Prostate Cancer (OD04410) | 18.6 | 23 |
| 84141 Prostate NAT (OD04410) | 9.1 | 22 |
| 87073 Prostate Cancer (OD04720-01) | 20 | 14.5 |
| 87074 Prostate NAT (OD04720-02) | 21.3 | 38.1 |
| Normal Lung GENPAK 061010 | 69.7 | 85.9 |
| 83239 Lung Met to Muscle (ODO4286) | 11.1 | 6.3 |
| 83240 Muscle NAT (ODO4286) | 15.1 | 23.3 |
| 84136 Lung Malignant Cancer (OD03126) | 30.6 | 30.8 |
| 84137 Lung NAT (OD03126) | 8 | 16.3 |
| 84871 Lung Cancer (OD04404) | 41.8 | 45.2 |
| 84872 Lung NAT (OD04404) | 15.4 | 27.5 |
| 84875 Lung Cancer (OD04565) | 7.4 | 8.5 |
| 84876 Lung NAT (OD04565) | 5.3 | 1.2 |
| 85950 Lung Cancer (OD04237-01) | 77.9 | 72.9 |
| 85970 Lung NAT (OD04237-02) | 14.3 | 12.9 |
| 83255 Ocular Mel Met to Liver (ODO4310) | 2.7 | 6 |
| 83256 Liver NAT (ODO4310) | 2.3 | 5.1 |
| 84139 Melanoma Mets to Lung (OD04321) | 1.3 | 6.2 |
| 84138 Lung NAT (OD04321) | 36.9 | 22.4 |
| Normal Kidney GENPAK 061008 | 9.2 | 19.2 |
| 83786 Kidney Ca, Nuclear grade 2 (OD04338) | 4.2 | 6.9 |
| 83787 Kidney NAT (OD04338) | 7 | 8.3 |
| 83788 Kidney Ca Nuclear grade 1/2 (OD04339) | 18.7 | 11.3 |
| 83789 Kidney NAT (OD04339) | 4.9 | 8.5 |
| 83790 Kidney Ca, Clear cell type (OD04340) | 6.3 | 14.8 |
| 83791 Kidney NAT (OD04340) | 10.7 | 14.6 |
| 83792 Kidney Ca, Nuclear grade 3 (OD04348) | 6 | 7.5 |
| 83793 Kidney NAT (OD04348) | 14.5 | 16.5 |
| 87474 Kidney Cancer (OD04622-01) | 15.4 | 25 |
| 87475 Kidney NAT (OD04622-03) | 2.3 | 2.8 |
| 85973 Kidney Cancer (OD04450-01) | 3.6 | 7.3 |
| 85974 Kidney NAT (OD04450-03) | 1.3 | 13.3 |
| Kidney Cancer Clontech 8120607 | 7.3 | 5.2 |
| Kidney NAT Clontech 8120608 | 1.1 | 3.6 |
| Kidney Cancer Clontech 8120613 | 2.8 | 7.5 |
| Kidney NAT Clontech 8120614 | 3.7 | 8 |
| Kidney Cancer Clontech 9010320 | 17.4 | 14.1 |
| Kidney NAT Clontech 9010321 | 16.4 | 11.3 |
| Normal Uterus GENPAK 061018 | 4.6 | 5.2 |
| Uterus Cancer GENPAK 064011 | 17.2 | 32.4 |
| Normal Thyroid Clontech A+ 6570-1 | 17.3 | 18.1 |
| Thyroid Cancer GENPAK 064010 | 6.5 | 1.1 |
| Thyroid Cancer INVITROGEN A302152 | 1.7 | 1.6 |
| Thyroid NAT INVITROGEN A302153 | 9.2 | 17.7 |
| Normal Breast GENPAK 061019 | 21.3 | 30.1 |
| 84877 Breast Cancer (OD04566) | 1.4 | 1.5 |
| 85975 Breast Cancer (OD04590-01) | 12.9 | 10.1 |
| 85976 Breast Cancer Mets (OD04590-03) | 100 | 88 |
| 87070 Breast Cancer Metastasis (OD04655-05) | 32.8 | 0 |
| GENPAK Breast Cancer 064006 | 25 | 28.5 |
| Breast Cancer Res. Gen. 1024 | 51.8 | 66.3 |
| Breast Cancer Clontech 9100266 | 25 | 25.1 |
| Breast NAT Clontech 9100265 | 18.2 | 31.7 |
| Breast Cancer INVITROGEN A209073 | 15 | 21 |
| Breast NAT INVITROGEN A2090734 | 16.2 | 12.3 |
| Normal Liver GENPAK 061009 | 5.6 | 4.9 |
| Liver Cancer GENPAK 064003 | 5.4 | 11.8 |
| Liver Cancer Research Genetics RNA 1025 | 3.7 | 3.6 |
| Liver Cancer Research Genetics RNA 1026 | 4.8 | 5.5 |
| Paired Liver Cancer Tissue Research Genetics RNA 6004-T | 10.6 | 7 |
| Paired Liver Tissue Research Genetics RNA 6004-N | 19.3 | 28.4 |
| Paired Liver Cancer Tissue Research Genetics RNA 6005-T | 4.6 | 6 |
| Paired Liver Tissue Research Genetics RNA 6005-N | 5 | 0 |
| Normal Bladder GENPAK 061001 | 18.3 | 11.5 |
| Bladder Cancer Research Genetics RNA 1023 | 7.9 | 21.4 |
| Bladder Cancer INVITROGEN A302173 | 33 | 18.9 |
| 87071 Bladder Cancer (OD04718-01) | 11 | 18.5 |
| 87072 Bladder Normal Adjacent (OD04718-03) | 9.4 | 9.7 |
| Normal Ovary Res. Gen. | 11.6 | 11.3 |
| Ovarian Cancer GENPAK 064008 | 23.3 | 22.9 |
| 87492 Ovary Cancer (OD04768-07) | 80.1 | 100 |
| 87493 Ovary NAT (OD04768-08) | 3.8 | 6.4 |
| Normal Stomach GENPAK 061017 | 15.9 | 17.1 |
| Gastric Cancer Clontech 9060358 | 36.3 | 45.8 |
| NAT Stomach Clontech 9060359 | 44.4 | 60.8 |
| Gastric Cancer Clontech 9060395 | 13.8 | 20.4 |
| NAT Stomach Clontech 9060394 | 78.5 | 93 |
| Gastric Cancer Clontech 9060397 | 5.8 | 11.5 |
| NAT Stomach Clontech 9060396 | 25.5 | 30.1 |
| Gastric Cancer GENPAK 064005 | 35.1 | 27.6 |

TABLE 14B
Panel 4.1D

| | Rel. Expr., % |
|---|---|
| | 4.1dx4tm5937f_{—} |
| Tissue Name | ag1605_a2 |
| 93768_Secondary Th1_anti-CD28/anti-CD3 | 1.3 |
| 93769_Secondary Th2_anti-CD28/anti-CD3 | 2 |
| 93770_Secondary Tr1_anti-CD28/anti-CD3 | 1 |
| 93573_Secondary Th1_resting day 4-6 in IL-2 | 1.3 |
| 93572_Secondary Th2_resting day 4-6 in IL-2 | 2.9 |
| 93571_Secondary Tr1_resting day 4-6 in IL-2 | 3 |
| 93568_primary Th1_anti-CD28/anti-CD3 | 1 |
| 93569_primary Th2_anti-CD28/anti-CD3 | 0.6 |
| 93570_primary Tr1_anti-CD28/anti-CD3 | 0.5 |
| 93565_primary Th1_resting dy 4-6 in IL-2 | 2 |
| 93566_primary Th2_resting dy 4-6 in IL-2 | 1.4 |
| 93567_primary Tr1_resting dy 4-6 in IL-2 | 2.6 |
| 93351_CD45RA CD4 lymphocyte_anti-CD28/anti-CD3 | 3.6 |
| 93352_CD45RO CD4 lymphocyte_anti-CD28/anti-CD3 | 3.6 |
| 93251_CD8 Lymphocytes_anti-CD28/anti-CD3 | 3.2 |
| 93353_chronic CD8 Lymphocytes 2ry_resting dy 4-6 in | 2 |
| IL-2 | |
| 93574_chronic CD8 Lymphocytes 2ry_activated | 0.9 |
| CD3/CD28 | |
| 93354_CD4_none | 2 |
| 93252_Secondary Th1/Th2/Tr1_anti-CD95 CH11 | 2.9 |
| 93103_LAK cells_resting | 7.8 |
| 93788_LAK cells_IL-2 | 5.5 |
| 93787_LAK cells_IL-2 + IL-12 | 3.7 |
| 93789_LAK cells_IL-2 + IFN gamma | 5.1 |
| 93790_LAK cells_IL-2 + IL-18 | 4.8 |
| 93104_LAK cells_PMA/ionomycin and IL-18 | 1.7 |
| 93578_NK Cells IL-2_resting | 4.5 |
| 93109_Mixed Lymphocyte Reaction_Two Way MLR | 10.5 |
| 93110_Mixed Lymphocyte Reaction_Two Way MLR | 4.5 |
| 93111_Mixed Lymphocyte Reaction_Two Way MLR | 2.4 |
| 93112_Mononuclear Cells (PBMCs)_resting | 2.9 |
| 93113_Mononuclear Cells (PBMCs)_PWM | 4.4 |
| 93114_Mononuclear Cells (PBMCs)_PHA-L | 10 |
| 93249_Ramos (B cell)_none | 94.1 |
| 93250_Ramos (B cell)_ionomycin | 100 |
| 93349_B lymphocytes_PWM | 16.9 |
| 93350_B lymphoytes_CD40L and IL-4 | 61.3 |
| 92665_EOL-1 (Eosinophil)_dbcAMP differentiated | 33.7 |
| 93248_EOL-1 (Eosinophil)_dbcAMP/PMAionomycin | 11.9 |
| 93356_Dendritic Cells_none | 2 |
| 93355_Dendritic Cells_LPS 100 ng/ml | 1.5 |
| 93775_Dendritic Cells_anti-CD40 | 1.6 |
| 93774_Monocytes_resting | 3.3 |
| 93776_Monocytes_LPS 50 ng/ml | 5.3 |
| 93581_Macrophages_resting | 2.4 |
| 93582_Macrophages_LPS 100 ng/ml | 1 |
| 93098_HUVEC (Endothelial)_none | 0.1 |
| 93099_HUVEC (Endothelial)_starved | 0.2 |
| 93100_HUVEC (Endothelial)_IL-1b | 0 |
| 93779_HUVEC (Endothelial)_IFN gamma | 0.2 |
| 93102_HUVEC (Endothelial)_TNF alpha + IFN gamma | 0.2 |
| 93101_HUVEC (Endothelial)_TNF alpha + IL4 | 0.2 |
| 93781_HUVEC (Endothelial)_IL-11 | 0.4 |
| 93583_Lung Microvascular Endothelial Cells_none | 0.7 |
| 93584_Lung Microvascular Endothelial Cells_TNFa (4 ng/ml) | 0 |
| and IL1b (1 ng/ml) | |
| 92662_Microvascular Dermal endothelium_none | 0 |
| 92663_Microsvasular Dermal endothelium_TNFa (4 ng/ml) | 0.9 |
| and IL1b (1 ng/ml) | |
| 93773_Bronchial epithelium_TNFa (4 ng/ml) and IL1b (1 ng/ml)** | 1.2 |
| 93347_Small Airway Epithelium_none | 0 |
| 93348_Small Airway Epithelium_TNFa (4 ng/ml) and | 0.2 |
| IL1b (1 ng/ml) | |
| 92668_Coronery Artery SMC_resting | 0.4 |
| 92669_Coronery Artery SMC_TNFa (4 ng/ml) and IL1b (1 ng/ml) | 0 |
| 93107_astrocytes_resting | 0.3 |
| 93108_astrocytes_TNFa (4 ng/ml) and IL1b (1 ng/ml) | 0.2 |
| 92666_KU-812 (Basophil)_resting | 0 |
| 92667_KU-812 (Basophil)_PMA/ionoycin | 0.3 |
| 93579_CCD1106 (Keratinocytes)_none | 0.2 |
| 93580_CCD1106 (Keratinocytes)_TNFa and IFNg** | 0.4 |
| 93791_Liver Cirrhosis | 0.4 |
| 93577_NCI-H292 | 0 |
| 93358_NCI-H292_IL-4 | 0 |
| 93360_NCI-H292_IL-9 | 0 |
| 93359_NCI-H292_IL-13 | 0.6 |
| 93357_NCI-H292_IFN gamma | 0.2 |
| 93777_HPAEC_- | 0 |
| 93778_HPAEC_IL-1beta/TNA alpha | 0 |
| 93254_Normal Human Lung Fibroblast_none | 1.9 |
| 93253_Normal Human Lung Fibroblast_TNFa (4 ng/ml) | 0.3 |
| and IL-1b (1 ng/ml) | |
| 93257_Normal Human Lung Fibroblast_IL-4 | 0.3 |
| 93256_Normal Human Lung Fibroblast_IL-9 | 0.5 |
| 93255_Normal Human Lung Fibroblast_IL-13 | 0 |
| 93258_Normal Human Lung Fibroblast_IFN gamma | 1.1 |
| 93106_Dermal Fibroblasts CCD1070_resting | 0 |
| 93361_Dermal Fibroblasts CCD1070_TNF alpha 4 ng/ml | 0.6 |
| 93105_Dermal Fibroblasts CCD1070_IL-1beta 1 ng/ml | 0 |
| 93772_dermal fibroblast_IFN gamma | 0 |
| 93771_dermal fibroblast_IL-4 | 0.2 |
| 93892_Dermal fibroblasts_none | 0 |
| 99202_Neutrophils_TNFa + LPS | 3.6 |
| 99203_Neutrophils_none | 15.8 |
| 735010_Colon_normal | 5.1 |
| 735019_Lung_none | 1.6 |
| 64028-1_Thymus_none | 12.2 |
| 64030-1_Kidney_none | 0.3 |

The RTQ-PCR analysis (Table 13 and 14) reveals that MOL1 is predominantly expressed in cell lines derived from lymphoma and leukemia, specifically Burkitt's lymphoma in panel 3D. This result is supported by the presence of GenBank ESTs coming from T cells from T cell leukemia (see Unigene http://www.ncbi.nlm.nih.gov/UniGene/clust.cgi?ORG=Hs&CID=87968). Recent report indicates that this receptor normally aids the immune cells to sense the presence of unmethylated CpG dinucleotides (Hemmi H, Takeuchi O, Kawai T, Kaisho T, Sato S, Sanjo H, Matsumoto M, Hoshino K, Wagner H, Takeda K, Akira S. A Toll-like receptor recognizes bacterial DNA. Nature Dec. 7, 2000;408(6813):740-5) and to induce proliferation of splenocytes, inflammatory cytokine production from macrophages and maturation of dendritic cells. The signaling pathway mediated by toll-like receptor 9 is through the activation of NF-kB. There is evidence that NF-kappaB activity is necessary for survival of lymphoma and leukemia cells (Constitutive activation of NF-kappaB in primary adult T-cell leukemia cells. Mori N, Fujii M, Ikeda S, Yamada Y, Tomonaga M, Ballard D W, Yamamoto N. Blood Apr. 1, 1999;93(7):2360-8; Inhibition of NF-kappaB induces apoptosis of KSHV-infected primary effusion lymphoma cells. Keller S A, Schattner E J, Cesarman E. Blood, Oct. 1, 2000, 96, No. 7, pp. 2537-2542). Overexpression of toll-like receptor 9 by lymphoma and leukemia cells is likely to mediate ligand-independent signaling, affecting the normal processes of activation, proliferation and tumorogenesis. Therefore the protein encoded (GM_{—}79960178) may serve as a potential marker for lymphoma and leukemia cells. In addition, human monoclonal antibodies directed against this protein could be therapeutics for the treatment of lymphoma and leukemia.

MOL2

Expression of gene MOL2 was assessed using the primer-probe set Ag743, described in Table 15. Results of the RTQ-PCR runs are shown in Table 16.

TABLE 15

Probe Name: Ag743

| | | | | Start | SEQID |
|---|---|---|---|---|---|
| Primers | Sequences | Tm | Length | Position | NO: |
| Forward | 5′-ATGTCTTGGTGGATGCAGAA-3′ | 59.1 | 20 | 1304 | 79 |
| Probe | TET-5′-CGGACTATAGCATTTCTAAGCGCCTCG-3′-TAMRA | 69.2 | 27 | 1340 | 80 |
| Reverse | 5′-CACATCCTCCTTGCAAATGT-3′ | 58.6 | 20 | 1370 | 81 |

TABLE 16
Panels 1.3D and 4D

| PANEL 1.3D | | PANEL 4D | |
|---|---|---|---|
| | Rel. Expr., % | | |
| | 1.3dx4tm5604t_{—} | | Rel. Expr., % |
| Tissue Name | ag743_a1 | Tissue Name | 4Dtm2477t_ag743 |
| Liver | 36.4 | 93768_Secondary Th1_anti- | 27.2 |
| adenocarcinoma | | CD28/anti-CD3 | |
| Pancreas | 4.6 | 93769_Secondary Th2_anti- | 19.6 |
| | | CD28/anti-CD3 | |
| Pancreatic ca. | 16.6 | 93770_Secondary Tr1_anti- | 23.2 |
| CAPAN 2 | | CD28/anti-CD3 | |
| Adrenal gland | 6.5 | 93573_Secondary Th1_resting | 7.2 |
| | | day 4-6 in IL-2 | |
| Thyroid | 5.2 | 93572_Secondary Th2_resting | 7.1 |
| | | day 4-6 in IL-2 | |
| Salivary gland | 12.0 | 93571_Secondary Tr1_resting | 11.9 |
| | | day 4-6 in IL-2 | |
| Pituitary gland | 22.7 | 93568_primary Th1_anti- | 43.2 |
| | | CD28/anti-CD3 | |
| Brain (fetal) | 8.4 | 93569_primary Th2_anti- | 32.1 |
| | | CD28/anti-CD3 | |
| Brain (whole) | 38.6 | 93570_primary Tr1_anti- | 50.0 |
| | | CD28/anti-CD3 | |
| Brain | 24.8 | 93565_primary Th1_resting dy | 43.8 |
| (amygdala) | | 4-6 in IL-2 | |
| Brain | 31.4 | 93566_primary Th2_resting dy | 16.4 |
| (cerebellum) | | 4-6 in IL-2 | |
| Brain | 22.0 | 93567_primary Tr1_resting dy | 22.4 |
| (hippocampus) | | 4-6 in IL-2 | |
| Brain | 11.8 | 93351_CD45RA CD4 | 23.0 |
| (substantia | | lymphocyte_anti-CD28/anti- | |
| nigra) | | CD3 | |
| Brain | 30.3 | 93352_CD45RO CD4 | 25.5 |
| (thalamus) | | lymphocyte_anti-CD28/anti- | |
| | | CD3 | |
| Cerebral | 22.0 | 93251_CD8 Lymphocytes_anti- | 16.0 |
| Cortex | | CD28/anti-CD3 | |
| Spinal cord | 20.6 | 93353_chronic CD8 | 15.0 |
| | | Lymphocytes 2ry_resting dy 4-6 | |
| | | in IL-2 | |
| CNS ca. | 28.1 | 93574_chronic CD8 | 10.2 |
| (glio/astro) | | Lymphocytes 2ry_activated | |
| U87-MG | | CD3/CD28 | |
| CNS ca. | 27.9 | 93354_CD4_none | 4.0 |
| (glio/astro) | | | |
| U-118-MG | | | |
| CNS ca. | 31.1 | 93252_Secondary | 9.6 |
| (astro) | | Th1/Th2/Tr1_anti-CD95 CH11 | |
| SW1783 | | | |
| CNS ca.* | 19.3 | 93103_LAK cells_resting | 12.9 |
| (neuro; met ) | | | |
| SK-N-AS | | | |
| CNS ca. | 47.2 | 93788_LAK cells_IL-2 | 14.9 |
| (astro) | | | |
| SF-539 | | | |
| CNS ca. | 10.2 | 93787_LAK cells_IL-2 + IL-12 | 12.5 |
| (astro) | | | |
| SNB-75 | | | |
| CNS ca. (glio) | 24.3 | 93789_LAK cells_IL-2 + IFN | 22.2 |
| SNB-19 | | gamma | |
| CNS ca. (glio) | 20.9 | 93790_LAK cells_IL-2 + IL-18 | 15.7 |
| U251 | | | |
| CNS ca. (glio) | 10.7 | 93104_LAK | 4.0 |
| SF-295 | | cells_PMA/ionomycin and IL-18 | |
| Heart (fetal) | 2.0 | 93578_NK Cells IL-2_resting | 12.0 |
| Heart | 6.5 | 93109_Mixed Lymphocyte | 9.8 |
| | | Reaction_Two Way MLR | |
| Fetal Skeletal | 0.7 | 93110_Mixed Lymphocyte | 14.3 |
| | | Reaction_Two Way MLR | |
| Skeletal | 18.3 | 93111_Mixed Lymphocyte | 11.7 |
| muscle | | Reaction_Two Way MLR | |
| Bone marrow | 11.2 | 93112_Mononuclear Cells | 5.8 |
| | | (PBMCs)_resting | |
| Thymus | 7.8 | 93113_Mononuclear Cells | 51.8 |
| | | (PBMCs)_PWM | |
| Spleen | 7.2 | 93114_Mononuclear Cells | 27.5 |
| | | (PBMCs)_PHA-L | |
| Lymph node | 7.5 | 93249_Ramos (B cell)_none | 0.2 |
| Colorectal | 28.4 | 93250_Ramos (B | 6.7 |
| | | cell)_ionomycin | |
| Stomach | 6.5 | 93349_B lymphocytes_PWM | 100.0 |
| Small intestine | 10.0 | 93350_B lymphoytes_CD40L | 23.7 |
| | | and IL-4 | |
| Colon ca. | 23.2 | 92665_EOL-1 | 15.5 |
| SW480 | | (Eosinophil)_dbcAMP | |
| | | differentiated | |
| Colon ca.* | 12.3 | 93248_EOL-1 | 10.4 |
| (SW480 | | (Eosinophil)_dbcAMP/PMAionomycin | |
| met)SW620 | | | |
| Colon ca. | 1.7 | 93356_Dendritic Cells_none | 15.3 |
| HT29 | | | |
| Colon ca. | 13.1 | 93355_Dendritic Cells_LPS 100 ng/ml | 6.7 |
| HCT-116 | | | |
| Colon ca. | 12.5 | 93775_Dendritic Cells_anti- | 16.7 |
| CaCo-2 | | CD40 | |
| 83219 CC | 10.4 | 93774_Monocytes_resting | 14.9 |
| Well to Mod | | | |
| Diff | | | |
| (ODO3866) | | | |
| Colon ca. | 11.3 | 93776_Monocytes_LPS 50 ng/ml | 4.0 |
| HCC-2998 | | | |
| Gastric ca.* | 17.6 | 93581_Macrophages_resting | 19.5 |
| (liver met) | | | |
| NCI-N87 | | | |
| Bladder | 17.6 | 93582_Macrophages_LPS 100 ng/ml | 7.5 |
| Trachea | 7.6 | 93098_HUVEC | 33.0 |
| | | (Endothelial)_none | |
| Kidney | 5.7 | 93099_HUVEC | 66.0 |
| | | (Endothelial)_starved | |
| Kidney (fetal) | 6.4 | 93100_HUVEC | 29.3 |
| | | (Endothelial)_IL-1b | |
| Renal ca. | 10.0 | 93779_HUVEC | 33.0 |
| 786-0 | | (Endothelial)_IFN gamma | |
| Renal ca. | 23.6 | 93102_HUVEC | 23.0 |
| A498 | | (Endothelial)_TNF alpha + IFN | |
| | | gamma | |
| Renal ca. | 37.8 | 93101_HUVEC | 19.9 |
| RXF 393 | | (Endothelial)_TNF alpha + IL4 | |
| Renal ca. | 18.1 | 93781_HUVEC | 22.4 |
| ACHN | | (Endothelial)_IL-11 | |
| Renal ca. | 26.1 | 93583_Lung Microvascular | 27.5 |
| UO-31 | | Endothelial Cells_none | |
| Renal ca. | 10.9 | 93584_Lung Microvascular | 25.2 |
| TK-10 | | Endothelial Cells_TNFa (4 ng/ml) | |
| | | and IL1b (1 ng/ml) | |
| Liver | 2.2 | 92662_Microvascular Dermal | 51.4 |
| | | endothelium_none | |
| Liver (fetal) | 4.4 | 92663_Microsvasular Dermal | 22.5 |
| | | endothelium_TNFa (4 ng/ml) | |
| | | and IL1b (1 ng/ml) | |
| Liver ca. | 28.1 | 93773_Bronchial | 17.0 |
| (hepatoblast) | | epithelium_TNFa (4 ng/ml) and | |
| HepG2 | | IL1b (1 ng/ml)** | |
| Lung | 12.4 | 93347_Small Airway | 20.9 |
| | | Epithelium_none | |
| Lung (fetal) | 5.2 | 93348_Small Airway | 84.7 |
| | | Epithelium_TNFa (4 ng/ml) and | |
| | | IL1b (1 ng/ml) | |
| Lung ca. | 10.8 | 92668_Coronery Artery | 37.9 |
| (small cell) | | SMC_resting | |
| LX-1 | | | |
| Lung ca. | 20.2 | 92669_Coronery Artery | 26.8 |
| (small cell) | | SMC_TNFa (4 ng/ml) and IL1b | |
| NCI-H69 | | (1 ng/ml) | |
| Lung ca. | 20.1 | 93107_astrocytes_resting | 47.6 |
| (s. cell var.) | | | |
| SHP-77 | | | |
| Lung ca. | 10.7 | 93108_astrocytes_TNFa (4 ng/ml) | 20.0 |
| (large | | and IL1b (1 ng/ml) | |
| cell)NCI- | | | |
| H460 | | | |
| Lung ca. (non- | 5.5 | 92666_KU-812 | 25.0 |
| sm. cell) A549 | | (Basophil)_resting | |
| Lung ca. (non- | 13.2 | 92667_KU-812 | 31.9 |
| s. cell) NCI- | | (Basophil)_PMA/ionoycin | |
| H23 | | | |
| Lung ca (non- | 18.3 | 93579_CCD1106 | 47.0 |
| s. cell) HOP- | | (Keratinocytes)_none | |
| 62 | | | |
| Lung ca. (non- | 13.8 | 93580_CCD1106 | 7.2 |
| s. cl) NCI- | | (Keratinocytes)_TNFa and IFNg** | |
| H522 | | | |
| Lung ca. | 17.2 | 93791_Liver Cirrhosis | 4.2 |
| (squam.) | | | |
| SW 900 | | | |
| Lung ca. | 20.9 | 93792_Lupus Kidney | 3.8 |
| (squam.) | | | |
| NCI-H596 | | | |
| Mammary | 17.8 | 93577_NCI-H292 | 55.1 |
| gland | | | |
| Breast ca.* | 20.4 | 93358_NCI-H292_IL-4 | 68.8 |
| (pl. effusion) | | | |
| MCF-7 | | | |
| Breast ca.* | 22.5 | 93360_NCI-H292_IL-9 | 59.5 |
| (pl. ef) MDA- | | | |
| MB-231 | | | |
| Breast ca.* | 15.5 | 93359_NCI-H292_IL-13 | 35.1 |
| (pl. effusion) | | | |
| T47D | | | |
| Breast ca. | 13.9 | 93357_NCI-H292_IFN gamma | 37.9 |
| BT-549 | | | |
| Breast ca. | 9.9 | 93777_HPAEC_- | 32.3 |
| MDA-N | | | |
| Ovary | 5.6 | 93778_HPAEC_IL-1beta/TNA | 23.2 |
| | | alpha | |
| Ovarian ca. | 15.4 | 93254_Normal Human Lung | 29.7 |
| OVCAR-3 | | Fibroblast none | |
| Ovarian ca. | 12.4 | 93253_Normal Human Lung | 16.8 |
| OVCAR-4 | | Fibroblast_TNFa (4 ng/ml) and | |
| | | IL-1b (1 ng/ml) | |
| Ovarian ca. | 27.2 | 93257_Normal Human Lung | 55.5 |
| OVCAR-5 | | Fibroblast_IL-4 | |
| Ovarian ca. | 9.0 | 93256_Normal Human Lung | 34.9 |
| OVCAR-8 | | Fibroblast_IL-9 | |
| Ovarian ca. | 22.6 | 93255_Normal Human Lung | 36.9 |
| IGROV-1 | | Fibroblast_IL-13 | |
| Ovarian ca.* | 100.0 | 93258_Normal Human Lung | 58.6 |
| (ascites) SK- | | Fibroblast_IFN gamma | |
| OV-3 | | | |
| Uterus | 6.1 | 93106_Dermal Fibroblasts | 74.2 |
| | | CCD1070_resting | |
| Plancenta | 15.8 | 93361_Dermal Fibroblasts | 64.2 |
| | | CCD1070_TNF alpha 4 ng/ml | |
| Prostate | 5.6 | 93105_Dermal Fibroblasts | 27.7 |
| | | CCD1070_IL-1beta 1 ng/ml | |
| Prostate ca.* | 50.1 | 93772_dermal fibroblast_IFN | 24.0 |
| (bone met)PC-3 | | gamma | |
| Testis | 8.3 | 93771_dermal fibroblast_IL-4 | 58.2 |
| Melanoma | 12.6 | 93259_IBD Colitis 1** | 2.8 |
| Hs688(A).T | | | |
| Melanoma* | 14.5 | 93260_IBD Colitis 2 | 1.1 |
| (met) | | | |
| Hs688(B).T | | | |
| Melanoma | 18.8 | 93261_IBD Crohns | 2.1 |
| UACC-62 | | | |
| Melanoma | 19.7 | 735010_Colon_normal | 14.3 |
| M14 | | | |
| Melanoma | 8.8 | 735019_Lung_none | 21.5 |
| LOX IMVI | | | |
| Melanoma* | 11.0 | 64028-1_Thymus_none | 27.0 |
| (met) SK- | | | |
| MEL-5 | | | |
| Adipose | 14.5 | 64030-1_Kidney_none | 31.2 |

MOL2 is widely expressed in tissues and cell lines represented in both panels 1.3D and 4D, with highest expression being in one ovarian cancer cell line (SK-OV-3). Thus, it could serve as a diagnostic marker for ovarian cancer.

MOL3

Expression of MOL3 was assessed using the primer-probe sets Ag474 and Ag770, described in Tables 17 and 18. Results of the RTQ-PCR runs are shown in Tables 19 and 20.

TABLE 17

Probe name: Ag474

| | | | Start | SEQ ID |
|---|---|---|---|---|
| Primers | Sequences | Length | Position | NO: |
| Forward | 5′-GGCACTGTCCCTCTGCACAT-3′ | 20 | 493 | 82 |
| Probe | FAM-5′-CCCTGAGAAAGATCTGCCACAAAGACATCTG-3′-TAMRA | 31 | 516 | 83 |
| Reverse | 5′-AACCTGCCCACAGAGCAATC-3′ | 20 | 549 | 84 |

TABLE 18

Probe name: Ag770

| | | | | |
|---|---|---|---|---|
| | | | | Start |
| Primers | Sequences | Tm | Length | Position |

| | | | | |
|---|---|---|---|---|
| Forward | 5′-ACAGTGCTGTCGCTGGTACTT-3′ | 60 | 21 | 717 |
| Probe | FAM-5′-TTCGTACTGAAAGGCGTACCTCTCCA-3′-TAMRA | 67.9 | 26 | 746 |
| Reverse | 5′-CTCAAACAGCTCACGAGTGAT-3′ | 58.1 | 21 | 773 |

TABLE 19
Ag474

| PANEL 1.3D | | PANEL 2D | |
|---|---|---|---|
| | Rel. Expr., % | | Rel. Expr., % |
| Tissue Name | 1.3Dtm3254f_ag474 | Tissue Name | 2Dtm3255f_ag474 |
| Liver | 0.0 | Normal Colon GENPAK | 0.3 |
| adenocarcinoma | | 061003 | |
| Pancreas | 0.0 | 83219 CC Well to Mod | 0.0 |
| | | Diff (ODO3866) | |
| Pancreatic ca. | 0.0 | 83220 CC NAT | 0.0 |
| CAPAN 2 | | (ODO3866) | |
| Adrenal gland | 0.3 | 83221 CC Gr.2 | 0.0 |
| | | rectosigmoid (ODO3868) | |
| Thyroid | 0.0 | 83222 CC NAT | 0.0 |
| | | (ODO3868) | |
| Salivary gland | 0.0 | 83235 CC Mod Diff | 0.0 |
| | | (ODO3920) | |
| Pituitary gland | 0.0 | 83236 CC NAT | 0.0 |
| | | (ODO3920) | |
| Brain (fetal) | 0.2 | 83237 CC Gr.2 ascend | 0.0 |
| | | colon (ODO3921) | |
| Brain (whole) | 0.0 | 83238 CC NAT | 0.0 |
| | | (ODO3921) | |
| Brain (amygdala) | 0.1 | 83241 CC from Partial | 3.8 |
| | | Hepatectomy (ODO4309) | |
| Brain (cerebellum) | 0.0 | 83242 Liver NAT | 100.0 |
| | | (ODO4309) | |
| Brain | 0.0 | 87472 Colon mets to lung | 0.0 |
| (hippocampus) | | (OD04451-01) | |
| Brain (substantia | 0.0 | 87473 Lung NAT | 0.0 |
| nigra) | | (OD04451-02) | |
| Brain (thalamus) | 0.0 | Normal Prostate Clontech | 0.0 |
| | | A+ 6546-1 | |
| Cerebral Cortex | 0.1 | 84140 Prostate Cancer | 0.0 |
| | | (OD04410) | |
| Spinal cord | 0.0 | 84141 Prostate NAT | 0.0 |
| | | (OD04410) | |
| CNS ca. (glio/astro) | 0.3 | 87073 Prostate Cancer | 0.0 |
| U87-MG | | (OD04720-01) | |
| CNS ca. | 0.0 | 87074 Prostate NAT | 0.0 |
| (glio/astro) U-118- | | (OD04720-02) | |
| MG | | | |
| CNS ca. (astro) | 0.0 | Normal Lung GENPAK | 0.0 |
| SW1783 | | 061010 | |
| CNS ca.* (neuro; | 0.0 | 83239 Lung Met to Muscle | 0.0 |
| met) SK-N-AS | | (ODO4286) | |
| CNS ca. (astro) | 0.0 | 83240 Muscle NAT | 0.0 |
| SF-539 | | (ODO4286) | |
| CNS ca. (astro) | 0.0 | 84136 Lung Malignant | 0.0 |
| SNB-75 | | Cancer (OD03126) | |
| CNS ca. (glio) | 0.0 | 84137 Lung NAT | 0.0 |
| SNB-19 | | (OD03126) | |
| CNS ca. (glio) | 0.0 | 84871 Lung Cancer | 0.0 |
| U251 | | (OD04404) | |
| CNS ca. (glio) | 0.0 | 84872 Lung NAT | 0.0 |
| SF-295 | | (OD04404) | |
| Heart (fetal) | 0.0 | 84875 Lung Cancer | 0.0 |
| | | (OD04565) | |
| Heart | 0.0 | 84876 Lung NAT | 0.0 |
| | | (OD04565) | |
| Fetal Skeletal | 0.2 | 85950 Lung Cancer | 0.0 |
| | | (OD04237-01) | |
| Skeletal muscle | 0.0 | 85970 Lung NAT | 0.0 |
| | | (OD04237-02) | |
| Bone marrow | 0.0 | 83255 Ocular Mel Met to | 0.0 |
| | | Liver (ODO4310) | |
| Thymus | 0.0 | 83256 Liver NAT | 64.2 |
| | | (ODO4310) | |
| Spleen | 0.2 | 84139 Melanoma Mets to | 0.0 |
| | | Lung (OD04321) | |
| Lymph node | 0.0 | 84138 Lung NAT | 0.0 |
| | | (OD04321) | |
| Colorectal | 0.0 | Normal Kidney GENPAK | 0.0 |
| | | 061008 | |
| Stomach | 0.1 | 83786 Kidney Ca, | 0.0 |
| | | Nuclear grade 2 | |
| | | (OD04338) | |
| Small intestine | 0.0 | 83787 Kidney NAT | 0.0 |
| | | (OD04338) | |
| Colon ca. | 0.3 | 83788 Kidney Ca Nuclear | 0.0 |
| SW480 | | grade 1/2 (OD04339) | |
| Colon ca.* (SW480 | 0.0 | 83789 Kidney NAT | 0.0 |
| met)SW620 | | (OD04339) | |
| Colon ca. | 0.0 | 83790 Kidney Ca, Clear | 0.1 |
| HT29 | | cell type (OD04340) | |
| Colon ca. | 0.0 | 83791 Kidney NAT | 0.0 |
| HCT-116 | | (OD04340) | |
| Colon ca. | 0.3 | 83792 Kidney Ca, Nuclear | 0.0 |
| CaCo-2 | | grade 3 (OD04348) | |
| 83219 CC Well to | 0.3 | 83793 Kidney NAT | 0.0 |
| Mod Diff | | (OD04348) | |
| (ODO3866) | | | |
| Colon ca. | 0.5 | 87474 Kidney Cancer | 0.0 |
| HCC-2998 | | (OD04622-01) | |
| Gastric ca.* (liver | 0.0 | 87475 Kidney NAT | 0.0 |
| met) NCI-N87 | | (OD04622-03) | |
| Bladder | 0.0 | 85973 Kidney Cancer | 0.0 |
| | | (OD04450-01) | |
| Trachea | 0.0 | 85974 KidneyNAT | 0.1 |
| | | (OD04450-03) | |
| Kidney | 0.0 | Kidney Cancer Clontech | 0.0 |
| Breast ca.* (pl. | 0.0 | Liver Cancer GENPAK | 49.0 |
| effusion) MCF-7 | | 064003 | |
| Breast ca.* (pl. ef) | 0.0 | Liver Cancer Research | 62.4 |
| MDA-MB-231 | | Genetics RNA 1025 | |
| Breast ca.* (pl. | 0.0 | Liver Cancer Research | 12.9 |
| effusion) T47D | | Genetics RNA 1026 | |
| Breast ca. | 0.0 | Paired Liver Cancer Tissue | 74.2 |
| BT-549 | | Research Genetics RNA | |
| | | 6004-T | |
| Breast ca. | 0.1 | Paired Liver Tissue | 8.5 |
| MDA-N | | Research Genetics RNA | |
| | | 6004-N | |
| Ovary | 0.0 | Paired Liver Cancer Tissue | 5.0 |
| | | Research Genetics RNA | |
| | | 6005-T | |
| Ovarian ca. | 0.5 | Paired Liver Tissue | 18.6 |
| OVCAR-3 | | Research Genetics RNA | |
| | | 6005-N | |
| Ovarian ca. | 0.0 | Normal Bladder GENPAK | 0.0 |
| OVCAR-4 | | 061001 | |
| Ovarian ca. | 0.0 | Bladder Cancer Research | 0.0 |
| OVCAR-5 | | Genetics RNA 1023 | |
| Ovarian ca. | 0.0 | Bladder Cancer | 0.0 |
| OVCAR-8 | | INVITROGEN A302173 | |
| Ovarian ca. | 0.0 | 87071 Bladder Cancer | 0.0 |
| IGROV-1 | | (OD04718-01) | |
| Ovarian ca.* | 0.0 | 87072 Bladder Normal | 0.0 |
| (ascites) SK-OV-3 | | Adjacent (OD04718-03) | |
| Uterus | 0.0 | Normal Ovary Res. Gen. | 0.0 |
| Placenta | 0.0 | Ovarian Cancer GENPAK | 0.0 |
| | | 064008 | |
| Prostate | 0.1 | 87492 Ovary Cancer | 0.1 |
| | | (OD04768-07) | |
| Prostate ca.* (bone | 0.0 | 87493 Ovary NAT | 0.0 |
| met)PC-3 | | (OD04768-08) | |
| Testis | 0.3 | Normal Stomach GENPAK | 0.0 |
| | | 061017 | |
| Melanoma | 0.0 | Gastric Cancer Clontech | 0.0 |
| Hs688(A).T | | 9060358 | |
| Melanoma* (met) | 0.0 | NAT Stomach Clontech | 0.0 |
| Hs688(B).T | | 9060359 | |
| Melanoma | 0.0 | Gastric Cancer Clontech | 0.4 |
| UACC-62 | | 9060395 | |
| Melanoma | 0.0 | NAT Stomach Clontech | 0.0 |
| M14 | | 9060394 | |
| Melanoma | 0.0 | Gastric Cancer Clontech | 0.3 |
| LOX IMVI | | 9060397 | |
| Melanoma* (met) | 0.0 | NAT Stomach Clontech | 0.0 |
| SK-MEL-5 | | 9060396 | |
| Adipose | 0.0 | Gastric Cancer GENPAK | 0.0 |
| | | 064005 | |

TABLE 20
Ag770

| PANEL 1.3D | | PANEL 4D | | |
|---|---|---|---|---|
| | Rel. Expr., % | | | Rel. Expr., % |
| | 1.3dx4tm5495 | | Rel. Expr., % | 4Dtm1910f_{—} |
| Tissue Name | f_ag770_b2 | Tissue Name | 4dtm1843f_ag770 | ag770 |
| Liver | 0.0 | 93768_Secondary | 0.0 | 0.0 |
| adenocarcinoma | | Th1_anti-CD28/anti-CD3 | | |
| Pancreas | 0.0 | 93769_Secondary | 6.4 | 0.0 |
| | | Th2_anti-CD28/anti-CD3 | | |
| Pancreatic ca. | 0.0 | 93770_Secondary | 3.1 | 0.0 |
| CAPAN 2 | | Tr1_anti-CD28/anti-CD3 | | |
| Adrenal gland | 0.0 | 93573_Secondary | 0.0 | 0.0 |
| | | Th1_resting day 4-6 in IL-2 | | |
| Thyroid | 0.0 | 93572_Secondary | 0.0 | 0.0 |
| | | Th2_resting day 4-6 in IL-2 | | |
| Salivary gland | 0.0 | 93571_Secondary | 0.0 | 0.0 |
| | | Tr1_resting day 4-6 in IL-2 | | |
| Pituitary gland | 0.0 | 93568_primary Th1_anti- | 3.7 | 0.0 |
| | | CD28/anti-CD3 | | |
| Brain (fetal) | 0.0 | 93569_primary Th2_anti- | 0.0 | 0.0 |
| | | CD28/anti-CD3 | | |
| Brain (whole) | 0.0 | 93570_primary Tr1_anti- | 0.0 | 0.0 |
| | | CD28/anti-CD3 | | |
| Brain | 0.0 | 93565_primary | 0.0 | 4.8 |
| (amygdala) | | Th1_resting dy 4-6 in IL-2 | | |
| Brain | 0.2 | 93566_primary | 0.0 | 2.2 |
| (cerebellum) | | Th2_resting dy 4-6 in IL-2 | | |
| Brain | 0.1 | 93567_primary Tr1_resting | 0.0 | 7.0 |
| (hippocampus) | | dy 4-6 in IL-2 | | |
| Brain | 0.0 | 93351_CD45RA CD4 | 3.1 | 0.0 |
| (substantia | | lymphocyte_anti- | | |
| nigra) | | CD28/anti-CD3 | | |
| Brain | 0.0 | 93352_CD45RO CD4 | 0.0 | 0.0 |
| (thalamus) | | lymphocyte_anti-1 | | |
| | | CD28/anti-CD3 | | |
| Cerebral Cortex | 0.0 | 93251_CD8 | 1.4 | 0.0 |
| | | Lymphocytes_anti- | | |
| | | CD28/anti-CD3 | | |
| Spinal cord | 0.0 | 93353_chronic CD8 | 0.0 | 0.0 |
| | | Lymphocytes 2ry_resting | | |
| | | day 4-6 in IL-2 | | |
| CNS ca. | 0.0 | 93574_chronic CD8 | 0.0 | 0.0 |
| (glio/astro) | | Lymphocytes | | |
| U87-MG | | 2ry_activated CD3/CD28 | | |
| CNS ca. | 0.0 | 93354_CD4_none | 0.0 | 0.0 |
| (glio/astro) U- | | | | |
| 118-MG | | | | |
| CNS ca. (astro) | 0.0 | 93252_Secondary | 0.0 | 41.2 |
| SW1783 | | Th1/Th2/Tr1_anti-CD95 | | |
| | | CH11 | | |
| CNS ca.* | 0.0 | 93103_LAK cells_resting | 0.0 | 0.0 |
| (neuro; met) | | | | |
| SK-N-AS | | | | |
| CNS ca. (astro) | 0.0 | 93788_LAK cells_IL-2 | 0.0 | 0.0 |
| SF-539 | | | | |
| CNS ca. (astro) | 0.0 | 93787_LAK cells_IL- | 0.0 | 0.0 |
| SNB-75 | | 2 + IL-12 | | |
| CNS ca. (glio) | 0.0 | 93789_LAK cells_IL- | 0.0 | 0.0 |
| SNB- 19 | | 2 + IFN gamma | | |
| CNS ca. (glio) | 0.0 | 93790_LAK cells_IL-2 + | 0.0 | 0.0 |
| U251 | | IL-18 | | |
| CNS ca. (glio) | 0.0 | 93104_LAK | 0.0 | 5.9 |
| SF-295 | | cells_PMA/ionomycin and | | |
| | | IL-18 | | |
| Heart (fetal) | 0.0 | 93578_NK Cells IL- | 0.0 | 0.0 |
| | | 2_resting | | |
| Heart | 0.3 | 93109_Mixed Lymphocyte | 6.0 | 0.0 |
| | | Reaction_Two Way MLR | | |
| Fetal Skeletal | 0.0 | 93110_Mixed Lymphocyte | 0.0 | 0.0 |
| | | Reaction_Two Way MLR | | |
| Skeletal muscle | 0.0 | 93111_Mixed Lymphocyte | 0.0 | 1.3 |
| | | Reaction_Two Way MLR | | |
| Bone marrow | 0.0 | 93112_Mononuclear Cells | 0.0 | 18.4 |
| | | (PBMCs)_resting | | |
| Thymus | 0.0 | 93113_Mononuclear Cells | 0.0 | 0.0 |
| | | (PBMCs)_PWM | | |
| Spleen | 0.0 | 93114_Mononuclear Cells | 0.0 | 6.6 |
| | | (PBMCs)_PHA-L | | |
| Lymph node | 0.0 | 93249_Ramos (B | 5.7 | 0.0 |
| | | cell)_none | | |
| Colorectal | 0.1 | 93250_Ramos (B | 0.0 | 0.0 |
| | | cell)_ionomycin | | |
| Stomach | 0.0 | 93349_B | 0.0 | 0.0 |
| | | lymphocytes_PWM | | |
| | | 8120607 | | |
| Kidney (fetal) | 0.0 | Kidney NAT Clontech | 0.0 | |
| | | 8120608 | | |
| Renal ca. | 0.0 | Kidney Cancer Clontech | 0.0 | |
| 786-0 | | 8120613 | | |
| Renal ca. | 0.0 | Kidney NAT Clontech | 0.0 | |
| A498 | | 8120614 | | |
| Renal ca. | 0.0 | Kidney Cancer Clontech | 0.0 | |
| RXF 393 | | 9010320 | | |
| Renal ca. | 0.0 | Kidney NAT Clontech | 0.0 | |
| ACHN | | 9010321 | | |
| Renal ca. | 0.3 | Normal Uterus GENPAK | 0.0 | |
| UO-31 | | 061018 | | |
| Renal ca. | 0.1 | Uterus Cancer GENPAK | 0.0 | |
| TK-10 | | 064011 | | |
| Liver | 100.0 | Normal Thyroid Clontech | 0.0 | |
| | | A+ 6570-1 | | |
| Liver (fetal) | 1.8 | Thyroid Cancer GENPAK | 0.0 | |
| | | 064010 | | |
| Liver ca. | 0.0 | Thyroid Cancer | 0.0 | |
| (hepatoblast) | | INVITROGEN A302152 | | |
| HepG2 | | | | |
| Lung | 0.0 | Thyroid NAT | 0.0 | |
| | | INVITROGEN A302153 | | |
| Lung (fetal) | 0.0 | Normal Breast GENPAK | 0.0 | |
| | | 061019 | | |
| Lung ca. (small | 0.0 | 84877 Breast Cancer | 0.0 | |
| cell) LX-1 | | (OD04566) | | |
| Lung ca. (small | 0.0 | 85975 Breast Cancer | 0.0 | |
| cell) NCI-H69 | | (OD04590-01) | | |
| Lung ca. (s. cell | 0.0 | 85976 Breast Cancer Mets | 0.0 | |
| var.) SHP-77 | | (OD04590-03) | | |
| Lung ca. (large | 0.0 | 87070 Breast Cancer | 0.0 | |
| cell)NCI-H460 | | Metastasis (OD04655-05) | | |
| Lung ca. (non-sm. | 0.1 | GENPAK Breast Cancer | 0.2 | |
| cell) A549 | | 064006 | | |
| Lung ca. (non- | 0.2 | Breast Cancer Res. Gen. | 0.2 | |
| s. cell) NCI-H23 | | 1024 | | |
| Lung ca (non-s. | 0.0 | Breast Cancer Clontech | 0.0 | |
| cell) | | | | |
| HOP-62 | | 9100266 | | |
| Lung ca. (non-s.cl) | 0.0 | Breast NAT Clontech | 0.0 | |
| NCI-H522 | | 9100265 | | |
| Lung ca. (squam.) | 0.0 | Breast Cancer | 0.0 | |
| SW 900 | | INVITROGEN A209073 | | |
| Lung ca. (squam.) | 0.0 | Breast NAT | 0.0 | |
| NCI-H596 | | INVITROGEN A2090734 | | |
| Mammary gland | 0.0 | Normal Liver GENPAK | 38.4 | |
| | | 061009 | | |
| Small intestine | 0.0 | 93350_B | 0.0 | 0.0 |
| | | lymphoytes_CD40L and | | |
| | | IL-4 | | |
| Colon ca. | 0.0 | 92665_EOL-1 | 0.0 | 3.2 |
| SW480 | | (Eosinophil)_dbcAMP | | |
| | | differentiated | | |
| Colon ca.* | 0.2 | 93248_EOL-1 | 0.0 | 0.0 |
| (SW480 | | (Eosinophil)_dbcAMP/PM | | |
| met)SW620 | | Aionomycin | | |
| Colon ca. | 0.0 | 93356_Dendritic | 0.0 | 0.0 |
| HT29 | | Cells_none | | |
| Colon ca. | 0.0 | 93355_Dendritic | 0.0 | 0.0 |
| HCT-116 | | Cells_LPS 100 ng/ml | | |
| Colon ca. | 0.0 | 93775_Dendritic | 0.0 | 0.0 |
| CaCo-2 | | Cells_anti-CD40 | | |
| 83219 CC Well | 0.0 | 93774_Monocytes resting | 0.0 | 0.0 |
| to Mod Diff | | | | |
| (ODO3866) | | | | |
| Colon ca. | 1.3 | 93776_Monocytes_LPS 50 ng/ml | 0.0 | 0.0 |
| HCC-2998 | | | | |
| Gastric ca.* | 0.2 | 93581_Macrophages_resting | 1.1 | 0.0 |
| (liver met) NCI- | | | | |
| N87 | | | | |
| Bladder | 0.0 | 93582_Macrophages_LPS | 0.0 | 5.9 |
| | | 100 ng/ml | | |
| Trachea | 0.0 | 93098_HUVEC | 0.0 | 0.0 |
| | | (Endothelial)_none | | |
| Kidney | 0.0 | 93099_HUVEC | 0.0 | 0.0 |
| | | (Endothelial)_starved | | |
| Kidney (fetal) | 0.0 | 93100_HUVEC | 0.0 | 0.0 |
| | | (Endothelial)_IL- 1b | | |
| Renal ca. | 0.0 | 93779_HUVEC | 0.0 | 0.0 |
| 786-0 | | (Endothelial)_IFN gamma | | |
| Renal ca. | 0.5 | 93102_HUVEC | 0.0 | 0.0 |
| A498 | | (Endothelial)_TNF alpha + | | |
| | | IFN gamma | | |
| Renal ca. | 0.0 | 93101_HUVEC | 0.0 | 0.0 |
| RXF 393 | | (Endothelial)_TNF alpha + | | |
| | | IL4 | | |
| Renal ca. | 0.0 | 93781_HUVEC | 0.0 | 0.0 |
| ACHN | | (Endothelial)_IL- 11 | | |
| Renal ca. | 0.0 | 93583_Lung | 0.0 | 0.0 |
| UO-31 | | Microvascular Endothelial | | |
| | | Cells_none | | |
| Renal ca. | 0.0 | 93584_Lung | 0.0 | 0.0 |
| TK-10 | | Microvascular Endothelial | | |
| | | Cells_TNFa (4 ng/ml) and | | |
| | | IL1b (1 ng/ml) | | |
| Liver | 100.0 | 92662_Microvascular | 0.0 | 0.0 |
| | | Dermal endothelium_none | | |
| Liver (fetal) | 0.3 | 92663_Microsvasular | 0.0 | 0.0 |
| | | Dermal endothelium_TNFa | | |
| | | (4 ng/ml) and IL1b (1 ng/ml) | | |
| Liver ca. | 0.0 | 93773_Bronchial | 0.0 | 2.2 |
| (hepatoblast) | | epithelium_TNFa (4 ng/ml) | | |
| HepG2 | | and IL1b (1 ng/ml)** | | |
| Lung | 0.0 | 93347_Small Airway | 0.0 | 0.0 |
| | | Epithelium_none | | |
| Lung (fetal) | 0.0 | 93348_Small Airway | 0.0 | 3.7 |
| | | Epithelium_TNFa (4 ng/ml) | | |
| | | and IL1b (1 ng/ml) | | |
| Lung ca. (small | 0.0 | 92668_Coronery Artery | 0.0 | 0.0 |
| cell) LX-1 | | SMC_resting | | |
| Lung ca. (small | 0.0 | 92669_Coronery Artery | 0.0 | 0.0 |
| cell) NCI-H69 | | SMC_TNFa (4 ng/ml) and | | |
| | | IL1b (1 ng/ml) | | |
| Lung ca. (s. cell | 0.0 | 93107_astrocytes_resting | 0.0 | 3.4 |
| var.) SHP-77 | | | | |
| Lung ca. (large | 0.0 | 93108_astrocytes TNFa (4 ng/ml) | 0.0 | 0.0 |
| cell)NCI-H460 | | and IL1b (1 ng/ml) | | |
| Lung ca. (non- | 0.0 | 92666_KU-812 | 0.0 | 1.6 |
| sm. cell) A549 | | (Basophil)_resting | | |
| Lung ca. (non- | 0.0 | 92667_KU-812 | 0.0 | 0.0 |
| s. cell) NCI-H23 | | (Basophil)_PMA/ionoycin | | |
| Lung ca. (non- | 0.0 | 93579_CCD1106 | 0.0 | 0.0 |
| s. cell) HOP-62 | | (Keratinocytes)_none | | |
| Lung ca. (non- | 0.0 | 93580_CCD1106 | 0.0 | 0.0 |
| s.cl) NCI-H522 | | (Keratinocytes)_TNFa and | | |
| | | IFNg** | | |
| Lung ca. | 0.5 | 93791_Liver Cirrhosis | 100.0 | 100.0 |
| (squam.) SW | | | | |
| 900 | | | | |
| Lung ca. | 0.0 | 93792_Lupus Kidney | 0.0 | 0.0 |
| (squam.) NCI- | | | | |
| H596 | | | | |
| Mammary gland | 0.0 | 93577_NCI-H292 | 0.0 | 0.0 |
| Breast ca.* (pl. | 0.0 | 93358_NCI-H292_IL-4 | 0.0 | 0.0 |
| effusion) MCF-7 | | | | |
| Breast ca.* | 0.0 | 93360_NCI-H292_IL-9 | 0.0 | 0.0 |
| (pl. ef) MDA- | | | | |
| MB-231 | | | | |
| Breast ca.* (pl. | 0.0 | 93359_NCI-H292_IL-13 | 0.0 | 0.0 |
| effusion) | | | | |
| T47D | | | | |
| Breast ca. | 0.0 | 93357_NCI-H292_IFN | 0.0 | 0.0 |
| BT-549 | | gamma | | |
| Breast ca. | 0.0 | 93777_HPAEC_- | 2.3 | 3.4 |
| MDA-N | | | | |
| Ovary | 0.0 | 93778_HPAEC_IL- | 0.0 | 0.0 |
| | | 1beta/TNA alpha | | |
| Ovarian ca. | 0.0 | 93254_Normal Human | 3.4 | 0.0 |
| OVCAR-3 | | Lung Fibroblast_none | | |
| Ovarian ca. | 0.0 | 93253_Normal Human | 0.0 | 0.0 |
| OVCAR-4 | | Lung Fibroblast_TNFa (4 ng/ml) | | |
| | | and IL-1b (1 ng/ml) | | |
| Ovarian ca. | 0.0 | 93257_Normal Human | 0.0 | 0.0 |
| OVCAR-5 | | Lung Fibroblast_IL-4 | | |
| Ovarian ca. | 0.0 | 93256_Normal Human | 0.0 | 0.0 |
| OVCAR-8 | | Lung Fibroblast_IL-9 | | |
| Ovarian ca. | 0.0 | 93255_Normal Human | 0.0 | 0.0 |
| IGROV-1 | | Lung Fibroblast_IL-13 | | |
| Ovarian ca.* | 0.0 | 93258_Normal Human | 0.0 | 0.0 |
| (ascites) SK- | | Lung Fibroblast_IFN | | |
| OV-3 | | gamma | | |
| Uterus | 0.5 | 93106_Dermal Fibroblasts | 0.0 | 0.0 |
| | | CCD1070_resting | | |
| Plancenta | 0.0 | 93361_Dermal Fibroblasts | 0.0 | 0.0 |
| | | CCD1070_TNF alpha 4 ng/ml | | |
| Prostate | 0.0 | 93105_Dermal Fibroblasts | 0.0 | 4.3 |
| | | CCD1070_IL- 1beta 1 ng/ml | | |
| Prostate ca.* | 0.0 | 93772_dermal | 0.0 | 7.9 |
| (bone met)PC-3 | | fibroblast_IFN gamma | | |
| Testis | 0.4 | 93771_dermal | 0.0 | 0.0 |
| | | fibroblast_IL-4 | | |
| Melanoma | 0.0 | 93259_IBD Colitis 1** | 6.0 | 13.1 |
| Hs688(A).T | | | | |
| Melanoma* | 0.0 | 93260_IBD Colitis 2 | 2.8 | 5.2 |
| (met) | | | | |
| Hs688(B).T | | | | |
| Melanoma | 0.3 | 93261_IBD Crohns | 0.0 | 1.9 |
| UACC-62 | | | | |
| Melanoma | 0.1 | 735010_Colon_normal | 1.3 | 3.2 |
| M14 | | | | |
| Melanoma | 0.0 | 735019_Lung_none | 14.9 | 0.0 |
| LOX IMVI | | | | |
| Melanoma* | 0.4 | 64028-1_Thymus_none | 2.3 | 7.3 |
| (met) SK- | | | | |
| MEL-5 | | | | |
| Adipose | 0.0 | 64030-1_Kidney_none | 0.0 | 0.0 |

Both probe/primer sets are specific for the sequence of gene Acc. No. MOL3. Unigene data at http://www.ncbi.nlm.nih.gov/UniGene/clust.cgi?ORG=Hs&CID=8509 and our RTQ-PCR panels 1.3D,2D and 4D indicate that this gene is specifically expressed by the liver and upregulated in some hepatocellular carcinomas (HCCs). There is evidence suggesting that the examination of the serum complements may be a useful tool for the detection of HCCs in liver cirrosis (LC) patients. (Takezaki E, Murakami S, Nishibayashi H, Kagawa K, Ohmori H. Gan No Rinsho October 1990;36(12):2119-22). Therefore the serum level of this protein can be used as a diagnostic marker to detect LC and HCC and antibodies directed against this protein can be a potential therapeutic agent against LC and HCC. In addition, this molecule can also serve as a specific marker for differentiating liver from other tissues.

MOL4

Expression of gene MOL4 was assessed using the primer-probe set Ag1611, described in Table 21

TABLE 21

Probe Name: Ag1611

| | | | | | |
|---|---|---|---|---|---|
| | | | | Start | SEQ ID |
| Primers | Sequences | Tm | Length | Position | NO: |

| | | | | | |
|---|---|---|---|---|---|
| Forward | 5′-ATATGCTGTGCTGCATTCAGT-3′ | 58.4 | 21 | 40 | 85 |
| Probe | TET-5′-CTGCCTGGTCAGTGAACAATTTCCTG-3′-TAMRA | 68.9 | 26 | 65 | 86 |
| Reverse | 5′-CAAGGCCACACTAGTCGTGTAG-3′ | 59.9 | 22 | 117 | 87 |

Expression of this gene in panels 1.3D, 4D and 2 is at very low to undetectable levels (Ct values>35) in a number of tissues.

MOL6

Expression of gene MOL6a was assessed using the primer-probe set Ag1167, described in Table 22. Results of the RTQ-PCR runs are shown in Tables 23 and 24.

TABLE 22

Probe Name: Ag1167

| | | | | | |
|---|---|---|---|---|---|
| | | | | Start | SEQ ID |
| Primers | Sequences | Tm | Length | Position | NO: |

| | | | | | |
|---|---|---|---|---|---|
| Forward | 5′-TCTTTGCTGACTCATCTGTTCA-3′ | 58.7 | 22 | 51 | 88 |
| Probe | TET-5′-AAGAAGACCCTGCTCCCTATTTGGTG-3′-TAMRA | 67.5 | 26 | 75 | 89 |
| Reverse | 5′-AGGGGTTGAAGTGAGACTTGAG-3′ | 59.8 | 22 | 104 | 90 |

TABLE 23
Panels 1.3D and 4D

| PANEL 1.3D | | PANEL 4D | |
|---|---|---|---|
| | Rel. Expr., % | | |
| | 1.3dx4tm5586t_{—} | | Rel. Expr., % |
| | ag1167_{—} | | 4Dtm1937t_{—} |
| Tissue Name | a1 | Tissue Name | ag1167 |
| Liver | 1.1 | 93768_Secondary Th1_anti-CD28/anti- | 1.7 |
| adenocarcinoma | | CD3 | |
| Pancreas | 0.0 | 93769_Secondary Th2_anti-CD28/anti- | 0.0 |
| | | CD3 | |
| Pancreatic ca. | 1.0 | 93770_Secondary Tr1_anti-CD28/anti- | 0.9 |
| CAPAN 2 | | CD3 | |
| Adrenal gland | 0.0 | 93573_Secondary Th1_resting day 4-6 | 2.7 |
| | | in IL-2 | |
| Thyroid | 0.0 | 93572_Secondary Th2_resting day 4-6 | 0.0 |
| | | in IL-2 | |
| Salivary gland | 0.0 | 93571_Secondary Tr1_resting day 4-6 | 0.0 |
| | | in IL-2 | |
| Pituitary gland | 2.1 | 93568_primary Th1_anti-CD28/anti- | 0.0 |
| | | CD3 | |
| Brain (fetal) | 4.1 | 93569_primary Th2_anti-CD28/anti- | 3.0 |
| | | CD3 | |
| Brain (whole) | 1.1 | 93570_primary Tr1_anti-CD28/anti- | 2.4 |
| | | CD3 | |
| Brain (amygdala) | 4.1 | 93565_primary Th1_resting dy 4-6 in | 5.3 |
| | | IL-2 | |
| Brain (cerebellum) | 6.7 | 93566_primary Th2_resting dy 4-6 in | 3.7 |
| | | IL-2 | |
| Brain (hippocampus) | 4.3 | 93567_primary Tr1_resting dy 4-6 in | 3.7 |
| | | IL-2 | |
| Brain (substantia | 5.4 | 93351_CD45RA CD4 | 1.6 |
| nigra) | | lymphocyte_anti-CD28/anti-CD3 | |
| Brain (thalamus) | 4.7 | 93352_CD45RO CD4 | 2.6 |
| | | lymphocyte_anti-CD28/anti-CD3 | |
| Cerebral Cortex | 1.3 | 93251_CD8 Lymphocytes_anti- | 0.9 |
| | | CD28/anti-CD3 | |
| Spinal cord | 1.6 | 93353_chronic CD8 Lymphocytes | 0.4 |
| | | 2ry_resting dy 4-6 in IL-2 | |
| CNS ca. (glio/astro) | 0.0 | 93574_chronic CD8 Lymphocytes | 0.9 |
| U87-MG | | 2ry_activated CD3/CD28 | |
| CNS ca. (glio/astro) | 0.0 | 93354_CD4_none | 1.0 |
| U-118-MG | | | |
| CNS ca. (astro) | 0.0 | 93252_Secondary Th1/Th2/Tr1_anti- | 2.5 |
| SW1783 | | CD95 CH11 | |
| CNS ca.* (neuro; met) | 0.0 | 93103_LAK cells_resting | 7.0 |
| SK-N-AS | | | |
| CNS ca. (astro) | 0.0 | 93788_LAK cells_IL-2 | 0.9 |
| SF-539 | | | |
| CNS ca. (astro) | 2.4 | 93787_LAK cells_IL-2 + IL-12 | 1.6 |
| SNB-75 | | | |
| CNS ca. (glio) | 4.3 | 93789_LAK cells_IL-2 + IFN gamma | 5.8 |
| SNB-19 | | | |
| CNS ca. (glio) | 1.3 | 93790_LAK cells_IL-2 + IL-18 | 1.7 |
| U251 | | | |
| CNS ca. (glio) | 0.0 | 93104_LAK cells_PMA/ionomycin | 0.0 |
| SF-295 | | and IL-18 | |
| Heart (fetal) | 0.0 | 93578_NK Cells IL-2_resting | 2.2 |
| Heart | 0.0 | 93109_Mixed Lymphocyte | 2.4 |
| | | Reaction_Two Way MLR | |
| Fetal Skeletal | 0.0 | 93110_Mixed Lymphocyte | 1.6 |
| | | Reaction_Two Way MLR | |
| Skeletal muscle | 1.0 | 93111_Mixed Lymphocyte | 0.0 |
| | | Reaction_Two Way MLR | |
| Bone marrow | 0.0 | 93112_Mononuclear Cells | 0.0 |
| | | (PBMCs)_resting | |
| Thymus | 0.0 | 93113_Mononuclear Cells | 6.0 |
| | | (PBMCs)_PWM | |
| Spleen | 16.0 | 93114_Mononuclear Cells | 0.9 |
| | | (PBMCs)_PHA-L | |
| Lymph node | 1.4 | 93249_Ramos (B cell)_none | 6.8 |
| Colorectal | 0.0 | 93250_Ramos (B cell)_ionomycin | 5.9 |
| Stomach | 0.0 | 93349_B lymphocytes_PWM | 3.8 |
| Small intestine | 2.9 | 93350_B lymphoytes_CD40L and IL-4 | 2.4 |
| Colon ca. | 0.0 | 92665_EOL-1 (Eosinophil)_dbcAMP | 82.9 |
| SW480 | | differentiated | |
| Colon ca.* (SW480 | 1.7 | 93248_EOL-1 | 100.0 |
| met)SW620 | | (Eosinophil)_dbcAMP/PMAionomycin | |
| Colon ca. | 0.0 | 93356_Dendritic Cells_none | 2.5 |
| HT29 | | | |
| Colon ca. | 3.9 | 93355_Dendritic Cells_LPS 100 ng/ml | 0.9 |
| HCT-116 | | | |
| Colon ca. | 1.0 | 93775_Dendritic Cells_anti-CD40 | 2.5 |
| CaCo-2 | | | |
| 83219 CC Well to | 0.0 | 93774_Monocytes_resting | 0.0 |
| Mod Diff (ODO3866) | | | |
| Colon ca. | 1.9 | 93776_Monocytes_LPS 50 ng/ml | 0.8 |
| HCC-2998 | | | |
| Gastric ca.* (liver | 2.3 | 93581_Macrophages_resting | 0.8 |
| met) NCI-N87 | | | |
| Bladder | 4.9 | 93582_Macrophages_LPS 100 ng/ml | 0.4 |
| Trachea | 0.0 | 93098_HUVEC (Endothelial)_none | 0.9 |
| Kidney | 1.8 | 93099_HUVEC (Endothelial)_starved | 1.5 |
| Kidney (fetal) | 2.3 | 93100_HUVEC (Endothelial)_IL-1b | 0.0 |
| Renal ca. | 0.0 | 93779_HUVEC (Endothelial)_IFN | 2.4 |
| 786-0 | gamma | | |
| Renal ca. | 0.9 | 93102_HUVEC (Endothelial)_TNF | 0.0 |
| A498 | | alpha + IFN gamma | |
| Renal ca. | 0.0 | 93101_HUVEC (Endothelial)_TNF | 0.0 |
| RXF 393 | | alpha + IL4 | |
| Renal ca. | 1.5 | 93781_HUVEC (Endothelial)_IL-11 | 0.9 |
| ACHN | | | |
| Renal ca. | 0.0 | 93583_Lung Microvascular Endothelial | 2.1 |
| UO-31 | | Cells_none | |
| Renal ca. | 4.0 | 93584_Lung Microvascular Endothelial | 1.3 |
| TK-10 | | Cells_TNFa (4 ng/ml) and IL1b (1 ng/ml) | |
| Liver | 0.0 | 92662_Microvascular Dermal | 1.2 |
| | | endothelium_none | |
| Liver (fetal) | 0.0 | 92663_Microsvasular Dermal | 1.5 |
| | | endothelium_TNFa (4 ng/ml) and IL1b | |
| | | (1 ng/ml) | |
| Liver ca. | 0.5 | 93773_Bronchial epithelium_TNFa (4 ng/ml) | 44.1 |
| (hepatoblast) HepG2 | | and IL1b (1 ng/ml)** | |
| Lung | 0.0 | 93347_Small Airway Epithelium_none | 5.6 |
| Lung (fetal) | 2.2 | 93348_Small Airway | 25.7 |
| | | Epithelium_TNFa (4 ng/ml) and IL1b | |
| | | (1 ng/ml) | |
| Lung ca. (small cell) | 5.2 | 92668_Coronery Artery SMC_resting | 0.7 |
| LX-1 | | | |
| Lung ca. (small cell) | 2.8 | 92669_Coronery Artery SMC_TNFa (4 ng/ml) | 0.0 |
| NCI-H69 | | and IL1b (1 ng/ml) | |
| Lung ca. (s. cell var.) | 0.8 | 93107_astrocytes_resting | 3.5 |
| SHP-77 | | | |
| Lung ca. (large | 2.0 | 93108_astrocytes_TNFa (4 ng/ml) and | 1 .4 |
| cell)NCI-H460 | | IL1b (1 ng/ml) | |
| Lung ca. (non-sm. | 1.8 | 92666_KU-812 (Basophil)_resting | 20.4 |
| cell) A549 | | | |
| Lung ca. (non-s. cell) | 4.7 | 92667_KU-812 | 34.2 |
| NCI-H23 | | (Basophil)_PMA/ionoycin | |
| Lung ca (non-s. cell) | 1.3 | 93579_CCD1106 (Keratinocytes)_none | 0.7 |
| HOP-62 | | | |
| Lung ca. (non-s.cl) | 0.0 | 93580_CCD1106 | 19.1 |
| NCI-H522 | | (Keratinocytes)_TNFa and IFNg** | |
| Lung ca. (squam.) | 0.0 | 93791_Liver Cirrhosis | 3.2 |
| SW 900 | | | |
| Lung ca. (squam.) | 2.2 | 93792_Lupus Kidney | 5.2 |
| NCI-H596 | | | |
| Mammary gland | 0.8 | 93577_NCI-H292 | 6.3 |
| Breast ca.* (pl. | 3.8 | 93358_NCI-H292_IL-4 | 7.4 |
| effusion) MCF-7 | | | |
| Breast ca.* (pl. ef) | 0.0 | 93360_NCI-H292_IL-9 | 6.5 |
| MDA-MB-231 | | | |
| Breast ca.* (pl. | 0.0 | 93359_NCI-H292_IL-13 | 8.8 |
| effusion) T47D | | | |
| Breast ca. | 0.0 | 93357_NCI-H292_IFN gamma | 4.2 |
| BT-549 | | | |
| Breast ca. | 0.0 | 93777_HPAEC_- | 0.9 |
| MDA-N | | | |
| Ovary | 0.0 | 93778_HPAEC_IL- 1beta/TNA alpha | 1.4 |
| Ovarian ca. | 0.0 | 93254_Normal Human Lung | 0.0 |
| OVCAR-3 | | Fibroblast_none | |
| Ovarian ca. | 1.6 | 93253_Normal Human Lung | 0.0 |
| OVCAR-4 | | Fibroblast_TNFa (4 ng/ml) and IL-1b | |
| | | (1 ng/ml) | |
| Ovarian ca. | 1.9 | 93257_Normal Human Lung | 1.5 |
| OVCAR-5 | | Fibroblast_IL-4 | |
| Ovarian ca. | 2.8 | 93256_Normal Human Lung | 0.0 |
| OVCAR-8 | | Fibroblast_IL-9 | |
| Ovarian ca. | 2.7 | 93255_Normal Human Lung | 19 |
| IGROV-1 | | Fibroblast_IL-13 | |
| Ovarian ca.* (ascites) | 5.6 | 93258_Normal Human Lung | 2.2 |
| SK-OV-3 | | Fibroblast_IFN gamma | |
| Uterus | 0.6 | 93106_Dermal Fibroblasts | 0.8 |
| | | CCD1070_resting | |
| Plancenta | 1.0 | 93361_Dermal Fibroblasts | 1.6 |
| | | CCD1070_TNF alpha 4 ng/ml | |
| Prostate | 0.0 | 93105_Dermal Fibroblasts | 0.0 |
| | | CCD1070_IL- 1beta 1 ng/ml | |
| Prostate ca.* (bone | 0.0 | 93772_dermal fibroblast_IFN gamma | 2.0 |
| met)PC-3 | | | |
| Testis | 100.0 | 93771_dermal fibroblast_IL-4 | 0.9 |
| Melanoma | 0.0 | 93259_IBD Colitis 1** | 21.0 |
| Hs688(A).T | | | |
| Melanoma* (met) | 0.0 | 93260_IBD Colitis 2 | 1.3 |
| Hs688(B).T | | | |
| Melanoma | 0.0 | 93261_IBD Crohns | 0.7 |
| UACC-62 | | | |
| Melanoma | 0.9 | 735010_Colon_normal | 0.7 |
| M14 | | | |
| Melanoma | 0.0 | 735019_Lung_none | 2.2 |
| LOX IMVI | | | |
| Melanoma* (met) | 0.0 | 64028-1_Thymus_none | 41.8 |
| SK-MEL-5 | | | |
| Adipose | 1.9 | 64030-1_Kidney_none | 6.0 |

TABLE 24
Panels 2D and 3D

| PANEL 2D | | PANEL 3D | |
|---|---|---|---|
| | Rel. Expr., % | | Rel. Expr., % |
| | 2Dtm2324t_{—} | | 3dtm5309t_{—} |
| Tissue Name | ag1167 | Tissue Name | ag1167 |
| Normal Colon | 2.9 | 94905_Daoy_Medulloblastoma/Cerebellum_sscDNA | 4.0 |
| GENPAK 061003 | | | |
| 83219 CC Well to | 2.5 | 94906_TE671_Medulloblastom/Cerebellum_sscDNA | 0.0 |
| Mod Diff(ODO3866) | | | |
| 83220 CC NAT | 18.9 | 94907_D283 | 8.4 |
| (ODO3866) | | Med_Medulloblastoma/Cerebellum_sscDNA | |
| 83221 CC Gr.2 | 2.2 | 94908_PFSK-1_Primitive | 7.0 |
| rectosigmoid | | Neuroectodermal/Cerebellum_sscDNA | |
| (ODO3868) | | | |
| 83222 CC NAT | 0.0 | 94909_XF-498_CNS_sscDNA | 0.0 |
| (ODO3868) | | | |
| 83235 CC Mod Diff | 6.3 | 94910_SNB-78_CNS/glioma_sscDNA | 10.2 |
| (ODO3920) | | | |
| 83236 CC NAT | 12.6 | 94911_SF- | 0.0 |
| (ODO3920) | | 268_CNS/glioblastoma_sscDNA | |
| 83237 CC Gr.2 | 0.0 | 94912_T98G_Glioblastoma_sscDNA | 0.0 |
| ascend colon | | | |
| (ODO3921) | | | |
| 83238 CC NAT | 7.7 | 96776_SK-N-SH_Neuroblastoma | 0.0 |
| (ODO3921) | | (metastasis)_sscDNA | |
| 83241 CC from | 2.9 | 94913_SF- | 0.0 |
| Partial Hepatectomy | | 295_CNS/glioblastoma_sscDNA | |
| (ODO4309) | | | |
| 83242 Liver NAT | 0.0 | 94914_Cerebellum_sscDNA | 66.0 |
| (ODO4309) | | | |
| 87472 Colon mets to | 5.0 | 96777_Cerebellum_sscDNA | 4.4 |
| lung (ODO4451-01) | | | |
| 87473 Lung NAT | 9.9 | 94916_NCI-H292_Mucoepidermoid | 7.3 |
| (OD04451-02) | | lung carcinoma_sscDNA | |
| Normal Prostate | 17.4 | 94917_DMS-114_Small cell lung | 0.0 |
| Clontech A+ 6546-1 | | cancer_sscDNA | |
| 84140 Prostate | 2.7 | 94918_DMS-79_Small cell lung | 100.0 |
| Cancer (OD04410) | | cancer/neuroendocrine_sscDNA | |
| 84141 Prostate NAT | 26.2 | 94919_NCI-H146_Small cell lung | 16.4 |
| (OD04410) | | cancer/neuroendocrine_sscDNA | |
| 87073 Prostate | 20.6 | 94920_NCI-H526_Small cell lung | 2.1 |
| Cancer (OD04720-01) | | cancer/neuroendocrine_sscDNA | |
| 87074 Prostate NAT | 13.9 | 94921_NCI-N417_Small cell lung | 0.0 |
| (ODO4720-02) | | cancer/neuroendocrine_sscDNA | |
| Normal Lung | 17.7 | 94923_NCI-H82_Small cell lung | 3.5 |
| GENPAK 061010 | | cancer/neuroendocrine_sscDNA | |
| 83239 Lung Met to | 5.0 | 94924_NCI-H157_Squamous cell lung | 0.0 |
| Muscle (ODO4286) | | cancer (metastasis)_sscDNA | |
| 83240 Muscle NAT | 2.6 | 94925_NCI-H1155_Large cell lung | 0.0 |
| (ODO4286) | | cancer/neuroendocrine_sscDNA | |
| 84136 Lung | 4.9 | 94926_NCI-H1299_Large cell lung | 11.3 |
| Malignant Cancer | | cancer/neuroendocrine_sscDNA | |
| (OD03126) | | | |
| 84137 Lung NAT | 0.0 | 94927_NCI-H727_Lung | 3.0 |
| (OD03126) | | carcinoid_sscDNA | |
| 84871 Lung Cancer | 5.5 | 94928_NCI-UMC-11_Lung | 10.1 |
| (OD04404) | | carcinoid_sscDNA | |
| 84872 Lung NAT | 2.4 | 94929_LX-1_Small cell lung | 0.0 |
| (OD04404) | | cancer_sscDNA | |
| 84875 Lung Cancer | 2.5 | 94930_Colo-205_Colon | 0.0 |
| (OD04565) | | cancer_sscDNA | |
| 84876 Lung NAT | 7.7 | 94931_KM12_Colon cancer_sscDNA | 4.9 |
| (OD04565) | | | |
| 85950 Lung Cancer | 18.4 | 94932_KM20L2_Colon | 0.0 |
| (OD04237-01) | | cancer_sscDNA | |
| 85970 Lung NAT | 7.8 | 94933_NCI-H716_Colon | 0.0 |
| (OD04237-02) | | cancer_sscDNA | |
| 83255 Ocular Mel | 0.0 | 94935_SW-48_Colon | 0.0 |
| Met to Liver | | adenocarcinoma_sscDNA | |
| (ODO4310) | | | |
| 83256 Liver NAT | 0.0 | 94936_SW1116_Colon | 1.6 |
| (ODO4310) | | adenocarcinoma_sscDNA | |
| 84139 Melanoma | 3.0 | 94937_LS 174T_Colon | 0.0 |
| Mets to Lung | | adenocarcinoma_sscDNA | |
| (OD04321) | | | |
| 84138 Lung NAT | 2.6 | 94938_SW-948_Colon | 4.4 |
| (OD04321) | | adenocarcinoma_sscDNA | |
| Normal Kidney | 100.0 | 94939_SW-480_Colon | 0.0 |
| GENPAK 061008 | | adenocarcinoma_sscDNA | |
| 83786 Kidney Ca, | 56.6 | 94940_NCI-SNU-5_Gastric | 0.0 |
| Nuclear grade 2 | | carcinoma_sscDNA | |
| (OD04338) | | | |
| 83787 Kidney NAT | 23.2 | 94941_KATO III_Gastric | 2.6 |
| (OD04338) | | carcinoma_sscDNA | |
| 83788 Kidney Ca | 0.0 | 94943_NCI-SNU-16_Gastric | 3.6 |
| Nuclear grade 1/2 | | carcinoma_sscDNA | |
| (OD04339) | | | |
| 83789 Kidney NAT | 46.0 | 94944_NCI-SNU-1_Gastric | 0.0 |
| (OD04339) | | carcinoma_sscDNA | |
| 83790 Kidney Ca, | 12.9 | 94946_RF-1_Gastric | 0.0 |
| Clear cell type | | adenocarcinoma_sscDNA | |
| (OD04340) | | | |
| 83791 Kidney NAT | 24.5 | 94947_RF-48_Gastric | 0.0 |
| (OD04340) | | adenocarcinoma_sscDNA | |
| 83792 Kidney Ca, | 4.5 | 96778_MKN-45_Gastric | 0.0 |
| Nuclear grade 3 | | carcinoma_sscDNA | |
| (OD04348) | | | |
| 83793 Kidney NAT | 44.4 | 94949_NCI-N87_Gastric | 0.0 |
| (OD04348) | | carcinoma_sscDNA | |
| 87474 Kidney Cancer | 2.8 | 94951_OVCAR-5_Ovarian | 0.0 |
| (OD04622-01) | | carcinoma_sscDNA | |
| 87475 Kidney NAT | 0.0 | 94952_RL95-2_Uterine | 0.0 |
| (OD04622-03) | | carcinoma_sscDNA | |
| 85973 Kidney Cancer | 4.4 | 94953_HelaS3_Cervical | 0.0 |
| (OD04450-01) | | adenocarcinoma_sscDNA | |
| 85974 Kidney NAT | 24.8 | 94954_Ca Ski_Cervical epidermoid | 5.8 |
| (OD04450-03) | | carcinoma (metastasis)_sscDNA | |
| Kidney Cancer | 0.0 | 94955_ES-2_Ovarian clear cell | 0.0 |
| Clontech 8120607 | | carcinoma_sscDNA | |
| Kidney NAT | 0.0 | 94957_Ramos/6 h stim_”; Stimulated | 0.0 |
| Clontech 8120608 | | with PMA/ionomycin 6 h_sscDNA | |
| Kidney Cancer | 0.0 | 94958_Ramos/14 h stim_”; Stimulated | 0.0 |
| Clontech 8120613 | | with PMA/ionomycin 14 h_sscDNA | |
| Kidney NAT | 0.0 | 94962_MEG-01_Chronic myelogenous | 28.3 |
| Clontech 8120614 | | leukemia (megokaryoblast)_sscDNA | |
| Kidney Cancer | 2.6 | 94963_Raji_Burkitt's | 0.0 |
| Clontech 9010320 | | lymphoma_sscDNA | |
| Kidney NAT | 8.4 | 94964_Daudi_Burkitt's | 2.9 |
| Clontech 9010321 | | lymphoma_sscDNA | |
| Normal Uterus | 0.0 | 94965_U266_B-cell | 6.4 |
| GENPAK 061018 | | plasmacytoma/myeloma_sscDNA | |
| Uterus Cancer | 16.2 | 94968_CA46_Burkitt's | 0.0 |
| GENPAK 064011 | | lymphoma_sscDNA | |
| Normal Thyroid | 1.8 | 94970_RL_non-Hodgkin's B-cell | 0.0 |
| Clontech A+ 6570-1 | | lymphoma_sscDNA | |
| Thyroid Cancer | 27.2 | 94972_JM1_pre-B-cell | 6.7 |
| GENPAK 064010 | | lymphoma/leukemia_sscDNA | |
| Thyroid Cancer | 5.2 | 94973_Jurkat_T cell leukemia_sscDNA | 4.2 |
| INVITROGEN | | | |
| A302152 | | | |
| Thyroid NAT | 19.9 | 94974_TF-1_Erythroleukemia_sscDNA | 2.4 |
| INVITROGEN | | | |
| A302153 | | | |
| Normal Breast | 47.3 | 94975_HUT 78_T-cell | 2.8 |
| GENPAK 061019 | | lymphoma_sscDNA | |
| 84877 Breast Cancer | 7.0 | 94977_U937_Histiocytic | 6.6 |
| (OD04566) | | lymphoma_sscDNA | |
| 85975 Breast Cancer | 15.7 | 94980_KU-812_Myelogenous | 18.2 |
| (OD04590-01) | | leukemia_sscDNA | |
| 85976 Breast Cancer | 17.7 | 94981_769-P_Clear cell renal | 2.6 |
| Mets (OD04590-03) | | carcinoma_sscDNA | |
| 87070 Breast Cancer | 76.8 | 94983_Caki-2_Clear cell renal | 0.0 |
| Metastasis | | carcinoma_sscDNA | |
| (OD04655-05) | | | |
| GENPAK Breast | 42.6 | 94984_SW 839_Clear cell renal | 1.9 |
| Cancer 064006 | | carcinoma_sscDNA | |
| Breast Cancer Res. | 47.6 | 94986_G401_Wilms' tumor_sscDNA | 0.0 |
| Gen. 1024 | | | |
| Breast Cancer | 2.6 | 94987_Hs766T_Pancreatic carcinoma | 0.0 |
| Clontech 9100266 | | (LN metastasis)_sscDNA | |
| Breast NAT Clontech | 5.1 | 94988_CAPAN-1_Pancreatic | 0.0 |
| 9100265 | | adenocarcinoma (liver | |
| | | metastasis)_sscDNA | |
| Breast Cancer | 10.7 | 94989_SU86.86_Pancreatic carcinoma | 3.2 |
| INVITROGEN | | (liver metastasis)_sscDNA | |
| A209073 | | | |
| Breast NAT | 12.9 | 94990_BxPC-3_Pancreatic | 0.0 |
| INVITROGEN | | adenocarcinoma_sscDNA | |
| A2090734 | | | |
| Normal Liver | 13.1 | 94991_HPAC_Pancreatic | 3.6 |
| GENPAK 061009 | | adenocarcinoma_sscDNA | |
| Liver Cancer | 4.8 | 94992_MIA PaCa-2_Pancreatic | 0.0 |
| GENPAK 064003 | | carcinoma_sscDNA | |
| Liver Cancer | 2.5 | 94993_CFPAC-1_Pancreatic ductal | 13.6 |
| Research Genetics | | adenocarcinoma_sscDNA | |
| RNA 1025 | | | |
| Liver Cancer | 0.0 | 94994_PANC-1_Pancreatic epithelioid | 0.0 |
| Research Genetics | | ductal carcinoma_sscDNA | |
| RNA 1026 | | | |
| Paired Liver Cancer | 5.4 | 94996_T24_Bladder carcinma | 3.1 |
| Tissue Research | | (transitional cell)_sscDNA | |
| Genetics RNA 6004-T | | | |
| Paired Liver Tissue | 2.5 | 94997_5637_Bladder | 0.0 |
| Research Genetics | | carcinoma_sscDNA | |
| RNA 6004-N | | | |
| Paired Liver Cancer | 0.0 | 94998_HT-1197_Bladder | 20.2 |
| Tissue Research | | carcinoma_sscDNA | |
| Genetics RNA 6005-T | | | |
| Paired Liver Tissue | 0.0 | 94999_UM-UC-3_Bladder carcinoma | 0.0 |
| Research Genetics | | (transitional cell)_sscDNA | |
| RNA 6005-N | | | |
| Normal Bladder | 3.1 | 95000_A204_Rhabdomyosarcoma_ssc | 3.3 |
| GENPAK 061001 | | DNA | |
| Bladder Cancer | 0.0 | 95001_HT- | 5.2 |
| Research Genetics | | 1080_Fibrosarcoma_sscDNA | |
| RNA 1023 | | | |
| Bladder Cancer | 16.7 | 95002_MG-63_Osteosarcoma | 0.0 |
| INVITROGEN | | (bone)_sscDNA | |
| A302173 | | | |
| 87071 Bladder | 0.0 | 95003_SK-LMS-1_Leiomyosarcoma | 7.9 |
| Cancer (OD04718- | | (vulva)_sscDNA | |
| 01) | | | |
| 87072 Bladder | 2.9 | 95004_SJRH30_Rhabdomyosarcoma | 0.0 |
| Normal Adjacent | | (met to bone marrow)_sscDNA | |
| (OD04718-03) | | | |
| Normal Ovary Res. | 0.0 | 95005_A431_Epidermoid | 0.0 |
| Gen. | | carcinoma_sscDNA | |
| Ovarian Cancer | 12.6 | 95007_WM266-4_Melanoma_sscDNA | 0.0 |
| GENPAK 064008 | | | |
| 87492 Ovary Cancer | 2.9 | 95010_DU 145_Prostate carcinoma | 0.0 |
| (OD04768-07) | | (brain metastasis)_sscDNA | |
| 87493 Ovary NAT | 0.0 | 95012_MDA-MB-468_Breast | 0.0 |
| (OD04768-08) | | adenocarcinoma_sscDNA | |
| Normal Stomach | 5.9 | 95013_SCC-4_Squamous cell | 0.0 |
| GENPAK 061017 | | carcinoma of tongue_sscDNA | |
| Gastric Cancer | 0.0 | 95014_SCC-9_Squamous cell | 0.0 |
| Clontech 9060358 | | carcinoma of tongue_sscDNA | |
| NAT Stomach | 0.0 | 95015_SCC-15_Squamous cell | 0.0 |
| Clontech 9060359 | | carcinoma of tongue_sscDNA | |
| Gastric Cancer | 2.9 | 95017_CAL 27_Squamous cell | 0.0 |
| Clontech 9060395 | | carcinoma of tongue_sscDNA | |
| NAT Stomach | 0.0 | | |
| Clontech 9060394 | | | |
| Gastric Cancer | 0.0 | | |
| Clontech 9060397 | | | |
| NAT Stomach | 0.0 | | |
| Clontech 9060396 | | | |
| Gastric Cancer | 10.5 | | |
| GENPAK 064005 | | | |

Expression of gene MOL6 in panel 1.3D is detected in the testes and at very low levels in the spleen, but not in any other tissues. Expression in panel 2D indicates higher expression in normal kidney and markedly lower expression in kidney cancer. This indicates a potential role for this gene as a protein therapeutic in cases of kidney cancer. In panel 3D, expression is seen to be highest in a specimen of small cell lung cancer, chronic myelogenous leukemia and bladder cancer.

Expression of gene MOL6 in Panel 4D is up-regulated in keratinocytes and small airway epithelium after treatment with TNF alpha and IL-1 beta. It is also upregulated in eosinophils regardless of treatment and in normal thymus.

Potential Role(s) of MOL6 in Inflammation: The expression pattern of GM_{—}87760758_A shows that it is induced in keratinocytes and small airway epithelium in response to pro-inflammatory cytokines. Thus the protein in question may contribute to tissue destruction in the airways, recruitment of leukocytes, and tissue remodeling (Reichart et al., 1996 J. Pathol. 178 (2): 215-20).

Impact of Therapeutic Targeting of MOL6: Antibodies or small molecule therapeutics to MOL6 may reduce or inhibit tissue damage due to inflammation in psoriasis, asthma and other mast cell-mediated diseases both in the skin and in the airways. The results are also suggestive of a potential role for MOL6 in the treatment for emphysema (Rice et al., 1998 Curr Pharm Des 4(5): 381-96).

MOL7

Expression of gene MOL7 was assessed using the primer-probe sets Ag1876, described in Table 25. Results of the RTQ-PCR runs are shown in Table 26.

TABLE 25

Probe name: Ag1876

| | | | | Start | SEQ ID |
|---|---|---|---|---|---|
| Primers | Sequences | Tm | Length | Position | NO: |
| Forward | 5′-AGCAAGATTGCTCACACAGAGT-3′ | 59.2 | 22 | 668 | 91 |
| Probe | TET-5′-CCAGTCAATACCATCATCATCCATGAGG-3′-TAMRA | 69.1 | 28 | 692 | 92 |
| Reverse | 5′-TATGTTGTTGCTCATGGAGTTG-3′ | 58.7 | 22 | 730 | 93 |

TABLE 26
Panel 1.3D

| | Rel. Expr., % | | Rel. Expr., % |
|---|---|---|---|
| | 1.3dx4tm5422t_{—} | | 1.3dx4tm5422t_{—} |
| Tissue Name | ag1876_al | Tissue Name | ag1876_al |
| Liver | 0.0 | Kidney (fetal) | 0.0 |
| adenocarcinoma | | | |
| Pancreas | 0.0 | Renal ca. 786-0 | 0.0 |
| Pancreatic ca. | 0.4 | Renal ca. A498 | 0.5 |
| CAPAN 2 | | | |
| Adrenal gland | 0.7 | Renal ca. RXF 393 | 0.0 |
| Thyroid | 0.0 | Renal ca. ACHN | 0.6 |
| Salivary gland | 0.4 | Renal ca. UO-31 | 0.0 |
| Pituitary gland | 0.0 | Renal ca. TK-10 | 0.4 |
| Brain (fetal) | 0.3 | Liver | 0.0 |
| Brain (whole) | 4.2 | Liver (fetal) | 0.0 |
| Brain (amygdala) | 2.5 | Liver ca. (hepatoblast) HepG2 | 0.0 |
| Brain (cerebellum) | 0.8 | Lung | 0.0 |
| Brain (hippocampus) | 2.7 | Lung (fetal) | 0.0 |
| Brain (substantia | 1.4 | Lung ca. (small cell) LX-1 | 1.3 |
| nigra) | | | |
| Brain (thalamus) | 1.6 | Lung ca. (small cell) NCI- | 0.0 |
| | | H69 | |
| Cerebral Cortex | 0.6 | Lung ca. (s. cell var.) SHP-77 | 0.0 |
| Spinal cord | 0.4 | Lung ca. (large cell)NCI-H460 | 0.4 |
| CNS ca. (glio/astro) | 0.0 | Lung ca. (non-sm. cell) A549 | 0.5 |
| U87-MG | | | |
| CNS ca. (glio/astro) | 0.2 | Lung ca. (non-s. cell) NCI-H23 | 0.0 |
| U-118-MG | | | |
| CNS ca. (astro) | 0.0 | Lung ca. (non-s. cell) HOP-62 | 0.0 |
| SW1783 | | | |
| CNS ca.* (neuro; met) | 0.7 | Lung ca. (non-s.cl) NCI-H522 | 0.3 |
| SK-N-AS | | | |
| CNS ca. (astro) | 0.0 | Lung ca. (squam.) SW 900 | 0.8 |
| SF-539 | | | |
| CNS ca. (astro) | 0.0 | Lung ca. (squam.) NCI-H596 | 0.0 |
| SNB-75 | | | |
| CNS ca. (glio) | 2.2 | Mammary gland | 0.0 |
| SNB-19 | | | |
| CNS ca. (glio) | 1.3 | Breast ca.* (pl. effusion) | 2.9 |
| U251 | | MCF-7 | |
| CNS ca. (glio) | 0.4 | Breast ca.* (pl. ef) MDA-MB- | 0.0 |
| SF-295 | | 231 | |
| Heart (fetal) | 0.0 | Breast ca.* (pl. effusion) | 0.0 |
| | | T47D | |
| Heart | 0.0 | Breast ca. BT- | 0.6 |
| | | 549 | |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 1.0 | Ovarian ca. OVCAR-4 | 0.0 |
| Spleen | 0.0 | Ovarian ca. OVCAR-5 | 0.3 |
| Lymph node | 0.5 | Ovarian ca. OVCAR-8 | 0.8 |
| Colorectal | 0.0 | Ovarian ca. IGROV-1 | 0.0 |
| Stomach | 0.4 | Ovarian ca.* (ascites) SK-OV-3 | 0.6 |
| Small intestine | 0.7 | Uterus | 0.0 |
| Colon ca. | 0.0 | Plancenta | 5.5 |
| SW480 | | | |
| Colon ca.* (SW480 | 0.6 | Prostate | 0.0 |
| met) SW620 | | | |
| Colon ca. | 0.0 | Prostate ca.* (bone met) PC-3 | 0.0 |
| HT29 | | | |
| Colon ca. | 0.3 | Testis | 100.0 |
| HCT-116 | | | |
| Colon ca. | 0.0 | Melanoma Hs688(A).T | 0.0 |
| CaCo-2 | | | |
| 83219 CC Well to | 0.0 | Melanoma* (met) Hs688(B).T | 0.0 |
| Mod Diff (ODO3866) | | | |
| Colon ca. | 0.2 | Melanoma UACC-62 | 1.4 |
| HCC-2998 | | | |
| Gastric ca.* (liver | 0.0 | Melanoma M14 | 0.0 |
| met) NCI-N87 | | | |
| Bladder | 0.0 | Melanoma LOX IMVI | 0.0 |
| Trachea | 32.3 | Melanoma* (met) SK-MEL-5 | 0.0 |
| Kidney | 0.0 | Adipose | 8.2 |

Expression of MOL7 in panel 1.3D was highest in testis, followed by trachea. Expression in the brain is at much lower levels. This molecule may therefore have a role in male fertility. There was low to undetectable expression in the samples of panel 4D (CT values>35).

MOL8b

Expression of gene MOL8b was assessed using the primer-probe sets Ag3183, described in Table 27. Results of the RTQ-PCR runs are shown in Table 28.

TABLE 27

Probe name: Ag3183

| | | | | Start | SEQ ID |
|---|---|---|---|---|---|
| Primers | Sequences | Tm | Length | Position | NO: |
| Forward | 5′-AAGGGGACGAGTGTGGGATT-3′ | 62 | 20 | 211 | 94 |
| Probe | TET-5′-TGGCACCGAAGTAGCCGTGGCG-3′-TAMRA | 74 | 22 | 301 | 95 |
| Reverse | 5′-GCGGGCACTTGGTGTCGCA-3′ | 64 | 19 | 325 | 96 |

TABLE 28
Panel 4D

| | Rel. Expr., % | | Rel. Expr., % |
|---|---|---|---|
| | 4dx4tm4998t_{—} | | 4dx4tm4998t_{—} |
| Tissue Name | ag3183_a2 | Tissue Name | ag3183_a2 |
| 93768_Secondary Th1_anti- | 0.0 | 93100_HUVEC | 5.7 |
| CD28/anti-CD3 | | (Endothelial)_IL-1b | |
| 93769_Secondary Th2_anti- | 0.0 | 93779_HUVEC | 18.4 |
| CD28/anti-CD3 | | (Endothelial)_IFN | |
| | | gamma | |
| 93770_Secondary Tr1_anti- | 0.0 | 93102_HUVEC | 9.6 |
| CD28/anti-CD3 | | (Endothelial)_TNF | |
| | | alpha + IFN gamma | |
| 93573_Secondary Th1_resting | 0.0 | 93101_HUVEC | 11.2 |
| day 4-6 in IL-2 | | (Endothelial)_TNF | |
| | | alpha + IL4 | |
| 93572_Secondary Th2_resting | 0.0 | 93781_HUVEC | 19.3 |
| day 4-6 in IL-2 | | (Endothelial)_IL-11 | |
| 93571_Secondary Tr1_resting | 2.5 | 93583_Lung | 21.6 |
| day 4-6 in IL-2 | | Microvascular | |
| | | Endothelial Cells_none | |
| 93568_primary Th1_anti- | 0.0 | 93584_Lung | 32.1 |
| CD28/anti-CD3 | | Microvascular | |
| | | Endothelial | |
| | | Cells_TNFa (4 ng/ml) | |
| | | and IL1b (1 ng/ml) | |
| 93569_primary Th2_anti- | 0.0 | 92662_Microvascular | 10.7 |
| CD28/anti-CD3 | | Dermal | |
| | | endothelium_none | |
| 93570_primary Tr1_anti- | 0.0 | 92663_Microsvasular | 5.0 |
| CD28/anti-CD3 | | Dermal | |
| | | endothelium_TNFa (4 ng/ml) | |
| | | and IL1b (1 ng/ml) | |
| 93565_primary Th1_resting dy | 0.0 | 93773_Bronchial | 11.6 |
| 4-6 in IL-2 | | epithelium_TNFa (4 ng/ml) | |
| | | and IL1b (1 ng/ml)** | |
| 93566_primary Th2_resting dy | 2.3 | 93347_Small Airway | 5.6 |
| 4-6 in IL-2 | | Epithelium_none | |
| 93567_primary Tr1_resting dy | 0.7 | 93348_Small Airway | 4.8 |
| 4-6 in IL-2 | | Epithelium_TNFa (4 ng/ml) | |
| | | and IL1b (1 ng/ml) | |
| 93351_CD45RA CD4 | 29.9 | 92668_Coronery | 46.8 |
| lymphocyte_anti-CD28/anti- | | Artery SMC_resting | |
| CD3 | | | |
| 93352_CD45RO CD4 | 0.0 | 92669_Coronery | 45.1 |
| lymphocyte_anti-CD28/anti- | | Artery SMC_TNFa (4 ng/ml) | |
| CD3 | | and IL1b (1ng/ml) | |
| 93251_CD8 | 0.0 | 93107_astrocytes_resting | 82.4 |
| Lymphocytes_anti-CD28/anti- | | | |
| CD3 | | | |
| 93353_chronic CD8 | 0.0 | 93108_astrocytes_TNFa | 74.9 |
| Lymphocytes 2ry_resting dy 4-6 | | (4 ng/ml) and IL1b (1 ng/ml) | |
| in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.4 | 92667_KU-812 | 0.0 |
| | | (Basophil)_PMA/ionoycin | |
| 93252_Secondary | 2.1 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa | |
| | | and IFNg** | |
| 93788_LAK cells_IL-2 | 0.0 | 93791_Liver Cirrhosis | 5.6 |
| 93787_LAK cells_IL-2 + IL-12 | 0.0 | 93792_Lupus Kidney | 9.3 |
| 93789_LAK cells_IL-2 + IFN | 0.0 | 93577_NCI-H292 | 2.5 |
| gamma | | | |
| 93790_LAK cells_IL-2 + IL-18 | 0.0 | 93358_NCI-H292_IL-4 | 0.0 |
| 93104_LAK | 0.0 | 93360_NCI-H292_IL-9 | 0.0 |
| cells_PMA/ionomycin and IL- | | | |
| 18 | | | |
| 93578_NK Cells IL-2_resting | 0.0 | 93359_NCI-H292_IL- | 3.8 |
| | | 13 | |
| 93109_Mixed Lymphocyte | 0.0 | 93357_NCI-H292_IFN | 0.0 |
| Reaction_Two Way MLR | | gamma | |
| 93110_Mixed Lymphocyte | 0.0 | 93777_HPAEC_- | 31.9 |
| Reaction_Two Way MLR | | | |
| 93111_Mixed Lymphocyte | 0.0 | 93778_HPAEC_IL- | 22.4 |
| Reaction_Two Way MLR | | 1beta/TNA alpha | |
| 93112_Mononuclear Cells | 0.0 | 93254_Normal Human | 44.4 |
| (PBMCs)_resting | | Lung Fibroblast_none | |
| 93113_Mononuclear Cells | 1.8 | 93253_Normal Human | 28.3 |
| (PBMCs)_PWM | | Lung Fibroblast_TNFa | |
| | | (4 ng/ml) and IL-1b (1 ng/ml) | |
| 93114_Mononuclear Cells | 0.0 | 93257_Normal Human | 62.6 |
| (PBMCs)_PHA-L | | Lung Fibroblast_IL-4 | |
| 93249_Ramos (B cell)_none | 0.0 | 93256_Normal Human | 100.0 |
| | | Lung Fibroblast_IL-9 | |
| 93250_Ramos (B | 0.0 | 93255_Normal Human | 73.3 |
| cell)_ionomycin | | Lung Fibroblast_IL-13 | |
| 93349_B lymphocytes_PWM | 0.0 | 93258_Normal Human | 57.1 |
| | | Lung Fibroblast_IFN | |
| | | gamma | |
| 93350_B lymphoytes_CD40L | 0.0 | 93106_Dermal | 91.5 |
| and IL-4 | | Fibroblasts | |
| | | CCD1070_resting | |
| 92665_EOL-1 | 0.0 | 93361_Dermal | 31.1 |
| (Eosinophil)_dbcAMP | | Fibroblasts | |
| differentiated | | CCD1070_TNF alpha 4 ng/ml | |
| 93248_EOL-1 | 1.2 | 93105_Dermal | 69.7 |
| (Eosinophil)_dbcAMP/PMAionomycin | | Fibroblasts | |
| | | CCD1070_IL- 1beta 1 ng/ml | |
| 93356_Dendritic Cells_none | 1.8 | 93772_dermal | 18.3 |
| | | fibroblast_IFN gamma | |
| 93355_Dendritic Cells_LPS | 0.0 | 93771_dermal | 22.4 |
| 100 ng/ml | | fibroblast_IL-4 | |
| 93775_Dendritic Cells_anti- | 0.4 | 93259_IBD Colitis 1** | 3.2 |
| CD40 | | | |
| 93774_Monocytes_resting | 2.8 | 93260_IBD Colitis 2 | 0.0 |
| 93776_Monocytes_LPS 50 ng/ml | 0.0 | 93261_IBD Crohns | 0.8 |
| 93581_Macrophages_resting | 0.0 | 735010_Colon_normal | 8.9 |
| 93582_Macrophages_LPS 100 ng/ml | 0.0 | 735019_Lung_none | 18.5 |
| 93098_HUVEC | 8.4 | 64028-1_Thymus_none | 9.4 |
| (Endothelial)_none | | | |
| 93099_HUVEC | 29.9 | 64030-1_Kidney_none | 3.0 |
| (Endothelial)_starved | | | |

Expression of gene CG50889_{—}02 in two runs of panel 1.3D was not reproducible and is not considered further.

Expression of gene CG50889_{—}02 in Panel 4D: There is 30-fold increase in the expression of CG50889_{—}02 in activated naive T cells (CD4+ CD45RA cells) as compared to resting CD4 cells. This protein is expressed both in resting and activated fibroblasts, endothelium, and epithelium.

Potential Role(s) of CG50889-02 in Inflammation

MOL8b may be important in the initial activation of naïve T cells. Activated T cells initiate the inflammatory process by secreting cytokines and chemokines, which in turn induce B cell antibody production leading to the extravasation of leukocytes into inflammatory sites.

Impact of Therapeutic Targeting of MOL8b:

Antibody or small molecule therapeutics to MOL8b may block T cell activation in response to tissue transplant and reduce or block rejection. These therapeutic drugs may also reduce or prevent inflammation in asthma/allergy, psoriasis, arthritis and diabetes in which activated T cells play a pivotal role. Antibodies to MOL8b may also serve as a diagnostic or experimental tool to identify and differentiate naïve activated T cells from more differentiated T cell population (memory T cells).

MOL9a

Expression of gene MOL9a was assessed using the primer-probe set Ag673, described in Table 29. Results of the RTQ-PCR runs are shown in Tables 30 and 31.

TABLE 29

Probe name: Ag673

| | | | | | |
|---|---|---|---|---|---|
| | | | | Start | SEQ |
| | | | | Posi- | ID |
| Primers | Sequences | Tm | Length | tion | NO: |

| | | | | | |
|---|---|---|---|---|---|
| Forward | 5′- | 59.9 | 22 | 6 | 97 |
| | TAGAGTTGGTGGTTCCAGGAT | | | | |
| | T-3′ | | | | |
| Probe | FAM-5′- | 66.6 | 26 | 56 | 98 |
| | TGATGTCTCCTCTTCAGGCAA | | | | |
| | TGTCT-3′-TAMRA | | | | |
| Reverse | 5′- | 58.9 | 20 | 83 | 99 |
| | TCTGCCAGCCACAGTATAGG- | | | | |
| | 3′ | | | | |

TABLE 30
Panels 1.3D and 2D

| PANEL 1.3D | | | |
|---|---|---|---|
| | Rel. Expr., % | PANEL 2D | |
| | 1.3dx4tm5796f_{—} | | Rel. Expr., % |
| Tissue Name | ag673_b1 | Tissue Name | 2Dtm2310f_ag673 |
| Liver adenocarcinoma | 23.4 | Normal Colon GENPAK | 39.2 |
| | | 061003 | |
| Pancreas | 0.9 | 83219 CC Well to Mod Diff | 15.0 |
| | | (ODO3866) | |
| Pancreatic ca. | 8.4 | 83220 CC NAT (ODO3866) | 15.1 |
| CAPAN 2 | | | |
| Adrenal gland | 0.2 | 83221 CC Gr.2 rectosigmoid | 13.1 |
| | | (ODO3868) | |
| Thyroid | 0.9 | 83222 CC NAT (ODO3868) | 3.9 |
| Salivary gland | 0.2 | 83235 CC Mod Diff | 20.3 |
| | | (ODO3920) | |
| Pituitary gland | 0.4 | 83236 CC NAT (ODO3920) | 7.6 |
| Brain (fetal) | 8.2 | 83237 CC Gr.2 ascend colon | 36.3 |
| | | (ODO3921) | |
| Brain (whole) | 4.0 | 83238 CC NAT (ODO3921) | 4.8 |
| Brain (amygdala) | 1.5 | 83241 CC from Partial | 30.6 |
| | | Hepatectomy (ODO4309) | |
| Brain (cerebellum) | 2.2 | 83242 Liver NAT (ODO4309) | 20.3 |
| Brain (hippocampus) | 2.9 | 87472 Colon mets to lung | 41.5 |
| | | (OD04451-01) | |
| Brain (substantia nigra) | 1.6 | 87473 Lung NAT (OD04451- | 12.1 |
| | | 02) | |
| Brain (thalamus) | 1.6 | Normal Prostate Clontech A+ | 25.5 |
| | | 6546-1 | |
| Cerebral Cortex | 5.3 | 84140 Prostate Cancer | 22.5 |
| | | (OD04410) | |
| Spinal cord | 1.7 | 84141 Prostate NAT | 23.2 |
| | | (OD04410) | |
| CNS ca. (glio/astro) | 6.5 | 87073 Prostate Cancer | 10.7 |
| U87-MG | | (OD04720-01) | |
| CNS ca. (glio/astro) | 8.5 | 87074 Prostate NAT | 24.5 |
| U-118-MG | | (OD04720-02) | |
| CNS ca. (astro) | 21.6 | Normal Lung GENPAK | 27.0 |
| SW1783 | | 061010 | |
| CNS ca.* (neuro; met) | 12.6 | 83239 Lung Met to Muscle | 63.3 |
| SK-N-AS | | (ODO4286) | |
| CNS ca. (astro) | 3.9 | 83240 Muscle NAT | 50.7 |
| SF-539 | | (ODO4286) | |
| CNS ca. (astro) | 18.7 | 84136 Lung Malignant Cancer | 42.3 |
| SNB-75 | | (OD03126) | |
| CNS ca. (glio) | 4.3 | 84137 Lung NAT (OD03126) | 29.1 |
| SNB-19 | | | |
| CNS ca. (glio) | 17.7 | 84871 Lung Cancer | 69.3 |
| U251 | | (OD04404) | |
| CNS ca. (glio) | 12.6 | 84872 Lung NAT (OD04404) | 26.8 |
| SF-295 | | | |
| Heart (fetal) | 0.2 | 84875 Lung Cancer | 19.1 |
| | | (OD04565) | |
| Heart | 0.6 | 84876 Lung NAT (OD04565) | 12.2 |
| Fetal Skeletal | 0.8 | 85950 Lung Cancer | 94.0 |
| | | (OD04237-01) | |
| Skeletal muscle | 6.5 | 85970 Lung NAT (OD04237-02) | 20.3 |
| Bone marrow | 0.7 | 83255 Ocular Mel Met to | 74.2 |
| | | Liver (ODO4310) | |
| Thymus | 0.4 | 83256 Liver NAT (ODO4310) | 40.1 |
| Spleen | 0.3 | 84139 Melanoma Mets to | 36.3 |
| | | Lung (OD04321) | |
| Lymph node | 0.8 | 84138 Lung NAT (OD04321) | 29.7 |
| Colorectal | 1.2 | Normal Kidney GENPAK | 62.4 |
| | | 061008 | |
| Stomach | 0.2 | 83786 Kidney Ca, Nuclear | 66.0 |
| | | grade 2 (OD04338) | |
| Small intestine | 0.4 | 83787 Kidney NAT | 23.3 |
| | | (OD04338) | |
| Colon ca. | 10.2 | 83788 Kidney Ca Nuclear | 34.9 |
| SW480 | | grade 1/2 (OD04339) | |
| Colon ca.* (SW480 | 59.0 | 83789 Kidney NAT | 31.0 |
| met) SW620 | | (OD04339) | |
| Colon ca. | 8.4 | 83790 Kidney Ca, Clear cell | 45.7 |
| HT29 | | type (OD04340) | |
| Colon ca. | 14.7 | 83791 Kidney NAT | 33.0 |
| HCT-116 | | (OD04340) | |
| Colon ca. | 18.6 | 83792 Kidney Ca, Nuclear | 18.3 |
| CaCo-2 | | grade 3 (OD04348) | |
| 83219 CC Well to Mod | 2.2 | 83793 Kidney NAT | 42.9 |
| Diff (ODO3866) | | (OD04348) | |
| Colon ca. | 16.6 | 87474 Kidney Cancer | 14.5 |
| HCC-2998 | | (OD04622-01) | |
| Gastric ca.* (liver met) | 12.3 | 87475 Kidney NAT | 3.9 |
| NCI-N87 | | (OD04622-03) | |
| Bladder | 5.9 | 85973 Kidney Cancer | 70.2 |
| | | (OD04450-01) | |
| Trachea | 0.3 | 85974 Kidney NAT | 31.6 |
| | | (OD04450-03) | |
| Kidney | 1.8 | Kidney Cancer Clontech | 11.4 |
| | | 8120607 | |
| Kidney (fetal) | 7.8 | Kidney NAT Clontech | 7.1 |
| | | 8120608 | |
| Renal ca. | 8.5 | Kidney Cancer Clontech | 8.2 |
| 786-0 | | 8120613 | |
| Renal ca. | 7.0 | Kidney NAT Clontech | 8.1 |
| A498 | | 8120614 | |
| Renal ca. | 17.6 | Kidney Cancer Clontech | 18.3 |
| RXF 393 | | 9010320 | |
| Renal ca. | 3.9 | Kidney NAT Clontech | 33.4 |
| ACHN | | 9010321 | |
| Renal ca. | 12.8 | Normal Uterus GENPAK | 11.8 |
| UO-31 | | 061018 | |
| Renal ca. | 23.4 | Uterus Cancer GENPAK | 30.1 |
| TK-10 | | 064011 | |
| Liver | 1.4 | Normal Thyroid Clontech A+ | 17.3 |
| | | 6570-1 | |
| Liver (fetal) | 1.0 | Thyroid Cancer GENPAK | 39.2 |
| | | 064010 | |
| Liver ca. (hepatoblast) | 9.3 | Thyroid Cancer | 18.7 |
| HepG2 | | INVITROGEN A302152 | |
| Lung | 77.4 | Thyroid NAT INVITROGEN | 18.7 |
| | | A302153 | |
| Lung (fetal) | 1.7 | Normal Breast GENPAK | 32.3 |
| | | 061019 | |
| Lung ca. (small cell) | 57.5 | 84877 Breast Cancer | 40.6 |
| LX-1 | | (OD04566) | |
| Lung ca. (small cell) | 11.7 | 85975 Breast Cancer | 100.0 |
| NCI-H69 | | (OD04590-01) | |
| Lung ca. (s. cell var.) | 100.0 | 85976 Breast Cancer Mets | 76.3 |
| SHP-77 | | (OD04590-03) | |
| Lung ca. (large | 6.3 | 87070 Breast Cancer | 73.2 |
| cell) NCI-H460 | | Metastasis (OD04655-05) | |
| Lung ca. (non-sm. cell) | 28.7 | GENPAK Breast Cancer | 46.0 |
| A549 | | 064006 | |
| Lung ca. (non-s. cell) | 4.8 | Breast Cancer Res. Gen. 1024 | 31.2 |
| NCI-H23 | | | |
| Lung ca (non-s. cell) | 3.1 | Breast Cancer Clontech | 41.5 |
| HOP-62 | | 9100266 | |
| Lung ca. (non-s.cl) | 31.7 | Breast NAT Clontech | 13.4 |
| NCI-H522 | | 9100265 | |
| Lung ca. (squam.) | 19.4 | Breast Cancer INVITROGEN | 39.2 |
| SW 900 | | A209073 | |
| Lung ca. (squam.) | 27.4 | Breast NAT INVITROGEN | 39.8 |
| NCI-H596 | | A2090734 | |
| Mammary gland | 3.7 | Normal Liver GENPAK | 20.9 |
| | | 061009 | |
| Breast ca.* (pl. | 10.6 | Liver Cancer GENPAK | 15.6 |
| effusion) MCF-7 | | 064003 | |
| Breast ca.* (pl. ef) | 6.6 | Liver Cancer Research | 17.2 |
| MDA-MB-231 | | Genetics RNA 1025 | |
| Breast ca.* (pl. | 17.8 | Liver Cancer Research | 9.6 |
| effusion) T47D | | Genetics RNA 1026 | |
| Breast ca. | 15.0 | Paired Liver Cancer Tissue | 42.9 |
| BT-549 | | Research Genetics RNA | |
| | | 6004-T | |
| Breast ca. | 5.6 | Paired Liver Tissue Research | 18.6 |
| MDA-N | | Genetics RNA 6004-N | |
| Ovary | 1.4 | Paired Liver Cancer Tissue | 12.2 |
| | | Research Genetics RNA | |
| | | 6005-T | |
| Ovarian ca. | 8.4 | Paired Liver Tissue Research | 8.1 |
| OVCAR-3 | | Genetics RNA 6005-N | |
| Ovarian ca. | 2.6 | Normal Bladder GENPAK | 33.7 |
| OVCAR-4 | | 061001 | |
| Ovarian ca. | 44.9 | Bladder Cancer Research | 5.7 |
| OVCAR-5 | | Genetics RNA 1023 | |
| Ovarian ca. | 5.0 | Bladder Cancer | 23.5 |
| OVCAR-8 | | INVITROGEN A302173 | |
| Ovarian ca. | 5.2 | 87071 Bladder Cancer | 69.7 |
| IGROV-1 | | (OD04718-01) | |
| Ovarian ca.* (ascites) | 50.1 | 87072 Bladder Normal | 20.6 |
| SK-OV-3 | | Adjacent (OD04718-03) | |
| Uterus | 0.7 | Normal Ovary Res. Gen. | 2.9 |
| Placenta | 0.0 | Ovarian Cancer GENPAK | 18.6 |
| | | 064008 | |
| Prostate | 0.4 | 87492 Ovary Cancer | 53.2 |
| | | (OD04768-07) | |
| Prostate ca.* (bone | 1.9 | 87493 Ovary NAT | 13.5 |
| met) PC-3 | | (OD04768-08) | |
| Testis | 0.3 | Normal Stomach GENPAK | 15.0 |
| | | 061017 | |
| Melanoma | 2.1 | Gastric Cancer Clontech | 2.5 |
| Hs688(A).T | | 9060358 | |
| Melanoma* (met) | 2.3 | NAT Stomach Clontech | 8.3 |
| Hs688(B).T | | 9060359 | |
| Melanoma | 5.7 | Gastric Cancer Clontech | 19.1 |
| UACC-62 | | 9060395 | |
| Melanoma | 5.6 | NAT Stomach Clontech | 4.6 |
| M14 | | 9060394 | |
| Melanoma | 12.5 | Gastric Cancer Clontech | 29.5 |
| LOX IMVI | | 9060397 | |
| Melanoma* (met) SK- | 11.3 | NAT Stomach Clontech | 3.7 |
| MEL-5 | | 9060396 | |
| Adipose | 3.2 | Gastric Cancer GENPAK | 9.9 |
| | | 064005 | |

TABLE 31
Panels 3D and 4D

| PANEL 3D | | PANEL 4D | |
|---|---|---|---|
| | Rel. Expr., % | | Rel. Expr., % |
| | 3dx4tm5137f_{—} | | 4dtm4833f_{—} |
| Tissue Name | ag673_b1 | Tissue Name | ag673 |
| 94905_Daoy_Medulloblastoma/ | 13.6 | 93768_Secondary Th1_anti- | 11.9 |
| Cerebellum_sscDNA | | CD28/anti-CD3 | |
| 94906_TE671_Medulloblastom/ | 11.6 | 93769_Secondary Th2_anti- | 8.2 |
| Cerebellum_sscDNA | | CD28/anti-CD3 | |
| 94907_D283 | 53.3 | 93770_Secondary Tr1_anti- | 6.6 |
| Med_Medulloblastoma/Cerebellum_{—} | | CD28/anti-CD3 | |
| sscDNA | | | |
| 94908_PFSK-1_Primitive | 9.3 | 93573_Secondary Th1_resting | 0.0 |
| Neuroectodermal/Cerebellum_{—} | | day 4-6 in IL-2 | |
| sscDNA | | | |
| 94909_XF- | 7.8 | 93572_Secondary Th2_resting | 0.8 |
| 498_CNS_sscDNA | | day 4-6 in IL-2 | |
| 94910_SNB- | 8.6 | 93571_Secondary Tr1_resting | 0.5 |
| 78_CNS/glioma_sscDNA | | day 4-6 in IL-2 | |
| 94911_SF- | 14.1 | 93568_primary Th1_anti- | 17.7 |
| 268_CNS/glioblastoma_sscDNA | | CD28/anti-CD3 | |
| 94912_T98G_Glioblastoma_{—} | 15.3 | 93569_primary Th2_anti- | 9.1 |
| sscDNA | | CD28/anti-CD3 | |
| 96776_SK-N- | 15.1 | 93570_primary Tr1_anti- | 16.3 |
| SH_Neuroblastoma | | CD28/anti-CD3 | |
| (metastasis)_sscDNA | | | |
| 94913_SF- | 13.6 | 93565_primary Th1_resting dy | 7.9 |
| 295_CNS/glioblastoma_sscDNA | | 4-6 in IL-2 | |
| 94914_Cerebellum_sscDNA | 5.5 | 93566_primary Th2_resting dy | 2.6 |
| | | 4-6 in IL-2 | |
| 96777_Cerebellum_sscDNA | 0.9 | 93567_primary Tr1_resting dy | 4.3 |
| | | 4-6 in IL-2 | |
| 94916_NCI- | 36.5 | 93351_CD45RA CD4 | 13.4 |
| H292_Mucoepidermoid lung | | lymphocyte_anti-CD28/anti- | |
| carcinoma_sscDNA | | CD3 | |
| 94917_DMS-114_Small cell | 8.1 | 93352_CD45RO CD4 | 8.9 |
| lung cancer_sscDNA | | lymphocyte_anti-CD28/anti- | |
| | | CD3 | |
| 94918_DMS-79_Small cell | 100.0 | 93251_CD8 | 12.9 |
| lung | | Lymphocytes_anti-CD28/anti- | |
| cancer/neuroendocrine_sscDNA | | CD3 | |
| 94919_NCI-H146_Small cell | 32.1 | 93353_chronic CD8 | 10.5 |
| lung | | Lymphocytes 2ry_resting dy 4-6 | |
| cancer/neuroendocrine_sscDNA | | in IL-2 | |
| 94920_NCI-H526_Small cell | 30.0 | 93574_chronic CD8 | 5.2 |
| lung | | Lymphocytes 2ry_activated | |
| cancer/neuroendocrine_sscDNA | | CD3/CD28 | |
| 94921_NCI-N417_Small cell | 30.6 | 93354_CD4_none | 1.4 |
| lung | | | |
| cancer/neuroendocrine_sscDNA | | | |
| 94923_NCI-H82_Small cell | 22.6 | 93252_Secondary | 0.8 |
| lung | | Th1/Th2/Tr1_anti-CD95 CH11 | |
| cancer/neuroendocrine_sscDNA | | | |
| 94924_NCI- | 67.1 | 93103_LAK cells_resting | 18.0 |
| H157_Squamous cell lung | | | |
| cancer (metastasis)_sscDNA | | | |
| 94925_NCI-H1155_Large | 19.8 | 93788_LAK cells_IL-2 | 7.8 |
| cell lung | | | |
| cancer/neuroendocrine_sscDNA | | | |
| 94926_NCI-H1299_Large | 36.3 | 93787_LAK cells_IL-2 + IL-12 | 8.1 |
| cell lung | | | |
| cancer/neuroendocrine_sscDNA | | | |
| 94927_NCI-H727_Lung | 20.2 | 93789_LAK cells_IL-2 + IFN | 13.6 |
| carcinoid_sscDNA | | gamma | |
| 94928_NCI-UMC-11_Lung | 73.3 | 93790_LAK cells_IL-2 + IL-18 | 10.9 |
| carcinoid_sscDNA | | | |
| 94929_LX-1_Small cell lung | 20.2 | 93104_LAK | 4.8 |
| cancer_sscDNA | | cells_PMA/ionomycin and IL- | |
| | | 18 | |
| 94930_Colo-205_Colon | 11.6 | 93578_NK Cells IL-2_resting | 1.7 |
| cancer_sscDNA | | | |
| 94931_KM12_Colon | 31.6 | 93109_Mixed Lymphocyte | 13.0 |
| cancer_sscDNA | | Reaction_Two Way MLR | |
| 94932_KM20L2_Colon | 8.5 | 93110_Mixed Lymphocyte | 11.4 |
| cancer_sscDNA | | Reaction_Two Way MLR | |
| 94933_NCI-H716_Colon | 36.9 | 93111_Mixed Lymphocyte | 3.2 |
| cancer_sscDNA | | Reaction_Two Way MLR | |
| 94935_SW-48_Colon | 12.5 | 93112_Mononuclear Cells | 1.4 |
| adenocarcinoma_sscDNA | | (PBMCs)_resting | |
| 94936_SW1116_Colon | 9.0 | 93113_Mononuclear Cells | 23.7 |
| adenocarcinoma_sscDNA | | (PBMCs)_PWM | |
| 94937_LS 174T_Colon | 32.6 | 93114_Mononuclear Cells | 8.7 |
| adenocarcinoma_sscDNA | | (PBMCs)_PHA-L | |
| 94938_SW-948_Colon | 1.5 | 93249_Ramos (B cell)_none | 19.9 |
| adenocarcinoma_sscDNA | | | |
| 94939_SW-480_Colon | 6.7 | 93250_Ramos (B | 100.0 |
| adenocarcinoma_sscDNA | | cell)_ionomycin | |
| 94940_NCI-SNU-5_Gastric | 12.4 | 93349_B lymphocytes_PWM | 65.1 |
| carcinoma_sscDNA | | | |
| 94941_KATO III_Gastric | 55.9 | 93350_B lymphoytes_CD40L | 6.0 |
| carcinoma_sscDNA | | and IL-4 | |
| 94943_NCI-SNU-16_Gastric | 12.2 | 92665_EOL-1 | 12.5 |
| carcinoma_sscDNA | | (Eosinophil)_dbcAMP | |
| | | differentiated | |
| 94944_NCI-SNU-1_Gastric | 58.1 | 93248_EOL-1 | 4.4 |
| carcinoma_sscDNA | | (Eosinophil)_dbcAMP/PMAionomycin | |
| 94946_RF-1_Gastric | 11.4 | 93356_Dendritic Cells_none | 9.5 |
| adenocarcinoma_sscDNA | | | |
| 94947_RF-48_Gastric | 15.3 | 93355_Dendritic Cells_LPS | 8.7 |
| adenocarcinoma_sscDNA | | 100 ng/ml | |
| 96778_MKN-45_Gastric | 33.2 | 93775_Dendritic Cells_anti- | 12.2 |
| carcinoma_sscDNA | | CD40 | |
| 94949_NCI-N87_Gastric | 16.7 | 93774_Monocytes_resting | 14.8 |
| carcinoma_sscDNA | | | |
| 94951_OVCAR-5_Ovarian | 10.0 | 93776_Monocytes_LPS 50 ng/ml | 15.5 |
| carcinoma_sscDNA | | | |
| 94952_RL95-2_Uterine | 7.6 | 93581_Macrophages_resting | 35.4 |
| carcinoma_sscDNA | | | |
| 94953_HelaS3_Cervical | 11.1 | 93582_Macrophages_LPS 100 ng/ml | 4.4 |
| adenocarcinoma_sscDNA | | | |
| 94954_Ca Ski_Cervical | 28.9 | 93098_HUVEC | 27.0 |
| epidermoid carcinoma | | (Endothelial)_none | |
| (metastasis)_sscDNA | | | |
| 94955_ES-2_Ovarian clear | 15.5 | 93099_HUVEC | 39.2 |
| cell carcinoma_sscDNA | | (Endothelial)_starved | |
| 94957_Ramos/6 h stim_”; | 15.8 | 93100_HUVEC | 12.7 |
| Stimulated with | | (Endothelial)_IL-1b | |
| PMA/ionomycin 6 h_sscDNA | | | |
| 94958_Ramos/14 h stim_”; | 12.4 | 93779_HUVEC | 17.1 |
| Stimulated with | | (Endothelial)_IFN gamma | |
| PMA/ionomycin | | | |
| 14 h_sscDNA | | | |
| 94962_MEG-01_Chronic | 25.5 | 93102_HUVEC | 14.0 |
| myelogenous leukemia | | (Endothelial)_TNF alpha + | |
| (megokaryoblast)_sscDNA | | IFN gamma | |
| 94963_Raji_Burkitt's | 8.4 | 93101_HUVEC | 22.4 |
| lymphoma_sscDNA | | (Endothelial)_TNF alpha + IL4 | |
| 94964_Daudi_Burkitt's | 29.1 | 93781_HUVEC | 11.7 |
| lymphoma_sscDNA | | (Endothelial)_IL-11 | |
| 94965_U266_B-cell | 8.0 | 93583_Lung Microvascular | 14.7 |
| plasmacytoma/myeloma_sscDNA | | Endothelial Cells_none | |
| 94968_CA46_Burkitt's | 7.2 | 93584_Lung Microvascular | 19.2 |
| lymphoma_sscDNA | | Endothelial Cells_TNFa (4 ng/ml) | |
| | | and IL1b (1 ng/ml) | |
| 94970_RL_non-Hodgkin's B- | 10.7 | 92662_Microvascular Dermal | 26.8 |
| cell lymphoma_sscDNA | | endothelium_none | |
| 94972_JM1_pre-B-cell | 5.5 | 92663_Microsvasular Dermal | 18.2 |
| lymphoma/leukemia_sscDNA | | endothelium_TNFa (4 ng/ml) | |
| | | and IL1b (1 ng/ml) | |
| 94973_Jurkat_T cell | 15.5 | 93773_Bronchial | 2.4 |
| leukemia_sscDNA | | epithelium_TNFa (4 ng/ml) | |
| | | and IL1b (1 ng/ml)** | |
| 94974_TF- | 33.9 | 93347_Small Airway | 8.7 |
| 1_Erythroleukemia_sscDNA | | Epithelium_none | |
| 94975_HUT 78_T-cell | 23.7 | 93348_Small Airway | 37.9 |
| lymphoma_sscDNA | | Epithelium_TNFa (4 ng/ml) | |
| | | and IL1b (1 ng/ml) | |
| 94977_U937_Histiocytic | 29.4 | 92668_Coronery Artery | 17.4 |
| lymphoma_sscDNA | | SMC_resting | |
| 94980_KU- | 27.2 | 92669_Coronery Artery | 7.4 |
| 812_Myelogenous | | SMC_TNFa (4 ng/ml) and | |
| leukemia_sscDNA | | IL1b (1 ng/ml) | |
| 94981_769-P_Clear cell | 17.3 | 93107_astrocytes_resting | 13.1 |
| renal carcinoma_sscDNA | | | |
| 94983_Caki-2_Clear cell | 19.9 | 93108_astrocytes_TNFa (4 ng/ml) | 9.3 |
| renal carcinoma_sscDNA | | and IL1b (1 ng/ml) | |
| 94984_SW 839_Clear cell | 15.4 | 92666_KU-812 | 13.0 |
| renal carcinoma_sscDNA | | (Basophil)_resting | |
| 94986_G401_Wilms' | 19.5 | 92667_KU-812 | 26.8 |
| tumor_sscDNA | | (Basophil)_PMA/ionoycin | |
| 94987_Hs766T_Pancreatic | 12.7 | 93579_CCD1106 | 14.9 |
| carcinoma (LN | | (Keratinocytes)_none | |
| metastasis)_sscDNA | | | |
| 94988_CAPAN-1_Pancreatic | 11.1 | 93580_CCD1106 | 1.4 |
| adenocarcinoma (liver | | (Keratinocytes)_TNFa and | |
| metastasis)_sscDNA | | IFNg** | |
| 94989_SU86.86_Pancreatic | 38.5 | 93791_Liver Cirrhosis | 2.3 |
| carcinoma (liver | | | |
| metastasis)_sscDNA | | | |
| 94990_BxPC-3_Pancreatic | 8.2 | 93792_Lupus Kidney | 1.0 |
| adenocarcinoma_sscDNA | | | |
| 94991_HPAC_Pancreatic | 24.3 | 93577_NCI-H292 | 12.3 |
| adenocarcinoma_sscDNA | | | |
| 94992_MIA PaCa- | 5.6 | 93358_NCI-H292_IL-4 | 20.0 |
| 2_Pancreatic | | | |
| carcinoma_sscDNA | | | |
| 94993_CFPAC-1_Pancreatic | 32.2 | 93360_NCI-H292_IL-9 | 31.4 |
| ductal | | | |
| adenocarcinoma_sscDNA | | | |
| 94994_PANC-1_Pancreatic | 29.0 | 93359_NCI-H292_IL-13 | 13.7 |
| epithelioid ductal | | | |
| carcinoma_sscDNA | | | |
| 94996_T24_Bladder | 13.0 | 93357_NCI-H292_IFN gamma | 15.2 |
| carcinma (transitional | | | |
| cell)_sscDNA | | | |
| 94997_5637_Bladder | 18.4 | 93777_HPAEC_- | 14.7 |
| carcinoma_sscDNA | | | |
| 94998_HT-1197_Bladder | 25.1 | 93778_HPAEC_IL-1 | 12.7 |
| carcinoma_sscDNA | | beta/TNA alpha | |
| 94999_UM-UC-3_Bladder | 3.3 | 93254_Normal Human Lung | 8.0 |
| carcinma (transitional | | Fibroblast_none | |
| cell)_sscDNA | | | |
| 95000_A204_{—} | 16.1 | 93253_Normal Human Lung | 3.6 |
| Rhabdomyosarcoma_sscDNA | | Fibroblast_TNFa (4 ng/ml) and | |
| | | IL-1b (1 ng/ml) | |
| 95001_HT- | 16.7 | 93257_Normal Human Lung | 18.7 |
| 1080_Fibrosarcoma_sscDNA | | Fibroblast_IL-4 | |
| 95002_MG- | 12.9 | 93256_Normal Human Lung | 13.5 |
| 63_Osteosarcoma | | Fibroblast_IL-9 | |
| (bone)_sscDNA | | | |
| 95003_SK-LMS- | 34.7 | 93255_Normal Human Lung | 10.2 |
| 1_Leiomyosarcoma | | Fibroblast_IL-13 | |
| (vulva)_sscDNA | | | |
| 95004_SJRH30_Rhabdomyo | 16.9 | 93258_Normal Human Lung | 17.2 |
| sarcoma (met to bone | | Fibroblast_IFN gamma | |
| marrow)_sscDNA | | | |
| 95005_A431_Epidermoid | 9.4 | 93106_Dermal Fibroblasts | 39.0 |
| carcinoma_sscDNA | | CCD1070_resting | |
| 95007_WM266- | 6.9 | 93361_Dermal Fibroblasts | 47.0 |
| 4_Melanoma_sscDNA | | CCD1070_TNF alpha 4 ng/ml | |
| 95010_DU 145_Prostate | 0.3 | 93105_Dermal Fibroblasts | 15.7 |
| carcinoma (brain | | CCD1070_IL-1beta 1 ng/ml | |
| metastasis)_sscDNA | | | |
| 95012_MDA-MB- | 13.4 | 93772_dermal fibroblast_IFN | 5.6 |
| 468_Breast | | gamma | |
| adenocarcinoma_sscDNA | | | |
| 95013_SCC-4_Squamous | 0.1 | 93771_dermal fibroblast_IL-4 | 11.7 |
| cell carcinoma of | | | |
| tongue_sscDNA | | | |
| 95014_SCC-9_Squamous | 0.2 | 93259_IBD Colitis 1** | 0.1 |
| cell carcinoma of | | | |
| tongue_sscDNA | | | |
| 95015_SCC-15_Squamous | 0.8 | 93260_IBD Colitis 2 | 0.3 |
| cell carcinoma of | | | |
| tongue_sscDNA | | | |
| 95017_CAL 27_Squamous | 29.0 | 93261_IBD Crohns | 0.5 |
| cell carcinoma of | | | |
| tongue_sscDNA | | | |
| | | 735010_Colon_normal | 3.1 |
| | | 735019_Lung_none | 7.1 |
| | | 64028-1_Thymus_none | 9.4 |
| | | 64030-1_Kidney_none | 3.0 |

Panel 1.3D Description: The gene MOL9a is expressed in a number of tissues, including the central nervous system, lung, mammary gland and kidney. Moreover, its eexpression seems to be enhanced in tumor cell lines as compared to normal tissue in most cases with a good therapeutic window.

Panel 2D Description: Tissue distribution of this gene MOL9a in panel 2D confirms the results obtained in panel 1.3 D. There is enhanced expression of this gene MOL9a in tumor tissue as against the normal adjacent tissue, particularly in lung and kidney cancer, but also in cases of colon cancer, ovarian cancer, breast cancer, gastric cancer, bladder cancer, liver cancer and thyroid cancer. Some metastases, particularly the lung and those from melanoma express high levels of MOL9a. Corroborative information about the expression of this molecule is available in the form of ESTs, mostly from enodothelial cells, colon, ovarian tumors, pancreas and brain regions. Panel 3D demonstrates increased expression of this gene MOL9a in a variety of carcinomas, supporting the results of panels 1.3D and 2D thus demonstrating utility for this protein as an antibody target. Therefore antibodies specific to this protein may be used as a therapeutic in the treatment of various types of cancer.

Panel 4D Description: This gene MOL9a is upregulated in endothelium, and epithelium regardless of stimulus. There is also high level expression of this protein in ionomycin-treated B cell line and mitogen (pokeweed mitogen, PWM) treated B cells. Consistent with this finding Peripheral blood mononuclear cells (PBMC) treated with PWM also demonstrate increased expression of this molecule. Further, induction of MOL9a is seen in activated T cells. In PBMC the T cell specific mitogen (Phytohemagglutinin, PHA) induces the expression of this transcript and in acute and chronically activated T cells the expression of this transcript is increased as compared to untreated or resting T cells.

The MOL9a is induced in activated B and T lymphocytes and may thus have a potential role in inflammation by regulating lymphocyte trafficking, or activation, or increasing tissue destruction. This molecule may also serve as a marker for activated T or B cells.

Impact of Therapeutic Targeting of MOL9a: Small molecule or antibody therapies to the molecule encoded by MOL9a may inhibit tissue damage due to T or B cell activation and the bioactive molecules produced by these cell types. These diseases would include asthma/allergy, colitis, Crohn's disease, lupus, and arthritis. Alternatively, protein therapeutics based on this molecule could serve as an adjuvant and help boost the effectiveness of vaccines or regulate immune status during organ transplant. 19506719_B_EXT may also serve as a marker for activated T cells and serve as a diagnostic tool in determining the extent of inflammation in autoimmune diseases such as asthma/allergy, colitis, Crohn's disease, lupus, and arthritis.

MOL9b

Expression of gene MOL9b was assessed using the primer-probe set Ag2458, described in Table 32. Results of the RTQ-PCR runs are shown in Tables 33 and 34.

TABLE 32

Probe name: Ag2458

| | | | | | |
|---|---|---|---|---|---|
| | | | | Start | SEQ |
| | | | | Posi- | ID |
| Primers | Sequences | Tm | Lenght | tion | NO: |

| | | | | | |
|---|---|---|---|---|---|
| Forward | 5′-GTTGGTGGTTCCAGGAT | 58.7 | 20 | 58 | 100 |
| | TTT-3′ | | | | |
| Probe | TET-5′- | 66.6 | 26 | 104 | 101 |
| | TGATGTCTCCTCTTCAGGCA | | | | |
| | ATGTCT-3′-TAMRA | | | | |
| Reverse | 5′-CTGCCAGCCACAGTATA | 58.9 | 20 | 130 | 102 |
| | GGA-3′ | | | | |

TABLE 33
Panels 1.3D and 2D

| PANEL 1.3D | | | PANEL 2D | |
|---|---|---|---|---|
| | Rel. Expr., % | Rel. Expr., % | | Rel. Expr., % |
| | 1.3dtm4270t_{—} | 1.3dx4tm5407t_{—} | | 2dtm4271t_{—} |
| Tissue Name | ag2458 | ag2458_a2 | Tissue Name | ag2458 |
| Liver | 33.9 | 41.4 | Normal Colon GENPAK | 62.4 |
| adenocarcinoma | | | 061003 | |
| Pancreas | 1.3 | 0.8 | 83219 CC Well to Mod Diff | 16.8 |
| | | | (ODO3866) | |
| Pancreatic ca. | 6.9 | 44.3 | 83220 CC NAT (ODO3866) | 11.7 |
| CAPAN 2 | | | | |
| Adrenal gland | 0.7 | 1.1 | 83221 CC Gr.2 rectosigmoid | 18.6 |
| | | | (ODO3868) | |
| Thyroid | 2.8 | 3.3 | 83222 CC NAT (ODO3868) | 8.4 |
| Salivary gland | 0.6 | 2.0 | 83235 CC Mod Diff | 34.2 |
| | | | (ODO3920) | |
| Pituitary gland | 1.6 | 0.4 | 83236 CC NAT (ODO3920) | 11.3 |
| Brain (fetal) | 9.2 | 19.7 | 83237 CC Gr.2 ascend colon | 74.7 |
| | | | (ODO3921) | |
| Brain (whole) | 5.4 | 17.7 | 83238 CC NAT (ODO3921) | 9.6 |
| Brain (amygdala) | 6.7 | 9.0 | 83241 CC from Partial | 35.6 |
| | | | Hepatectomy (ODO4309) | |
| Brain (cerebellum) | 2.7 | 10.2 | 83242 Liver NAT | 32.3 |
| | | | (ODO4309) | |
| Brain | 23.0 | 13.1 | 87472 Colon mets to lung | 29.7 |
| (hippocampus) | | | (OD04451-01) | |
| Brain (substantia | 1.6 | 7.2 | 87473 Lung NAT | 6.4 |
| nigra) | | | (OD04451-02) | |
| Brain (thalamus) | 4.9 | 16.6 | Normal Prostate Clontech | 8.8 |
| | | | A+ 6546-1 | |
| Cerebral Cortex | 26.2 | 8.2 | 84140 Prostate Cancer | 25.9 |
| | | | (OD04410) | |
| Spinal cord | 2.4 | 9.8 | 84141 Prostate NAT | 27.9 |
| | | | (OD04410) | |
| CNS ca. (glio/astro) | 11.0 | 19.7 | 87073 Prostate Cancer | 15.8 |
| U87-MG | | | (OD04720-01) | |
| CNS ca. | 45.1 | 82.4 | 87074 Prostate NAT | 28.1 |
| (glio/astro) U-118- | | | (OD04720-02) | |
| MG | | | | |
| CNS ca. (astro) | 21.9 | 46.0 | Normal Lung GENPAK | 24.8 |
| SW1783 | | | 061010 | |
| CNS ca.* (neuro; | 73.7 | 40.2 | 83239 Lung Met to Muscle | 100.0 |
| met) SK-N-AS | | | (ODO4286) | |
| CNS ca. (astro) | 9.9 | 12.0 | 83240 Muscle NAT | 31.9 |
| SF-539 | | | (ODO4286) | |
| CNS ca. (astro) | 22.5 | 71.5 | 84136 Lung Malignant | 29.5 |
| SNB-75 | | | Cancer (OD03126) | |
| CNS ca. (glio) | 6.2 | 18.1 | 84137 Lung NAT | 27.9 |
| SNB-19 | | | (OD03126) | |
| CNS ca. (glio) | 5.9 | 45.0 | 84871 Lung Cancer | 71.7 |
| U251 | | | (OD04404) | |
| CNS ca. (glio) | 13.0 | 26.4 | 84872 Lung NAT | 15.1 |
| SF-295 | | | (OD04404) | |
| Heart (fetal) | 1.7 | 0.0 | 84875 Lung Cancer | 28.5 |
| | | | (OD04565) | |
| Heart | 1.2 | 4.4 | 84876 Lung NAT | 13.1 |
| | | | (OD04565) | |
| Fetal Skeletal | 11.4 | 1.2 | 85950 Lung Cancer | 90.1 |
| | | | (OD04237-01) | |
| Skeletal muscle | 3.8 | 41.7 | 85970 Lung NAT | 16.8 |
| | | | (OD04237-02) | |
| Bone marrow | 1.5 | 2.2 | 83255 Ocular Mel Met to | 76.3 |
| | | | Liver (ODO4310) | |
| Thymus | 0.8 | 0.4 | 83256 Liver NAT | 26.8 |
| | | | (ODO4310) | |
| Spleen | 0.9 | 0.8 | 84139 Melanoma Mets to | 36.3 |
| | | | Lung (OD04321) | |
| Lymph node | 1.3 | 10.4 | 84138 Lung NAT | 22.5 |
| | | | (OD04321) | |
| Colorectal | 4.8 | 3.1 | Normal Kidney GENPAK | 33.7 |
| | | | 061008 | |
| Stomach | 0.0 | 3.1 | 83786 Kidney Ca, Nuclear | 68.8 |
| | | | grade 2 (OD04338) | |
| Small intestine | 1.1 | 2.3 | 83787 Kidney NAT | 33.4 |
| | | | (OD04338) | |
| Colon ca. | 19.5 | 18.9 | 83788 Kidney Ca Nuclear | 30.6 |
| SW480 | | | grade 1/2 (OD04339) | |
| Colon ca.* (SW480 | 29.9 | 29.8 | 83789 Kidney NAT | 27.2 |
| met)SW620 | | | (OD04339) | |
| Colon ca. | 17.7 | 9.7 | 83790 Kidney Ca, Clear cell | 49.3 |
| HT29 | | | type (OD04340) | |
| Colon ca. | 22.1 | 43.8 | 83791 Kidney NAT | 32.1 |
| HCT-116 | | | (OD04340) | |
| Colon ca. | 13.5 | 18.4 | 83792 Kidney Ca, Nuclear | 16.0 |
| CaCo-2 | | | grade 3 (OD04348) | |
| 83219 CC Well to | 10.6 | 7.0 | 83793 Kidney NAT | 35.4 |
| Mod Diff | | | (OD04348) | |
| (ODO3866) | | | | |
| Colon ca. | 45.7 | 21.1 | 87474 Kidney Cancer | 13.2 |
| HCC-2998 | | | (OD04622-01) | |
| Gastric ca.* (liver | 38.4 | 69.5 | 87475 Kidney NAT | 4.0 |
| met) NCI-N87 | | | (OD04622-03) | |
| Bladder | 7.4 | 13.6 | 85973 Kidney Cancer | 48.6 |
| | | | (OD04450-01) | |
| Trachea | 2.9 | 3.0 | 85974 Kidney NAT | 30.4 |
| | | | (OD04450-03) | |
| Kidney | 1.3 | 3.5 | Kidney Cancer Clontech | 20.4 |
| | | | 8120607 | |
| Kidney (fetal) | 3.6 | 3.9 | Kidney NAT Clontech | 7.3 |
| | | | 8120608 | |
| Renal ca. | 10.0 | 19.0 | Kidney Cancer Clontech | 14.4 |
| 786-0 | | | 8120613 | |
| Renal ca. | 29.7 | 28.6 | Kidney NAT Clontech | 8.8 |
| A498 | | | 8120614 | |
| Renal ca. | 5.6 | 53.0 | Kidney Cancer Clontech | 12.2 |
| RXF 393 | | | 9010320 | |
| Renal ca. | 5.5 | 13.3 | Kidney NAT Clontech | 22.1 |
| ACHN | | | 9010321 | |
| Renal ca. | 21.6 | 45.5 | Normal Uterus GENPAK | 8.3 |
| UO-31 | | | 061018 | |
| Renal ca. | 20.4 | 27.0 | Uterus Cancer GENPAK | 15.3 |
| TK-10 | | | 064011 | |
| Liver | 2.9 | 2.4 | Normal Thyroid Clontech | 15.1 |
| | | | A+ 6570-1 | |
| Liver (fetal) | 3.8 | 5.0 | Thyroid Cancer GENPAK | 33.0 |
| | | | 064010 | |
| Liver ca. | 17.3 | 28.2 | Thyroid Cancer | 21.6 |
| (hepatoblast) | | | INVITROGEN A302152 | |
| HepG2 | | | | |
| Lung | 1.9 | 2.5 | Thyroid NAT | 14.4 |
| | | | INVITROGEN A302153 | |
| Lung (fetal) | 1.4 | 5.6 | Normal Breast GENPAK | 33.2 |
| | | | 061019 | |
| Lung ca. (small | 11.8 | 40.6 | 84877 Breast Cancer | 44.8 |
| cell) LX-1 | | | (OD04566) | |
| Lung ca. (small | 31.4 | 44.0 | 85975 Breast Cancer | 95.9 |
| cell) NCI-H69 | | | (OD04590-01) | |
| Lung ca. (s.cell | 69.3 | 90.5 | 85976 Breast Cancer Mets | 61.1 |
| var.) SHP-77 | | | (OD04590-03) | |
| Lung ca. (large | 9.9 | 63.6 | 87070 Breast Cancer | 38.4 |
| cell)NCI-H460 | | | Metastasis (OD04655-05) | |
| Lung ca. (non-sm. | 20.9 | 25.9 | GENPAK Breast Cancer | 33.2 |
| cell) A549 | | | 064006 | |
| Lung ca. (non- | 11.3 | 10.7 | Breast Cancer Res. Gen. | 23.0 |
| s.cell) NCI-H23 | | | 1024 | |
| Lung ca (non-s.cell) | 4.1 | 9.1 | Breast Cancer Clontech | 33.4 |
| HOP-62 | | | 9100266 | |
| Lung ca. (non-s.cl) | 29.1 | 30.0 | Breast NAT Clontech | 19.5 |
| NCI-H522 | | | 9100265 | |
| Lung ca. (squam.) | 9.7 | 20.1 | Breast Cancer | 47.0 |
| SW 900 | | | INVITROGEN A209073 | |
| Lung ca. (squam.) | 9.5 | 43.2 | Breast NAT INVITROGEN | 37.6 |
| NCI-H596 | | | A2090734 | |
| Mammary gland | 5.3 | 9.0 | Normal Liver GENPAK | 15.5 |
| | | | 061009 | |
| Breast ca.* (pl. | 14.3 | 30.8 | Liver Cancer GENPAK | 14.0 |
| effusion) MCF-7 | | | 064003 | |
| Breast ca.* (pl.ef) | 42.3 | 41.1 | Liver Cancer Research | 20.4 |
| MDA-MB-231 | | | Genetics RNA 1025 | |
| Breast ca.* (pl. | 9.6 | 13.8 | Liver Cancer Research | 7.1 |
| effusion) T47D | | | Genetics RNA 1026 | |
| Breast ca. | 100.0 | 100.0 | Paired Liver Cancer Tissue | 24.8 |
| BT-549 | | | Research Genetics RNA | |
| | | | 6004-T | |
| Breast ca. | 17.2 | 8.8 | Paired Liver Tissue Research | 18.0 |
| MDA-N | | | Genetics RNA 6004-N | |
| Ovary | 4.8 | 0.9 | Paired Liver Cancer Tissue | 13.4 |
| | | | Research Genetics RNA | |
| | | | 6005-T | |
| Ovarian ca. | 11.3 | 20.9 | Paired Liver Tissue Research | 7.3 |
| OVCAR-3 | | | Genetics RNA 6005-N | |
| Ovarian ca. | 2.5 | 10.6 | Normal Bladder GENPAK | 59.9 |
| OVCAR-4 | | | 061001 | |
| Ovarian ca. | 20.0 | 26.3 | Bladder Cancer Research | 5.2 |
| OVCAR-5 | | | Genetics RNA 1023 | |
| Ovarian ca. | 18.6 | 16.5 | Bladder Cancer | 30.1 |
| OVCAR-8 | | | INVITROGEN A302173 | |
| Ovarian ca. | 6.9 | 6.8 | 87071 Bladder Cancer | 65.5 |
| IGROV-1 | | | (OD04718-01) | |
| Ovarian ca.* | 32.3 | 64.3 | 87072 Bladder Normal | 18.4 |
| (ascites) SK-OV-3 | | | Adjacent (OD04718-03) | |
| Uterus | 1.8 | 4.3 | Normal Ovary Res. Gen. | 5.1 |
| Plancenta | 2.2 | 1.1 | Ovarian Cancer GENPAK | 29.1 |
| | | | 064008 | |
| Prostate | 2.0 | 2.4 | 87492 Ovary Cancer | 66.0 |
| | | | (OD04768-07) | |
| Prostate ca.* (bone | 3.1 | 3.2 | 87493 Ovary NAT | 7.7 |
| met)PC-3 | | | (OD04768-08) | |
| Testis | 2.0 | 1.5 | Normal Stomach GENPAK | 16.4 |
| | | | 061017 | |
| Melanoma | 4.6 | 5.4 | Gastric Cancer Clontech | 3.5 |
| Hs688(A).T | | | 9060358 | |
| Melanoma* (met) | 2.4 | 6.9 | NAT Stomach Clontech | 10.5 |
| Hs688(B).T | | | 9060359 | |
| Melanoma | 1.3 | 12.6 | Gastric Cancer Clontech | 29.9 |
| UACC-62 | | | 9060395 | |
| Melanoma | 5.9 | 56.2 | NAT Stomach Clontech | 12.9 |
| M14 | | | 9060394 | |
| Melanoma | 14.7 | 8.1 | Gastric Cancer Clontech | 42.3 |
| LOX IMVI | | | 9060397 | |
| Melanoma* (met) | 16.2 | 24.7 | NAT Stomach Clontech | 6.2 |
| SK-MEL-5 | | | 9060396 | |
| Adipose | 4.4 | 3.4 | Gastric Cancer GENPAK | 31.0 |
| | | | 064005 | |

TABLE 34
Panels 3D and 4D

| PANEL 3D | | PANEL 4D | |
|---|---|---|---|
| | Rel. Expr., % | | Rel. Expr., % |
| | 3dx4tm5121t_{—} | | 4dtm4272t_{—} |
| Tissue Name | ag2458_b2 | Tissue Name | ag2458 |
| 94905_Daoy_Medulloblastoma/ | 13.6 | 93768_Secondary Th1_anti- | 14.5 |
| Cerebellum_sscDNA | | CD28/anti-CD3 | |
| 94906_TE671_Medulloblastom/ | 7.4 | 93769_Secondary Th2_anti- | 7.4 |
| Cerebellum_sscDNA | | CD28/anti-CD3 | |
| 94907_D283 | 53.0 | 93770_Secondary Tr1_anti- | 9.5 |
| Med_Medulloblastoma/Cerebellum | | CD28/anti-CD3 | |
| _sscDNA | | | |
| 94908_PFSK-1_Primitive | 6.5 | 93573_Secondary Th1_resting day | 0.2 |
| Neuroectodermal/Cerebellum | | 4-6 in IL-2 | |
| _sscDNA | | | |
| 94909_XF- | 6.8 | 93572_Secondary Th2_resting day | 0.6 |
| 498_CNS_sscDNA | | 4-6 in IL-2 | |
| 94910_SNB- | 9.8 | 93571_Secondary Tr1_resting day | 0.8 |
| 78_CNS/glioma_sscDNA | | 4-6 in IL-2 | |
| 94911_SF- | 10.2 | 93568_primary Th1_anti- | 19.8 |
| 268_CNS/glioblastoma_sscDNA | | CD28/anti-CD3 | |
| 94912_T98G_Glioblastoma | 15.7 | 93569_Primary Th2_anti- | 13.0 |
| _sscDNA | | CD28/anti-CD3 | |
| 96776_SK-N- | 16.5 | 93570_primary Tr1_anti- | 19.2 |
| SH_Neuroblastoma | | CD28/anti-CD3 | |
| (metastasis)_sscDNA | | | |
| 94913_SF- | 7.4 | 93565_primary Th1_resting dy 4-6 | 8.8 |
| 295_CNS/glioblastoma_sscDNA | | in IL-2 | |
| 94914_Cerebellum_sscDNA | 3.9 | 93566_primary Th2_resting dy 4-6 | 2.2 |
| | | in IL-2 | |
| 96777_Cerebellum_sscDNA | 0.8 | 93567_primary Tr1_resting dy 4-6 | 3.6 |
| | | in IL-2 | |
| 94916_NCI- | 46.9 | 93351_CD45RA CD4 | 19.5 |
| H292_Mucoepidermoid | | lymphocyte_anti-CD28/anti-CD3 | |
| lung carcinoma_sscDNA | | | |
| 94917_DMS-114_Small | 10.9 | 93352_CD45RO CD4 | 17.7 |
| cell lung cancer_sscDNA | | lymphocyte_anti-CD28/anti-CD3 | |
| 94918_DMS-79_Small cell | 100.0 | 93251_CD8 Lymphocytes_anti- | 9.3 |
| lung | | CD28/anti-CD3 | |
| cancer/neuroendocrine_sscDNA | | | |
| 94919_NCI-H146_Small | 33.4 | 93353_chronic CD8 Lymphocytes | 11.0 |
| cell lung | | 2ry_resting dy 4-6 in IL-2 | |
| cancer/neuroendocrine_sscDNA | | | |
| 94920_NCI-H526_Small | 30.2 | 93574_chronic CD8 Lymphocytes | 5.5 |
| cell lung | | 2ry_activated CD3/CD28 | |
| cancer/neuroendocrine_sscDNA | | | |
| 94921_NCI-N417_Small | 26.1 | 93354_CD4_none | 0.7 |
| cell lung | | | |
| cancer/neuroendocrine_sscDNA | | | |
| 94923_NCI-H82_Small | 28.4 | 93252_Secondary | 0.9 |
| cell lung | | Th1/Th2/Tr1_anti-CD95 CH11 | |
| cancer/neuroendocrine_sscDNA | | | |
| 94924_NCI- | 88.0 | 93103_LAK cells_resting | 15.8 |
| H157_Squamous cell lung | | | |
| cancer | | | |
| (metastasis)_sscDNA | | | |
| 94925_NCI-H1155_Large | 31.3 | 93788_LAK cells_IL-2 | 6.1 |
| cell lung | | | |
| cancer/neuroendocrine_sscDNA | | | |
| 94926_NCI-H1299_Large | 32.4 | 93787_LAK cells_IL-2 + IL-12 | 8.8 |
| cell lung | | | |
| cancer/neuroendocrine_sscDNA | | | |
| 94927_NCI-H727_Lung | 23.0 | 93789_LAK cells_IL-2 + IFN | 11.7 |
| carcinoid_sscDNA | | gamma | |
| 94928_NCI-UMC- | 75.0 | 93790_LAK cells_IL-2 + IL-18 | 13.2 |
| 11_Lung | | | |
| carcinoid_sscDNA | | | |
| 94929_LX-1_Small cell | 17.0 | 93104_LAK | 8.5 |
| lung cancer_sscDNA | | cells_PMA/ionomycin and IL-18 | |
| 94930_Colo-205_Colon | 11.1 | 93578_NK Cells IL-2_resting | 2.5 |
| cancer_sscDNA | | | |
| 94931_KM12_Colon | 37.3 | 93109_Mixed Lymphocyte | 17.0 |
| cancer_sscDNA | | Reaction_Two Way MLR | |
| 94932_KM20L2_Colon | 7.8 | 93110_Mixed Lymphocyte | 10.2 |
| cancer_sscDNA | | Reaction_Two Way MLR | |
| 94933_NCI-H716_Colon | 32.3 | 93111_Mixed Lymphocyte | 7.4 |
| cancer_sscDNA | | Reaction_Two Way MLR | |
| 94935_SW-48_Colon | 7.8 | 93112_Mononuclear Cells | 2.4 |
| adenocarcinoma_sscDNA | | (PBMCs)_resting | |
| 94936_SW1116_Colon | 10.9 | 93113_Mononuclear Cells | 23.7 |
| adenocarcinoma_sscDNA | | (PBMCs)_PWM | |
| 94937_LS 174T_Colon | 29.3 | 93114_Mononuclear Cells | 9.6 |
| adenocarcinoma_sscDNA | | (PBMCs)_PHA-L | |
| 94938_SW-948_Colon | 1.9 | 93249_Ramos (B cell)_none | 30.4 |
| adenocarcinoma_sscDNA | | | |
| 94939_SW-480_Colon | 4.9 | 93250_Ramos (B cell)_ionomycin | 100.0 |
| adenocarcinoma_sscDNA | | | |
| 94940_NCI-SNU- | 8.3 | 93349_B lymphocytes_PWM | 70.2 |
| 5_Gastric | | | |
| carcinoma_sscDNA | | | |
| 94941_KATO III_Gastric | 53.1 | 93350_B lymphoytes_CD40L and | 5.5 |
| carcinoma_sscDNA | | IL-4 | |
| 94943_NCI-SNU- | 7.3 | 92665_EOL-1 | 11.7 |
| 16_Gastric | | (Eosinophil)_dbcAMP | |
| carcinoma_sscDNA | | differentiated | |
| 94944_NCI-SNU- | 64.4 | 93248_EOL-1 | 6.3 |
| 1_Gastric | | (Eosinophil)_dbcAMP/PMAionomycin | |
| carcinoma_sscDNA | | | |
| 94946_RF-1_Gastric | 11.4 | 93356_Dendritic Cells_none | 12.4 |
| adenocarcinoma_sscDNA | | | |
| 94947_RF-48_Gastric | 15.4 | 93355_Dendritic Cells_LPS 100 ng/ml | 9.0 |
| adenocarcinoma_sscDNA | | | |
| 96778_MKN-45_Gastric | 28.8 | 93775_Dendritic Cells_anti-CD40 | 12.5 |
| carcinoma_sscDNA | | | |
| 94949_NCI-N87_Gastric | 19.5 | 93774_Monocytes_resting | 15.2 |
| carcinoma_sscDNA | | | |
| 94951_OVCAR-5_Ovarian | 11.7 | 93776_Monocytes_LPS 50 ng/ml | 11.7 |
| carcinoma_sscDNA | | | |
| 94952_RL95-2_Uterine | 4.5 | 93581_Macrophages_resting | 41.8 |
| carcinoma_sscDNA | | | |
| 94953_HelaS3_Cervical | 11.3 | 93582_Macrophages_LPS 100 ng/ml | 6.8 |
| adenocarcinoma_sscDNA | | | |
| 94954_Ca Ski_Cervical | 24.3 | 93098_HUVEC | 35.8 |
| epidermoid carcinoma | | (Endothelial)_none | |
| (metastasis)_sscDNA | | | |
| 94955_ES-2_Ovarian clear | 16.1 | 93099_HUVEC | 58.2 |
| cell carcinoma_sscDNA | | (Endothelial)_starved | |
| 94957_Ramos/6 h stim_”; | 8.7 | 93100_HUVEC (Endothelial)_IL- | 16.5 |
| Stimulated with | | 1b | |
| PMA/ionomycin | | | |
| 6 h_sscDNA | | | |
| 94958_Ramos/14 h stim_”; | 8.3 | 93779_HUVEC (Endothelial)_IFN | 26.1 |
| Stimulated with | | gamma | |
| PMA/ionomycin | | | |
| 14 h_sscDNA | | | |
| 94962_MEG-01_Chronic | 22.1 | 93102_HUVEC | 16.0 |
| myelogenous leukemia | | (Endothelial)_TNF alpha + IFN | |
| (megokaryoblast)_sscDNA | | gamma | |
| 94963_Raji_Burkitt's | 8.3 | 93101_HUVEC | 23.2 |
| lymphoma_sscDNA | | (Endothelial)_TNF alpha + IL4 | |
| 94964_Daudi_Burkitt's | 19.8 | 93781_HUVEC (Endothelial)_IL- | 13.6 |
| lymphoma_sscDNA | | 11 | |
| 94965_U266_B-cell | 6.4 | 93583_Lung Microvascular | 21.0 |
| plasmacytoma/myeloma_sscDNA | | Endothelial Cells_none | |
| 94968_CA46_Burkitt's | 6.5 | 93584_Lung Microvascular | 18.3 |
| lymphoma_sscDNA | | Endothelial Cells_TNFa (4 ng/ml) | |
| | | and IL1b (1 ng/ml) | |
| 94970_RL_non-Hodgkin's | 9.0 | 92662_Microvascular Dermal | 35.4 |
| B-cell lymphoma_sscDNA | | endothelium_none | |
| 94972_JM1_pre-B-cell | 4.1 | 92663_Microsvasular Dermal | 20.0 |
| lymphoma/leukemia_sscDNA | | endothelium_TNFa (4 ng/ml) and | |
| | | IL1b (1 ng/ml) | |
| 94973_Jurkat_T cell | 9.8 | 93773_Bronchial | 14.9 |
| leukemia_sscDNA | | epithelium_TNFa (4 ng/ml) and | |
| | | IL1b (1 ng/ml)** | |
| 94974_TF- | 31.5 | 93347_Small Airway | 7.2 |
| 1_Erythroleukemia_sscDNA | | Epithelium_none | |
| 94975_HUT 78_T-cell | 15.7 | 93348_Small Airway | 45.4 |
| lymphoma_sscDNA | | Epithelium_TNFa (4 ng/ml) and | |
| | | IL1b (1 ng/ml) | |
| 94977_U937_Histiocytic | 30.5 | 92668_Coronery Artery | 17.7 |
| lymphoma_sscDNA | | SMC_resting | |
| 94980_KU- | 21.7 | 92669_Coronery Artery | 10.8 |
| 812_Myelogenous | | SMC_TNFa (4 ng/ml) and IL1b (1 ng/ml) | |
| leukemia_sscDNA | | | |
| 94981_769-P_Clear cell | 16.6 | 93107_astrocytes_resting | 11.1 |
| renal carcinoma_sscDNA | | | |
| 94983_Caki-2_Clear cell | 16.8 | 93108_astrocytes_TNFa (4 ng/ml) | 10.5 |
| renal carcinoma_sscDNA | | and IL1b (1 ng/ml) | |
| 94984_SW 839_Clear cell | 11.8 | 92666_KU-812 (Basophil)_resting | 18.8 |
| renal carcinoma_sscDNA | | | |
| 94986_G401_Wilms' | 16.5 | 92667_KU-812 | 27.9 |
| tumor_sscDNA | | (Basophil)_PMA/ionoycin | |
| 94987_Hs766T_Pancreatic | 12.9 | 93579_CCD1106 | 20.9 |
| carcinoma (LN | | (Keratinocytes)_none | |
| metastasis)_sscDNA | | | |
| 94988_CAPAN- | 12.0 | 93580_CCD1106 | 7.4 |
| 1_Pancreatic | | (Keratinocytes)_TNFa and IFNg** | |
| adenocarcinoma (liver | | | |
| metastasis)_sscDNA | | | |
| 94989_SU86.86_Pancreatic | 32.6 | 93791_Liver Cirrhosis | 3.7 |
| carcinoma (liver | | | |
| metastasis)_sscDNA | | | |
| 94990_BxPC-3_Pancreatic | 4.0 | 93792_Lupus Kidney | 1.9 |
| adenocarcinoma_sscDNA | | | |
| 94991_HPAC_Pancreatic | 26.4 | 93577_NCI-H292 | 18.6 |
| adenocarcinoma_sscDNA | | | |
| 94992_MIA PaCa- | 8.2 | 93358_NCI-H292_IL-4 | 33.7 |
| 2_Pancreatic | | | |
| carcinoma_sscDNA | | | |
| 94993_CFPAC- | 30.3 | 93360_NCI-H292_IL-9 | 36.9 |
| 1_Pancreatic ductal | | | |
| adenocarcinoma_sscDNA | | | |
| 94994_PANC-1_Pancreatic | 26.7 | 93359_NCI-H292_IL-13 | 20.0 |
| epithelioid ductal | | | |
| carcinoma_sscDNA | | | |
| 94996_T24_Bladder | 8.2 | 93357_NCI-H292_IFN gamma | 20.4 |
| carcinma (transitional | | | |
| cell)_sscDNA | | | |
| 94997_5637_Bladder | 20.5 | 93777_HPAEC_- | 18.8 |
| carcinoma_sscDNA | | | |
| 94998_HT-1197_Bladder | 18.0 | 93778_HPAEC_IL-1beta/TNA | 18.9 |
| carcinoma_sscDNA | | alpha | |
| 94999_UM-UC-3_Bladder | 4.6 | 93254_Normal Human Lung | 9.5 |
| carcinma (transitional | | Fibroblast_none | |
| cell)_sscDNA | | | |
| 95000_A204_Rhabdomyos | 20.0 | 93253_Normal Human Lung | 3.7 |
| arcoma_sscDNA | | Fibroblast_TNFa (4 ng/ml) and IL- | |
| | | 1b (1 ng/ml) | |
| 95001_HT- | 15.4 | 93257_Normal Human Lung | 24.7 |
| 1080_Fibrosarcoma_sscDNA | | Fibroblast_IL-4 | |
| 95002_MG- | 16.2 | 93256_Normal Human Lung | 19.2 |
| 63_Osteosarcoma | | Fibroblast_IL-9 | |
| (bone)_sscDNA | | | |
| 95003_SK-LMS- | 27.5 | 93255_Normal Human Lung | 14.3 |
| 1_Leiomyosarcoma | | Fibroblast_IL-13 | |
| (vulva)_sscDNA | | | |
| 95004_SJRH30_Rhabdom | 12.5 | 93258_Normal Human Lung | 23.2 |
| yosarcoma (met to bone | | Fibroblast_IFN gamma | |
| marrow)_sscDNA | | | |
| 95005_A431_Epidermoid | 6.9 | 93106_Dermal Fibroblasts | 47.0 |
| carcinoma_sscDNA | | CCD1070_resting | |
| 95007_WM266- | 7.5 | 93361_Dermal Fibroblasts | 42.3 |
| 4_Melanoma_sscDNA | | CCD1070 _TNF alpha 4 ng/ml | |
| 95010_DU 145_Prostate | 0.1 | 93105_Dermal Fibroblasts | 20.2 |
| carcinoma (brain | | CCD1070_IL-1beta 1 ng/ml | |
| metastasis)_sscDNA | | | |
| 95012_MDA-MB- | 13.5 | 93772_dermal fibroblast_IFN | 9.2 |
| 468_Breast | | gamma | |
| adenocarcinoma_sscDNA | | | |
| 95013_SCC-4_Squamous | 1.3 | 93771_dermal fibroblast_IL-4 | 22.1 |
| cell carcinoma of | | | |
| tongue_sscDNA | | | |
| 95014_SCC-9_Squamous | 0.3 | 93259_IBD Colitis 1** | 1.4 |
| cell carcinoma of | | | |
| tongue_sscDNA | | | |
| 95015_SCC-15_Squamous | 0.3 | 93260_IBD Colitis 2 | 1.1 |
| cell carcinoma of | | | |
| tongue_sscDNA | | | |
| 95017_CAL 27_Squamous | 24.5 | 93261_IBD Crohns | 1.0 |
| cell carcinoma of | | | |
| tongue_sscDNA | | | |
| | | 735010_Colon_normal | 4.1 |
| | | 735019_Lung_none | 8.9 |
| | | 64028-1_Thymus_none | 10.5 |
| | | 64030-1_Kidney_none | 3.6 |

Panel 4D Description: The gene MOL9b is upregulated in endothelium, and epithelium regardless of stimulus. There is up regulation of this molecule in an ionomycin treated B cell line and it is highly expressed in mitogen (PWM) treated B cells. Consistent with this finding, PBMCs treated with PWM also up regulate this molecule. Induction of this gene MOL9b is also seen in activated T cells. In PBMC the T cell specific mitogen PHA induces the expression of this transcript and in acute and chronically activated T cells the expression of this transcript is increased as compared to untreated or resting T cells. This transcript is also expressed in resting macrophages.

Potential Role(s) of in Inflammation: The molecule MOL9b is induced by B and T lymphocytes and may potentiate inflammation by regulating lymphocyte trafficking, or activation, or increasing tissue destruction. This molecule may also serve as a marker for activated T or B cells and may also be involved in the differentiation of monocytes into macrophages (see reference). Macrophages also participate in inflammation by producing multiple biologically active proteins like cytokines, activating other cells within the local microenvironment, and ingesting dead and dying cells.

Impact of Therapeutic Targeting of MOL9b: Small molecule or antibody therapies to the molecule encoded for by MOL9b may reduce or eliminate inflammation and tissue damage due to T or B cell activation or macrophages and the bioactive molecules produced by these cell types. These diseases would include asthma/allergy, colitis, Crohn's disease, lupus, and arthritis. Alternatively, protein therapeutics based on this molecule could serve as an adjuvant and help boost the effectiveness of vaccines or regulate immune status during organ transplant. MOL9b also serves as a marker for activated T and B cells and serve as a diagnostic tool for determining indirectly measuring the extent of inflammation due to autoimmune diseases which induce T or B cells activation such as asthma/allergy, colitis, Crohn's disease, lupus, and arthritis. Elevated expression of the gene MOL9b in macrophages may help in distinguishing, resting macrophages from monocytes , dendritic cells.

MOL10a

Expression of gene MOL10a was assessed using the primer-probe set Ag1129, described in Table 35. Results of the RTQ-PCR runs are shown in Table 36.

TABLE 35

Probe name: Ag1129

| | | | | | |
|---|---|---|---|---|---|
| | | | | Start | SEQ |
| | | | | Posi- | ID |
| Primers | Sequences | Tm | Length | tion | NO: |

| | | | | | |
|---|---|---|---|---|---|
| Forward | 5- | 59.5 | 21 | 1518 | 103 |
| | CTAGACCAGCAGCTGGATGA | | | | |
| | T-3′ | | | | |
| Probe | TET-5′- | 68.7 | 26 | 1539 | 104 |
| | CTACAGACCAAGTTTGCTCG | | | | |
| | CCTCCT-3′-TAMRA | | | | |
| Reverse | 5′- | 59 | 22 | 1587 | 105 |
| | CAATGCGGTAAGCAATCTTA | | | | |
| | AG-3′ | | | | |

TABLE 36
Panels 1.3D and 4D

| PANEL 1.3D | | PANEL 4D | |
|---|---|---|---|
| | Rel. Expr., % | | Rel. Expr., % |
| | 1.3dx4tm5771t_{—} | | 4Dtm1984t_{—} |
| Tissue Name | ag1129_a2 | Tissue Name | ag1129 |
| Liver | 3.9 | 93768_Secondary Th1_anti-CD28/anti- | 0.0 |
| adenocarcinoma | | CD3 | |
| Pancreas | 3.7 | 93769_Secondary Th2_anti-CD28/anti- | 9.0 |
| | | CD3 | |
| Pancreatic ca. | 2.8 | 93770_Secondary Tr1_anti-CD28/anti- | 9.0 |
| CAPAN 2 | | CD3 | |
| Adrenal gland | 1.8 | 93573_Secondary Th1_resting day 4-6 | 0.0 |
| | | in IL-2 | |
| Thyroid | 3.2 | 93572_Secondary Th2_resting day 4-6 | 0.0 |
| | | in IL-2 | |
| Salivary gland | 3.8 | 93571_Secondary Tr1_resting day 4-6 in | 0.0 |
| | | IL-2 | |
| Pituitary gland | 2.6 | 93568_primary Th1_anti-CD28/anti- | 0.0 |
| | | CD3 | |
| Brain (fetal) | 21.9 | 93569_primary Th2_anti-CD28/anti- | 0.0 |
| | | CD3 | |
| Brain (whole) | 14.6 | 93570_primary Tr1_anti-CD28/anti- | 8.9 |
| | | CD3 | |
| Brain (amygdala) | 13.0 | 93565_primary Th1_resting dy 4-6 in | 6.8 |
| | | IL-2 | |
| Brain (cerebellum) | 1.8 | 93566_primary Th2_resting dy 4-6 in | 14.6 |
| | | IL-2 | |
| Brain (hippocampus) | 13.7 | 93567_primary Tr1_resting dy 4-6 in IL-2 | 0.0 |
| Brain (substantia | 21.4 | 93351_CD45RA CD4 lymphocyte_anti- | 5.9 |
| nigra) | | CD28/anti-CD3 | |
| Brain (thalamus) | 10.3 | 93352_CD45RO CD4 lymphocyte_anti- | 14.3 |
| | | CD28/anti-CD3 | |
| Cerebral Cortex | 10.5 | 93251_CD8 Lymphocytes_anti- | 33.0 |
| | | CD28/anti-CD3 | |
| Spinal cord | 8.1 | 93353_chronic CD8 Lymphocytes | 4.2 |
| | | 2ry_resting dy 4-6 in IL-2 | |
| CNS ca. (glio/astro) | 6.0 | 93574_chronic CD8 Lymphocytes | 0.0 |
| U87-MG | | 2ry_activated CD3/CD28 | |
| CNS ca. (glio/astro) | 0.0 | 93354_CD4_none | 1.4 |
| U-118-MG | | | |
| CNS ca. (astro) | 3.5 | 93252_Secondary Th1/Th2/Tr1_anti- | 20.2 |
| SW1783 | | CD95 CH11 | |
| CNS ca.* (neuro; met) | 0.0 | 93103_LAK cells_resting | 8.7 |
| SK-N-AS | | | |
| CNS ca. (astro) | 1.7 | 93788_LAK cells_IL-2 | 30.8 |
| SF-539 | | | |
| CNS ca. (astro) | 0.0 | 93787_LAK cells_IL-2 + IL-12 | 15.6 |
| SNB-75 | | | |
| CNS ca. (glio) | 0.9 | 93789_LAK cells_IL-2 + IFN gamma | 53.2 |
| SNB-19 | | | |
| CNS ca. (glio) | 1.4 | 93790_LAK cells_IL-2 + IL-18 | 20.6 |
| U251 | | | |
| CNS ca. (glio) | 0.0 | 93104_LAK cells_PMA/ionomycin and | 0.0 |
| SF-295 | | IL-18 | |
| Heart (fetal) | 0.0 | 93578_NK Cells IL-2_resting | 5.9 |
| Heart | 3.3 | 93109_Mixed Lymphocyte | 12.9 |
| | | Reaction_Two Way MLR | |
| Fetal Skeletal | 2.0 | 93110_Mixed Lymphocyte | 3.5 |
| | | Reaction_Two Way MLR | |
| Skeletal muscle | 2.3 | 93111_Mixed Lymphocyte | 6.8 |
| | | Reaction_Two Way MLR | |
| Bone marrow | 4.0 | 93112_Mononuclear Cells | 17.2 |
| | | (PBMCs)_resting | |
| Thymus | 3.9 | 93113_Mononuclear Cells | 32.5 |
| | | (PBMCs)_PWM | |
| Spleen | 0.0 | 93114_Mononuclear Cells | 20.3 |
| | | (PBMCs)_PHA-L | |
| Lymph node | 1.7 | 93249_Ramos (B cell)_none | 22.2 |
| Colorectal | 1.4 | 93250_Ramos (B cell)_ionomycin | 68.3 |
| Stomach | 0.0 | 93349_B lymphocytes_PWM | 33.7 |
| Small intestine | 0.0 | 93350_B lymphoytes_CD40L and IL-4 | 9.4 |
| Colon ca. | 1.5 | 92665_EOL-1 (Eosinophil)_dbcAMP | 15.3 |
| SW480 | | differentiated | |
| Colon ca.* (SW480 | 2.0 | 93248_EOL-1 | 0.0 |
| met)SW620 | | (Eosinophil)_dbcAMP/PMAionomycin | |
| Colon ca. | 2.2 | 93356_Dendritic Cells_none | 0.0 |
| HT29 | | | |
| Colon ca. | 3.7 | 93355_Dendritic Cells_LPS 100 ng/ml | 6.7 |
| HCT-116 | | | |
| Colon ca. | 0.6 | 93775_Dendritic Cells_anti-CD40 | 0.0 |
| CaCo-2 | | | |
| 83219 CC Well to | 5.2 | 93774_Monocytes_resting | 20.6 |
| Mod Diff (ODO3866) | | | |
| Colon ca. | 5.1 | 93776_Monocytes_LPS 50 ng/ml | 15.8 |
| HCC-2998 | | | |
| Gastric ca.* (liver | 10.0 | 93581_Macrophages_resting | 15.9 |
| met) NCI-N87 | | | |
| Bladder | 4.6 | 93582_Macrophages_LPS 100 ng/ml | 64.2 |
| Trachea | 30.4 | 93098_HUVEC (Endothelial)_none | 3.0 |
| Kidney | 3.3 | 93099_HUVEC (Endothelial)_starved | 0.0 |
| Kidney (fetal) | 7.3 | 93100_HUVEC (Endothelial)_IL-1b | 3.5 |
| Renal ca. | 0.0 | 93779_HUVEC (Endothelial)_IFN | 6.7 |
| 786-0 | | gamma | |
| Renal ca. | 0.0 | 93102_HUVEC (Endothelial)_TNF | 0.0 |
| A498 | | alpha + IFN gamma | |
| Renal ca. | 4.4 | 93101_HUVEC (Endothelial)_TNF | 8.1 |
| RXF 393 | | alpha + IL4 | |
| Renal ca. | 0.0 | 93781_HUVEC (Endothelial)_IL-11 | 0.0 |
| ACHN | | | |
| Renal ca. | 0.0 | 93583_Lung Microvascular Endothelial | 24.0 |
| UO-31 | | Cells_none | |
| Renal ca. | 5.9 | 93584_Lung Microvascular Endothelial | 17.8 |
| TK-10 | | Cells_TNFa (4 ng/ml) and IL1b (1 ng/ml) | |
| Liver | 3.4 | 92662_Microvascular Dermal | 5.2 |
| | | endothelium_none | |
| Liver (fetal) | 0.0 | 92663_Microsvasular Dermal | 0.0 |
| | | endothelium_TNFa (4 ng/ml) and IL1b | |
| | | (1 ng/ml) | |
| Liver ca. | 0.0 | 93773_Bronchial epithelium_TNFa (4 ng/ml) | 79.6 |
| (hepatoblast) HepG2 | | and IL1b (1 ng/ml)** | |
| Lung | 30.2 | 93347_Small Airway Epithelium_none | 0.0 |
| Lung (fetal) | 100.0 | 93348_Small Airway Epithelium_TNFa | 53.6 |
| | | (4 ng/ml) and IL1b (1 ng/ml) | |
| Lung ca. (small cell) | 1.9 | 92668_Coronery Artery SMC_resting | 0.0 |
| LX-1 | | | |
| Lung ca. (small cell) | 9.8 | 92669_Coronery Artery SMC_TNFa (4 ng/ml) | 0.0 |
| NCI-H69 | | and IL1b (1 ng/ml) | |
| Lung ca. (s.cell var.) | 9.5 | 93107_astrocytes_resting | 0.0 |
| SHP-77 | | | |
| Lung ca. (large | 0.0 | 93108_astrocytes_TNFa (4 ng/ml) and | 19.6 |
| cell)NCI-H460 | | IL1b (1 ng/ml) | |
| Lung ca. (non-sm. | 1.5 | 92666_KU-812 (Basophil)_resting | 0.0 |
| cell) A549 | | | |
| Lung ca. (non-s.cell) | 1.8 | 92667_KU-812 | 15.1 |
| NCI-H23 | | (Basophil)_PMA/ionoycin | |
| Lung ca (non-s.cell) | 0.8 | 93579_CCD1106 (Keratinocytes)_none | 0.0 |
| HOP-62 | | | |
| Lung ca. (non-s.cl) | 8.7 | 93580_CCD1106 | 4.7 |
| NCI-H522 | | (Keratinocytes)_TNFa and IFNg** | |
| Lung ca. (squam.) | 1.8 | 93791_Liver Cirrhosis | 47.6 |
| SW 900 | | | |
| Lung ca. (squam.) | 6.7 | 93792_Lupus Kidney | 25.0 |
| NCI-H596 | | | |
| Mammary gland | 8.0 | 93577_NCI-H292 | 17.0 |
| Breast ca.* (pl. | 0.0 | 93358_NCI-H292_IL-4 | 4.8 |
| effusion) MCF-7 | | | |
| Breast ca.* (pl.ef) | 0.0 | 93360_NCI-H292_IL-9 | 12.9 |
| MDA-MB-231 | | | |
| Breast ca.* (pl. | 0.0 | 93359_NCI-H292_IL-13 | 0.0 |
| effusion) T47D | | | |
| Breast ca. | 0.0 | 93357_NCI-H292_IFN gamma | 4.4 |
| BT-549 | | | |
| Breast ca. | 0.0 | 93777_HPAEC_- | 0.0 |
| MDA-N | | | |
| Ovary | 2.3 | 93778_HPAEC_IL-1beta/TNA alpha | 6.9 |
| Ovarian ca. | 0.0 | 93254_Normal Human Lung | 0.0 |
| OVCAR-3 | | Fibroblast_none | |
| Ovarian ca. | 1.2 | 93253_Normal Human Lung | 6.2 |
| OVCAR-4 | | Fibroblast_TNFa (4 ng/ml) and IL-1b (1 ng/ml) | |
| Ovarian ca. | 7.7 | 93257_Normal Human Lung | 6.9 |
| OVCAR-5 | | Fibroblast_IL-4 | |
| Ovarian ca. | 0.0 | 93256_Normal Human Lung | 0.0 |
| OVCAR-8 | | Fibroblast_IL-9 | |
| Ovarian ca. | 0.0 | 93255_Normal Human Lung | 0.0 |
| IGROV-1 | | Fibroblast_IL-13 | |
| Ovarian ca.* (ascites) | 0.0 | 93258_Normal Human Lung | 0.0 |
| SK-OV-3 | | Fibroblast_IFN gamma | |
| Uterus | 1.9 | 93106_Dermal Fibroblasts | 12.9 |
| | | CCD1070_resting | |
| Plancenta | 0.0 | 93361_Dermal Fibroblasts | 28.5 |
| | | CCD1070_TNF alpha 4 ng/ml | |
| Prostate | 4.9 | 93105_Dermal Fibroblasts | 0.0 |
| | | CCD1070_IL-1beta 1 ng/ml | |
| Prostate ca.* (bone | 4.4 | 93772_dermal fibroblast_IFN gamma | 9.8 |
| met)PC-3 | | | |
| Testis | 57.0 | 93771_dermal fibroblast_IL-4 | 2.6 |
| Melanoma | 0.0 | 93259_IBD Colitis 1** | 88.3 |
| Hs688(A).T | | | |
| Melanoma* (met) | 0.0 | 93260_IBD Colitis 2 | 0.0 |
| Hs688(B).T | | | |
| Melanoma | 0.0 | 93261_IBD Crohns | 0.0 |
| UACC-62 | | | |
| Melanoma | 0.0 | 735010_Colon_normal | 20.2 |
| M14 | | | |
| Melanoma | 3.6 | 735019_Lung_none | 29.7 |
| LOX IMVI | | | |
| Melanoma* (met) | 3.3 | 64028-1_Thymus_none | 100.0 |
| SK-MEL-5 | | | |
| Adipose | 3.2 | 64030-1_Kidney_none | 10.0 |

The gene MOL10a shows high levels in the testis and in fetal lung in panel 1.3D. This indicates that this gene may be used for regeneration therapy in the lung and may also play a role in male fertility. The profile in panel 4D shows high expression in thymus with low to undetectable expression in other tissues and cell lines (Ct values >35). Therefore this gene may be involved in T-cell development and may be a marker for immature T cells.

### Example 2

SNP Analysis of MOL6A

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (See Alderborn et al. Determination of Single Nucleotide Polymorphisms by Real-time Pyrophosphate DNA Sequencing. (2000). Genome Research. 10, Issue 8, August. 1249-1265). In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PP₁) that is released from each dNTP upon DNA chain elongation. Following Klenow polymerase-mediated base incorporation, PP₁ is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

The DNA and protein sequences for the novel single nucleotide polymorphic variants of the Trypsin-like gene of MOL6a (GM87760758_A) are reported in Table 37. Variants are reported individually but any combination of all or a select subset of variants are also included. In Table 37, the positions of the variant bases and the variant amino acid residues are underlined.

TABLE 37

A. Variant 13373750 of MOL6a nucleotide sequence (SEQ ID NO. 11).
C to A at position 360.
B. Nucleotide sequence of variant at position 360.

| | | |
|---|---|---|
| 1 | TACCATGAAATATGTCTTCTATTTGGGTGTCCTCGCTGGGACATTTTTCTTTGCTGACTCATCTGTCCAGAAAGAAGAC | (SEQ ID NO.106) |
| | C | |
| 81 | CTGCTCCCTATTTGGTCTACCTCAAGTCTCACTTCAACCCCTGTGTGGGCGTCCTCATCAAACCCAGCTGGCTCCTGG | |
| | CC | |
| 161 | CCAGCTCACTGCTATTTACCAAATCTGAAACTGATGCTGGGAAATTTCAAGAGCAGAGTCAGAGACGGTACTGAACA | |
| | GAC | |
| 241 | AATTAACCCCATTCAGATCGTCCGCTACTGGAACTACAGTCATAGCGCCCCACAGGATGACCTCATGCTCATCAAGC | |
| | TGG | |
| 321 | CTAAGCCTGCCATGCTCAATCCCAAAGTCCAGCCCCTTACCCTCGCCACCACCAATGTCAGGCCAGGCACTGTCTGT | |
| | CTA | |
| 401 | CTCTCAGGTTTGGACTGGAGCCAAGAAAACAGTGGCCGACACCCTGACTTGCGGCAGAACCTGGAGGCCCCCGTGAT | |
| | GTC | |
| 481 | TGATCGAGAATGCCAAAAAAACAGAACAAGGAAAAAGCCACAGGAATTCCTTATGTGTGAAATTTGTGAAAGTATTC | |
| | AGC | |
| 561 | CGAATTTTTGGGGAGGTGGCCGTTGCTACTGTCATCTGCAAAGACAAGCTCCAGGGAATCGAGGTGGGGCACTTCAT | |
| | GGG | |
| 641 | AGGGGACGTCGGCATCTACACCAATGTTTACAAATATGTATCCTGGATTGAGAACACTGCTAAGGACAAGTGAGACC | |
| | CTA | |
| 721 | A | |

C. Protein Sequence of Variant at Position 119.

C. Protein Sequences of variant at position 119.

| | | |
|---|---|---|
| 1 | MKYVFYLGVLAGTFFFADSSVQKEDPAPYLVYLKSHFNPCVGVLIKPSWVLAPAHCYLPNLKVMLGNFKSRVRDGTEQT | (SEQ ID NO. 107) |
| | I | |
| 81 | NPIQTVRYWNYSHSAPQDDLMLIKLAKPAMLNPKVQPLTLATTNVRPGTVCLLSGLDWSQENSGRHPDLRQNLEAPVM | |
| | SD | |
| 161 | RECQKNRTRKKPQEFLMCETCESIQPNFWGGGRCYCHLQRQAPGNRGGALHGRGRRHLHQCLQICILD | |

D. Effect of Variant on Amino Acid Residue

Pro to Thr

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177;
(b) a variant of a mature form of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177; and
(d) a variant of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177;
(b) a variant of a mature form of an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177;
(d) a variant of an amino acid sequence selected from the group consisting SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 21, 23, 25, 27, 29, 114, 116, 118, 120, 123, 125, 128, 130, 132, 134, 136, 138, 140, 142, 144, 146, 148, 150, 152, 154, and 156, 158, 160, 162, 164, 166, 168, 170, 172, 174, and 176, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a MOLX-associated disorder, said method comprising administering to a subject in which such treatment or prevention is desired the polypeptide of claim 1 in an amount sufficient to treat or prevent said MOLX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a MOLX-associated disorder, said method comprising administering to a subject in which such treatment or prevention is desired the nucleic acid of claim 5 in an amount sufficient to treat or prevent said MOLX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a MOLX-associated disorder, said method comprising administering to a subject in which such treatment or prevention is desired the antibody of claim 15 in an amount sufficient to treat or prevent said MOLX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, or a biologically active fragment thereof.

49. A method of treating a pathological state in a mammal, the method comprising administering to the mammal the antibody of claim 15 in an amount sufficient to alleviate the pathological state.

50. A method for the screening of a candidate substance interacting with an olfactory receptor polypeptide selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, or fragments or variants thereof, comprises the following steps:
a) providing a polypeptide selected from the group consisting of the sequences of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, or a peptide fragment or a variant thereof;
b) obtaining a candidate substance;
c) bringing into contact said polypeptide with said candidate substance; and
d) detecting the complexes formed between said polypeptide and said candidate substance.

51. A method for the screening of ligand molecules interacting with an olfactory receptor polypeptide selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein said method comprises:
a) providing a recombinant eukaryotic host cell containing a nucleic acid encoding a polypeptide selected from the group consisting of the polypeptides comprising the amino acid sequences SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177;
b) preparing membrane extracts of said recombinant eukaryotic host cell;
c) bringing into contact the membrane extracts prepared at step b) with a selected ligand molecule; and
d) detecting the production level of second messengers metabolites.

52. A method for the screening of ligand molecules interacting with an olfactory receptor polypeptide selected from the group consisting of SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177, wherein said method comprises:
a) providing an adenovirus containing a nucleic acid encoding a polypeptide selected from the group consisting of polypeptides comprising the amino acid sequences SEQ ID NOS:2, 4, 6, 8, 10, 12, 14, 16, 18, 20, 22, 24, 26, 28, 30, 115, 117, 119, 122, 124, 127, 129, 131, 133, 135, 137, 139, 141, 143, 145, 147, 149, 151, 153, 155, 157, 159, 161, 163, 165, 167, 169, 171, 173, 175, and 177;
b) infecting an olfactory epithelium with said adenovirus;
c) bringing into contact the olfactory epithelium b) with a selected ligand molecule; and
d) detecting the increase of the response to said ligand molecule.
